(19) United States
(12) Patent Application Publication
(10) Pub. No.: US 2023/0183377 A1
(43) Pub. Date: Jun. 15, 2023

(54) ACTIVATABLE ANTIGEN BINDING PROTEINS WITH UNIVERSAL MASKING MOIETIES

(71) Applicant: SORRENTO THERAPEUTICS, INC., San Diego, CA (US)

(72) Inventors: Yanliang ZHANG, San Diego, CA (US); Gunnar F. KAUFMANN, San Diego, CA (US); Chin-I PAI, San Diego, CA (US)

(73) Assignee: SORRENTO THERAPEUTICS, INC., San Diego, CA (US)

(21) Appl. No.: 17/802,433

(22) PCT Filed: Feb. 24, 2021
(86) PCT No.: PCT/US2021/019331

§ 371 Date: Aug. 25, 2022

Related U.S. Application Data
(60) Provisional application No. 62/981,782, filed on Feb. 26, 2020.

(51) Int. Cl. C07K 16/46; C07K 14/725; C07K 16/28

(52) U.S. Cl. CPC C07K 16/46; C07K 14/7051; C07K 16/2863; C07K 16/2896; C07K 2317/31; C07K 2317/52; C07K 2317/94; C07K 2317/732; C07K 2319/03; C07K 2319/50

(57) ABSTRACT

The present disclosure provides activatable masked antigen binding proteins comprising an antigen binding protein attached to universal masking moieties by peptide linkers. The universal masking moieties dimerize with each other to form a dimerized masking complex that blocks binding between the antigen binding domain and its target antigen. The individual masking moieties and the dimerized masking complex do not bind specifically to the antigen binding domain. The masking moieties form stable dimers because their association with each other mimics homodimers or heterodimers found in naturally-occurring immunoglobulin or cell receptor molecules. The dimerization of the masking moieties does not involve covalent bonding and can be optimized by engineering interchain association through structure complementarity such as knob-in-hole.

### CROSS-REFERENCE TO RELATED APPLICATIONS

This application claims priority to U.S. Provisional Application No. 62/981,782, filed Feb. 26, 2020, the contents of which is incorporated by reference herein in its entirety.

Throughout this application various publications, patents, and/or patent applications are referenced. The disclosures of the publications, patents and/or patent applications are hereby incorporated by reference in their entireties into this application in order to more fully describe the state of the art to which this disclosure pertains.

### TECHNICAL FIELD

The present disclosure provides activatable antigen binding proteins having immunoglobulin-like antigen binding activity, wherein the activatable antigen binding proteins comprise universal masking moieties that block binding between the antigen binding domain and its target antigen until the universal masking moieties are removed from blocking the antigen binding domain.

### INTRODUCTION AND SUMMARY

Antibodies have been successfully used as therapeutic reagents for various cancers. When administered to a subject, antibodies bind to both healthy and diseased tissues leading to deleterious side effects. To overcome the off-target effects, antibodies have been engineered to include masking moieties attached to a peptide linker. The masking moiety prevents antibody binding to the target antigen wherein the masking moiety either binds directly to the antigen binding domain or sterically hinders binding between the antigen binding domain and its target antigen. The peptide linkers are designed to be cleaved by proteases secreted by the diseased tissue which unmasks the antigen binding domain at the disease site and decreases off-target activity.

Described herein are activatable masked antigen binding proteins which offer many advantages over other masked antibody-like molecules (sometimes called “probodies”). The activatable masked antigen binding proteins described herein comprise an antigen binding protein attached to universal masking moieties by peptide linkers. The universal masking moieties dimerize with each other to form a dimerized masking complex that blocks binding between the antigen binding domain and its target antigen. The individual masking moieties and the dimerized masking complex do not bind specifically to the antigen binding domain. The masking moieties form stable dimers because their association with each other mimics homodimers or heterodimers found in naturally-occurring immunoglobulin or cell receptor molecules. The dimerization of the masking moieties does not involve covalent bonding and can be optimized by engineering interchain association through structure complementarity such as knob-in-hole. The masking moieties can potentially exhibit reduced immunogenicity because they are derived from the constant domains of immunoglobulin-like molecules (e.g., CH1, CH3 kappa, CH3 lambda) or T cell receptors (e.g., TCR alpha constant and TCR beta constant domains).

The masking moieties are attached to peptide linkers that can be cleaved by a protease, for example a protease localized at a disease tissue site. The masking moieties and peptide linkers operate together acting as a mask to sterically hinder binding between the antigen binding domain of the antibody and its target antigen. The peptide linkers having amino acid lengths and sequences that can be modified for improved flexibility and/or cleavage susceptibility. By modifying the peptide linker length and/or sequences, the steric hinderance of the masking moieties and cleavage susceptibility of the peptide linkers can be increased or decreased (e.g., tunable cleavability).

The length of the peptide linkers can be lengthened or shortened to obtain an optimal balance between linker flexibility to permit steric hinderance by the masking moieties and optimal cleavage susceptibility by the tumor-associated protease. For example, shorter peptide linkers may exhibit improved steric hinderance (improved masking) but may also exhibit reduced susceptibility to protease digestion. The peptide linkers can be designed to include amino acids (e.g., glycines) that confer linker flexibility. The cleavable site within the peptide linkers can be designed to confer modulated protease susceptibility. In one example, the peptide linkers can be designed to include 1-5 glycines on the C-terminal and/or N-terminal end of the peptide linker to increase or decrease protease susceptibility. In another example, certain amino acid sequences are readily cleavable with MMP9 protease but exhibit reduced levels of cleavage with MMP2 protease. Additionally, the junction sequence between the C-terminal end of the peptide linker and the N-terminal end of the heavy chain or light chain variable region can be mutated to include one or more amino acid substitutions, deletions and/or insertions to modulate protease susceptibility.

In the inactive state, the activatable masked antigen binding proteins exhibit reduced binding to healthy tissue that expresses low levels of tumor-associated protease. The activatable masked antigen binding proteins are converted from the inactive state to activated state upon cleavage of the peptide linkers at the diseased tissue site which expresses higher levels of the tumor-associated protease, thereby greatly reducing off-target activity and reducing toxicity. The activatable masked antigen binding proteins described herein have the potential to widen the therapeutic window.

The activatable masked antigen binding protein can be joined to a toxin by a chemical linker thereby forming an immunoconjugate. The toxin can be cytotoxic to cells and tissue. The immunoconjugate can be used to deliver the toxin to the target tumor.

### DESCRIPTION OF THE FIGURES

FIG. 1 is a schematic showing a non-limiting embodiment of an activatable masked antigen binding protein having an IgG type structure. In one embodiment, the activatable masked antigen binding protein binds one target antigen (e.g., monospecific).

FIG. 2 is a schematic showing a non-limiting embodiment of an activatable masked antigen binding protein having an IgG type structure. In one embodiment, the activatable masked antigen binding protein binds two different target antigens (e.g., bispecific).

FIG. 3A is a schematic showing a non-limiting embodiment of an activatable masked antigen binding protein having a dimeric antigen receptor (DAR) type structure.

FIG. 3B is a schematic showing a non-limiting embodiment of an activatable masked antigen binding protein having a dimeric antigen receptor (DAR) type structure.

FIG. 4A is a schematic showing a non-limiting embodiment of a precursor polypeptide of an activatable masked antigen binding protein wherein the precursor can be processed to become a mature dimeric antigen receptor (DAR) type structure.

FIG. 4B is a schematic showing a non-limiting embodiment of a precursor polypeptide of an activatable masked antigen binding protein wherein the precursor can be processed to become a mature dimeric antigen receptor (DAR) type structure.

FIG. 4C is a schematic showing a non-limiting embodiment of two polypeptides that can form an activatable masked antigen binding protein having a dimeric antigen receptor (DAR) type structure.

FIG. 4D is a schematic showing a non-limiting embodiment of two polypeptides that can form an activatable masked antigen binding protein having a dimeric antigen receptor (DAR) type structure.

FIG. 5A shows an SDS-PAGE gel of cleavage products of various anti-EGFR activatable masked IgG-type antibodies comprising MMP2/9 or uPA1 peptide linkers resulting from digestion with MMP2 protease as described in Example 3 and Table 14.

FIG. 5B shows an SDS-PAGE gel of cleavage products of various anti-EGFR activatable masked IgG-type antibodies comprising MMP2/9 or uPA1 peptide linkers resulting from digestion with MMP9 protease as described in Example 3 and Table 15.

FIG. 6A shows an SDS-PAGE gel of cleavage products of an anti-EGFR activatable masked IgG-type antibody [HC-CH3 hole MMP2/9, LC-CH knob MMP2/9] digested with MMP9 or MMP2 protease for 0.5, 1 2, 6 or 24 hours as described in Example 4.

FIG. 6B shows an SDS-PAGE gel of cleavage products of an anti-EGFR activatable masked IgG-type antibody [HC-CH3 hole MMP2/9, LC-CH knob deleted] digested with MMP9 or MMP2 protease for 0.5, 1 2, 6 or 24 hours as described in Example 4.

FIG. 6C shows an SDS-PAGE gel of cleavage products of an anti-EGFR activatable masked IgG-type antibody [HC-CH3 hole MMP2/9, LC-CH knob non-cleavable] digested with MMP9 or MMP2 protease for 0.5, 1 2, 6 or 24 hours as described in Example 4.

FIG. 6D shows an SDS-PAGE gel of cleavage products of an anti-EGFR activatable masked IgG-type antibody [HC-CH3 hole MMP2/9, LC-CH knob extended] digested with MMP9 or MMP2 protease for 0.5, 1 2, 6 or 24 hours as described in Example 4.

FIG. 6E shows an SDS-PAGE gel of cleavage products of an anti-EGFR activatable masked IgG-type antibody [HC-CH3 hole deleted, LC-CH knob deleted] digested with MMP9 or MMP2 protease for 0.5, 1 2, 6 or 24 hours as described in Example 4.

FIG. 7A shows an SDS-PAGE gel of cleavage products of an anti-EGFR activatable masked IgG-type antibody [HC-CH3 hole MMP2/9, LC-CH knob MMP2/9] digested with MMP9, MMP2 or uPA protease for 1, 3, 5 or 20 hours as described in Example 5.

FIG. 7B shows an SDS-PAGE gel of cleavage products of an anti-EGFR activatable masked IgG-type antibody [HC-CH3 hole MMP9, LC-CH knob GS] digested with MMP9, MMP2 or uPA protease for 1, 3, 5 or 20 hours as described in Example 5.

FIG. 7C shows an SDS-PAGE gel of cleavage products of an anti-EGFR activatable masked IgG-type antibody [HC-CH3 hole MMP9, LC-CH knob uPA1] digested with MMP9, MMP2 or uPA protease for 1, 3, 5 or 20 hours as described in Example 5.

FIG. 8A shows a peptide trace from an LC-MS analysis of undigested anti-EGFR activatable masked IgG-type antibody [HC-CH3 hole MMP2/9, LC-CH3 knob MMP2/9] as described in Example 6 and Table 16. The antibody was de-glycosylated prior to analysis.

FIG. 8B shows a peptide trace from an LC-MS analysis of anti-EGFR activatable masked IgG-type antibody [HC-CH3 hole MMP2/9, LC-CH3 knob MMP2/9] digested with MMP9 as described in Example 6 and Table 17. The antibody was de-glycosylated prior to analysis.

FIG. 8C shows a peptide trace from an LC-MS analysis of anti-EGFR activatable masked IgG-type antibody [HC-CH3 hole MMP2/9, LC-CH3 knob MMP2/9] digested with MMP9 as described in Example 6 and Table 17 The antibody was de-glycosylated prior to analysis.

FIG. 9A shows a peptide trace from an LC-MS analysis of undigested anti-EGFR activatable masked IgG-type antibody [HC-CH1 MMP2/9, LC-CL lambda MMP2/9] as described in Example 6 and Table 18. The antibody was de-glycosylated prior to analysis.

FIG. 9B shows a peptide trace from an LC-MS analysis of anti-EGFR activatable masked IgG-type antibody [HC-CH1 MMP2/9, LC-CL lambda MMP2/9] digested with MMP9 as described in Example 6 and Table 19. The antibody was de-glycosylated prior to analysis.

FIG. 9C shows a peptide trace from an LC-MS analysis of anti-EGFR activatable masked IgG-type antibody [HC-CH1 MMP2/9, LC-CL lambda MMP2/9] digested with MMP9 as described in Example 6 and Table 19. The antibody was de-glycosylated prior to analysis.

FIG. 10A shows a dose response comparing binding of EGFR-expressing cells (MDA-MB-231) to uncut (solid circles) or cut with MMP9 (solid squares) anti-EGFR activatable masked antibody [HC-CH3 hole MMP2/9, LC-CH3 knob MMP2/9] or binding to a control anti-EGFR antibody (solid triangles), as described in Example 7 and Table 20.

FIG. 10B shows a dose response comparing binding of EGFR-expressing cells (A431) to uncut (solid circles) or cut with MMP9 (solid squares) anti-EGFR activatable masked antibody [HC-CH3 hole MMP2/9, LC-CH3 knob MMP2/9] or binding to a control anti-EGFR antibody (solid triangles), as described in Example 7 and Table 20.

FIG. 10C shows a dose response comparing binding of EGFR-expressing cells (MDA-MB-468) to uncut (solid circles) or cut with MMP9 (solid squares) anti-EGFR activatable masked antibody [HC-CH3 hole MMP2/9, LC-CH3 knob MMP2/9] or binding to a control anti-EGFR antibody (solid triangles), as described in Example 7 and Table 20.

FIG. 11A shows a binding curve from an ELISA assay which compares the binding activities of uncut activatable masked anti-EGFR antibodies [HC-CH3 hole MMP2/9, LC-CH3 knob MMP2/9] (solid diamonds) and [HC-CH1 MMP2/9, LC-CH1 lambda MMP2/9] (solid triangles) and control anti-EGFR antibody (open triangles), with recombinant EGFR protein, as described in Example 8. The EC50 values are listed in Table 21.

FIG. 11B shows a binding curve from an ELISA assay which compares the binding activities of uncut activatable masked anti-EGFR antibodies [HC-CH3 hole MMP2/9, LC-CH3 knob deleted] (solid circles) and [HC-CH3 hole MMP2/9, LC-CH3 knob non-cleavable] (solid squares) and [HC-CH3 hole MMP2/9, LC-CH3 knob extended] (solid triangles) and [HC-CH3 hole MMP2/9, LC-CH3 knob MMP2/9] (solid upside-down triangles) and control anti-EGFR antibody (solid diamonds), with recombinant EGFR protein, as described in Example 8. The binding curve for [HC-CH3 hole MMP2/9, LC-CH3 knob non-cleavable] cut with MMP9 is also shown (“cut”). The EC50 values are listed in Table 22.

FIG. 11C shows a binding curve from an ELISA assay which compares the binding activities of uncut activatable masked anti-EGFR antibodies [HC-CH3 hole non-cleavable, LC-CH3 knob deleted] (solid circles) and [HC-CH3 hole non-cleavable, LC-CH3 knob non-cleavable] (solid squares) and [HC-CH3 hole non-cleavable, LC-CH3 knob extended] (solid triangles) and [HC-CH3 hole MMP2/9, LC-CH3 knob MMP2/9] (solid upside-down triangles) and control anti-EGFR antibody (solid diamonds), with recombinant EGFR protein, as described in Example 8. The EC50 values are listed in Table 23.

FIG. 11D shows a binding curve from an ELISA assay which compares the binding activities of uncut activatable masked anti-EGFR antibodies [HC-CH3 hole deletion, LC-CH3 knob deleted] (solid circles) and [HC-CH3 hole deletion, LC-CH3 knob non-cleavable] (solid squares) and [HC-CH3 hole deletion, LC-CH3 knob extended] (solid triangles) and [HC-CH3 hole MMP2/9, LC-CH3 knob MMP2/9] (solid upside-down triangles) and control anti-EGFR antibody (solid diamonds), with recombinant EGFR protein, as described in Example 8. The EC50 values are listed in Table 24.

FIG. 11E shows a binding curve from an ELISA assay which compares the binding activities of an activatable masked anti-EGFR antibody [HC-CH3 hole MMP2/9, LC-CH3 knob MMP2/9] in uncut state (solid circles) or cut with MMP9 (solid up-side down triangles), and control anti-EGFR antibody (solid triangles), with recombinant EGFR protein, as described in Example 8. The EC50 values are listed in Table 25.

FIG. 11F shows a binding curve from an ELISA assay which compares the binding activities of an activatable masked anti-EGFR antibody [HC-CH1 MMP2/9, LC-CL lambda MMP2/9] in uncut state (solid squares) or cut with MMP9 (solid diamonds), and control anti-EGFR antibody (solid triangles), with recombinant EGFR protein, as described in Example 8. The EC50 values are listed in Table 26.

FIG. 11G shows a binding curve from an ELISA assay which compares the binding activities of an activatable masked anti-EGFR antibody [HC-CH3 hole MMP2/9, LC-CH3 knob non-cleavable] in uncut state (solid squares) or cut with MMP9 (“cut”, solid circles), and control anti-EGFR antibody (solid diamonds), with recombinant EGFR protein, as described in Example 8. The EC50 values are listed in Table 27.

FIG. 11H shows a binding curve from an ELISA assay which compares the binding activities of an activatable masked anti-EGFR antibody [HC-CH3 hole non-cleavable, LC-CH3 knob extended] in uncut state (solid triangles) or cut with MMP9 (“cut”, solid circles), and control anti-EGFR antibody (solid diamonds), with recombinant EGFR protein, as described in Example 8. The EC50 values are listed in Table 28.

FIG. 12A shows a bio-layer interferometry trace of human EGFR antigen binding to control anti-EGFR antibody, or activatable masked anti-EGFR antibody [HC-CH3 hole MMP2/9, LC-CH3 knob MMP2/9] in either uncut or cut state, as described in Example 9.

FIG. 12B shows a bio-layer interferometry trace of human EGFR antigen binding to control anti-EGFR antibody, or activatable masked anti-EGFR antibody [HC-CH1 MMP2/9, LC-CL lambda MMP2/9] in either uncut or cut state, as described in Example 9.

FIG. 12C shows a bio-layer interferometry trace of human EGFR antigen binding to control anti-EGFR antibody, or activatable masked anti-EGFR antibody [HC-CH1 MMP2/9, LC-CL kappa MMP2/9] in either uncut or cut state, as described in Example 9. The binding kinetic values are listed in Table 29.

FIG. 13A shows the result of FACs analysis for binding transgenic HeLa cells expressing an anti-EGFR DAR mimic having activatable masking moieties [HC-CH3(IgGl) MMP2/9, LC-CH3(IgGl) MMP2/9] in an uncut state, to anti-human kappa APC and fluorophore-labeled EGFR antigen, as described in Example 14. The positive control HeLa cells express anti-EGFR DAR with no mask and negative control is non-transgenic HeLa cells.

FIG. 13B shows the result of FACs analysis of transgenic HeLa cells expressing an anti-EGFR DAR mimic having activatable masking moieties [HC-CH3(IgG4) MMP2/9, LC-CH3(IgG4) MMP2/9] in an uncut state, binding to anti-human kappa APC and fluorophore-labeled EGFR antigen, as described in Example 14. The positive control HeLa cells express anti-EGFR DAR with no mask and negative control is non-transgenic HeLa cells.

FIG. 13C shows the result of FACs analysis of transgenic HeLa cells expressing an anti-EGFR DAR mimic having activatable masking moieties [HC-CH3 hole MMP2/9, LC-CH3 knob MMP2/9] in an uncut state, binding to anti-human kappa APC and fluorophore-labeled EGFR antigen, as described in Example 14. The positive control HeLa cells express anti-EGFR DAR with no mask and negative control is non-transgenic HeLa cells.

FIG. 13D shows the result of FACs analysis of transgenic HeLa cells expressing an anti-EGFR DAR mimic having activatable masking moieties [HC-CH1 MMP2/9, LC-CL kappa MMP2/9] in an uncut state, binding to anti-human kappa APC and fluorophore-labeled EGFR antigen, as described in Example 14. The positive control HeLa cells express anti-EGFR DAR with no mask and negative control is non-transgenic HeLa cells.

FIG. 13E shows the result of FACs analysis of transgenic HeLa cells expressing an anti-EGFR DAR mimic having activatable masking moieties [HC-38C2-VH MMP2/9, LC-38C2-VL MMP2/9] in an uncut state, binding to anti-human kappa APC and fluorophore-labeled EGFR antigen, as described in Example 14. The positive control HeLa cells express anti-EGFR DAR with no mask and negative control is non-transgenic HeLa cells.

FIG. 14A shows the result of FAC analysis of transgenic HeLa cells expressing an anti-EGFR DAR mimic having activatable masking moieties [HC-CH1 MMP2/9, LC-CL kappa MP2/9] in an uncut state, binding to anti-human kappa APC and fluorophore-labeled EGFR antigen, as described in Example 13.

FIG. 14B shows the result of FAC analysis of transgenic HeLa cells expressing an anti-EGFR DAR mimic having activatable masking moieties [HC-CH1 MMP2/9, LC-CL kappa MP2/9] digested with MMP9, binding to anti-human kappa APC and fluorophore-labeled EGFR antigen, as described in Example 13.

FIG. 14C shows the result of FAC analysis of positive control transgenic HeLa cells expressing an anti-EGFR DAR mimic with no activatable masking moieties, binding to anti-human kappa APC and fluorophore-labeled EGFR antigen, as described in Example 14.

FIG. 14D shows the result of FAC analysis of negative control non-transgenic HeLa cells that do not express an anti-EGFR DAR mimic and have no activatable masking moieties, binding to anti-human kappa APC and fluorophore-labeled EGFR antigen, as described in Example 13.

FIG. 15A shows the result of FAC analysis of transgenic HeLa cells expressing an anti-CD38 DAR mimic having activatable masking moieties [HC-CH3(IgG4) MMP2/9, LC-CH3(IgG4) MMP2/9] in an uncut state, binding to CD38 Fc labeled with PE and anti-hinge antibody labeled with APC, as described in Example 14.

FIG. 15B shows the result of FAC analysis of transgenic HeLa cells expressing an anti-CD38 DAR mimic lacking activatable masking moieties, binding to binding to CD38 Fc labeled with PE and anti-hinge antibody labeled with APC, as described in Example 14.

FIG. 15C shows the result of FAC analysis of transgenic HeLa cells expressing an anti-CD38 CAR mimic that lacks activatable masking moieties, binding to CD38 Fc labeled with PE and anti-hinge antibody labeled with APC, as described in Example 14.

FIG. 15D shows the result of FAC analysis of non-transgenic HeLa cells binding to CD38 Fc labeled with PE and anti-hinge antibody labeled with APC, as described in Example 14.

FIG. 15E shows the result of FAC analysis of transgenic HeLa cells expressing an anti-CD38 DAR mimic having activatable masking moieties [HC-CH3 hole MMP2/9, LC-CH3 knob MMP2/9] in an uncut state, binding to CD38 Fc labeled with PE and anti-hinge antibody labeled with APC, as described in Example 14.

FIG. 15F shows the same FAC result shown in FIG. 15B which is transgenic HeLa cells expressing an anti-CD38 DAR mimic lacking activatable masking moieties, binding to binding to CD38 Fc labeled with PE and anti-hinge antibody labeled with APC, as described in Example 14.

FIG. 15G shows the same FAC result shown in FIG. 15C which is transgenic HeLa cells expressing anti-CD38 CAR mimic that lacks activatable masking moieties, binding to CD38 Fc labeled with PE and anti-hinge antibody labeled with APC, as described in Example 14.

FIG. 15H shows the same FAC result shown in FIG. 15D which is non-transgenic HeLa cells binding to CD38 Fc labeled with PE and anti-hinge antibody labeled with APC, as described in Example 14.

FIG. 16A shows the result of FAC analysis of transgenic HeLa cells expressing an anti-CD38 DAR mimic lacking activatable masking moieties, binding to anti-hinge antibody labeled with APC and CD38 Fc labeled with PE, as described in Example 14.

FIG. 16B shows the result of FAC analysis of transgenic HeLa cells expressing an anti-CD38 CAR mimic lacking activatable masking moieties, binding to anti-hinge antibody labeled with APC and CD38 Fc labeled with PE, as described in Example 14.

FIG. 16C shows the result of FAC analysis of transgenic HeLa cells expressing an anti-BCMA CAR mimic lacking activatable masking moieties, binding to anti-hinge antibody labeled with APC and CD38 Fc labeled with PE, as described in Example 14.

FIG. 16D shows the result of FAC analysis of non-transgenic HeLa cells binding to CD38 Fc labeled with PE and anti-hinge antibody labeled with APC, binding to anti-hinge antibody labeled with APC and CD38 Fc labeled with PE, as described in Example 14.

FIG. 16E shows the result of FAC analysis of transgenic HeLa cells expressing an anti-CD38 DAR mimic having activatable masking moieties [HC-CH3(IgG4) MMP2/9, LC-CH3(IgG4) MMP2/9] in an uncut state, binding to anti-hinge antibody labeled with APC and CD38 Fc labeled with PE, as described in Example 14.

FIG. 16F shows the result of FAC analysis of transgenic HeLa cells expressing an anti-CD38 DAR mimic having activatable masking moieties [HC-CH3 hole MMP2/9, LC-CH3 knob MMP2/9] in an uncut state, binding to anti-hinge antibody labeled with APC and CD38 Fc labeled with PE, as described in Example 14.

FIG. 17 shows microscopy images of fixed cell imaging assays of detecting binding of target cells to anti-EGFR DAR mimics having activatable masking moieties [HC-CH MMP2/9, LC-CL kappa MMP2/9] in the presence or absence of conditioned media from MM1R multiple myeloma cells.

FIG. 18 shows the results of an ADCC assay comparing control anti-EGFR antibody with no masking moieties with various anti-EGFR activatable masked IgG-type antibodies, as described in Example 16. The EC50 values are listed in Table 34.

FIG. 19 shows the results of a flow cytometry assay used detect human T cells expressing an anti-EGFR DAR having activatable masking moieties [HC-CH3(IgG4) MMP2/9, LC-CH3(IgG4) MMP2/9] and carrying an intracellular signaling domain having 4-1BB and CD3zeta, in an uncut state, and binding to anti-human kappa APC and fluorophore-labeled EGFR antigen, as described in Example 18. The test cells express anti-EGFR DAR carry linkers MMP2/9, non-cleavable, or uPA. The positive control T cells express anti-EGFR DAR with no mask and negative control is non-transgenic GFP T cells.

FIG. 20A shows the results of a cytotoxicity assay comparing control anti-EGFR DAR T cells (A), activated T cells (E), and no effector (F), with anti-EGFR masked DAR T cells carrying linkers cleavable with MMP9 (B) or uPA (C), or non-cleavable linkers (D). The target cells are A549. The E:T ratio is 5:1.

FIG. 20B shows a bar graph of the cytotoxicity data shown in FIG. 20A above, comparing cell killing levels at 8 and 48 hours.

FIG. 21A shows the results of a cytotoxicity assay comparing control anti-EGFR DAR T cells (A), activated T cells (E), and no effector (F), with anti-EGFR masked DAR T cells carrying linkers cleavable with MMP9 (B) or uPA (C), or non-cleavable linkers (D). The target cells are A549. The E:T ratio is 20:1.

FIG. 21B shows a bar graph of the cytotoxicity data shown in FIG. 21A above, comparing cell killing levels at 8 and 48 hours.

FIG. 22A shows a standard curve of an interferon gamma release assay, described in Example 20.

FIG. 22B is a bar graph of the interferon gamma release assay, comparing anti-EGFR masked DAR T cells carrying linkers cleavable with MMP9 or uPA, or non-cleavable linkers. The negative controls included anti-EGFR DAR T cells, activated T cells (GFP), and no DAR T cells.

FIG. 23 shows the amino acid sequence of a precursor polypeptide chain of a control anti-EGFR DAR containing a first polypeptide chain, a self-cleaving sequence (e.g., T2A), and a second polypeptide chain, where the precursor polypeptide chain lacks Ig gamma-1 CH3 masking moieties and lacks cleavable linkers, and where the precursor polypeptide chain includes an intracellular signaling domain from 4-1BB and CD3zeta. The amino acid sequence of the predicted first and second polypeptide chains are also shown. Preparation of transgenic T cells expressing the precursor, and first and second polypeptide chains, are described in Example 17. Analysis of these transgenic T cells are shown in FIGS. 19-22B.

FIG. 24 shows the amino acid sequence of a precursor polypeptide chain of an anti-EGFR DAR containing a first polypeptide chain, a self-cleaving sequence (e.g., T2A), and a second polypeptide chain, where the precursor polypeptide chain includes Ig gamma-1 CH3 masking moieties linked to MMP2/9 cleavable linkers, and where the precursor polypeptide chain includes an intracellular signaling domain from 4-1BB and CD3zeta. The amino acid sequence of the predicted first and second polypeptide chains are also shown. Preparation of transgenic T cells expressing the precursor, and first and second polypeptide chains, are described in Example 17. Analysis of these transgenic T cells are shown in FIGS. 19-22B.

FIG. 25 shows the amino acid sequence of a precursor polypeptide chain of an anti-EGFR DAR containing a first polypeptide chain, a self-cleaving sequence (e.g., T2A), and a second polypeptide chain, where the precursor polypeptide chain includes Ig gamma-1 CH3 masking moieties linked to uPA cleavable linkers, and where the precursor polypeptide chain includes an intracellular signaling domain from 4-1BB and CD3zeta. The amino acid sequence of the predicted first and second polypeptide chains are also shown. Preparation of transgenic T cells expressing the precursor, and first and second polypeptide chains, are described in Example 17. Analysis of these transgenic T cells are shown in FIGS. 19-22B.

FIG. 26 shows the amino acid sequence of a precursor polypeptide chain of an anti-EGFR DAR containing a first polypeptide chain, a self-cleaving sequence (e.g., T2A), and a second polypeptide chain, where the precursor polypeptide chain includes Ig gamma-1 CH3 masking moieties linked to non-cleavable linkers, and where the precursor polypeptide chain includes an intracellular signaling domain from 4-1BB and CD3zeta. The amino acid sequence of the predicted first and second polypeptide chains are also shown. Preparation of transgenic T cells expressing the precursor, and first and second polypeptide chains, are described in Example 17. Analysis of these transgenic T cells are shown in FIGS. 19-22B.

### DETAILED DESCRIPTION

### Definitions

Unless defined otherwise, technical and scientific terms used herein have meanings that are commonly understood by those of ordinary skill in the art unless defined otherwise. Generally, terminologies pertaining to techniques of cell and tissue culture, molecular biology, immunology, microbiology, genetics, transgenic cell production, protein chemistry and nucleic acid chemistry and hybridization described herein are well known and commonly used in the art. The methods and techniques provided herein are generally performed according to conventional procedures well known in the art and as described in various general and more specific references that are cited and discussed herein unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992). A number of basic texts describe standard antibody production processes, including, Borrebaeck (ed) Antibody Engineering, 2nd Edition Freeman and Company, NY, 1995; McCafferty et al. Antibody Engineering, A Practical Approach IRL at Oxford Press, Oxford, England, 1996; and Paul (1995) Antibody Engineering Protocols Humana Press, Towata, N.J., 1995; Paul (ed.), Fundamental Immunology, Raven Press, N.Y, 1993; Coligan (1991) Current Protocols in Immunology Wiley/Greene, NY; Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Coding Monoclonal Antibodies: Principles and Practice (2nd ed.) Academic Press, New York, N.Y., 1986, and Kohler and Milstein Nature 256: 495-497, 1975. All of the references cited herein are incorporated herein by reference in their entireties. Enzymatic reactions and enrichment/purification techniques are also well known and are performed according to manufacturer’s specifications, as commonly accomplished in the art or as described herein. The terminology used in connection with, and the laboratory procedures and techniques of, analytical chemistry, synthetic organic chemistry, and medicinal and pharmaceutical chemistry described herein are well known and commonly used in the art. Standard techniques can be used for chemical syntheses, chemical analyses, pharmaceutical preparation, formulation, and delivery, and treatment of patients.

The headings provided herein are not limitations of the various aspects of the disclosure, which aspects can be understood by reference to the specification as a whole.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative (e.g., “or”) herein is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition.

The terms “peptide”, “polypeptide” and “protein” and other related terms used herein are used interchangeably and refer to a polymer of amino acids and are not limited to any particular length. Polypeptides may comprise natural and non-natural amino acids. Polypeptides include recombinant or chemically-synthesized forms. Polypeptides also include precursor molecules that have not yet been subjected to cleavage, for example cleavage by a secretory signal peptide or by non-enzymatic cleavage at certain amino acid residues. Polypeptides include mature molecules that have undergone cleavage. These terms encompass native and artificial proteins, protein fragments and polypeptide analogs (such as muteins, variants, chimeric proteins and fusion proteins) of a protein sequence as well as post-translationally, or otherwise covalently or non-covalently, modified proteins. Two or more polypeptides (e.g., 3 polypeptide chains) can associate with each other, via covalent and/or non-covalent association, to form a polypeptide complex. Association of the polypeptide chains can also include peptide folding. Thus, a polypeptide complex can be dimeric, trimeric, tetrameric, or higher order complexes depending on the number of polypeptide chains that form the complex.

The terms “nucleic acid”, “polynucleotide” and “oligonucleotide” and other related terms used herein are used interchangeably and refer to polymers of nucleotides and are not limited to any particular length. Nucleic acids include recombinant and chemically-synthesized forms. Nucleic acids include DNA molecules (cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. Nucleic acid molecule can be single-stranded or double-stranded. In one embodiment, the nucleic acid molecules of the disclosure comprise a contiguous open reading frame encoding an antibody, or a fragment or scFv, derivative, mutein, or variant thereof. In one embodiment, nucleic acids comprise one type of polynucleotide or a mixture of two or more different types of polynucleotides. Nucleic acids encoding activatable masked IgG-type antibodies and dimeric antigen receptor (DAR) having activatable masking moieties or antigen-binding portions thereof, are described herein.

The term “recover” or “recovery” or “recovering”, and other related terms, refers to obtaining a protein (e.g., an antibody or an antigen binding portion thereof), from host cell culture medium or from host cell lysate or from the host cell membrane. In one embodiment, the protein is expressed by the host cell as a recombinant protein fused to a secretion signal peptide (leader peptide sequence) sequence which mediates secretion of the expressed protein from a host cell (e.g., from a mammalian host cell). The secreted protein can be recovered from the host cell medium. In one embodiment, the protein is expressed by the host cell as a recombinant protein that lacks a secretion signal peptide sequence which can be recovered from the host cell lysate. In one embodiment, the protein is expressed by the host cell as a membrane-bound protein which can be recovered using a detergent to release the expressed protein from the host cell membrane. In one embodiment, irrespective of the method used to recover the protein, the protein can be subjected to procedures that remove cellular debris from the recovered protein. For example, the recovered protein can be subj ected to chromatography, gel electrophoresis and/or dialysis. In one embodiment, the chromatography comprises any one or any combination or two or more procedures including affinity chromatography, hydroxyapatite chromatography, ion-exchange chromatography, reverse phase chromatography and/or chromatography on silica. In one embodiment, affinity chromatography comprises protein A or G (cell wall components from Staphylococcus aureus).

The term “isolated” refers to a protein (e.g., an antibody or an antigen binding portion thereof) or polynucleotide that is substantially free of other cellular material. A protein may be rendered substantially free of naturally associated components (or components associated with a cellular expression system or chemical synthesis methods used to produce the antibody) by isolation, using protein purification techniques well known in the art. The term isolated also refers in some embodiment to protein or polynucleotides that are substantially free of other molecules of the same species, for example other protein or polynucleotides having different amino acid or nucleotide sequences, respectively. The purity of homogeneity of the desired molecule can be assayed using techniques well known in the art, including low resolution methods such as gel electrophoresis and high resolution methods such as HPLC or mass spectrometry. In one embodiment, the activatable masked IgG-type antibodies and dimeric antigen receptor (DAR) having activatable masking moieties or antigen-binding portions thereof, of the present disclosure are isolated.

The term “leader sequence” or “leader peptide” or “peptide signal sequence” or “signal peptide” or “secretion signal peptide” refers to a peptide sequence that is located at the N-terminus of a polypeptide. A leader sequence directs a polypeptide chain to a cellular secretory pathway and can direct integration and anchoring of the polypeptide into the lipid bilayer of the cellular membrane. Typically, a leader sequence is about 10-50 amino acids in length. A leader sequence can direct transport of a precursor polypeptide from the cytosol to the endoplasmic reticulum. In one embodiment, a leader sequence includes signal sequences comprising CD8α, CD28 or CD16 leader sequences. In one embodiment, the signal sequence comprises a mammalian sequence, including for example mouse or human Ig gamma secretion signal peptide. In one embodiment, a leader sequence comprises a mouse Ig gamma leader peptide sequence MEWSWVFLFFLSVTTGVHS.

An “antigen binding protein” and related terms used herein refers to a protein comprising a portion that binds to an antigen and, optionally, a scaffold or framework portion that allows the antigen binding portion to adopt a conformation that promotes binding of the antigen binding protein to the antigen. Examples of antigen binding proteins include antibodies, antibody fragments (e.g., an antigen binding portion of an antibody), antibody derivatives, and antibody analogs. The antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, for example, Korndorfer et al., 2003, Proteins: Structure, Function, and Bioinformatics, Volume 53, Issue 1: 121-129; Roque et al., 2004, Biotechnol. Prog. 20:639-654. In addition, peptide antibody mimetics (“PAMs”) can be used, as well as scaffolds based on antibody mimetics utilizing fibronection components as a scaffold. Antigen binding proteins comprising activatable masked IgG-type antibodies and dimeric antigen receptor (DAR) having activatable masking moieties or antigen-binding portions thereof, are described herein.

An antigen binding protein can have, for example, the structure of a naturally occurring immunoglobulin. In one embodiment, an “immunoglobulin” refers to a naturally-occurring tetrameric molecule composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa or lambda light chains. Heavy chains are classified as mu, delta, gamma, alpha, or epsilon, and define the antibody’s isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The heavy and/or light chains may or may not include a leader sequence for secretion. The variable regions of each light/heavy chain pair form the antibody binding site such that an intact immunoglobulin has two antigen binding sites. In one embodiment, an antigen binding protein can be a synthetic molecule having a structure that differs from a tetrameric immunoglobulin molecule but still binds a target antigen or binds two or more target antigens. For example, a synthetic antigen binding protein can comprise antibody fragments, 1-6 or more polypeptide chains, asymmetrical assemblies of polypeptides, or other synthetic molecules. Antigen binding proteins having immunoglobulin-like properties that bind specifically to EGFR or CD38 are described herein.

The variable regions of immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4.

One or more CDRs may be incorporated into a molecule either covalently or noncovalently to make it an antigen binding protein. An antigen binding protein may incorporate the CDR(s) as part of a larger polypeptide chain, may covalently link the CDR(s) to another polypeptide chain, or may incorporate the CDR(s) noncovalently. The CDRs permit the antigen binding protein to specifically bind to a particular antigen of interest.

The assignment of amino acids to each domain is in accordance with the definitions of Kabat et al. in Sequences of Proteins of Immunological Interest, 5^{th} Ed., US Dept. of Health and Human Services, PHS, NIH, NIH Publication no. 91-3242, 1991 (“Kabat numbering”). Other numbering systems for the amino acids in immunoglobulin chains include IMGT.RTM. (international ImMunoGeneTics information system; Lefranc et al, Dev. Comp. Immunol. 29:185-203; 2005) and AHo (Honegger and Pluckthun, J. Mol. Biol. 309(3):657-670; 2001); Chothia (Al-Lazikani et al., 1997 Journal of Molecular Biology 273:927-948; Contact (Maccallum et al., 1996 Journal of Molecular Biology 262:732-745, and Aho (Honegger and Pluckthun 2001 Journal of Molecular Biology 309:657-670.

An “antibody” and “antibodies” and related terms used herein refers to an intact immunoglobulin or to an antigen binding portion thereof that binds specifically to an antigen. Antigen binding portions may be produced by recombinant DNA techniques or by enzymatic or chemical cleavage of intact antibodies. Antigen binding portions include, inter alia, Fab, Fab′, F(ab′)₂, Fv, domain antibodies (dAbs), and complementarity determining region (CDR) fragments, single-chain antibodies (scFv), chimeric antibodies, diabodies, triabodies, tetrabodies, and polypeptides that contain at least a portion of an immunoglobulin that is sufficient to confer specific antigen binding to the polypeptide.

Antibodies include recombinantly produced antibodies and antigen binding portions. Antibodies include non-human, chimeric, humanized and fully human antibodies. Antibodies include monospecific, multispecific (e.g., bispecific, trispecific and higher order specificities). Antibodies include tetrameric antibodies, light chain monomers, heavy chain monomers, light chain dimers, heavy chain dimers. Antibodies include F(ab′)₂ fragments, Fab′ fragments and Fab fragments. Antibodies include single domain antibodies, monovalent antibodies, single chain antibodies, single chain variable fragment (scFv), camelized antibodies, affibodies, disulfide-linked Fvs (sdFv), anti-idiotypic antibodies (anti-Id), minibodies. Antibodies include monoclonal and polyclonal populations. Antibodies-like molecules comprising activatable masked IgG-type antibodies and dimeric antigen receptor (DAR) having activatable masking moieties or antigen-binding portions thereof, are described herein.

An “antigen binding domain,” “antigen binding region,” or “antigen binding site” and other related terms used herein refer to a portion of an antigen binding protein that contains amino acid residues (or other moieties) that interact with an antigen and contribute to the antigen binding protein’s specificity and affinity for the antigen. For an antibody that specifically binds to its antigen, this will include at least part of at least one of its CDR domains. Antigen binding domains from anti-EGFR or anti-CD38 antibodies are described herein.

The terms “specific binding”, “specifically binds” or “specifically binding” and other related terms, as used herein in the context of an antibody or antigen binding protein or antibody fragment, refer to non-covalent or covalent preferential binding to an antigen relative to other molecules or moieties (e.g., an antibody specifically binds to a particular antigen relative to other available antigens). In one embodiment, an antibody specifically binds to a target antigen if it binds to the antigen with a dissociation constant K_{D} of 10⁻⁵ M or less, or 10⁻⁶ M or less, or 10⁻⁷ M or less, or 10⁻⁸ M or less, or 10⁻⁹ M or less, or 10⁻¹⁰ M or less, or 10⁻¹¹ M or less. In one embodiment, activatable masked IgG-type antibodies and dimeric antigen receptor (DAR) having activatable masking moieties or antigen-binding portions thereof that bind specifically to their target antigens (e.g., EGFR or CD38 antigen) are described herein.

In one embodiment, binding specificity can be measure by ELISA, radioimmune assay (RIA), electrochemiluminescence assays (ECL), immunoradiometric assay (IRMA), or enzyme immune assay (EIA).

In one embodiment, a dissociation constant (K_{D}) can be measured using a BIACORE surface plasmon resonance (SPR) assay. Surface plasmon resonance refers to an optical phenomenon that allows for the analysis of real-time interactions by detection of alterations in protein concentrations within a biosensor matrix, for example using the BIACORE system (Biacore Life Sciences division of GE Healthcare, Piscataway, NJ).

An “epitope” and related terms as used herein refers to a portion of an antigen that is bound by an antigen binding protein (e.g., by an antibody or an antigen binding portion thereof). An epitope can comprise portions of two or more antigens that are bound by an antigen binding protein. An epitope can comprise non-contiguous portions of an antigen or of two or more antigens (e.g., amino acid residues that are not contiguous in an antigen’s primary sequence but that, in the context of the antigen’s tertiary and quaternary structure, are near enough to each other to be bound by an antigen binding protein). Generally, the variable regions, particularly the CDRs, of an antibody interact with the epitope. In one embodiment, activatable masked IgG-type antibodies and dimeric antigen receptor (DAR) having activatable masking moieties or antigen-binding portions thereof that bind an epitope of EGFR or CD38 antigen are described herein.

An “antibody fragment”, “antibody portion”, “antigen-binding fragment of an antibody”, or “antigen-binding portion of an antibody” and other related terms used herein refer to a molecule other than an intact antibody that comprises a portion of an intact antibody that binds the antigen to which the intact antibody binds. Examples of antibody fragments include, but are not limited to, Fv, Fab, Fab′, Fab′—SH, F(ab′)₂; Fd; and Fv fragments, as well as dAb; diabodies; linear antibodies; single-chain antibody molecules (e.g. scFv); polypeptides that contain at least a portion of an antibody that is sufficient to confer specific antigen binding to the polypeptide. Antigen binding portions of an antibody may be produced by recombinant DNA techniques or by enzymatic or chemical cleavage of intact antibodies. Antigen binding portions include, inter alia, Fab, Fab′, F(ab′)2, Fv, domain antibodies (dAbs), and complementarity determining region (CDR) fragments, chimeric antibodies, diabodies, triabodies, tetrabodies, and polypeptides that contain at least a portion of an immunoglobulin that is sufficient to confer antigen binding properties to the antibody fragment. In one embodiment, antigen-binding fragments include fragments of activatable masked IgG-type antibodies and dimeric antigen receptor (DAR) having activatable masking moieties or antigen-binding portions thereof that bind specifically to their target antigens (e.g., EGFR or CD38 antigen) which are described herein.

The terms “Fab”, “Fab fragment” and other related terms refers to a monovalent fragment comprising a variable light chain region (V_{L}), constant light chain region (C_{L}), variable heavy chain region (V_{H}), and first constant region (C_{H1}). A Fab is capable of binding an antigen. An F(ab′)₂ fragment is a bivalent fragment comprising two Fab fragments linked by a disulfide bridge at the hinge region. A F(Ab′)₂ has antigen binding capability. An Fd fragment comprises V_{H} and C_{H1} regions. An Fv fragment comprises V_{L} and V_{H} regions. An Fv can bind an antigen. A dAb fragment has a V_{H} domain, a V_{L} domain, or an antigen-binding fragment of a V_{H} or VL domain (U.S. Pat. 6,846,634 and 6,696,245; U.S. published Application Nos. 2002/02512, 2004/0202995, 2004/0038291, 2004/0009507, 2003/0039958; and Ward et al., Nature 341:544-546, 1989). Fab fragments comprising antigen binding portions from anti-EGFR or anti-CD38 antibodies are described herein.

A single-chain antibody (scFv) is an antibody in which a V_{L} and a V_{H} region are joined via a linker (e.g., a synthetic sequence of amino acid residues) to form a continuous protein chain. Preferably the linker is long enough to allow the protein chain to fold back on itself and form a monovalent antigen binding site (see, e.g., Bird et al., 1988, Science 242:423-26 and Huston et al., 1988, Proc. Natl. Acad. Sci. USA 85:5879-83). Single chain antibodies comprising antigen binding portions from anti-EGFR or anti-CD38 antibodies are described herein.

Diabodies are bivalent antibodies comprising two polypeptide chains, wherein each polypeptide chain comprises V_{H} and V_{L} domains joined by a linker that is too short to allow for pairing between two domains on the same chain, thus allowing each domain to pair with a complementary domain on another polypeptide chain (see, e.g., Holliger et al., 1993, Proc. Natl. Acad. Sci. USA 90:6444-48, and Poljak et al., 1994, Structure 2:1121-23). If the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different. Diabody, tribody and tetrabody constructs can be prepared using antigen binding portions from any of the anti-EGFR or anti-CD38 antibodies described herein.

The term “human antibody” refers to antibodies that have one or more variable and constant regions derived from human immunoglobulin sequences. In one embodiment, all of the variable and constant domains are derived from human immunoglobulin sequences (e.g., a fully human antibody). These antibodies may be prepared in a variety of ways, examples of which are described below, including through recombinant methodologies or through immunization with an antigen of interest of a mouse that is genetically modified to express antibodies derived from human heavy and/or light chain-encoding genes. Fully human anti-EGFR or anti-CD38 antibodies and antigen binding proteins, or portions thereof are described herein.

A “humanized” antibody refers to an antibody having a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

The term “chimeric antibody” and related terms used herein refers to an antibody that contains one or more regions from a first antibody and one or more regions from one or more other antibodies. In one embodiment, one or more of the CDRs are derived from a human antibody. In another embodiment, all of the CDRs are derived from a human antibody. In another embodiment, the CDRs from more than one human antibody are mixed and matched in a chimeric antibody. For instance, a chimeric antibody may comprise a CDR1 from the light chain of a first human antibody, a CDR2 and a CDR3 from the light chain of a second human antibody, and the CDRs from the heavy chain from a third antibody. In another example, the CDRs originate from different species such as human and mouse, or human and rabbit, or human and goat. One skilled in the art will appreciate that other combinations are possible.

Further, the framework regions may be derived from one of the same antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody (-ies) from another species or belonging to another antibody class or subclass. Also included are fragments of such antibodies that exhibit the desired biological activity (i.e., the ability to specifically bind a target antigen). Chimeric antibodies can be prepared from portions of any of the activatable masked IgG-type antibodies and dimeric antigen receptor (DAR) having activatable masking moieties or antigen-binding portions thereof are described herein.

As used herein, the term “variant” polypeptides and “variants” of polypeptides refers to a polypeptide comprising an amino acid sequence with one or more amino acid residues inserted into, deleted from and/or substituted into the amino acid sequence relative to a reference polypeptide sequence. Polypeptide variants include fusion proteins. In the same manner, a variant polynucleotide comprises a nucleotide sequence with one or more nucleotides inserted into, deleted from and/or substituted into the nucleotide sequence relative to another polynucleotide sequence. Polynucleotide variants include fusion polynucleotides.

As used herein, the term “derivative” of a polypeptide is a polypeptide (e.g., an antibody) that has been chemically modified, e.g., via conjugation to another chemical moiety such as, for example, polyethylene glycol, albumin (e.g., human serum albumin), phosphorylation, and glycosylation. Unless otherwise indicated, the term “antibody” includes, in addition to antibodies comprising two full-length heavy chains and two full-length light chains, derivatives, variants, fragments, and muteins thereof, examples of which are described below.

The term “Fc” or “Fc region” as used herein refers to the portion of an antibody heavy chain constant region beginning in or after the hinge region and ending at the C-terminus of the heavy chain. The Fc region comprises at least a portion of the CH and CH3 regions, and may or may not include a portion of the hinge region. Two polypeptide chains each carrying a half Fc region can dimerize to form an Fc region. An Fc region can bind Fc cell surface receptors and some proteins of the immune complement system. An Fc region exhibits effector function, including any one or any combination of two or more activities including complement-dependent cytotoxicity (CDC), antibody-dependent cell-mediated cytotoxicity (ADCC), antibody-dependent phagocytosis (ADP), opsonization and/or cell binding. An Fc region can bind an Fc receptor, including FcyRI (e.g., CD64), FcyRII (e.g., CD32) and/or FcyRIII (e.g., CD16a).

The term “labeled antibody” or related terms as used herein refers to antibodies and their antigen binding portions thereof that are unlabeled or joined to a detectable label or moiety for detection, wherein the detectable label or moiety is radioactive, colorimetric, antigenic, enzymatic, a detectable bead (such as a magnetic or electrodense (e.g., gold) bead), biotin, streptavidin or protein A. A variety of labels can be employed, including, but not limited to, radionuclides, fluorescers, enzymes, enzyme substrates, enzyme cofactors, enzyme inhibitors and ligands (e.g., biotin, haptens). Any of the activatable masked IgG-type antibodies and dimeric antigen receptor (DAR) having activatable masking moieties or antigen-binding portions thereof that described herein can be unlabeled or can be joined to a detectable label or moiety.

The “percent identity” or “percent homology” and related terms used herein refers to a quantitative measurement of the similarity between two polypeptide or between two polynucleotide sequences. The percent identity between two polypeptide sequences is a function of the number of identical amino acids at aligned positions that are shared between the two polypeptide sequences, taking into account the number of gaps, and the length of each gap, which may need to be introduced to optimize alignment of the two polypeptide sequences. In a similar manner, the percent identity between two polynucleotide sequences is a function of the number of identical nucleotides at aligned positions that are shared between the two polynucleotide sequences, taking into account the number of gaps, and the length of each gap, which may need to be introduced to optimize alignment of the two polynucleotide sequences. A comparison of the sequences and determination of the percent identity between two polypeptide sequences, or between two polynucleotide sequences, may be accomplished using a mathematical algorithm. For example, the “percent identity” or “percent homology” of two polypeptide or two polynucleotide sequences may be determined by comparing the sequences using the GAP computer program (a part of the GCG Wisconsin Package, version 10.3 (Accelrys, San Diego, Calif.)) using its default parameters. Expressions such as “comprises a sequence with at least X% identity to Y” with respect to a test sequence mean that, when aligned to sequence Y as described above, the test sequence comprises residues identical to at least X% of the residues of Y.

In one embodiment, the amino acid sequence of a test antibody may be similar but not identical to any of the amino acid sequences of the polypeptides that make up the activatable masked IgG-type antibodies and dimeric antigen receptor (DAR) having activatable masking moieties or antigen-binding portions thereof that are described herein. The similarities between the test antibody and the polypeptides can be at least 95%, or at or at least 96% identical, or at least 97% identical, or at least 98% identical, or at least 99% identical, to any of the polypeptides that make up the activatable masked IgG-type antibodies and dimeric antigen receptor (DAR) having activatable masking moieties or antigen-binding portions thereof that are described herein. In one embodiment, similar polypeptides can contain amino acid substitutions within a heavy and/or light chain. In one embodiment, the amino acid substitutions comprise one or more conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference in its entirety. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine.

Antibodies can be obtained from sources such as serum or plasma that contain immunoglobulins having varied antigenic specificity. If such antibodies are subjected to affinity purification, they can be enriched for a particular antigenic specificity. Such enriched preparations of antibodies usually are made of less than about 10% antibody having specific binding activity for the particular antigen. Subjecting these preparations to several rounds of affinity purification can increase the proportion of antibody having specific binding activity for the antigen. Antibodies prepared in this manner are often referred to as “monospecific.” Monospecific antibody preparations can be made up of about 10%, 20%, 30%, 40%, 50%, 60%, 70%, 75%, 80%, 85%, 90%, 95%, 97%, 99%, or 99.9% antibody having specific binding activity for the particular antigen. Antibodies can be produced using recombinant nucleic acid technology as described below.

A “vector” and related terms used herein refers to a nucleic acid molecule (e.g., DNA or RNA) which can be operably linked to foreign genetic material (e.g., nucleic acid transgene). Vectors can be used as a vehicle to introduce foreign genetic material into a cell (e.g., host cell). Vectors can include at least one restriction endonuclease recognition sequence for insertion of the transgene into the vector. Vectors can include at least one gene sequence that confers antibiotic resistance or a selectable characteristic to aid in selection of host cells that harbor a vector-transgene construct. Vectors can be single-stranded or double-stranded nucleic acid molecules. Vectors can be linear or circular nucleic acid molecules. A donor nucleic acid used for gene editing methods employing zinc finger nuclease, TALEN or CRISPR/Cas can be a type of a vector. One type of vector is a “plasmid,” which refers to a linear or circular double stranded extrachromosomal DNA molecule which can be linked to a transgene, and is capable of replicating in a host cell, and transcribing and/or translating the transgene. A viral vector typically contains viral RNA or DNA backbone sequences which can be linked to the transgene. The viral backbone sequences can be modified to disable infection but retain insertion of the viral backbone and the co-linked transgene into a host cell genome. Examples of viral vectors include retroviral, lentiviral, adenoviral, adeno-associated, baculoviral, papovaviral, vaccinia viral, herpes simplex viral and Epstein Barr viral vectors. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome.

An “expression vector” is a type of vector that can contain one or more regulatory sequences, such as inducible and/or constitutive promoters and enhancers. Expression vectors can include ribosomal binding sites and/or polyadenylation sites. Expression vectors can include one or more origin of replication sequence. Regulatory sequences direct transcription, or transcription and translation, of a transgene linked to the expression vector which is transduced into a host cell. The regulatory sequence(s) can control the level, timing and/or location of expression of the transgene. The regulatory sequence can, for example, exert its effects directly on the transgene, or through the action of one or more other molecules (e.g., polypeptides that bind to the regulatory sequence and/or the nucleic acid). Regulatory sequences can be part of a vector. Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-3606. An expression vector can comprise nucleic acids that encode at least a portion of any of the activatable masked IgG-type antibodies and dimeric antigen receptor (DAR) having activatable masking moieties or antigen-binding portions thereof that are described herein.

A transgene is “operably linked” to a vector when there is linkage between the transgene and the vector to permit functioning or expression of the transgene sequences contained in the vector. In one embodiment, a transgene is “operably linked” to a regulatory sequence when the regulatory sequence affects the expression (e.g., the level, timing, or location of expression) of the transgene.

The terms “transfected” or “transformed” or “transduced” or other related terms used herein refer to a process by which exogenous nucleic acid (e.g., transgene) is transferred or introduced into a host cell. A “transfected” or “transformed” or “transduced” host cell is one which has been transfected, transformed or transduced with exogenous nucleic acid (transgene). The host cell includes the primary subject cell and its progeny. Exogenous nucleic acids encoding at least a portion of any of the activatable masked IgG-type antibodies and dimeric antigen receptor (DAR) having activatable masking moieties or antigen-binding portions thereof that are described herein can be introduced into a host cell. Expression vectors comprising at least a portion of any of the activatable masked IgG-type antibodies and dimeric antigen receptor (DAR) having activatable masking moieties or antigen-binding portions thereof that are described herein can be introduced into a host cell, and the host cell can express polypeptides comprising at least a portion of the activatable masked IgG-type antibodies and dimeric antigen receptor (DAR) having activatable masking moieties or antigen-binding portions thereof that are described herein.

The terms “host cell” or “or a population of host cells” or related terms as used herein refer to a cell (or a population thereof) into which foreign (exogenous or transgene) nucleic acids have been introduced. The foreign nucleic acids can include an expression vector operably linked to a transgene, and the host cell can be used to express the nucleic acid and/or polypeptide encoded by the foreign nucleic acid (transgene). A host cell (or a population thereof) can be a cultured cell or can be extracted from a subject. The host cell (or a population thereof) includes the primary subject cell and its progeny without any regard for the number of passages. Progeny cells may or may not harbor identical genetic material compared to the parent cell. Host cells encompass progeny cells. In one embodiment, a host cell describes any cell (including its progeny) that has been modified, transfected, transduced, transformed, and/or manipulated in any way to express an antibody, as disclosed herein. In one example, the host cell (or population thereof) can be introduced with an expression vector operably linked to a nucleic acid encoding the desired antibody, or an antigen binding portion thereof, described herein. Host cells and populations thereof can harbor an expression vector that is stably integrated into the host’s genome, or can harbor an extrachromosomal expression vector. In one embodiment, host cells and populations thereof can harbor an extrachromosomal vector that is present after several cell divisions or is present transiently and is lost after several cell divisions.

Transgenic host cells can be prepared using non-viral methods, including well-known designer nucleases including zinc finger nucleases, TALENS or CRISPR/Cas. A transgene can be introduced into a host cell’s genome using genome editing technologies such as zinc finger nuclease. A zinc finger nuclease includes a pair of chimeric proteins each containing a non-specific endonuclease domain of a restriction endonuclease (e.g., FokI) fused to a DNA-binding domain from an engineered zinc finger motif. The DNA-binding domain can be engineered to bind a specific sequence in the host’s genome and the endonuclease domain makes a double-stranded cut. The donor DNA carries the transgene, for example any of the nucleic acids encoding a CAR or DAR construct described herein, and flanking sequences that are homologous to the regions on either side of the intended insertion site in the host cell’s genome. The host cell’s DNA repair machinery enables precise insertion of the transgene by homologous DNA repair. Transgenic mammalian host cells have been prepared using zinc finger nucleases (U.S. patent Nos. 9,597,357, 9,616,090, 9,816,074 and 8,945,868). A transgenic host cell can be prepared using TALEN (Transcription Activator-Like Effector Nucleases) which are similar to zinc finger nucleases in that they include a non-specific endonuclease domain fused to a DNA-binding domain which can deliver precise transgene insertion. Like zinc finger nucleases, TALEN also introduce a double-strand cut into the host’s DNA. Transgenic host cells can be prepared using CRISPR (Clustered Regularly Interspaced Short Palindromic Repeats). CRISPR employs a Cas endonuclease coupled to a guide RNA for target specific donor DNA integration. The guide RNA includes a conserved multi-nucleotide containing protospacer adjacent motif (PAM) sequence upstream of the gRNA-binding region in the target DNA and hybridizes to the host cell target site where the Cas endonuclease cleaves the double-stranded target DNA. The guide RNA can be designed to hybridize to a specific target site. Similar to zinc finger nuclease and TALEN, the CRISPR/Cas system can be used to introduce site specific insertion of donor DNA having flanking sequences that have homology to the insertion site. Examples of CRISPR/Cas systems used to modify genomes are described for example in U.S. Pat. Nos. 8,697,359, 10,000,772, 9,790,490, and U. S. Pat. Application Publication No. US 2018/0346927. In one embodiment, transgenic host cells can be prepared using zinc finger nuclease, TALEN or CRISPR/Cas system, and the host target site can be a TRAC gene (T Cell Receptor Alpha Constant). The donor DNA can include for example any of the nucleic acids encoding a CAR or DAR construct described herein. Electroporation, nucleofection or lipofection can be used to co-deliver into the host cell the donor DNA with the zinc finger nuclease, TALEN or CRISPR/Cas system.

A host cell can be a prokaryote, for example, E. coli, or it can be a eukaryote, for example, a single-celled eukaryote (e.g., a yeast or other fungus), a plant cell (e.g., a tobacco or tomato plant cell), an mammalian cell (e.g., a human cell, a monkey cell, a hamster cell, a rat cell, a mouse cell, or an insect cell) or a hybridoma. In one embodiment, a host cell can be introduced with an expression vector operably linked to a nucleic acid encoding a desired antibody thereby generating a transfected/transformed host cell which is cultured under conditions suitable for expression of the antibody by the transfected/transformed host cell, and optionally recovering the antibody from the transfected/transformed host cells (e.g., recovery from host cell lysate) or recovery from the culture medium. In one embodiment, host cells comprise non-human cells including CHO, BHK, NS0, SP2/0, and YB2/0. In one embodiment, host cells comprise human cells including HEK293, HT-1080, Huh-7 and PER.C6. Examples of host cells include the COS-7 line of monkey kidney cells (ATCC CRL 1651) (see Gluzman et al., 1981, Cell 23: 175), L cells, C127 cells, 3T3 cells (ATCC CCL 163), Chinese hamster ovary (CHO) cells or their derivatives such as Veggie CHO and related cell lines which grow in serum- free media (see Rasmussen et al., 1998, Cytotechnology 28:31) or CHO strain DX-B 11, which is deficient in DHFR (see Urlaub et al., 1980, Proc. Natl. Acad. Sci. USA 77:4216-20), HeLa cells, BHK (ATCC CRL 10) cell lines, the CV⅟EBNA cell line derived from the African green monkey kidney cell line CV1 (ATCC CCL 70) (see McMahan et al., 1991, EMBO J. 10:2821), human embryonic kidney cells such as 293, 293 EBNA or MSR 293, human epidermal A431 cells, human Colo 205 cells, other transformed primate cell lines, normal diploid cells, cell strains derived from in vitro culture of primary tissue, primary explants, HL-60, U937, HaK or Jurkat cells. In one embodiment, host cells include lymphoid cells such as Y0, NS0 or Sp20. In one embodiment, a host cell is a mammalian host cell, but is not a human host cell. Typically, a host cell is a cultured cell that can be transformed or transfected with a polypeptide-encoding nucleic acid, which can then be expressed in the host cell. The phrase “transgenic host cell” or “recombinant host cell” can be used to denote a host cell that has been transformed or transfected with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but also to the progeny or potential progeny of such a cell. Because certain modifications may occur in succeeding generations due to, e.g., mutation or environmental influence, such progeny may not, in fact, be identical to the parent cell, but are still included within the scope of the term as used herein. A host cell, or a population of host cells, harboring a vector (e.g., an expression vector) operably linked to at least one nucleic acid encoding one or more activatable masked IgG-type antibodies and dimeric antigen receptor (DAR) having activatable masking moieties or antigen-binding portions thereof are described herein.

Polypeptides of the present disclosure (e.g., antibodies and antigen binding proteins) can be produced using any method known in the art. In one example, the polypeptides are produced by recombinant nucleic acid methods by inserting a nucleic acid sequence (e.g., DNA) encoding the polypeptide into a recombinant expression vector which is introduced into a host cell and expressed by the host cell under conditions promoting expression.

General techniques for recombinant nucleic acid manipulations are described for example in Sambrook et al., in Molecular Cloning: A Laboratory Manual, Vols. 1-3, Cold Spring Harbor Laboratory Press, 2 ed., 1989, or F. Ausubel et al., in Current Protocols in Molecular Biology (Green Publishing and Wiley-Interscience: New York, 1987) and periodic updates, herein incorporated by reference in their entireties. The nucleic acid (e.g., DNA) encoding the polypeptide is operably linked to an expression vector carrying one or more suitable transcriptional or translational regulatory elements derived from mammalian, viral, or insect genes. Such regulatory elements include a transcriptional promoter, an optional operator sequence to control transcription, a sequence encoding suitable mRNA ribosomal binding sites, and sequences that control the termination of transcription and translation. The expression vector can include an origin or replication that confers replication capabilities in the host cell. The expression vector can include a gene that confers selection to facilitate recognition of transgenic host cells (e.g., transformants).

The recombinant DNA can also encode any type of protein tag sequence that may be useful for purifying the protein. Examples of protein tags include but are not limited to a histidine tag, a FLAG tag, a myc tag, an HA tag, or a GST tag. Appropriate cloning and expression vectors for use with bacterial, fungal, yeast, and mammalian cellular hosts can be found in Cloning Vectors: A Laboratory Manual, (Elsevier, N.Y., 1985).

The expression vector construct can be introduced into the host cell using a method appropriate for the host cell. A variety of methods for introducing nucleic acids into host cells are known in the art, including, but not limited to, electroporation; transfection employing calcium chloride, rubidium chloride, calcium phosphate, DEAE-dextran, or other substances; viral transfection; non-viral transfection; microprojectile bombardment; lipofection; and infection (e.g., where the vector is an infectious agent). Suitable host cells include prokaryotes, yeast, mammalian cells, or bacterial cells.

Suitable bacteria include gram negative or gram positive organisms, for example, E. coli or Bacillus spp. Yeast, preferably from the Saccharomyces species, such as S. cerevisiae, may also be used for production of polypeptides. Various mammalian or insect cell culture systems can also be employed to express recombinant proteins. Baculovirus systems for production of heterologous proteins in insect cells are reviewed by Luckow and Summers, (Bio/Technology, 6:47, 1988). Examples of suitable mammalian host cell lines include endothelial cells, COS-7 monkey kidney cells, CV-1, L cells, C127, 3T3, Chinese hamster ovary (CHO), human embryonic kidney cells, HeLa, 293, 293T, and BHK cell lines. Purified polypeptides are prepared by culturing suitable host/vector systems to express the recombinant proteins. For many applications, the small size of many of the polypeptides disclosed herein would make expression in E. coli as the preferred method for expression. The protein is then purified from culture media or cell extracts. Any of the activatable masked IgG-type antibodies and dimeric antigen receptor (DAR) having activatable masking moieties or antigen-binding portions thereof, can be expressed by transgenic host cells.

Antibodies and antigen binding proteins disclosed herein can also be produced using cell-translation systems. For such purposes the nucleic acids encoding the polypeptide must be modified to allow in vitro transcription to produce mRNA and to allow cell-free translation of the mRNA in the particular cell-free system being utilized (eukaryotic such as a mammalian or yeast cell-free translation system or prokaryotic such as a bacterial cell-free translation system.

Nucleic acids encoding any of the various polypeptides disclosed herein may be synthesized chemically. Codon usage may be selected so as to improve expression in a cell. Such codon usage will depend on the cell type selected. Specialized codon usage patterns have been developed for E. coli and other bacteria, as well as mammalian cells, plant cells, yeast cells and insect cells. See for example: Mayfield et al., Proc. Natl. Acad. Sci. USA. 2003 100(2):438-42; Sinclair et al. Protein Expr. Purif. 2002 (1):96-105; Connell N D. Curr. Opin. Biotechnol. 2001 12(5):446-9; Makrides et al. Microbiol. Rev. 1996 60(3):512-38; and Sharp et al. Yeast. 1991 7(7):657-78.

Antibodies and antigen binding proteins described herein can also be produced by chemical synthesis (e.g., by the methods described in Solid Phase Peptide Synthesis, 2nd ed., 1984, The Pierce Chemical Co., Rockford, Ill.). Modifications to the protein can also be produced by chemical synthesis.

Antibodies and antigen binding proteins described herein can be purified by isolation/purification methods for proteins generally known in the field of protein chemistry. Non-limiting examples include extraction, recrystallization, salting out (e.g., with ammonium sulfate or sodium sulfate), centrifugation, dialysis, ultrafiltration, adsorption chromatography, ion exchange chromatography, hydrophobic chromatography, normal phase chromatography, reversed-phase chromatography, gel filtration, gel permeation chromatography, affinity chromatography, electrophoresis, countercurrent distribution or any combinations of these. After purification, polypeptides may be exchanged into different buffers and/or concentrated by any of a variety of methods known to the art, including, but not limited to, filtration and dialysis.

The purified antibodies and antigen binding proteins described herein are preferably at least 65% pure, at least 75 % pure, at least 85% pure, more preferably at least 95% pure, and most preferably at least 98% pure. Regardless of the exact numerical value of the purity, the polypeptide is sufficiently pure for use as a pharmaceutical product. Any of the activatable masked IgG-type antibodies and dimeric antigen receptor (DAR) having activatable masking moieties or antigen-binding portions thereof that are described herein can be expressed by transgenic host cells and then purified to about 65-98% purity or high level of purity using any art-known method.

In certain embodiments, the antibodies and antigen binding proteins herein can further comprise post-translational modifications. Exemplary post-translational protein modifications include phosphorylation, acetylation, methylation, ADP-ribosylation, ubiquitination, glycosylation, carbonylation, sumoylation, biotinylation or addition of a polypeptide side chain or of a hydrophobic group. As a result, the modified polypeptides may contain non-amino acid elements, such as lipids, poly- or mono-saccharide, and phosphates. A preferred form of glycosylation is sialylation, which conjugates one or more sialic acid moieties to the polypeptide. Sialic acid moieties improve solubility and serum half-life while also reducing the possible immunogenicity of the protein. See Raju et al. Biochemistry. 2001 31; 40(30):8868-76.

In one embodiment, the antibodies and antigen binding proteins described herein can be modified to become soluble polypeptides which comprises linking the Antibodies and antigen binding proteins to non-proteinaceous polymers. In one embodiment, the non-proteinaceous polymer comprises polyethylene glycol (“PEG”), polypropylene glycol, or polyoxyalkylenes, in the manner as set forth in U.S. Pat. Nos. 4,640,835; 4,496,689; 4,301,144; 4,670,417; 4,791,192 or 4,179,337.

PEG is a water soluble polymer that is commercially available or can be prepared by ring-opening polymerization of ethylene glycol according to methods well known in the art (Sandler and Karo, Polymer Synthesis, Academic Press, New York, Vol. 3, pages 138-161). The term “PEG” is used broadly to encompass any polyethylene glycol molecule, without regard to size or to modification at an end of the PEG, and can be represented by the formula: X—O(CH₂CH₂O)ₙ—CH₂CH₂OH (1), where n is 20 to 2300 and X is H or a terminal modification, e.g., a C₁₋₄ alkyl. In one embodiment, the PEG terminates on one end with hydroxy or methoxy, i.e., X is H or CH₃ (“methoxy PEG”). A PEG can contain further chemical groups which are necessary for binding reactions; which results from the chemical synthesis of the molecule; or which is a spacer for optimal distance of parts of the molecule. In addition, such a PEG can consist of one or more PEG side-chains which are linked together. PEGs with more than one PEG chain are called multiarmed or branched PEGs. Branched PEGs can be prepared, for example, by the addition of polyethylene oxide to various polyols, including glycerol, pentaerythriol, and sorbitol. For example, a four-armed branched PEG can be prepared from pentaerythriol and ethylene oxide. Branched PEG are described in, for example, EP-A 0 473 084 and U.S. Pat. No. 5,932,462. One form of PEGs includes two PEG side-chains (PEG2) linked via the primary amino groups of a lysine (Monfardini et al., Bioconjugate Chem. 6 (1995) 62-69).

The serum clearance rate of PEG-modified polypeptide may be modulated (e.g., increased or decreased) by about 10%, 20%, 30%, 40%, 50%, 60%, 70%, 80%, or even 90%, relative to the clearance rate of the unmodified antibodies and antigen binding proteins binding polypeptides. The PEG-modified antibodies and antigen binding proteins may have a half-life (t_{½}) which is enhanced relative to the half-life of the unmodified polypeptide. The half-life of PEG-modified polypeptide may be enhanced by at least 10%, 20%, 30%, 40%, 50%, 60%, 70%, 80%, 90%, 100%, 125%, 150%, 175%, 200%, 250%, 300%, 400% or 500%, or even by 1000% relative to the half-life of the unmodified antibodies and antigen binding proteins. In one embodiment, the protein half-life is determined in vitro, such as in a buffered saline solution or in serum. In other embodiments, the protein half-life is an in vivo half-life, such as the half-life of the protein in the serum or other bodily fluid of an animal.

The present disclosure provides therapeutic compositions comprising any of the activatable masked IgG-type antibodies and dimeric antigen receptor (DAR) having activatable masking moieties or antigen-binding portions thereof that are described herein in an admixture with a pharmaceutically-acceptable excipient. An excipient encompasses carriers, stabilizers and excipients. Excipients of pharmaceutically acceptable excipients includes for example inert diluents or fillers (e.g., sucrose and sorbitol), lubricating agents, glidants, and anti-adhesives (e.g., magnesium stearate, zinc stearate, stearic acid, silicas, hydrogenated vegetable oils, or talc). Additional examples include buffering agents, stabilizing agents, preservatives, non-ionic detergents, anti-oxidants and isotonifiers.

Therapeutic compositions and methods for preparing them are well known in the art and are found, for example, in “Remington: The Science and Practice of Pharmacy” (20th ed., ed. A. R. Gennaro A R., 2000, Lippincott Williams & Wilkins, Philadelphia, Pa.). Therapeutic compositions can be formulated for parenteral administration may, and can for example, contain excipients, sterile water, saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the antibody (or antigen binding protein thereof) described herein. Nanoparticulate formulations (e.g., biodegradable nanoparticles, solid lipid nanoparticles, liposomes) may be used to control the biodistribution of the antibody (or antigen binding protein thereof). Other potentially useful parenteral delivery systems include ethylenevinyl acetate copolymer particles, osmotic pumps, implantable infusion systems, and liposomes. The concentration of the antibody (or antigen binding protein thereof) in the formulation varies depending upon a number of factors, including the dosage of the drug to be administered, and the route of administration.

Any of the activatable masked IgG-type antibodies and dimeric antigen receptor (DAR) having activatable masking moieties or antigen-binding portions thereof described herein may be optionally administered as a pharmaceutically acceptable salt, such as non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, and the like. In one example, the antibody (or antigen binding protein thereof) is formulated in the presence of sodium acetate to increase thermal stability.

Any of the activatable masked IgG-type antibodies and dimeric antigen receptor (DAR) having activatable masking moieties or antigen-binding portions thereof described herein may be formulated for oral use include tablets containing the active ingredient(s) in a mixture with non-toxic pharmaceutically acceptable excipients. Formulations for oral use may also be provided as chewable tablets, or as hard gelatin capsules wherein the active ingredient is mixed with an inert solid diluent, or as soft gelatin capsules wherein the active ingredient is mixed with water or an oil medium.

The term “subject” as used herein refers to human and non-human animals, including vertebrates, mammals and non-mammals. In one embodiment, the subject can be human, non-human primates, simian, ape, murine (e.g., mice and rats), bovine, porcine, equine, canine, feline, caprine, lupine, ranine or piscine.

The term “administering”, “administered” and grammatical variants refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In one embodiment, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods. Any of the activatable masked IgG-type antibodies and dimeric antigen receptor (DAR) having activatable masking moieties or antigen-binding portions thereof described herein can be administered to a subject using art-known methods and delivery routes.

The terms “effective amount”, “therapeutically effective amount” or “effective dose” or related terms may be used interchangeably and refer to an amount of an activatable masked IgG-type antibodies and dimeric antigen receptor (DAR) having activatable masking moieties or antigen-binding portions thereof that when administered to a subject, is sufficient to effect a measurable improvement or prevention of a disease or disorder associated with tumor or cancer antigen expression. Therapeutically effective amounts of antibodies provided herein, when used alone or in combination, will vary depending upon the relative activity of the antibodies and combinations (e.g. , in inhibiting cell growth) and depending upon the subject and disease condition being treated, the weight and age and sex of the subject, the severity of the disease condition in the subject, the manner of administration and the like, which can readily be determined by one of ordinary skill in the art.

In one embodiment, a therapeutically effective amount will depend on certain aspects of the subject to be treated and the disorder to be treated and may be ascertained by one skilled in the art using known techniques. In general, the polypeptide is administered at about 0.01 g/kg to about 50 mg/kg per day, preferably 0.01 mg/kg to about 30 mg/kg per day, most preferably 0.1 mg/kg to about 20 mg/kg per day. The polypeptide may be administered daily (e.g., once, twice, three times, or four times daily) or preferably less frequently (e.g., weekly, every two weeks, every three weeks, monthly, or quarterly). In addition, as is known in the art, adjustments for age as well as the body weight, general health, sex, diet, time of administration, drug interaction, and the severity of the disease may be necessary.

The present disclosure provides methods for treating a subject having a disease associated with expression of one or more tumor-associated antigens. The disease comprises cancer or tumor cells expressing the tumor-associated antigens, such as for example EGFR or CD38 antigen. In one embodiment, the cancer or tumor includes cancer of the prostate, breast, ovary, head and neck, bladder, skin, colorectal, anus, rectum, pancreas, lung (including non-small cell lung and small cell lung cancers), leiomyoma, brain, glioma, glioblastoma, esophagus, liver, kidney, stomach, colon, cervix, uterus, endometrium, vulva, larynx, vagina, bone, nasal cavity, paranasal sinus, nasopharynx, oral cavity, oropharynx, larynx, hypolarynx, salivary glands, ureter, urethra, penis and testis.

In one embodiment, the cancer comprises hematological cancers, including leukemias, lymphomas, myelomas and B cell lymphomas. Hematologic cancers include multiple myeloma (MM), non-Hodgkin’s lymphoma (NHL) including Burkitt’s lymphoma (BL), B chronic lymphocytic leukemia (B-CLL), systemic lupus erythematosus (SLE), B and T acute lymphocytic leukemia (ALL), acute myeloid leukemia (AML), chronic lymphocytic leukemia (CLL), diffuse large B cell lymphoma, chronic myelogenous leukemia (CML), hairy cell leukemia (HCL), follicular lymphoma, Waldenstrom’s Macroglobulinemia, mantle cell lymphoma, Hodgkin’s Lymphoma (HL), plasma cell myeloma, precursor B cell lymphoblastic leukemia/lymphoma, plasmacytoma, giant cell myeloma, plasma cell myeloma, heavy-chain myeloma, light chain or Bence-Jones myeloma, lymphomatoid granulomatosis, post-transplant lymphoproliferative disorder, an immunoregulatory disorder, rheumatoid arthritis, myasthenia gravis, idiopathic thrombocytopenia purpura, anti-phospholipid syndrome, Chagas’ disease, Grave’s disease, Wegener’s granulomatosis, poly-arteritis nodosa, Sjogren’s syndrome, pemphigus vulgaris, scleroderma, multiple sclerosis, anti-phospholipid syndrome, ANCA associated vasculitis, Goodpasture’s disease, Kawasaki disease, autoimmune hemolytic anemia, and rapidly progressive glomerulonephritis, heavy-chain disease, primary or immunocyte-associated amyloidosis, and monoclonal gammopathy of undetermined significance.

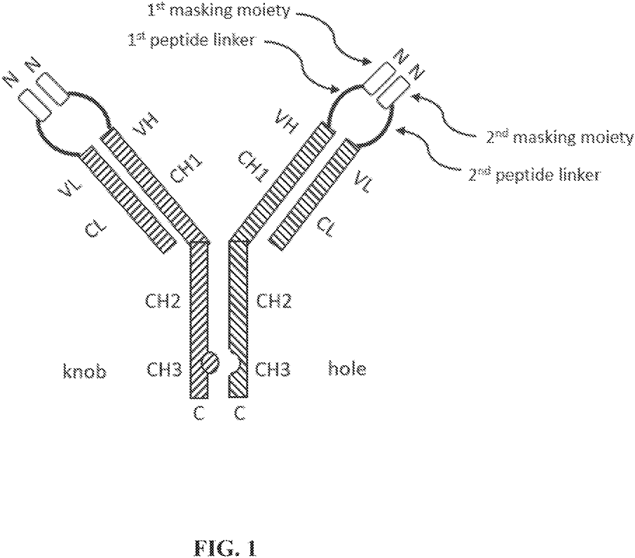

The term “in-tandem.” as used herein with respect to regions in a polypeptide chain or a protein complex, such as Fab regions, means that the regions are arranged head-to-tail without an intervening different region (e.g., Fc region) between the Fab regions. The in-tandem Fab regions may be separated by a linker. The exemplary structure illustrated in FIG. 1 comprises in-tandem Fab regions.

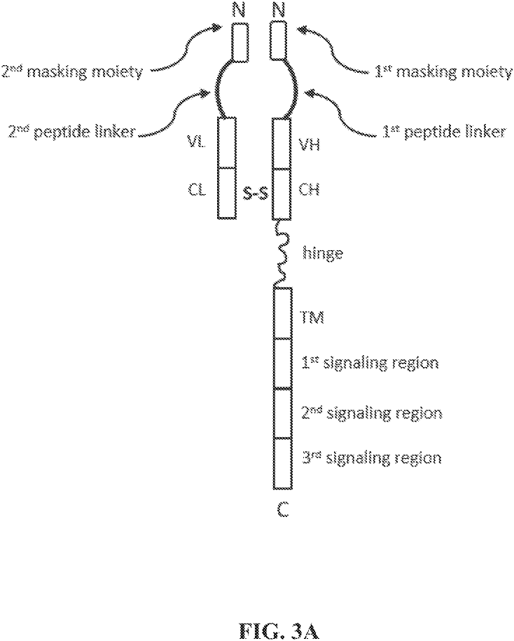

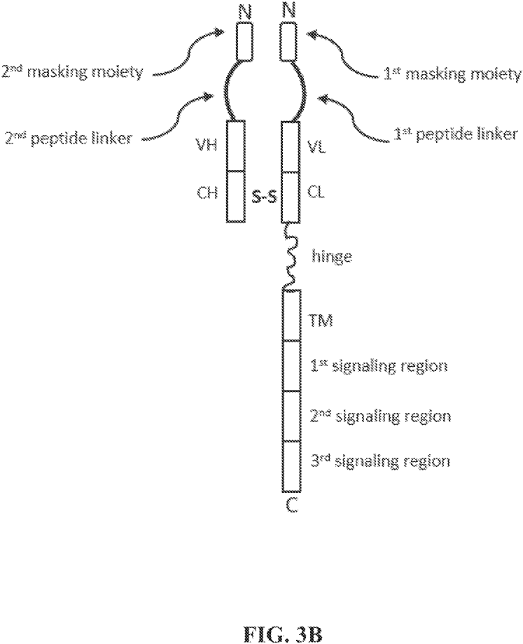

The term “non-tandem” as used herein with respect to regions in a polypeptide chain or a protein complex, such as Fab regions, means that the regions are not in an “in-tandem” arrangement. The exemplary structure illustrated in FIG. 3 comprises non-tandem Fab regions

### Activatable Masked Antigen Binding Proteins

The present disclosure provides activatable masked antigen binding proteins comprising at least a first antigen binding domain which comprises a first heavy chain variable region and a first light chain variable region, wherein (i) the first heavy chain variable region is j oined to a first masking moiety via a first peptide linker, and (ii) the first light chain variable region is joined to a second masking moiety via a second peptide linker, and (iii) the first and second masking moieties associate with each other to form a first dimerized masking complex without covalent bonding, and (iv) the individual first and second masking moieties do not specifically bind the first antigen binding domain, and (v) the first dimerized masking complex does not specifically bind the first antigen binding domain. In one embodiment, the first and second masking moieties associate with each other to reduce the first antigen binding domain from binding to its target antigen. In one embodiment, the first peptide linker includes a first cleavable site. In one embodiment, the second peptide linker includes a second cleavable site.

In one embodiment, the activatable masked antigen binding proteins further comprises a second antigen binding domain which comprises a second heavy chain variable region and a second light chain variable region, wherein (i) the second heavy chain variable region is joined to a third masking moiety via a third peptide linker, and (ii) the second light chain variable region is joined to a fourth masking moiety via a fourth peptide linker, and (iii) the third and fourth masking moieties associate with each other to form a second dimerized masking complex without covalent bonding, and (iv) the individual third and fourth masking moieties do not specifically bind the second antigen binding domain, and (v) the second dimerized masking complex does not specifically bind the second antigen binding domain. In one embodiment, the third and fourth masking moieties associate with each other to reduce the second antigen binding domain from binding to its target antigen which may be the same or different target antigen compared to the target antigen bound by the first antigen binding domain. In one embodiment, the third peptide linker includes a third cleavable site. In one embodiment, the fourth peptide linker includes a fourth cleavable site.

In one embodiment, the first, second, third and/or fourth cleavable sites are cleavable with a cleaving condition which includes a protease, esterase, reductive condition, or oxidative condition. In one embodiment, the first, second, third and/or fourth cleavable sites are cleavable with the same or different cleaving condition. In one embodiment, the first, second third and/or fourth cleavable sites are cleavable with a protease that is present in a tumor microenvironment or are cleavable with a reductive or oxidative condition that is present in a tumor microenvironment. In one embodiment, the first, second, third and/or fourth cleavable sites are cleavable with the same or different proteases.

In one embodiment, the activatable masked antigen binding protein is joined to a toxin by a chemical linker thereby forming an immunoconjugate. In one embodiment, the toxin is joined to the chemical linker which in turn is j oined to a lysine residue of the activatable masked antigen binding protein by forming an amide bond with lysine side chain. In one embodiment, the lysine residue on the activatable masked antigen binding protein is selectively joined to the linker according to methods and chemical reactions described in U.S. Pat. No. 9,981,046. In one embodiment, the toxin is cytotoxic to cells and tissue. In one embodiment, the immunoconjugate can be used to deliver the toxin to a target cell or tissue (e.g., target tumor).

The present disclosure provides activatable masked antigen binding proteins comprising at least a first antigen binding domain which comprises a first heavy chain variable region and a first light chain variable region, wherein (i) the N-terminal end of the first heavy chain variable region is joined to a first masking moiety via a first peptide linker having a first cleavable site, and (ii) the N-terminal end of the first light chain variable region is joined to a second masking moiety via a second peptide linker having a second cleavable site, and (iii) the first and second masking moieties associate with each other to form a first dimerized masking complex without disulfide bonding, and (iv) the first and second masking moieties do not specifically bind the at least first antigen binding domain, and (v) the first dimerized masking complex does not specifically bind the at least first antigen binding domain, and (vi) the first cleavable site is cleavable with a first protease (e.g., the first protease comprises a tumor-associated protease), and (vii) the second cleavable site is cleavable with a second protease (e.g., the second protease comprises a tumor-associated protease), wherein the first and second cleavable sites are cleavable with the same or different proteases. In one embodiment, the first and second masking moieties associate with each other to reduce the capability of the first antigen binding domain from binding to its target antigen. In one embodiment, a first recombinant polypeptide comprises the first heavy chain variable region joined to the first peptide linker which is joined to the first masking moiety. In one embodiment, a second recombinant polypeptide comprises the first light chain variable region joined to the second peptide linker which is joined to the second masking moiety.

In one embodiment, the activatable masked antigen binding proteins further comprise a second antigen binding domain which comprises a second heavy chain variable region and a second light chain variable region, wherein (i) the N-terminal end of the second heavy chain variable region is joined to a third masking moiety via a third peptide linker having a third cleavable site, and (ii) the N-terminal end of the second light chain variable region is joined to a fourth masking moiety via a fourth peptide linker having a fourth cleavable site, and (iii) the third and fourth masking moieties associate with each other to form a second dimerized masking complex without disulfide bonding, and (iv) the third and fourth masking moieties do not specifically bind the second antigen binding domain, and (v) the second dimerized masking complex does not specifically bind the second antigen binding domain, and (vi) the third cleavable site is cleavable with a third protease (e.g., the third protease comprises a tumor-associated protease), and (vii) the fourth cleavable site is cleavable with a fourth protease (e.g., the fourth protease comprises a tumor-associated protease), wherein the third and fourth cleavable sites are cleavable with the same or different proteases. In one embodiment, the third and fourth masking moieties associates with each other to reduce the capability of the second antigen binding domain from binding to its target antigen. In one embodiment, a third recombinant polypeptide comprises the second heavy chain variable region joined to the third peptide linker which is joined to the third masking moiety. In one embodiment, a fourth recombinant polypeptide comprises the second light chain variable region joined to the fourth peptide linker which is joined to the fourth masking moiety.

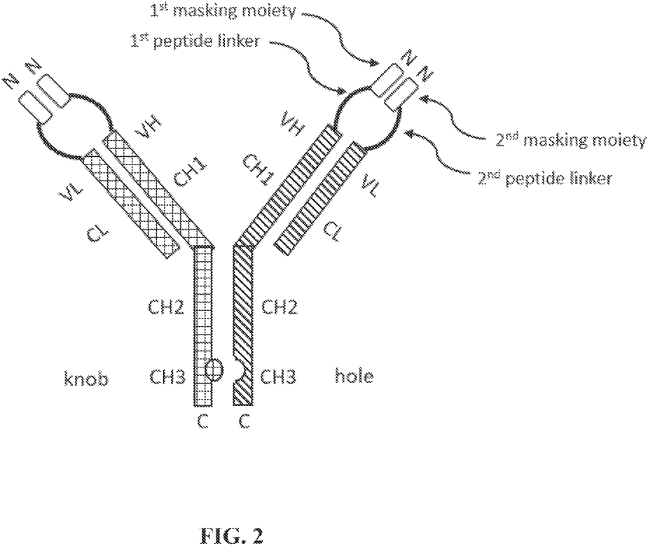

In one embodiment, the activatable masked antigen binding protein comprises an IgG class antibody (e.g., FIG. 1) that binds a target antigen (e.g., monospecific). In one embodiment, the activatable masked antigen binding proteins comprises an IgG class antibody (e.g., FIG. 2) that binds two target antigens (e.g., bispecific). In one embodiment, the bispecific antibody has an IgG-like structure (e.g., DVD-Ig from U.S. 2011/0263827). In one embodiment, the activatable masked antigen binding protein comprises a dimeric antigen receptor (DAR) (e.g., FIG. 3) (e.g., see PCT/US2019/21681, entitled “Dimeric Antigen Receptors (DAR)” and filed on Mar. 11, 2019.

In one embodiment, the activatable masked antigen binding protein is joined to a toxin by a chemical linker thereby forming an immunoconjugate. In one embodiment, the toxin is joined to the chemical linker which in turn is joined to a lysine residue of the activatable masked antigen binding protein by forming an amide bond with lysine side chain. In one embodiment, the lysine residue on the activatable masked antigen binding protein is selectively joined to the linker according to methods and chemical reactions described in U.S. Pat. No. 9,981,046. In one embodiment, the toxin is cytotoxic to cells and tissue. In one embodiment, the immunoconjugate can be used to deliver the toxin to a target cell or tissue (e.g., target tumor).

### Activatable Masked Antigen Binding Proteins Comprising IgG-Like Molecules

The present disclosure provides activatable masked antigen binding proteins comprising an IgG type antibody having (a) a first antigen binding domain which comprises a first heavy chain variable region and a first light chain variable region and (b) a second antigen binding domain which comprises a second heavy chain variable region and a second light chain variable region, wherein (i) the N-terminal end of the first heavy chain variable region is joined to a first masking moiety via a first peptide linker having a first cleavable site, (ii) the N-terminal end of the first light chain variable region is joined to a second masking moiety via a second peptide linker having a second cleavable site, (iii) the first and second masking moieties associate with each other without disulfide bonding, (iv) the first and second masking moieties do not specifically bind the first antigen binding domain, (v) the first cleavable site is cleavable with a first protease, and (vi) the second cleavable site is cleavable with a second protease, and wherein in the activatable masked antigen binding proteins further comprise an IgG type antibody having (vii) the N-terminal end of the second heavy chain variable region is joined to a third masking moiety via a third peptide linker having a third cleavable site, (viii) the N-terminal end of the second light chain variable region is joined to a fourth masking moiety via a fourth peptide linker having a fourth cleavable site, (ix) the third and fourth masking moieties associate with each other without disulfide bonding, (x) the third and fourth masking moieties do not specifically bind the second antigen binding domain, (xi) the third cleavable site is cleavable with a third protease, and (xii) the fourth cleavable site is cleavable with a fourth protease, wherein the first, second, third and fourth cleavable sites are cleavable with the same or different proteases.

In one embodiment, the activatable masked antigen binding protein comprises an IgG class antibody (e.g., FIG. 1) that binds a target antigen (e.g., monospecific). In one embodiment, the activatable masked antigen binding proteins comprises an IgG class antibody (e.g., FIG. 2) that binds two target antigens (e.g., bispecific).

In one embodiment, the C-terminal end of the first and third masking moiety is joined to the N-terminal end of the first and third peptide linkers, respectively. In one embodiment, the C-terminal end of the first and third peptide linkers are joined to the N-terminal end of the first and second heavy chain variable region, respectively.

In one embodiment, the C-terminal end of the second and fourth masking moiety is joined to the N-terminal end of the second and fourth peptide linkers, respectively. In one embodiment, the C-terminal end of the second and fourth peptide linkers are joined to the N-terminal end of the first and second light chain variable region, respectively.

In one embodiment, the activatable masked antigen binding proteins comprising an IgG type antibody can bind a target antigen (e.g., monospecific antibody) or can bind two different target antigens (e.g., bispecific antibody).

In one embodiment, the first and second masking moieties associates with each other to reduce the first antigen binding domain from binding to its target antigen. In one embodiment, the third and fourth masking moieties associates with each other to reduce the second antigen binding domain from binding to its target antigen.

In one embodiment, the first, second, third and/or fourth protease(s) is/are produced by the same tumor or by different tumors.

In one embodiment, the activatable masked antigen binding protein is joined to a toxin by a chemical linker.

The present disclosure provides activatable masked antigen binding proteins comprising an IgG type antibody and at least a first and second masking moiety. In one embodiment, the first and second masking moieties, and/or the third and fourth masking moieties, when associated (dimerized) with each other, interfere (obstruct) with the ability of the antigen binding protein to bind its target antigen. The first and second masking moieties, and/or the third and fourth masking moieties, when associated (dimerized) with each other, can block binding between the antigen binding protein and its target antigen by steric hinderance.

In one embodiment, the first and second masking moieties, and/or the third and fourth masking moieties, comprise polypeptides that associate (dimerize) with each other without forming a covalent bond (e.g., no disulfide or transglutamination bonding). The first and second masking moieties, and/or the third and fourth masking moieties, comprise polypeptides that can associate (dimerize) with each other by non-covalent interaction, including ionic interaction, hydrogen bonding, dipole-dipole interaction, hydrophilic interaction, hydrophobic interaction, affinity bonding, or bonds or associations involving van der Waals forces.

In one embodiment, the first and second masking moieties (paired first and second masking moieties), or the third and fourth masking moieties (paired third and fourth masking moieties), associate with each other as a homodimer or heterodimer.

In one embodiment, one of the masking moieties can be engineered to have a knob structure that forms an interchain steric complementarity structure to favor homo-dimerization or hetero-dimerization with another masking moiety which is engineered to have a hole structure.

The present disclosure provides activatable masked antigen binding proteins, wherein one or both of the masking moieties in a dimerized configuration includes one or more mutations that create a new interchain salt bridge. In one embodiment, a threonine residue in one masking moiety can be replaced with glutamic acid, and an asparagine at a corresponding position in another masking moiety is replaced with lysine, thereby creating an interchain salt bridge.

In one embodiment, the first, second, third and/or fourth masking moieties are derived from an immunoglobulin heavy chain constant region, for example from a mu, delta, gamma, alpha, or epsilon heavy chain. In one embodiment, the first, second, third and/or fourth masking moieties are derived from a gamma immunoglobulin constant region (e.g., CL, CH1, CH2 or CH3).

In one embodiment, die first or second, or third or fourth, masking moiety comprises a heavy chain constant region (CH1). In one embodiment, the second or first, or fourth or third, masking moiety comprises a kappa or lambda light chain constant region (CL kappa or CL lambda). In one embodiment, the first masking moiety comprises a heavy chain constant region (CH1) and the second masking moiety comprises a kappa or lambda light chain constant region (CL kappa or CL lambda). In one embodiment, the first masking moiety comprises a kappa or lambda light chain constant region (CL kappa or CL lambda) and the second masking moiety comprises a heavy chain constant region (CH1), In one embodiment, the third masking moiety comprises a heavy chain constant region (CH1) and the fourth masking moiety comprises a kappa or lambda light chain constant region (CL kappa or CL lambda). In one embodiment, the third masking moiety comprises a kappa or lambda light chain constant region (CL kappa or CL lambda) and the fourth masking moiety comprises a heavy chain constant region (CH1). In one embodiment, the first, second, third and/or fourth masking moiety comprises a CH1, CL (kappa) or CL (lambda) having the amino acid sequence of SEQ ID NO:21, 22 or 23. See also Tables 1 and 2 for a list of amino acid sequences of various masking moieties.

In one embodiment, the first or second, or third or fourth, masking moiety comprises an IgG1 or IgG4 gamma heavy chain constant region, for example CH3. In one embodiment, the first and second masking moieties comprise an IgG1 or IgG4 gamma heavy chain constant region CH3. In one embodiment, the third and fourth masking moieties comprise an IgG1 or IgG4 gamma heavy chain constant region CH3. In one embodiment, one of the masking moieties which comprises an IgG1 or IgG4 gamma heavy chain constant region CH3 is engineered to have a knob or hole structure that forms an interchain steric complementarity structure to favor dimerization (e.g., homo-dimerization) with another masking moiety which comprises an IgG1 or IgG4 gamma heavy chain constant region CH3 which is engineered to have hole or knob structure. In one embodiment, the first, second, third and/or fourth masking moiety comprises a CH3 (IgG1 or IgG4) having the amino acid sequence of SEQ ID NO:24, 25, 30 or 31. See also Tables 1 and 2 for a list of amino acid sequences of various masking moieties.

In one embodiment, at least one of the masking moieties is derived from an immunoglobulin heavy chain constant region and is joined to the heavy chain variable region of the activatable masked antibody. In one embodiment, at least one of the masking moieties is derived from an immunoglobulin heavy chain constant region and is joined to the light chain variable region of the activatable masked antibody.

In one embodiment, the first and second, or third and fourth, masking moieties are derived from T cell receptors alpha (α) and beta (β) constant regions In one embodiment, the first masking moiety comprises a T cell receptor alpha (α) constant regions and the second masking moiety comprises a T cell receptor beta (β) constant region. In one embodiment, the first masking moiety comprises a T cell receptor beta (β) constant regions and the second masking moiety comprises a T cell receptor alpha (α) constant region. In one embodiment, the third masking moiety comprises a T cell receptor alpha (α) constant regions and the fourth masking moiety comprises a T cell receptor beta (β) constant region. In one embodiment, the third masking moiety comprises a T cell receptor beta (β) constant regions and the fourth masking moiety comprises a T cell receptor alpha (α) constant region.

In one embodiment, at least one of the masking moieties is derived from a T cell receptor alpha (α) or beta (β) constant region and is joined to the heavy chain variable region of the activatable masked antibody. In one embodiment, at least one of the masking moieties is derived from a T cell receptor beta (β) or alpha (α) constant region and is joined to the light chain variable region of the activatable masked antibody. In one embodiment, the first, second, third and/or fourth masking moiety comprises a T cell receptor alpha (α) or beta (β) constant region having the amino acid sequence of SEQ ID NO:28 or 29. See Tables 1 and 2 for a list of amino acid sequences of various masking moieties.

In one embodiment, the first, second, third or fourth masking moiety comprises a variable heavy chain domain from a catalytic antibody (e.g., 38C2). In one embodiment, the first, second, third or fourth masking moiety comprises a variable light chain domain from a catalytic antibody (e.g., 38C2). In one embodiment, the first masking moiety comprises a variable heavy chain domain from 38C2 antibody and the second masking moiety comprise a variable light chain domain from 38C2 antibody. In one embodiment, the first masking moiety comprises a variable light chain domain from 38C2 antibody and the second masking moiety comprise a variable heavy chain domain from 38C2 antibody. In one embodiment, the third masking moiety comprises a variable heavy chain domain from 38C2 antibody and the fourth masking moiety comprise a variable light chain domain from 38C2 antibody. In one embodiment, the third masking moiety comprises a variable light chain domain from 38C2 antibody and the fourth masking moiety comprise a variable heavy chain domain from 38C2 antibody. In one embodiment, the first, second, third and/or fourth masking moiety comprises a variable heavy chain domain from a catalytic antibody (e.g., 38C2) having the amino acid sequence of SEQ ID NO:26 or 27. See Tables 1 and 2 for a list of amino acid sequences of various masking moieties.

In one embodiment, a masking moiety (e.g., first, second, third or fourth masking moiety) comprises two or more copies of a moiety arranged in-tandem along the same polypeptide chain. For example, a masking moiety comprises two antibody CH3 regions arranged in-tandem, or two antibody CH2 regions arranged in-tandem, or two antibody CH1 regions arranged in-tandem, or two antibody CL regions arranged in-tandem. Any of the CH3, CH2, CH1 or CL regions can include a knob or hole structure. For example, the masking moiety comprises two TCR alpha chain constant region arranged in-tandem, or two TCR beta chain constant region arranged in-tandem.

In one embodiment, a masking moiety (e.g., first, second, third or fourth masking moiety) comprises two or more different moieties arranged in-tandem along the same polypeptide chain. For example, the masking moiety comprises an antibody CH2 region and an antibody CH3 region arranged in-tandem.

The present disclosure provides activatable masked antigen binding proteins comprising an IgG type antibody and at least first and second peptide linkers have the same or different lengths (e.g., 5-10, 10-20, 20-30, 30-40 or 40-50 amino acids in length). In one embodiment, the first and/or second peptide linkers are glycine-rich (e.g., GS type linkers). In one embodiment, the first and the second peptide linkers have the same amino acid sequence or different amino acid sequences.

In one embodiment, the first peptide linker is cleavable with a first protease. In one embodiment, the first peptide linker has an amino acid sequence (first cleavable site) that is a substrate for cleavage by the first protease.

In one embodiment, the second peptide linker is cleavable with a second protease. In one embodiment, the second peptide linker has an amino acid sequence (second cleavable site) that is a substrate for cleavage by the second protease.

In one embodiment, the first peptide linker is cleavable with a first protease and the second peptide linker is cleavable with a second protease. In one embodiment, the first and second peptide linkers are cleavable with the same protease. In one embodiment, the first and second peptide linkers are cleavable with different proteases. In one embodiment, the first or the second peptide linker is not cleavable with a protease.

The present disclosure provides activatable masked antigen binding proteins comprising an IgG type antibody and further comprising third and fourth peptide linkers have the same or different lengths (e.g., 10-20, 20-30, 30-40 or 40-50 amino acids in length). In one embodiment, the third and/or fourth peptide linkers are glycine-rich (e.g., GS type linkers). In one embodiment, the third and the fourth peptide linkers have the same amino acid sequence or different amino acid sequences.

In one embodiment, the third peptide linker is cleavable with a third protease. In one embodiment, the third peptide linker has an amino acid sequence that is a substrate for cleavage by the third protease.

In one embodiment, the fourth peptide linker is cleavable with a fourth protease. In one embodiment, the fourth peptide linker has an amino acid sequence that is a substrate for cleavage by the fourth protease.

In one embodiment, the third peptide linker is cleavable with a third protease and the fourth peptide linker is cleavable with a fourth protease. In one embodiment, the third and fourth peptide linkers are cleavable with the same protease. In one embodiment, the third and fourth peptide linkers are cleavable with different proteases. In one embodiment, the third or the fourth peptide linker is not cleavable with a protease.

In one embodiment, the first, second, third and/or fourth proteases can be independently selected from: a matrix metalloprotease (MMP) including MMP1, MMP2, MMP3, MMP8, MMP9, MMP11, MMP13, MMP14, MT1-MMP (membrane type 1 matrix metalloproteinase); a disintegrin and metalloproteinase (ADAM) protease including ADAM10, ADAM12, ADAM17; a urokinase plasminogen activator (uPA) or uPAR; a serine protease; a cysteine protease; an aspartate protease; a threonine protease; a cathepsin (e.g., cysteine or aspartic cathepsins) including cathepsin B, cathepsin C, cathepsin D, cathepsin E, cathepsin K or cathepsin L.

In one embodiment, the first, second, third and/or fourth peptide linker comprises the amino acid sequence that is at least 90%, 91%, 92%, 93%, 94%, 95%, 96%, 97%, 98%, 99% or 100% identical the amino acid sequences of any one of SEQ ID NOS:32-40. See also the various peptide linker sequences listed in Tables 3-9.

The present disclosure provides activatable masked antigen binding proteins comprising an IgG type antibody having at least a first antigen binding domain which comprises a first heavy chain variable region and a first light chain variable region, wherein the first antigen binding domain binds a first target antigen (e.g., specifically binds a first target antigen). In one embodiment, the activatable masked antigen binding protein can further comprise a second antigen binding domain which comprises a second heavy chain variable region and a second light chain variable region, wherein the second antigen binding domain binds a second target antigen (e.g., specifically binds a second target antigen). In one embodiment, the first and second antigen binding domains bind the same target antigen. In another embodiment, the first and second antigen binding domains bind a different target antigen.

In one embodiment, the activatable masked antigen binding protein binds EGFR. antigen (SEQ ID NO:1) or CD38 antigen (SEQ ID NO:6).

In one embodiment, the antigen binding domain comprises a heavy chain variable region comprising an amino acid sequence having at least 92%, or at least 93%, or at least 94%, or at least 95%, or at least 96%, or at least 97%, or at least 98%, or at least 99% sequence identity to a portion or full-length of SEQ ID NO:2 or 4 (e.g., anti-EGFR) or to a portion or full-length of SEQ ID NO:7, 9, 11, 13, 14, 15, 16, 17, 18 or 20 (e.g., anti-CD38), or having 100% sequence identity to a portion or full-length of SEQ ID NO:2 or 4 (e.g., anti-EGFR) or a portion or full-length of SEQ ID NO: 7, 9, 11, 13, 14, 15, 16, 17, 18 or 20 (e.g., anti-CD38).

In one embodiment, the antigen binding domain comprises a light chain variable region comprising an amino acid sequence having at least 92%, or at least 93%), or at least 94%, or at least 95%, or at least 96%, or at least 97%, or at least 98%, or at least 99% sequence identity to a portion or full-length of SEQ ID NO:3 or 5 (anti-EGFR) or a portion or full-length of SEQ ID NO:8, 10, 12 or 19 (e.g., anti-CD38), or having 100% sequence identity to a portion or full-length of SEQ ID NO:3 or 5 (e.g., anti-EGFR) or a portion or full-length of SEQ ID NO:8, 10, 12 or 19 (e.g., anti-CD38).

The present disclosure provides activatable masked antigen binding proteins that exhibit inactive and activated states which depend upon the ability of the masking moieties to obstruct binding between the antigen binding domain and its target antigen. In one embodiment, individual masking moieties are tethered to the N-terminus of the heavy chain and light chain variable regions by intact peptide linkers, and the masking moieties form a dimerized complex which obstructs the antigen binding domain resulting in an inactive state. Cleavage of one or both peptide linkers allows the dimerized complex to move away from the obstructing position to permit binding between the antigen binding domain and its target antigen. In one embodiment, cleavage of both peptide linkers liberates the dimerized complex to migrate away from the antigen binding protein.

In one embodiment, the activatable masked antigen binding proteins comprising an IgG type antibody is inhibited from binding to its target antigen (e.g., inactive) when the first and second peptide linkers and/or the third and fourth peptide linkers are in the un-cleaved state. In one embodiment, the activatable masked antigen binding proteins exhibit reduced binding capability to its target antigen (e.g., inactive) when the first and second peptide linkers and/or the third and fourth peptide linkers are in the un-cleaved state. In one embodiment, in an inactive state masked antigen binding protein, the first and second masking moieties and/or the third and fourth masking moieties may or may not be associated with each other.

In one embodiment, a masked antigen binding protein having intact first and second peptide linkers, and/or intact third and fourth peptide linkers, the binding capacity to the target antigen can be reduced by at least 25-50%, or at least 50-75%, or at least 75-95%, or at least 95-99% or more.

In one embodiment, a masked antigen binding protein having intact first and second peptide linkers, and/or intact third and fourth peptide linkers, the binding capacity to the target antigen can be reduced by about 2-20 fold, or about 20-100 fold, or about 100-200 fold, or about 200-500 fold, or about 500-1000 fold, or about 1,000-10,000 fold, or about 10,000-100,000 fold, or higher fold-levels of reduced binding capacity.

In one embodiment, a masked antigen binding protein having intact first and second peptide linkers, and/or intact third and fourth peptide linkers, the binding affinity (e.g., K_{D}) of an inactive antigen binding protein is about 10⁻⁴ - 10⁻⁸ M. In one embodiment, the target antigen is a soluble antigen or a cell surface antigen.

The present disclosure provides activatable masked antigen binding proteins that are un-masked and capability of binding its target antigen (e.g., activated) when the first and second peptide linkers and/or the third and fourth peptide linkers are in the cleaved state. In one embodiment, the activatable masked antigen binding proteins exhibit increased binding capability to its target antigen (e.g., activated) when the first and second peptide linkers and/or the third and fourth peptide linkers are in the cleaved state. In one embodiment, in an activated state masked antigen binding protein, the first and second masking moieties and/or the third and fourth masking moieties may or may not be associated with each other.

In one embodiment, an un-masked antigen binding protein having cleaved first and/or second peptide linkers, and/or having cleaved third and/or fourth peptide linkers, the binding capacity to the target antigen can be increased by at least 25-50%, or at least 50-75%, or at least 75-95%, or at least 95-99% or more.

In one embodiment, an un-masked antigen binding protein having cleaved first and/or second peptide linkers, and/or having cleaved third and/or fourth peptide linkers, the binding capacity to the target antigen can be increased by about 2-20 fold, or about 20-100 fold, or about 100-200 fold, or about 200-500 fold, or about 500-1000 fold, or about 1,000-10,000 fold, or about 10,000-100,000 fold, or higher fold-levels of increased binding capacity.

In one embodiment, an un-masked antigen binding protein having cleaved first and/or second peptide linkers, and/or having cleaved third and/or fourth peptide linkers, the binding affinity (e.g., K_{D}) of an activated antigen binding protein is about 10⁻⁵ - 10⁻¹² M. In one embodiment, the target antigen is a soluble antigen or a cell surface antigen. In one embodiment, the binding affinity of the activated form of the antigen binding protein is higher compared to the inactive form.

In one embodiment, the difference in binding affinity between the inactive and activated antigen binding protein (e.g., K_{D} of inactive vs. K_{D} of activated) is about 10, 10², 10³, 10⁴ or 10⁵ or higher differences in binding affinity.

The present disclosure provides activatable masked antigen binding proteins comprising an IgG type antibody, in an inactive or activated state, comprising an Fc region that exhibits effector function, including complement-dependent cytotoxicity (CDC), antibody-dependent cell-mediated cytotoxicity (ADCC) and/or antibody-dependent phagocytosis (ADP). In one embodiment, a mutation in the Fc region can increase or decrease any one or any combination of these functions. In one embodiment, the Fc region comprises a LALA-PG mutation (L234A, L235A, P329G) which reduces effector function.

In one embodiment, the Fc region mediates serum half-life of the activatable masked antigen binding proteins, and a mutation in the Fc region can increase or decrease the serum half-life of the activatable masked antigen binding proteins.

In one embodiment, the Fc region affects thermal stability of the activatable masked antigen binding proteins, and mutation in the Fc region can increase or decrease the thermal stability of the activatable masked antigen binding proteins.

The present disclosure provides activatable masked antigen binding proteins comprising an IgG type antibody and having one or more amino acid mutations in an Fc region where the mutation(s) lead to introducing knob-in-hole structures that promote dimerization of the first and second heavy chains, (Ridgeway 1996 Protein Engineering 9(7):617-621), or lead to introducing additional interchain disulfide bonding (Carter 2011 Journal of Immunological Methods 248:7-15), and/or lead to introducing new salt bridges.

In one embodiment, the Fc region includes mutations that create a protrusion (e.g., knob) on one heavy chain and a socket (e.g., hole) on the other heavy chain so that the protrusion and socket associate with each other. In one embodiment, the protrusion and socket promote association between the heavy chains, for example to promote dimerization. In one embodiment, one of the heavy chains is mutated by substituting a small amino acid with a larger one to create a protrusion. In one embodiment, the other heavy chain is mutated by substituting a larger amino acid with a smaller one to create a socket. In one embodiment, Fc region knob-in-hole mutations comprise a substitute mutation at any one Fc location or any combination of two or more Fc locations selected from a group consisting of T366, L368, T394, F405, Y407 and K409 (numbering is based on Kabat system). In one embodiment, Fc region knob-in-hole mutations comprise any one or any combination of two or more of the following mutations: T366Y, T366W, T366S, L368A, T394S, T394W, F405A, F405W, Y407A, Y407V, Y407T (numbering based on Kabat system).

The present disclosure provides nucleic acids encoding any of the activatable masked antigen binding proteins comprising an IgGtype antibody. For example, a nucleic acid encodes an activatable masked antigen binding protein comprising an IgG class antibody that binds a target antigen (e.g., monospecific). In another example, a nucleic acid encodes an activatable masked antigen binding protein comprising an IgG class antibody that binds two target antigens (e.g., bispecific).

In one embodiment, a first nucleic acid encodes a heavy chain comprising (i) a first or third masking moiety, (ii) a first or third peptide linker and (iii) a heavy chain variable region. In one embodiment, the first nucleic acid further encodes a heavy chain comprising (iv) a heavy chain constant region (CH1). In one embodiment, the first nucleic acid further encodes a heavy chain comprising (v) a hinge region. In one embodiment, the first nucleic acid further encodes a heavy chain comprising (vi) a heavy chain constant region (CH2 and/or CH3). In one embodiment, the first nucleic acid encodes a heavy chain constant region (CH2 and/or CH3) with a knob or hole. In one embodiment, the first linker comprises a first cleavable site. In one embodiment, the third linker comprises a third cleavable site. In one embodiment, the first nucleic acid comprises a recombinant nucleic acid molecule.

In one embodiment, the first nucleic acid encodes a heavy chain comprising a heavy chain variable region which comprises an amino acid sequence having at least 92%, or at least 93%, or at least 94%, or at least 95%, or at least 96%, or at least 97%, or at least 98%, or at least 99% sequence identity to a portion or full-length of SEQ ID NO:2 or 4 (e.g., anti-EGFR) or to a portion or full-length of SEQ ID NO:7, 9, 11, 13, 14, 15, 16, 17, 18 or 20 (e.g., anti-CD38), or having 100% sequence identity to a portion or full-length of SEQ ID NO:2 or 4 (e.g., anti-EGFR) or a portion or full-length of SEQ ID NO: 7, 9, 11, 13, 14, 15, 16, 17, 18 or 20 (e.g., anti-CD38).

In one embodiment, the first nucleic acid encodes a heavy chain comprising a first or third masking moiety comprising the amino acid sequence of any one of SEQ ID NO:21-31 (see Tables 1 and 2).

In one embodiment, the first nucleic acid encodes a heavy comprising a first or third peptide linker comprising the amino acid sequence of any one of SEQ ID NO:32-40 (see also Tables 3-9 and 12).

In one embodiment, a second nucleic acid encodes a light chain comprising (i) a second or fourth masking moiety, (ii) a second or fourth peptide linker and (iii) a light chain variable region. In one embodiment, the second nucleic acid further encodes a light chain constant region (CL). In one embodiment, the second linker comprises a second cleavable site. In one embodiment, the fourth linker comprises a fourth cleavable site. In one embodiment, the second nucleic acid comprises a recombinant nucleic acid molecule.

In one embodiment, the second nucleic acid encodes a light chain comprising a light chain variable region which comprises an amino acid sequence having at least 92%, or at least 93%, or at least 94%, or at least 95%, or at least 96%, or at least 97%, or at least 98%, or at least 99% sequence identity to a portion or full-length of SEQ ID NO:3 or 5 (anti-EGFR) or a portion or full-length of SEQ ID NO:8, 10, 12 or 19 (e.g., anti-CD38), or having 100% sequence identity to a portion or full-length of SEQ ID NO:3 or 5 (e.g., anti-EGFR) or a portion or full-length of SEQ ID NO:8, 10, 12 or 19 (e.g., anti-CD38).

In one embodiment, the second nucleic acid encodes a light chain comprising a second or fourth masking moiety comprising the amino acid sequence of any one of SEQ ID NO:21-31 (see Tables 1 and 2).

In one embodiment, the second nucleic acid encodes a light chain comprising a second or fourth peptide linker comprising the amino acid sequence of any one of SEQ ID NO:32-40 (see also Tables 3-9 and 12).

The present disclosure provides individual vectors, including expression vectors, that are operably joined to nucleic acids encoding any of the activatable masked antigen binding proteins comprising an IgG type antibody. For example, the expression vector is operably joined to a nucleic acid encoding an activatable masked antigen binding protein comprising an IgG class antibody that binds a target antigen (e.g., monospecific). In another example, the expression vector is operably joined to a nucleic acid encoding an activatable masked antigen binding protein comprising an IgG class antibody that binds two target antigens (e.g., bispecific).

In one embodiment, the expression vector comprising one or more regulatory sequences (e.g., promoter and/or enhancer) that control transcription of the nucleic acids encoding any of the activatable masked antigen binding proteins comprising an IgG type antibody. In one embodiment, the expression vector comprises one or more regulatory sequences each operably joined to a nucleic acid encoding a heavy chain or light chain, and each regulatory sequence controls transcription of the nucleic acids encoding the heavy chain or light chain in a mono-cistronic manner. In one embodiment, the expression vector comprises a regulatory sequence operably joined to nucleic acids encoding a heavy chain and light chain, and the regulatory sequence controls transcription of the heavy chain and light chain in a poly-cistronic manner.

In one embodiment, the vector comprises multiple regulatory sequences that can be operably joined to individual nucleic acids each encoding a heavy chain or a light chain sequence.

In one embodiment, one expression vector is introduced into a host cell, wherein the expression vector within the host cell carries a promoter (and optionally an enhancer sequence), that is operably joined to a nucleic acid encoding a heavy chain and/or light chain sequence. Thus, the host cell can express the heavy chain and/or light chain that make up any of the activatable masked antigen binding proteins.

In one embodiment, multiple expression vectors are introduced into a host cell, wherein individual expression vectors within the host cell carry a promoter (and optionally an enhancer sequence), that is operably joined to a nucleic acid encoding a heavy chain or light chain sequence. Thus, the host cell can express the heavy chain and light chain that make up any of the activatable masked antigen binding proteins.

The vectors comprise promoters that are inducible or constitutive promoters. The vectors and host cells can be selected to generate transgenic host cells that transiently or stably express any of the heavy chains or light chains described herein.

The present disclosure provides host cells that harbor a single expression vector that is operably joined to one or more nucleic acids that encode the heavy chain and/or light chain that make up any of the activatable masked antigen binding proteins.

The present disclosure provides host cells that harbor two or more expression vectors each expression vector being operably joined to one or more nucleic acids that encode a heavy chain and/or light chain that make up any of the activatable masked antigen binding proteins.

The host cell can be a bacterial or mammalian cell. In one embodiment, the host cell comprises a Chinese hamster ovary (CHO) cell.

In one embodiment, at least one expression vector is introduced into the host cell via lipofection (e.g., using a lipid surfactant); electroporation; transfection employing calcium chloride, rubidium chloride, calcium phosphate, DEAE-dextran, or other substances; viral transfection; non-viral transfection; microprojectile bombardment; and infection (e.g., where the vector is an infectious agent).

In one embodiment, host cells harbor one expression vector which is operably joined to nucleic acids encoding the heavy chain and light chain. The host cells can express the heavy chain and light chain at a molar ratio for heavy chain:light chain of 1:1, 1:1.5, 1.5:1, 1:2, 2:1, 1:3, or 3:1. Other molar ratios are possible as well known in the art.

In one embodiment, host cells harbor expression two vectors, wherein the first expression vector is operably joined to nucleic acids encoding the heavy chain, and the second expression vector is operably joined to nucleic acid encoding the light chain. The host cells can express the heavy chain and light chain at a molar ratio for heavy chain:light chain of 1:1, 1:1.5, 1.5:1, 1:2, 2:1, 1:3, or 3:1. Other molar ratios are possible as well known in the art.

The present disclosure provide methods for preparing any of the activatable masked antigen binding proteins comprising an IgG type antibody described herein, the method comprising: culturing a population of host cells, wherein individual host cells in the population harbor at least one expression vector that is operably linked to any one or any combination of nucleic acids encoding any one or any combination of heavy chain and/or light chain described herein, wherein the culturing is conducted under conditions suitable for expressing the heavy chains and/or light chains by the population of host cells.

In one embodiment, the nucleic acids encoding the heavy chain and/or light chain further encodes a signal peptide for secretion of the expressed polypeptide chains. In one embodiment, the culturing is conducted under conditions suitable for secretion of the heavy chain and/or light chain by the population of host cells.

In one embodiment, the nucleic acids encoding any one or any combination of the heavy chain and/or light chain further encodes an affinity tag sequence for enriching the expressed polypeptides. Exemplary affinity tag sequences include histidine tag, FLAG tag, myc tag, HA tag, and GST tag.

In one embodiment, the methods for preparing any of the activatable masked antigen binding proteins comprising an IgG type antibody described herein further comprise isolating the expressed heavy chains and/or light chains.

In one embodiment, the culturing is conducted under conditions that are suitable for assembly or association of the heavy chain and light chain to form the activatable masked antigen binding proteins comprising an IgG type antibody.

In one embodiment, the method further comprises isolating or recovering the assembled activatable masked antigen binding proteins comprising an IgG type antibody. In one embodiment, the isolating is conducted using affinity chromatography. In one embodiment, the isolating is conducted using affinity chromatography with protein A or G from Staphylococcus aureus, glutathione S-transferase (GST), or immuno-affinity. In one embodiment, one or more additional isolating steps are conducted which includes cation exchange, anion exchange chromatography, hydrophobic interaction chromatography, mixed mode chromatography and/or hydroxyapatite chromatography.

In one embodiment, the assembled activatable masked antigen binding proteins comprising an IgG type antibody comprising heavy chains paired with light chains, wherein (1) the heavy chain comprises a first or third masking moiety, a first or third peptide linker, a heavy chain variable region, and optionally including heavy chain constant regions (CH1, CH2 and/or CH3), and (2) the light chain comprises a second or fourth masking moiety, a second or fourth peptide linker, a light chain variable region, and optionally including light chain constant regions (CL).

The activatable masked antigen binding proteins comprising an IgG type antibody can be prepared using transgenic host cell expression, phage display, yeast display and human antibody gene transgenic mice using methods that are well known in the art. In one embodiment, the yield of antigen binding proteins using transgenic host cell expression can be about 20-80%, or about 30-90%, or about 40-95%, or about 50-99% of the total activatable masked antigen binding proteins formed.

The present disclosure provide methods for cleaving at least one peptide linker of any of the activatable masked antigen binding proteins comprising an IgG type antibody described herein, the method comprising: (a) contacting at least one protease with an activatable masked antigen binding protein in an inactive form, wherein the first, second, third and fourth peptide linkers are in the un-cleaved state.

In one embodiment, the activatable masked antigen binding protein is contacted with an one or any combination of two or more proteases selected from: a matrix metalloprotease (MMP), MMP1, MMP2, MMP3, MMP8, MMP9, MMP11, MMP13, MMP14, MT1-MMP (membrane type 1 matrix metalloproteinase), a disintegrin and metalloproteinase (ADAM) protease, ADAM10, ADAM12, ADAM17, urokinase plasminogen activator (uPA), uPAR, serine proteases, cysteine proteases, aspartate proteases, threonine proteases, cathepsins (e.g., cysteine or aspartic cathepsins), cathepsin B, cathepsin C, cathepsin D, cathepsin E, cathepsin K or cathepsin L.

In one embodiment, the activatable masked antigen binding protein is contacted essentially simultaneously (at the same time) with two or more proteases, or sequentially contacted with two or more proteases in any order.

In one embodiment, at least one of the peptide linkers of the activatable masked antigen binding protein used in step (a) comprises a cleavable site that is cleavable with one or more proteases, including a matrix metalloprotease (MMP), MMP1, MMP2, MMP3, MMP8, MMP9, MMP11, MMP13, MMP14, MT1-MMP (membrane type 1 matrix metalloproteinase), a disintegrin and metalloproteinase (ADAM) protease, ADAM10, ADAM12, ADAM17, urokinase plasminogen activator (uPA), uPAR, serine proteases, cysteine proteases, aspartate proteases, threonine proteases, cathepsins (e.g., cysteine or aspartic cathepsins), cathepsin B, cathepsin C, cathepsin D, cathepsin E, cathepsin K or cathepsin L.

In one embodiment, the method further comprises: (b) cleaving at least one of the cleavable sites to convert the activatable masked antigen binding protein to an activated form. In one embodiment, the activated form can bind a target antigen.

In one embodiment, the method further comprises: (c) binding the activatable masked antigen binding protein (now in the activated state) to a target antigen.

In one embodiment, the steps of contacting, cleaving and binding are conducted in an in vitro or in vivo condition.

In one embodiment, the target antigen comprises a soluble antigen or a surface antigen expressed by a healthy or diseased cell.

In one embodiment, the diseased cell (e.g., tumor or cancer cell) that expresses the target antigen also expresses one or more proteases that cleaves the peptide linker. In one embodiment, the diseased cell expresses one or a combination of two or more proteases comprising a matrix metalloprotease (MMP), MMP1, MMP2, MMP3, MMP8, MMP9, MMP11, MMP13, MMP14, MT1-MMP (membrane type 1 matrix metalloproteinase), a disintegrin and metalloproteinase (ADAM) protease, ADAM10, ADAM12, ADAM17, urokinase plasminogen activator (uPA), serine proteases, cysteine proteases, aspartate proteases, threonine proteases, cathepsins, cathepsin B, cathepsin C, cathepsin D, cathepsin E, cathepsin K and/or cathepsin L.

In one embodiment, method further comprises: (d) killing the diseased cell (e.g., tumor or cancer cell).

In one embodiment, at least one of the peptide linkers is cleavable with a protease that is present in a tumor microenvironment.

In one embodiment, the tumor microenvironment comprise a protease selected from a group consisting of a matrix metalloprotease (MMP), MMP1, MMP2, MMP3, MMP8, MMP9, MMP11, MMP13, MMP14, MT1-MMP (membrane type 1 matrix metalloproteinase), a disintegrin and metalloproteinase (ADAM) protease, ADAM10, ADAM12, ADAM17, urokinase plasminogen activator (uPA), serine proteases, cysteine proteases, aspartate proteases, threonine proteases, cathepsins (e.g., cysteine or aspartic cathepsins), cathepsin B, cathepsin C, cathepsin D, cathepsin E, cathepsin K and cathepsin L.

The present disclosure provide methods treating a subject having a disease associated with expression or over-expression of a tumor-associated antigen, the method comprising: administering to the subject an effective amount of a therapeutic composition comprising one or any combination of two or more activatable masked antigen binding proteins comprising an IgG type antibody described herein. In one embodiment the activatable masked antigen binding proteins comprising an IgG type antibody (ies) are administered to the subject in an inactive form having the first, second, third and fourth peptide linkers in the un-cleaved state.

The present disclosure provides a method of treating a subject having a disease, disorder or condition associated with detrimental expression of a tumor antigen, wherein the disease is cancer, including, but not limited to hematologic cancer, breast cancer, ovarian cancer, prostate cancer, head and neck cancer, lung cancer, bladder cancer, melanoma, colorectal cancer, pancreatic cancer, lung cancer, liver cancer, renal cancer, esophageal cancer, leiomyoma, leiomyosarcoma, glioma, and glioblastoma, wherein the method for treating the subject comprises administering to the subject an effective amount of a therapeutic composition comprising one or any combination of two or more activatable masked antigen binding proteins comprising an IgG type antibody described herein.

In one embodiment, the cancer is a hematologic cancer selected from the group consisting of non-Hodgkin’s lymphoma (NHL), Burkitt’s lymphoma (BL), B chronic lymphocytic leukemia (B-CLL), B and T acute lymphocytic leukemia (ALL), T cell lymphoma (TCL), acute myeloid leukemia (AML), hairy cell leukemia (HCL), Hodgkin’s Lymphoma (HL), chronic myeloid leukemia (CML) and multiple myeloma (MM).

The present disclosure provides an in vitro cleavage-based method to detect protease activity and specificity for detecting, diagnosing, monitoring and/or staging a diseased tissue such as a cancer or tumor. A tumor or cancer mass can be extracted from a subject and contacted with one or any combination of two or more activatable masked antigen binding proteins comprising an IgG type antibody described herein, each having a known protease cleavage profile. The tumor or cancer mass from the subject produces one or more protease and is contacted with one or more activatable masked antigen binding proteins under conditions suitable for a protease(s) to cleave a peptide linker on the activatable masked antigen binding protein(s). The cleaved peptide linker product can be detected using any method. Thus, the type of protease produced by the tumor or cancer mass can be identified. In one embodiment, the peptide linker is cleavable with any one or any combination of two or more protease selected from a group consisting of a matrix metalloprotease (MMP), MMP1, MMP2, MMP3, MMP8, MMP9, MMP11, MMP13, MMP14, MT1-MMP (membrane type 1 matrix metalloproteinase), ADAM protease, urokinase plasminogen activator (uPA), serine proteases, cysteine proteases, aspartate proteases, threonine proteases, cathepsins (e.g., cysteine or aspartic cathepsins), cathepsin B, cathepsin C, cathepsin D, cathepsin E, cathepsin K and cathepsin L.

The present disclosure provides a method for detecting the presence of a protease produced by a tumor from a subject, the method comprising: (a) contacting (i) a tumor obtained from the subject with (ii) at least one of the activatable masked antigen binding proteins comprising an IgG type antibody described herein, wherein the tumor sample produces a protease.

In one embodiment, the activatable masked antigen binding proteins each comprise at least a first antigen binding domain which comprises a first heavy chain variable region and a first light chain variable region, wherein (i) the first heavy chain variable region is joined to a first masking moiety via a first peptide linker, and (ii) the first light chain variable region is joined to a second masking moiety via a second peptide linker. In one embodiment, the activatable masked antigen binding proteins further comprise a second antigen binding domain which comprises a second heavy chain variable region and a second light chain variable region, wherein (i) the second heavy chain variable region is joined to a third masking moiety via a third peptide linker, and (ii) the second light chain variable region is j oined to a fourth masking moiety via a fourth peptide linker.

In one embodiment, the first peptide linker includes a first cleavable site and the second peptide linker includes a second cleavable site. In one embodiment, the third peptide linker includes a third cleavable site and the fourth peptide linker includes a fourth cleavable site. In one embodiment, the amino acid sequence of the first, second, third and/or fourth cleavable sites may or may not be a substrate for cleavage by the protease produced by the tumor sample. In one embodiment, the contacting in step (a) is performed under conditions suitable for the protease to cleave the first, second, third and/or fourth cleavable site to generate one or more cleavage products when the protease cleaves the first, second, third and/or fourth cleavable site.

In one embodiment, the method further comprises: (b) detecting the first, second, third and/or fourth cleavage product from the first, second, third and/or fourth peptide linker. In one embodiment, the method further comprises: (c) identifying the type of protease produced by the tumor from the subject by detecting the first, second, third and/or fourth cleavage product and correlating any of the cleavage products with the amino acid sequence of the first, second, third and/or fourth cleavable site.

In one embodiment, by identifying the type of protease produced by the tumor in the subject, the cancer in the subject can be diagnosed. In one embodiment, the first, second, third and/or fourth cleavage product can be detected by gel electrophoresis, Western blot analysis, immunology, immunohistochemistry, colorimetrically, spectrophotometrically, mass spectrometry, liquid chromatography, or by any combination thereof. In one embodiment, the tumor or cancer mass can be obtained from a prostate, breast, ovary, head and neck, bladder, skin, colorectal, anus, rectum, pancreas, lung (including non-small cell lung and small cell lung cancers), leiomyoma, brain, glioma, glioblastoma, esophagus, liver, kidney, stomach, colon, cervix, uterus, endometrium, vulva, larynx, vagina, bone, nasal cavity, paranasal sinus, nasopharynx, oral cavity, oropharynx, larynx, hypolarynx, salivary glands, ureter, urethra, penis, and testis. In one embodiment, the subject is a human, non-human primate, simian, ape, murine (e.g., mice and rats), bovine, porcine, equine, canine, feline, caprine, lupine, ranine or piscine. In one embodiment, the in vitro cleavage-based method can be used for detecting, diagnosing, monitoring and/or staging a cancer in the subject.

The present disclosure provides a kit comprising: at least one of the activatable masked antigen binding proteins comprising an IgG type antibody, and/or at least one nucleic acid encoding an activatable masked antigen binding proteins comprising an IgG type antibody. In one embodiment, the kit comprises one or more adjunct compounds selected from a group consisting of Tris, phosphate, carbonate, stabilizers, excipients, biocides and bovine serum albumin. In one embodiment, the kit comprises one or more adjunct compounds selected from a group consisting of Tris, phosphate, carbonate, stabilizers, excipients, biocides and bovine serum albumin. In one embodiment, the kit comprises one container which contains at least one activatable masked antigen binding proteins comprising an IgG type antibody (or nucleic acid encoding the protein thereof) and optionally one or more adjunct compound. In one embodiment, the kit comprises two or more containers, wherein one container contains at least one activatable masked antigen binding proteins comprising an IgG type antibody (or nucleic acid encoding the protein thereof) and a separate container contains one or more adjunct compounds.

### Activatable Masked Antigen Binding Proteins Comprising Dimeric Antigen Receptors (DAR)

The present disclosure provides activatable masked antigen binding proteins comprising a dimeric antigen receptor (DAR) having a Fab fragment j oined to a transmembrane region and intracellular signaling regions, wherein the Fab fragment is joined to paired masking moieties. In one embodiment, the DAR construct includes an optional hinge region between the Fab fragment and the transmembrane region.

In one embodiment, the dimeric antigen receptors (DAR) comprise two polypeptide chains that dimerize to form a protein complex, wherein a first polypeptide chain comprises a heavy chain variable region and a second polypeptide chain comprises a light chain variable region, wherein the heavy chain variable region and the light chain variable region form an antigen binding domain. In one embodiment, the first polypeptide chain is linked to the second polypeptide chain by one or a plurality of disulfide bonds. The dimeric antigen receptors have antibody-like properties as they bind specifically to a target antigen.

In one embodiment, the dimeric antigen receptors (DAR) comprise two polypeptide chains that dimerize to form a protein complex, wherein a first polypeptide chain comprises a light chain variable region and a second polypeptide chain comprises a heavy chain variable region, wherein the light chain variable region and the heavy chain variable region form an antigen binding domain. In one embodiment, the first polypeptide chain is linked to the second polypeptide chain by one or a plurality of disulfide bonds. The dimeric antigen receptors have antibody-like properties as they bind specifically to a target antigen.

In one embodiment, the activatable masked antigen binding protein is joined to a toxin by a linker.

In one embodiment, the dimeric antigen receptors (DAR) lack a peptide linker and/or lack a masking moiety.

The present disclosure provides activatable masked antigen binding proteins comprising a dimeric antigen receptor (DAR) having a first and a second polypeptide chain, wherein the (a) first polypeptide chain comprises regions: (i) an antibody heavy chain variable region (VH), (ii) an antibody heavy chain constant region (CH), (iii) an optional hinge region, (iv) a transmembrane region (TM), and (v) an intracellular signaling region, wherein the (b) second polypeptide chain comprises regions: (i) an antibody light chain variable region (VL) (e.g., kappa or lambda), and (ii) an antibody light chain constant region (CL), wherein the antibody heavy chain variable region (VH) and the antibody light chain variable region (VL) form an antigen binding domain that binds a target antigen.

In one embodiment, (i) the N-terminal end of the first polypeptide chain is joined to a first masking moiety via a first peptide linker having a first cleavable site, and (ii) the N-terminal end of the second polypeptide chain is joined to a second masking moiety via a second peptide linker having a second cleavable site, and (iii) the first and second masking moieties associate with each other without disulfide bonding, and (iv) the first and second masking moieties do not selectively bind the antigen binding domain, and (v) the first cleavable site is cleavable with a first protease, and (vi) the second cleavable site is cleavable with a second protease, wherein the first and second protease cleavable sites are cleavable with the same or different proteases. In one embodiment, the dimeric antigen receptors (DAR) lack a peptide linker and/or lack a masking moiety.

In one embodiment, the activatable masked antigen binding proteins comprising the dimeric antigen receptor (DAR) comprises a first and a second polypeptide chain, wherein the (a) first polypeptide chain comprises: (i) a first masking moiety, (ii) a first peptide linker, (iii) an antibody heavy chain variable region (VH), (iv) an antibody heavy chain constant region (CH), (v) an optional hinge region, (vi) a transmembrane region (TM), and (vii) an intracellular signaling region, wherein the (b) second polypeptide chain comprises: (i) a second masking moiety, (ii) a second peptide linker, (iii) an antibody light chain variable region (VL) (e.g., kappa or lambda), and (iv) an antibody light chain constant region (CL), wherein the antibody heavy chain variable region (VH) and the antibody light chain variable region (VL) form an antigen binding domain that binds a target antigen.

In one embodiment, the first and second masking moieties associates with each other to reduce the antigen binding domain from binding to its target antigen.

In one embodiment, the first and/or second cleavable sites are cleavable with the same or different proteases.

In one embodiment, the first and second proteases are produced by the same tumor or by different tumors.

In one embodiment, the hinge region is about 10 to about 100 amino acids in length. In one embodiment, the hinge region is independently selected from the group consisting of a PDGFR (platelet-derived growth factor receptor) hinge region or a fragment thereof, a CD8 hinge region or a fragment thereof, a CD8α hinge region or a fragment thereof, a hinge region of an antibody (IgG, IgA, IgM, IgE, or IgD) joining the constant domains CH1 and CH2 of an antibody. The hinge region can be derived from an antibody and may or may not comprise one or more constant regions of the antibody.

In one embodiment, the transmembrane domain can be derived from a membrane protein sequence region selected from the group consisting of CD8α, CD8β, 4-1BB/CD137, CD28, CD34, CD4, FcsRIγ, CD16, OX40/CD134, CD3ζ, CD3ε, CD3γ, CD3δ, TCRα, TCRβ, TCRζ, CD32, CD64, CD64, CD45, CD5, CD9, CD22, CD33, CD37, CD64, CD80, CD86, CD137, CD154, LFA-1 T cell co-receptor, CD2 T cell co-receptor/adhesion molecule, CD40, CD4OL/CD154, VEGFR2, FAS, and FGFR2B.

In one embodiment, the intracellular signaling region comprises any one or any combination of two or more intracellular signaling sequences selected from the group consisting of signaling regions from CD3-zeta chain, 4-1BB, CD28, CD27, OX40, CD30, CD40, PD-1, ICOS, lymph oocyte function-associated antigen-1 (LFA-1), CD2, CD7, LIGHT, NKG2C, B7-H3, GITR (TNFRSF18), DR3 (TNFRSF25), TNFR2, CD226, and combinations thereof.

In one embodiment, the intracellular signaling region comprises 1, 2 or 3 different intracellular signaling sequences.

In one embodiment, the intracellular signaling region comprises any one or any combination of two or more signaling sequences selected from a group consisting of 4-1BB, CD28, CD3zeta (long) signaling sequence and/or CD3zeta (short) signaling sequence having an ITAM 3 motif.

In one embodiment, the activatable masked antigen binding protein is joined to a toxin by a linker.

The present disclosure provides activatable masked antigen binding proteins comprising a dimeric antigen receptor (DAR) having a first and a second polypeptide chain, wherein the (a) first polypeptide chain comprises regions: (i) an antibody light chain variable region (VL) (e.g., kappa or lambda), (ii) an antibody light chain constant region (CL), (iii) an optional hinge region, (iv) a transmembrane region (TM), and (v) an intracellular signaling region, wherein the (b) second polypeptide chain comprises regions: (i) an antibody heavy chain variable region (VH), and (ii) an antibody heavy chain constant region (CH), wherein the antibody heavy chain variable region (VH) and the antibody light chain variable region (VL) form an antigen binding domain that binds a target antigen.

In one embodiment, (i) the N-terminal end of the first polypeptide chain is joined to a first masking moiety via a first peptide linker having a first cleavable site, and (ii) the N-terminal end of the second polypeptide chain is joined to a second masking moiety via a second peptide linker having a second cleavable site, and (iii) the first and second masking moieties associate with each other without disulfide bonding, and (iv) the first and second masking moieties do not selectively bind the antigen binding domain, and (v) the first cleavable site is cleavable with a first protease, and (vi) the second cleavable site is cleavable with a second protease, wherein the first and second protease cleavable sites are cleavable with the same or different proteases. In one embodiment, the dimeric antigen receptors (DAR) lack a peptide linker and/or lack a masking moiety.

In one embodiment, the activatable masked antigen binding proteins comprising the dimeric antigen receptor (DAR) comprises a first and a second polypeptide chain, wherein the (a) first polypeptide chain comprises: (i) a first masking moiety, (ii) a first peptide linker, (iii) an antibody light chain variable region (VL) (e.g., kappa or lambda), (iv) an antibody light chain constant region (CL), (v) an optional hinge region, (vi) a transmembrane region (TM), and (vii) an intracellular signaling region, wherein the (b) second polypeptide chain comprises: (i) a second masking moiety, (ii) a second peptide linker, (iii) an antibody heavy chain variable region (VH), and (iv) an antibody heavy chain constant region (CH), wherein the antibody heavy chain variable region (VH) and the antibody light chain variable region (VL) form an antigen binding domain that binds a target antigen.

In one embodiment, the first and second masking moieties associates with each other to reduce the antigen binding domain from binding to its target antigen.

In one embodiment, the first and/or second cleavable sites are cleavable with the same or different proteases.

In one embodiment, the first and second proteases are produced by the same tumor or by different tumors.

In one embodiment, the hinge region is about 10 to about 100 amino acids in length. In one embodiment, the hinge region is independently selected from the group consisting of a CD8 hinge region or a fragment thereof, a CD8α hinge region or a fragment thereof, a hinge region of an antibody (IgG, IgA, IgM, IgE, or IgD) joining the constant domains CH1 and CH2 of an antibody. The hinge region can be derived from an antibody and may or may not comprise one or more constant regions of the antibody.

In one embodiment, the transmembrane domain can be derived from a membrane protein sequence region selected from the group consisting of CD8α, CD8β, 4-1BB/CD137, CD28, CD34, CD4, FcsRIγ, CD16, OX40/CD134, CD3ζ, CD3ε, CD3γ, CD3δ, TCRα, TCRβ, TCRζ, CD32, CD64, CD64, CD45, CD5, CD9, CD22, CD33, CD37, CD64, CD80, CD86, CD137, CD154, LFA-1 T cell co-receptor, CD2 T cell co-receptor/adhesion molecule, CD40, CD4OL/CD154, VEGFR2, FAS, and FGFR2B.

In one embodiment, the intracellular signaling region comprises any one or any combination of two or more intracellular signaling sequences selected from the group consisting of signaling regions from CD3-zeta chain, 4-1BB, CD28, CD27, OX40, CD30, CD40, PD-1, ICOS, lymph oocyte function-associated antigen-1 (LFA-1), CD2, CD7, LIGHT, NKG2C, B7-H3, GITR (TNFRSF 18), DR3 (TNFRSF25), TNFR2, CD226, and combinations thereof.

In one embodiment, the intracellular signaling region comprises 1, 2 or 3 different intracellular signaling sequences.

In one embodiment, the intracellular signaling region comprises any one or any combination of two or more signaling sequences selected from a group consisting of 4-1BB, CD28, CD3zeta (long) signaling sequence, and/or CD3zeta (short) signaling sequence having an ITAM 3 motif.

In one embodiment, the activatable masked antigen binding protein is joined to a toxin by a linker.

The present disclosure provides any of the activatable masked antigen binding proteins comprising a dimeric antigen receptor (DAR) described herein having at least a first and second masking moiety. In one embodiment, the first and second masking moieties when associated (dimerized) with each other, interfere (obstruct) with the ability of the antigen binding protein to bind its target antigen. The first and second masking moieties when associated (dimerized) with each other, can block binding between the antigen binding protein and its target antigen by steric hinderance.

In one embodiment, the first and second masking moieties comprise polypeptides that associate (dimerize) with each other without forming a covalent bond (e.g., no disulfide or transglutamination bonding). The first and second masking moieties comprise polypeptides that can associate (dimerize) with each other by non-covalent interaction, including ionic interaction, hydrogen bonding, dipole-dipole interaction, hydrophilic interaction, hydrophobic interaction, affinity bonding, or bonds or associations involving van der Waals forces.

In one embodiment, the first and second masking moieties associate with each other as a homodimer or heterodimer.

In one embodiment, one of the masking moieties can be engineered to have a knob structure that forms an interchain steric complementarity structure to favor homo-dimerization of, hetero-dimerization with another masking moiety which is engineered to have a hole structure.

The present disclosure provides activatable masked antigen binding proteins, wherein one or both of the masking moieties in a dimerized configuration includes one or more mutations that create a new interchain salt bridge. In one embodiment, a threonine residue in one masking moiety can be replaced with glutamic acid, and an asparagine at a corresponding position in another masking moiety is replaced with lysine, thereby creating an interchain salt bridge.

In one embodiment, the first and/or second masking moieties are derived from an immunoglobulin constant region (e.g., CL, CH1, CH2 or CH3).

In one embodiment, the first or second masking moiety comprises a heavy chain constant region (CH1). In one embodiment, the second or first masking moiety comprises a kappa or lambda light chain constant region (CL kappa or CL lambda). In one embodiment, the first masking moiety comprises a heavy chain constant region (CH1) and the second masking moiety comprises a kappa or lambda light chain constant region (CL kappa or CL lambda). In one embodiment, the first masking moiety comprises a kappa or lambda light chain constant region (CL kappa or CL lambda) and the second masking moiety comprises a heavy chain constant region (CH1). In one embodiment, the first and/or second masking moiety comprises a CH1, CL (lambda) or CL (kappa) having the amino acid sequence of SEQ ID NO:21, 22 or 23. See Tables 1 and 2 for a list of amino acid sequences of various masking moieties.

In one embodiment, the first or second masking moiety comprises an IgG1 or IgG4 gamma heavy chain constant region, for example CH3 In one embodiment, the first and second masking moieties comprise an IgG1 or IgG4 gamma heavy chain constant region CH3. In one embodiment, one of the masking moieties which comprises an IgGI or IgG4 gamma heavy chain constant region CH3 is engineered to have a knob or hole structure that forms an interchain steric complementarity structure to favor dimerization (e.g., homo-dimerization) with another masking moiety which comprises an IgG1 or IgG4 gamma heavy chain constant region CH3 which is engineered to have hole or knob structure. In one embodiment, the first and/or second masking moiety comprises a CH3 (IgG1 or (IgG4) having the amino acid sequence of SEQ ID NO:24, 25, 30 or 31. See Tables 1 and 2 for a list of amino acid sequences of various masking moieties.

In one embodiment, at least one of the masking moieties is derived from an immunoglobulin heavy chain constant region and is joined to the heavy chain variable region of the activatable masked antibody. In one embodiment, at least one of the masking moieties is derived from an immunoglobulin heavy chain constant region and is joined to the light chain variable region of the activatable masked antibody.

In one embodiment, die first and second masking moieties are derived from T cell receptors alpha (α) and beta (β) constant regions. In one embodiment, the first masking moiety comprises a T cell receptor alpha (α) constant regions and the second masking moiety comprises a T cell receptor beta (β) constant region. In one embodiment, the first masking moiety comprises a T cell receptor beta (β) constant regions and the second masking moiety comprises a T cell receptor alpha (α) constant region.

In one embodiment, at least one of the masking moieties is derived from a T cell receptor alpha (α) or beta (β) constant region and is joined to the heavy chain variable region of the activatable masked antibody. In one embodiment, at least one of the masking moieties is derived from a T cell receptor beta (β) or alpha (α) constant region and is joined to the light chain variable region of the activatable masked antibody. In one embodiment, the first and/or second masking moiety comprises a T cell receptor alpha (α) or beta (β) constant region having the amino acid sequence of SEQ ID NO:28 or 29. See Tables 1 and 2 for a list of amino acid sequences of various masking moieties.

In one embodiment, the first or second masking moiety comprises a variable heavy chain domain from a catalytic antibody (e.g., 38C2). In one embodiment, the first or second masking moiety comprises a variable light chain domain from a catalytic antibody (e.g., 38C2). In one embodiment, the first masking moiety comprises a variable heavy chain domain from 38C2 antibody and the second masking moiety comprise a variable light chain domain from 38C2 antibody. In one embodiment, the first and/or second masking moiety comprises a variable heavy chain domain from catalytic antibody 38C2 having the amino acid sequence of SEQ ID NO:26 or 27. See Tables 1 and 2 for a list of amino acid sequences of various masking moieties.

In one embodiment, a masking moiety (e.g., first or second masking moiety) comprises two or more copies of a moiety arranged in-tandem along the same polypeptide chain. For example, a masking moiety comprises two antibody CH3 regions arranged in-tandem, or two antibody CH2 regions arranged in-tandem, or two antibody CH1 regions arranged in-tandem, or two antibody CL regions arranged in-tandem. Any of the CH3, CH2, CH1 or CL regions can include a knob or hole structure. For example, the masking moiety comprises two TCR alpha chain constant region arranged in-tandem, or two TCR beta chain constant region arranged in-tandem.

In one embodiment, a masking moiety (e.g., first or second masking moiety) comprises two or more different moieties arranged in-tandem along the same polypeptide chain. For example, the masking moiety comprises an antibody CH2 region and an antibody CH3 region arranged in-tandem.

The present disclosure provides any of the activatable masked antigen binding proteins comprising a dimeric antigen receptor (DAR) described herein including at least first and second peptide linkers that have the same or different lengths (e.g., 10-20, 20-30, 30-40 or 40-50 amino acids in length). In one embodiment, the first and/or second peptide linkers are glycine-rich (e.g., GS type linkers). In one embodiment, the first and the second peptide linkers have the same amino acid sequence or different amino acid sequences.

In one embodiment, the first peptide linker is cleavable with a first protease. In one embodiment, the first peptide linker has an amino acid sequence (first cleavable site) that is a substrate for cleavage by the first protease.

In one embodiment, the second peptide linker is cleavable with a second protease. In one embodiment, the second peptide linker has an amino acid sequence (second cleavable site) that is a substrate for cleavage by the second protease.

In one embodiment, the first peptide linker is cleavable with a first protease and the second peptide linker is cleavable with a second protease. In one embodiment, the first and second peptide linkers are cleavable with the same protease. In one embodiment, the first and second peptide linkers are cleavable with different proteases. In one embodiment, the first or the second peptide linker is not cleavable with a protease.

In one embodiment, the first and second proteases can be independently selected from: a matrix metalloprotease (MMP) including MMP1, MMP2, MMP3, MMP8, MMP9, MMP11, MMP13, MMP14, MT1-MMP (membrane type 1 matrix metalloproteinase); a disintegrin and metalloproteinase (ADAM) protease including ADAM10, ADAM12, ADAM17; a urokinase plasminogen activator (uPA) or uPAR; a serine protease; a cysteine protease; an aspartate protease; a threonine protease; a cathepsin (e.g., cysteine or aspartic cathepsins) including cathepsin B, cathepsin C, cathepsin D, cathepsin E, cathepsin K or cathepsin L.

In one embodiment, the first and/or second peptide linkers comprise the amino acid sequence that is at least 90%, 91%, 92%, 93%, 94%, 95%, 96%, 97%, 98%, 99% or 100% identical to the amino acid sequences of any one of SEQ ID NOS: 32-40. See also the various peptide linker sequences listed in Tables 3-9.

The present disclosure provides activatable masked antigen binding proteins comprising a dimeric antigen receptor (DAR) having an antigen binding domain which comprises a first heavy chain variable region and a first light chain variable region, wherein the antigen binding domain binds a target antigen (e.g., specifically binds a target antigen).

In one embodiment, the activatable masked antigen binding protein binds EGFR antigen having the amino acid sequence of SEQ ID NO: 1 or bind CD38 antigen having the amino acid sequence of SEQ ID NO:6.

In one embodiment, the activatable masked antigen binding proteins binds an antigen from a human. In one embodiment, the activatable masked antigen binding proteins binds an antigen from a human and can bind (e.g., cross-react) with an antigen (e.g., homologous antigen) from any one or any combination of non-human animals such as dog, cat, mouse, rat, goat, rabbit, hamster and/or monkey (e.g., cynomolgus, rhesus or macaque).

The present disclosure provides activatable masked antigen binding proteins comprising a dimeric antigen receptor (DAR) having a first and a second polypeptide chain.

In one embodiment, the first polypeptide chain comprises a first masking moiety comprising a CH1, CL (lambda or kappa) region having the amino acid sequence of SEQ ID NO:21, 22 or 23. In one embodiment, the first polypeptide chain comprises a first masking moiety comprising a CH3 (IgG1 or IgG4) region having the amino acid sequence of SEQ ID NO:24, 25, 30 or 31. In one embodiment, the first polypeptide chain comprises a first masking moiety comprising a T cell receptor alpha (α) or beta (β) constant region having the amino acid sequence of SEQ ID NO:28 or 29. In one embodiment, the first polypeptide chain comprises a first masking moiety comprising a variable heavy chain domain from catalytic antibody 38C2 having the amino acid sequence of SEQ ID NO:26 or 27.

In one embodiment, the second polypeptide chain comprises a second masking moiety comprising a CH1, CL (lambda or kappa) region having the amino acid sequence of SEQ ID NO:21, 22 or 23. In one embodiment, the second polypeptide chain comprises a second masking moiety comprising a CH3 (IgG1 or IgG4) region having the amino acid sequence of SEQ ID NO:24, 25, 30 or 31. In one embodiment, the second polypeptide chain comprises a second masking moiety comprising a T cell receptor alpha (α) or beta (β) constant region having the amino acid sequence of SEQ ID NO:28 or 29. In one embodiment, the second polypeptide chain comprises a second masking moiety comprising a variable heavy chain domain from catalytic antibody 38C2 having the amino acid sequence of SEQ ID NO:26 or 27.

In one embodiment, the first polypeptide chain comprises a first peptide linker comprising the amino acid sequence of one of SEQ ID NO:32-40 or comprising any one of the amino acid sequences listed in Tables 3-9.

In one embodiment, the second polypeptide chain comprises a second peptide linker comprising the amino acid sequence of one of SEQ ID NO:32-40 or comprising any one of the amino acid sequences listed in Tables 3-9.

In one embodiment, the first polypeptide chain comprises an antibody heavy chain variable region (VH) comprising an anti-EGFR heavy chain variable region sequence having an amino acid sequence having at least 92%, or at least 93%, or at least 94%, or at least 95%, or at least 96%, or at least 97%, or at least 98%, or at least 99% sequence identity, or having 100% sequence identity, to a portion or full-length of SEQ ID NO:2 or 4. In one embodiment, the first polypeptide chain comprises an antibody heavy chain variable region (VH) comprising an anti-CD38 heavy chain variable region sequence having an amino acid sequence having at least 92%, or at least 93%, or at least 94%, or at least 95%, or at least 96%, or at least 97%, or at least 98%, or at least 99% sequence identity, or having 100% sequence identity, to a portion or full-length of SEQ ID NO:7, 9, 11, 13, 14, 15, 16, 17, 18 or 20.

In one embodiment, the first polypeptide chain comprises an antibody light chain variable region (VL) comprising an anti-EGFR light chain variable region sequence having an amino acid sequence having at least 92%, or at least 93%, or at least 94%, or at least 95%, or at least 96%, or at least 97%, or at least 98%, or at least 99% sequence identity, or having 100% sequence identity, to a portion or full-length of SEQ ID NO:3 or 5. In one embodiment, the first polypeptide chain comprises an antibody light chain variable region (VL) comprising an anti-CD38 light chain variable region sequence having an amino acid sequence having at least 92%, or at least 93%, or at least 94%, or at least 95%, or at least 96%, or at least 97%, or at least 98%, or at least 99% sequence identity, or having 100% sequence identity, to a portion or full-lengthof SEQ ID NO:8, 10, 12 or 19.

In one embodiment, the second polypeptide chain comprises an antibody light chain variable region (VL) comprising an anti-EGFR light chain variable region sequence having an amino acid sequence having at least 92%, or at least 93%, or at least 94%, or at least 95%, or at least 96%, or at least 97%, or at least 98%, or at least 99% sequence identity, or having 100% sequence identity, to a portion or full-length of SEQ ID NO:3 or 5. In one embodiment, the second polypeptide chain comprises an antibody light chain variable region (VL) comprising an anti-CD38 light chain variable region sequence having an amino acid sequence having at least 92%, or at least 93%, or at least 94%, or at least 95%, or at least 96%, or at least 97%, or at least 98%, or at least 99% sequence identity, or having 100% sequence identity, to a portion or full-length of SEQ ID NO:8, 10, 12 or 19.

In one embodiment, the second polypeptide chain comprises an antibody heavy chain variable region (VH) comprising an anti-EGFR heavy chain variable region sequence having an amino acid sequence having at least 92%, or at least 93%, or at least 94%, or at least 95%, or at least 96%, or at least 97%, or at least 98%, or at least 99% sequence identity, or having 100% sequence identity, to a portion or full-length of SEQ ID NO:2 or 4. In one embodiment, the second polypeptide chain comprises an antibody heavy chain variable region (VH) comprising an anti-CD38 heavy chain variable region sequence having an amino acid sequence having at least 92%, or at least 93%, or at least 94%, or at least 95%, or at least 96%, or at least 97%, or at least 98%, or at least 99% sequence identity, or having 100% sequence identity, to a portion or full-length of SEQ ID NO:7, 9, 11, 13, 14, 15, 16, 17, 18 or 20.

In one embodiment, the first polypeptide chain comprises an antibody heavy chain constant region (CH) comprising an anti-EGFR heavy chain constant region sequence (e.g., from SEQ ID NO:2 or 4). In one embodiment, the first polypeptide chain comprises an antibody heavy chain constant region (CH) comprising an anti-CD38 heavy chain constant region sequence (e.g., from SEQ ID NO: 7, 9, 11, 13, 14, 15, 16, 17, 18 or 20).

In one embodiment, the first polypeptide chain comprises an antibody light chain constant region (CL) comprising an anti-EGFR light chain constant region sequence (e.g., from SEQ ID NO:3 or 5). In one embodiment, the first polypeptide chain comprises an antibody light chain constant region (CL) comprising an anti-CD38 light chain constant region sequence (e.g., from SEQ ID NO:8, 10, 12 or 19).

In one embodiment, the second polypeptide chain comprises an antibody light chain constant region (CL) comprising an anti-EGFR light chain constant region sequence (e.g., from SEQ ID NO:3 or 5). In one embodiment, the second polypeptide chain comprises an antibody light chain constant region (CL) comprising an anti-CD38 light chain constant region sequence (e.g., from SEQ ID NO:8, 10, 12 or 19).

In one embodiment, the second polypeptide chain comprises an antibody heavy chain constant region (CH) comprising an anti-EGFR heavy chain constant region sequence (e.g., from SEQ ID NO:2 or 4). In one embodiment, the second polypeptide chain comprises an antibody heavy chain constant region (CH) comprising an anti-CD38 heavy chain constant region sequence (e.g., from SEQ ID NO: 7, 9, 11, 13, 14, 15, 16, 17, 18 or 20).

In one embodiment, the first polypeptide chain comprises a hinge region having the amino acid sequence of any one or any combination of two or more of: a CD28 hinge sequence (SEQ ID NO:44); a CD8 hinge sequence (SEQ ID NO:45); a long hinge comprising CD28 and CD8 hinge sequences (SEQ ID NO:46); and/or a PDGFR beta hinge sequence (SEQ ID NO:41).

In one embodiment, the first polypeptide chain comprises a transmembrane region having the amino acid sequence of a CD28 transmembrane sequence (SEQ ID NO:47) or a PDGFR beta transmembrane sequence (SEQ ID NO:42).

In one embodiment, the first polypeptide chain comprises 1 - 3 intracellular signaling sequences in any order and in any combination having the amino acid sequence(s) of: a 4-1BB signaling sequence (SEQ ID NO:48); a CD28 signaling sequence (SEQ ID NO:49); a CD3zeta signaling sequence (SEQ ID NO:50); and/or a CD3zeta short signaling sequence (SEQ ID NO:51).

The present disclosure provides activatable masked antigen binding proteins that exhibit inactive and activated states which depend upon the ability of the masking moieties to obstruct binding between the antigen binding domain and its target antigen. In one embodiment, individual masking moieties are tethered to the N-terminus of the heavy chain variable region (on the first polypeptide chain) and light chain variable region (on the second polypeptide chain) by intact peptide linkers, and the masking moieties form a dimerized complex which obstructs the antigen binding domain resulting in an inactive state. Cleavage of one or both peptide linkers allows the dimerized complex to move away from the obstructing position to permit binding between the antigen binding domain and its target antigen. In one embodiment, cleavage of both peptide linkers liberates the dimerized complex to migrate away from the antigen binding protein.

In one embodiment, the activatable masked antigen binding proteins comprising a dimeric antigen receptor (DAR) is inhibited from binding to its target antigen (e.g., inactive) when the first and second peptide linkers are in the un-cleaved state. In one embodiment, the activatable masked antigen binding proteins exhibit reduced binding capability to its target antigen (e.g., inactive) when the first and second peptide linkers are in the un-cleaved state. In one embodiment, in an inactive state masked antigen binding protein, the first and second masking moieties may or may not be associated with each other.

In one embodiment, a masked antigen binding protein having intact first and second peptide linkers, the binding capacity to the target antigen can be reduced by at least 25-50%, or at least 50-75%, or at least 75-95%, or at least 95-99% or more.

In one embodiment, a masked antigen binding protein having intact first and second peptide linkers, the binding capacity to the target antigen can be reduced by about 2-20 fold, or about 20-50 fold, or about 50-200 fold, or about 200-350 fold, or about 350-500 fold, or higher fold-levels of reduced binding capacity.

In one embodiment, a masked antigen binding protein having intact first and second peptide linkers, the binding affinity (e.g., K_{D}) of an inactive antigen binding protein is about 10⁻⁴ - 10⁻⁸ M. In one embodiment, the target antigen is a soluble antigen or a cell surface antigen.

The present disclosure provides activatable masked antigen binding proteins that are un-masked and capable of binding its target antigen (e.g., activated) when the first and/or second peptide linkers are in the cleaved state. In one embodiment, the activatable masked antigen binding proteins exhibit increased binding capability to its target antigen (e.g., activated) when the first and/or second peptide linkers are in the cleaved state. For example, the activatable masked antigen binding proteins can bind its target antigen when only the first or second peptide linker is cleaved, or when the first and second linkers are cleaved. In one embodiment, in an activated state masked antigen binding protein, the first and second masking moieties may or may not be associated with each other.

In one embodiment, an un-masked antigen binding protein having cleaved first and/or second peptide linkers, the binding capacity to the target antigen can be increased by at least 25-50%, or at least 50-75%, or at least 75-95%, or at least 95-99% or more.

In one embodiment, an un-masked antigen binding protein having cleaved first and/or second peptide linkers, the binding capacity to the target antigen can be increased by about 2-20 fold, or about 20-50 fold, or about 50-200 fold, or about 200-350 fold, or about 350-500 fold, or higher fold-levels of increased binding capacity.

In one embodiment, an un-masked antigen binding protein having cleaved first and/or second peptide linkers, the binding affinity (e.g., K_{D}) of an activated antigen binding protein is about 10⁻⁵- 10⁻¹² M. In one embodiment, the target antigen is a soluble antigen or a cell surface antigen. In one embodiment, the binding affinity of the activated form of the antigen binding protein is higher compared to the inactive form.

In one embodiment, the difference in binding affinity between the inactive and activated antigen binding protein (e.g., K_{D} of inactive vs. K_{D} of activated) is about 10, 10², 10³, 10⁴ or 10⁵ or higher differences in binding affinity.

The present disclosure provides a Version 1 (e.g., V1) dimeric antigen receptors (DAR) construct comprising a first polypeptide chain carrying heavy chain variable (VH) and heavy chain constant regions (CH), and a second polypeptide chain carrying light chain variable (VL) and light chain constant regions (CL), wherein (a) the first polypeptide chain comprising regions ordered from the amino terminus to the carboxyl terminus: (i) a first masking moiety, (ii) a first peptide linker, (iii) an antibody heavy chain variable region (VH), (iv) an antibody heavy chain constant region (CH), (v) a long hinge region comprising CD8 and CD28 hinge sequences (e.g., SEQ ID NO:46), (vi) a transmembrane region (TM) comprising CD28 transmembrane sequence (e.g., SEQ ID NO:47), and (vii) an intracellular signaling region comprising CD28 signaling sequence (e.g., SEQ ID NO:49) and CD3-zeta signaling sequence having ITAM motifs 1, 2 and 3 (e.g., SEQ ID NO:50); (b) a second polypeptide chain comprising regions ordered from the amino terminus to the carboxyl terminus: (i) a second masking moiety, (ii) a second peptide linker, (iii) an antibody light chain variable region (VL) (e.g., kappa or lambda), and (iv) an antibody light chain constant region (CL). In one embodiment, the V1 dimeric antigen receptors (DAR) lack a peptide linker and/or lack a masking moiety.

In one embodiment, the V1 DAR antibody heavy chain variable region (VH) comprises an anti-EGFR heavy chain variable region sequence (e.g., from SEQ ID NO:2 or 4) and the antibody heavy chain constant region (CH) comprises an anti-EGFR heavy chain constant region sequence (e.g., from SEQ ID NO:2 or 4). In one embodiment, the V1 DAR antibody heavy chain variable region (VH) comprises an anti-CD38 heavy chain variable region sequence (e.g., from SEQ ID NO:7, 9, 11, 13, 14, 15, 16, 17, 18 or 20) and the antibody heavy chain constant region (CH) comprises an anti-CD38 heavy chain constant region sequence (e.g., from SEQ ID NO: 7, 9, 11, 13, 14, 15, 16, 17, 18 or 20).

In one embodiment, the V1 DAR antibody light chain variable region (VL) comprises an anti-EGFR light chain variable region sequence (e.g., from SEQ ID NO:3 or 5) and the antibody light chain constant region (CL) comprises an anti-EGFR light chain constant region sequence (e.g., from SEQ ID NO:3 or 5). In one embodiment, the V1 DAR antibody light chain variable region (VL) comprises an anti-CD38 light chain variable region sequence (e.g., from SEQ ID NO:8, 10, 12 or 19) and the antibody light chain constant region (CL) comprises an anti-CD38 light chain constant region sequence (e.g., from SEQ ID NO:8, 10, 12 or 19).

In one embodiment, the first polypeptide chain of the Version 1 (V1) DAR construct comprises a PDGFR beta hinge sequence (SEQ ID NO:41) and a PDGFR beta transmembrane sequence (SEQ ID NO:42).

The present disclosure provides a Version 2 (e.g., V2) dimeric antigen receptors (DAR) construct comprising a first polypeptide chain carrying heavy chain variable (VH) and heavy chain constant regions (CH), and a second polypeptide chain carrying light chain variable (VL) and light chain constant regions (CL), wherein (a) the first polypeptide chain comprising regions ordered from the amino terminus to the carboxyl terminus: (i) a first masking moiety, (ii) a first peptide linker, (iii) an antibody heavy chain variable region (VH), (iv) an antibody heavy chain constant region (CH), (v) a short hinge region comprising a CD28 hinge sequence (e.g., SEQ ID NO:44), (vi) a transmembrane region (TM) comprising CD28 transmembrane sequence (e.g., SEQ ID NO:47), and (vii) an intracellular signaling region comprising either (1) a 4-1BB signaling sequence (e.g., SEQ ID NO:48) and CD3-zeta having ITAM motifs 1, 2 and 3 (e.g., SEQ ID NO:50), or (2) CD28 (e.g., SEQ ID NO:49) signaling sequence and CD3-zeta having ITAM motifs 1, 2 and 3 (e.g., SEQ ID NO:50), or (3) 4-1BB (e.g., SEQ ID NO:48) signaling sequence and CD28 (e.g., SEQ ID NO:49) signaling sequence and CD3-zeta having ITAM motifs 1, 2 and 3 (e.g., SEQ ID NO:50); (b) a second polypeptide chain comprising regions ordered from the amino terminus to the carboxyl terminus: (i) a second masking moiety, (ii) a second peptide linker, (iii) an antibody light chain variable region (VL) (e.g., kappa or lambda), and (iv) an antibody light chain constant region (CL). In one embodiment, the V2 dimeric antigen receptors (DAR) lack a peptide linker and/or lack a masking moiety.

In one embodiment, the Version 2a (V2a) DAR construct comprises the intracellular signaling region having the 4-1BB signaling sequence (e.g., SEQ ID NO:48) and CD3-zeta having ITAM motifs 1, 2 and 3 (e.g., SEQ ID NO:50).

In one embodiment, the Version 2b (V2b) DAR construct comprises the intracellular signaling region having the CD28 (e.g., SEQ ID NO:49) signaling sequence and CD3-zeta having ITAM motifs 1, 2 and 3 (e.g., SEQ ID NO:50).

In one embodiment, the Version 2c (V2c) DAR construct comprises the intracellular signaling region having the 4-1BB (e.g., SEQ ID NO:48) signaling sequence and CD28 (e.g., SEQ ID NO:49) signaling sequence and CD3-zeta having ITAM motifs 1, 2 and 3 (e.g., SEQ ID NO:50).

In one embodiment, the DAR V2a and V2b are second generation DAR constructs, while the DAR V2c is a third generation DAR construct. In one embodiment, the antibody heavy chain variable region (VH) comprises an anti-EGFR heavy chain variable region sequence (e.g., from SEQ ID NO:2 or 4) and the antibody heavy chain constant region (CH) comprises an anti-EGFR heavy chain constant region sequence (e.g., from SEQ ID NO:2 or 4). In one embodiment, the antibody heavy chain variable region (VH) comprises an anti-CD38 heavy chain variable region sequence (e.g., from SEQ ID NO:7, 9, 11, 13, 14, 15, 16, 17, 18 or 20) and the antibody heavy chain constant region (CH) comprises an anti-CD38 heavy chain constant region sequence (e.g., SEQ ID NO: 7, 9, 11, 13, 14, 15, 16, 17, 18 or 20).

In one embodiment, the V2 DAR antibody light chain variable region (VL) comprises an anti-EGFR light chain variable region sequence (e.g., from SEQ ID NO:3 or 5) and the antibody light chain constant region (CL) comprises an anti-EGFR light chain constant region sequence (e.g., from SEQ ID NO:3 or 5). In one embodiment, the V2 DAR antibody light chain variable region (VL) comprises an anti-CD38 light chain variable region sequence (e.g., from SEQ ID NO:8, 10, 12 or 19) and the antibody light chain constant region (CL) comprises an anti-CD38 light chain constant region sequence (e.g., from SEQ ID NO:8, 10, 12 or 19).

In one embodiment, the first polypeptide chain of the Version 2a, 2b or 2c DAR construct comprises a PDGFR beta hinge sequence (SEQ ID NO:41) and a PDGFR beta transmembrane sequence (SEQ ID NO:42).

The present disclosure provides a Version 3 (e.g., V3) dimeric antigen receptors (DAR) construct comprising a first polypeptide chain carrying heavy chain variable (VH) and heavy chain constant regions (CH), and a second polypeptide chain carrying light chain variable (VL) and light chain constant regions (CL), wherein (a) the first polypeptide chain comprising regions ordered from the amino terminus to the carboxyl terminus: (i) a first masking moiety, (ii) a first peptide linker, (iii) an antibody heavy chain variable region (VH), (iv) an antibody heavy chain constant region (CH), (v) a short hinge region comprising CD28 hinge sequences (e.g., SEQ ID NO:44), (vi) a transmembrane region (TM) comprising CD28 transmembrane sequence (e.g., SEQ ID NO:47), and (vii) an intracellular signaling region comprising 4-1BB signaling sequence (e.g., SEQ ID NO:48) and CD3-zeta signaling sequence having only ITAM motif3 (e.g., SEQ ID NO:51); (b) a second polypeptide chain comprising regions ordered from the amino terminus to the carboxyl terminus: (i) a second masking moiety, (ii) a second peptide linker, (iii) an antibody light chain variable region (VL) (e.g., kappa or lambda), and (iv) an antibody light chain constant region (CL). In one embodiment, the V3 dimeric antigen receptors (DAR) lack a peptide linker and/or lack a masking moiety.

In one embodiment, the antibody heavy chain variable region (VH) comprises an anti-EGFR heavy chain variable region sequence (e.g., from SEQ ID NO:2 or 4) and the antibody heavy chain constant region (CH) comprises an anti-EGFR heavy chain constant region sequence (e.g., from SEQ ID NO:2 or 4). In one embodiment, the antibody heavy chain variable region (VH) comprises an anti-CD38 heavy chain variable region sequence (e.g., from SEQ ID NO:7, 9, 11, 13, 14, 15, 16, 17, 18 or 20) and the antibody heavy chain constant region (CH) comprises an anti-CD38 heavy chain constant region sequence (e.g., from SEQ ID NO: 7, 9, 11, 13, 14, 15, 16, 17, 18 or 20).

In one embodiment, the V3 DAR antibody light chain variable region (VL) comprises an anti-EGFR light chain variable region sequence (e.g., from SEQ ID NO:3 or 5) and the antibody light chain constant region (CL) comprises an anti-EGFR light chain constant region sequence (e.g., from SEQ ID NO:3 or 5). In one embodiment, the V3 DAR antibody light chain variable region (VL) comprises an anti-CD38 light chain variable region sequence (e.g., from SEQ ID NO:8, 10, 12 or 19) and the antibody light chain constant region (CL) comprises an anti-CD38 light chain constant region sequence (e.g., from SEQ ID NO:8, 10, 12 or 19).

In one embodiment, the first polypeptide chain of the Version 3 (V3) DAR construct comprises a PDGFR beta hinge sequence (SEQ ID NO:41) and a PDGFR beta transmembrane sequence (SEQ ID NO:42).

The present disclosure provides a Version 4 (e.g., V4) dimeric antigen receptors (DAR) construct comprising a first polypeptide chain carrying heavy chain variable (VH) and heavy chain constant regions (CH), and a second polypeptide chain carrying light chain variable (VL) and light chain constant regions (CL), wherein (a) the first polypeptide chain comprising regions ordered from the amino terminus to the carboxyl terminus: (i) a first masking moiety, (ii) a first peptide linker, (iii) an antibody heavy chain variable region (VH), (iv) an antibody heavy chain constant region (CH), (v) a transmembrane region (TM) comprising CD28 transmembrane sequence (e.g., SEQ ID NO:47), and (vi) an intracellular signaling region comprising 4-1BB signaling sequence (e.g., SEQ ID NO:48) and CD3-zeta signaling sequence having only ITAM motif 3 (e.g., SEQ ID NO:51); (b) a second polypeptide chain comprising regions ordered from the amino terminus to the carboxyl terminus: (i) a second masking moiety, (ii) a second peptide linker, (iii) an antibody light chain variable region (VL) (e.g., kappa or lambda), and (iv) an antibody light chain constant region (CL). The DAR V4 construct lacks a hinge sequence. In one embodiment, the V4 dimeric antigen receptors (DAR) lack a peptide linker and/or lack a masking moiety.

In one embodiment, the antibody heavy chain variable region (VH) comprises an anti-EGFR heavy chain variable region sequence (e.g., from SEQ ID NO:2 or 4) and the antibody heavy chain constant region (CH) comprises an anti-EGFR heavy chain constant region sequence (e.g., SEQ ID NO:2 or 4). In one embodiment, the antibody heavy chain variable region (VH) comprises an anti-CD38 heavy chain variable region sequence (e.g., from SEQ ID NO:7, 9, 11, 13, 14, 15, 16, 17, 18 or 20) and the antibody heavy chain constant region (CH) comprises an anti-CD38 heavy chain constant region sequence (e.g., from SEQ ID NO: 7, 9, 11, 13, 14, 15, 16, 17, 18 or 20).

In one embodiment, the V4 DAR antibody light chain variable region (VL) comprises an anti-EGFR light chain variable region sequence (e.g., from SEQ ID NO:3 or 5) and the antibody light chain constant region (CL) comprises an anti-EGFR light chain constant region sequence (e.g., from SEQ ID NO:3 or 5). In one embodiment, the V4 DAR antibody light chain variable region (VL) comprises an anti-CD38 light chain variable region sequence (e.g., from SEQ ID NO:8, 10, 12 or 19) and the antibody light chain constant region (CL) comprises an anti-CD38 light chain constant region sequence (e.g., from SEQ ID NO:8, 10, 12 or 19).

In one embodiment, the first polypeptide chain of the Version 4 (V4) DAR construct comprises a PDGFR beta hinge sequence (SEQ ID NO:41) and a PDGFR beta transmembrane sequence (SEQ ID NO:42).

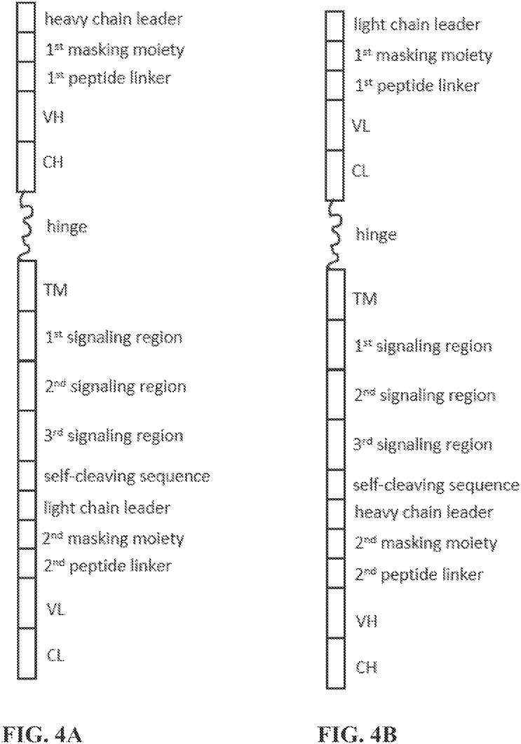

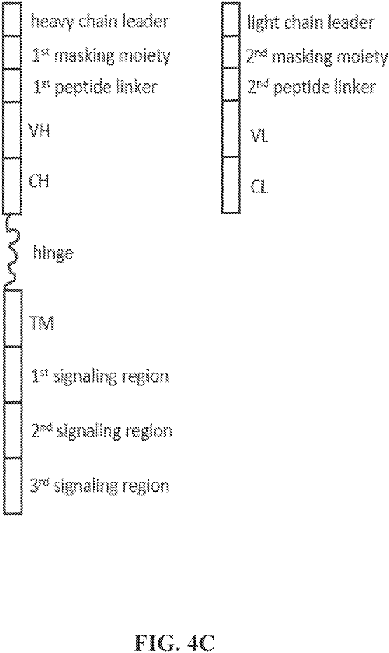

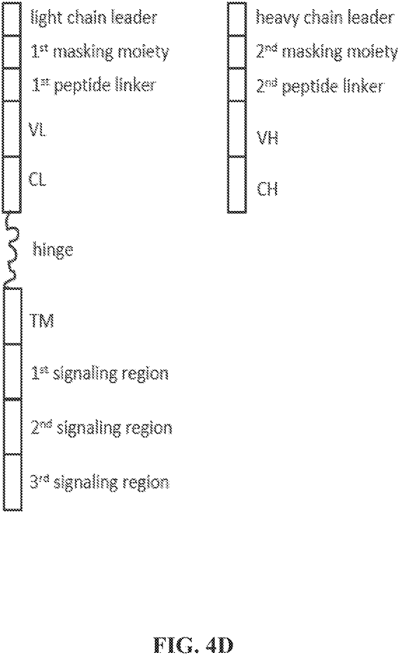

The present disclosure provides nucleic acids encoding a precursor polypeptide (e.g., FIG. 4A and B) that can be processed (cleaved) and assembled to become any of the activatable masked antigen binding proteins comprising a dimeric antigen receptor (DAR) described herein, including the second and third generation DAR constructs, and including the Versions V1, V2a, V2b, V2c, V3 and V4 DAR constructs, and including any of the activatable masked antigen binding proteins comprising a dimeric antigen receptor (DAR) that can bind EGFR or CD38.

In one embodiment, the nucleic acid encodes a precursor polypeptide chain comprising (i) heavy chain leader sequence, (ii) a first masking moiety, (iii) a first peptide linker, (iv) an antibody heavy chain variable region (VH), (v) an antibody heavy chain constant region (CH), (vi) an optional hinge region, (vii) a transmembrane region (TM), (viii) an intracellular signaling region, (ix) a self-cleaving sequence, (x) a light chain leader sequence, (xi) a second masking moiety, (xii) a second peptide linker, (xiii) an antibody light chain variable region (VL) (e.g., kappa or lambda), and (xiv) an antibody light chain constant region (CL). In one embodiment, the first peptide linker comprises a first cleavable site. In one embodiment, the second peptide linker comprises a second cleavable site. In one embodiment, the self-cleaving sequence comprises T2A (e.g., amino acid sequence EGRGSLLTCGDVEENPGP, P2A (e.g., amino acid sequence (GSG)ATNFSLLKQAGDVEENPG, PE2A (e.g., amino acid sequence (GSG)QCTNYALLKLAGDVESNPG, PF2A (e.g., amino acid sequence (GSG)VKQTLNFDLLKLAGDVESNPGP, wherein the amino acid sequence (GSG) is optional. In one embodiment, the nucleic acid encoding the precursor polypeptide chain comprises a recombinant nucleic acid molecule.

In one embodiment, the nucleic acid encodes a precursor polypeptide chain comprising (i) light chain leader sequence, (ii) a first masking moiety, (iii) a first peptide linker, (iv) an antibody light chain variable region (VL) (e.g., kappa or lambda), (v) an antibody light chain constant region (CL), (vi) an optional hinge region, (vii) a transmembrane region (TM), (viii) an intracellular signaling region, (ix) a self-cleaving sequence, (x) a heavy chain leader sequence, (xi) a second masking moiety, (xii) a second peptide linker, (xiii) an antibody heavy chain variable region (VH), and (xiv) an antibody heavy chain constant region (CH). In one embodiment, the first peptide linker comprises a first cleavable site. In one embodiment, the second peptide linker comprises a second cleavable site. In one embodiment, the second peptide linker comprises a second cleavable site. In one embodiment, the self-cleaving sequence comprises T2A (e.g., amino acid sequence EGRGSLLTCGDVEENPGP), P2A (e.g., amino acid sequence (GSG)ATNFSLLKQAGDVEENPG), PE2A (e.g., amino acid sequence (GSG)QCTNYALLKLAGDVESNPG), PF2A (e.g., amino acid sequence (GSG)VKQTLNFDLLKLAGDVESNPGP), wherein the amino acid sequence (GSG) is optional. In one embodiment, the nucleic acid encoding the precursor polypeptide chain comprises a recombinant nucleic acid molecule.

In one embodiment, the nucleic acid encodes a precursor polypeptide chain comprising a first and/or second masking moiety comprising a CH1, CL (lambda or kappa) region having the amino acid sequence of SEQ ID NO:21, 22 or 23. In one embodiment, the nucleic acid encodes a precursor polypeptide chain comprising a first and/or second masking moiety comprising a CH3 (IgG1 or IgG4) region having the amino acid sequence of SEQ ID NO:24, 25, 30 or 31. In one embodiment, the nucleic acid encodes a precursor polypeptide chain comprising a first and/or second masking moiety comprising a T cell receptor alpha (α) or beta (β) constant region having the amino acid sequence of SEQ ID NO:28 or 29. In one embodiment, the nucleic acid encodes a precursor polypeptide chain comprising a first and/or second masking moiety a variable heavy chain domain from catalytic antibody 38C2 having the amino acid sequence of SEQ ID NO:26 or 27.

In one embodiment, the nucleic acid encodes a precursor polypeptide chain comprising a first and/or second peptide linker comprising the amino acid sequence of one of SEQ ID NO:32-40 or comprising any one of the amino acid sequences listed in Tables 3-9.

In one embodiment, the nucleic acid encodes a precursor polypeptide chain comprising an antibody heavy chain variable region (VH) which comprises an anti-EGFR heavy chain variable region sequence having an amino acid sequence having at least 92%, or at least 93%, or at least 94%, or at least 95%), or at least 96%, or at least 97%, or at least 98%, or at least 99% sequence identity, or having 100% sequence identity, to a portion or full-length of SEQ ID NO:2 or 4. In one embodiment, the nucleic acid encodes a precursor polypeptide chain comprising an antibody heavy chain variable region (VH) which comprises an anti-CD38 heavy chain variable region sequence having an amino acid sequence having at least 92%, or at least 93%, or at least 94%, or at least 95%, or at least 96%, or at least 97%, or at least 98%, or at least 99% sequence identity, or having 100% sequence identity, to a portion or full-length of SEQ ID NO:7, 9, 11, 13, 14, 15, 16, 17, 18 or 20.

In one embodiment, the nucleic acid encodes a precursor polypeptide chain comprising an antibody heavy chain constant region (CH) which comprises an anti-EGFR heavy chain constant region sequence (e.g., from SEQ ID NO:2 or 4). In one embodiment, the nucleic acid encodes a precursor polypeptide chain comprising an antibody heavy chain constant region (CH) which comprises an anti-CD38 heavy chain constant region sequence (e.g., from SEQ ID NO: 7, 9, 11, 13, 14, 15, 16, 17, 18 or 20).

In one embodiment, the nucleic acid encodes a precursor polypeptide chain comprising an antibody light chain variable region (VL) which comprises an anti-EGFR light chain variable region sequence having an amino acid sequence having at least 92%, or at least 93%, or at least 94%, or at least 95%, or at least 96%, or at least 97%, or at least 98%, or at least 99% sequence identity, or having 100% sequence identity, to a portion or full-length of SEQ ID NO:3 or 5. In one embodiment, the nucleic acid encodes a precursor polypeptide chain comprising an antibody light chain variable region (VL) which comprises an anti-CD38 light chain variable region sequence having an amino acid sequence having at least 92%, or at least 93%, or at least 94%, or at least 95%, or at least 96%, or at least 97%, or at least 98%, or at least 99% sequence identity, or having 100% sequence identity, to a portion or full-length of SEQ ID NO:8, 10, 12 or 19.

In one embodiment, the nucleic acid encodes a precursor polypeptide chain comprising an antibody light chain constant region (CL) which comprises an anti-EGFR light chain constant region sequence (e.g., from SEQ ID NO:3 or 5). In one embodiment, the nucleic acid encodes a precursor polypeptide chain comprising an antibody light chain constant region (CL) which comprises an anti-CD38 light chain constant region sequence (e.g., from SEQ ID NO:8, 10, 12 or 19).

In one embodiment, the nucleic acid encodes a precursor polypeptide chain comprising a hinge region having the amino acid sequence of any one or any combination of two or more of: a CD28 hinge sequence (SEQ ID NO:44); a CD8 hinge sequence (SEQ ID NO:45); a long hinge comprising CD28 and CD8 hinge sequences (SEQ ID NO:46); and/or a PDGFR beta hinge sequence (SEQ ID NO:41).

In one embodiment, the nucleic acid encodes a precursor polypeptide chain comprising transmembrane region having the amino acid sequence of a CD28 transmembrane sequence (SEQ ID NO:47) or a PDGFR beta transmembrane sequence (SEQ ID NO:42).

In one embodiment, the nucleic acid encodes a precursor polypeptide chain comprising 1 - 3 intracellular signaling sequences in any order and in any combination having the amino acid sequence(s) of: a 4-1BB signaling sequence (SEQ ID NO:48); a CD28 signaling sequence (SEQ ID NO:49); a CD3zeta signaling sequence (SEQ ID NO:50); and/or a CD3zeta short signaling sequence (SEQ ID NO:51).

The present disclosure provides a first nucleic acid encoding a first polypeptide chain and a second nucleic acid encoding a second polypeptide chain (e.g., FIG. 4C and D), wherein the first and second polypeptide chains can associate/dimerize with each other to form any of the activatable masked antigen binding proteins comprising a dimeric antigen receptor (DAR) described herein, including the second and third generation DAR constructs, and including the Versions V1, V2a, V2b, V2c, V3 and V4 DAR constructs, and including any of the activatable masked antigen binding proteins comprising a dimeric antigen receptor (DAR) that can bind EGFR or CD38.

In one embodiment, the first nucleic acid encodes the first polypeptide chain comprising (i) heavy chain leader sequence, (ii) a first masking moiety, (iii) a first peptide linker, (iv) an antibody heavy chain variable region (VH), (v) an antibody heavy chain constant region (CH), (vi) an optional hinge region, (vii) a transmembrane region (TM), and (viii) an intracellular signaling region (e.g., FIG. 4C).

In one embodiment, the second nucleic acid encodes the second polypeptide chain comprising (i) a light chain leader sequence, (ii) a second masking moiety, (iii) a second peptide linker, (iv) an antibody light chain variable region (VL) (e.g., kappa or lambda), and (x) an antibody light chain constant region (CL) (e.g., FIG. 4C).

In one embodiment, the first peptide linker comprises a first cleavable site. In one embodiment, the second peptide linker comprises a second cleavable site. In one embodiment, the first and second nucleic acids which encode the first and second chains, respectively, comprise recombinant nucleic acid molecules.

In one embodiment, the first nucleic acid encodes the first polypeptide chain comprising (i) light chain leader sequence, (ii) a first masking moiety, (iii) a first peptide linker, (iv) an antibody light chain variable region (VL) (e.g., kappa or lambda), (v) an antibody light chain constant region (CL), (vi) an optional hinge region, (vii) a transmembrane region (TM), and (viii) an intracellular signaling region (e.g., FIG. 4D).

In one embodiment, the second nucleic acid encodes (i) a heavy chain leader sequence, (ii) a second masking moiety, (iii) a second peptide linker, (iv) an antibody heavy chain variable region (VH), and (x) an antibody heavy chain constant region (CH) (e.g., FIG. 4D).

In one embodiment, the first peptide linker comprises a first cleavable site. In one embodiment, the second peptide linker comprises a second cleavable site. In one embodiment, the first and second nucleic acids which encode the first and second chains, respectively, comprise recombinant nucleic acid molecules.

In one embodiment, the first nucleic acid encodes the first polypeptide chain comprising a first masking moiety comprising a CH1, CL (lambda or kappa) region having the amino acid sequence of SEQ ID NO:21, 22 or 23. In one embodiment, the first nucleic acid encodes the first polypeptide chain comprising a first masking moiety comprising a CH3 (IgG1 or IgG4) region having the amino acid sequence of SEQ ID NO:24, 25, 30 or 31. In one embodiment, the first nucleic acid encodes the first polypeptide chain comprising a first masking moiety comprising a T cell receptor alpha (a) or beta (β) constant region having the amino acid sequence of SEQ ID NO:28 or 29. In one embodiment, the first nucleic acid encodes the first polypeptide chain comprising a first masking moiety comprising a variable heavy chain domain from catalytic antibody 38C2 having the amino acid sequence of SEQ ID NO:26 or 27.

In one embodiment, the second nucleic acid encodes the second polypeptide chain comprising a second masking moiety comprising a CH1, CL (lambda or kappa) region having the amino acid sequence of SEQ ID NO:21, 22 or 23. In one embodiment, the second nucleic acid encodes the second polypeptide chain comprising a second masking moiety comprising a CH3 (IgG1 or IgG4) region having the amino acid sequence of SEQ ID NO:24, 25, 30 or 31. In one embodiment, the second nucleic acid encodes the second polypeptide chain comprising a second masking moiety comprising a T cell receptor alpha (α) or beta (β) constant region having the amino acid sequence of SEQ ID NO:28 or 29. In one embodiment, the second nucleic acid encodes the second polypeptide chain comprising a second masking moiety comprising a variable heavy chain domain from catalytic antibody 38C2 having the amino acid sequence of SEQ ID NO:26 or 27.

In one embodiment, the first nucleic acid encodes the first polypeptide chain comprising a first peptide linker comprising the amino acid sequence of one of SEQ ID NO:32-40 or comprising any one of the amino acid sequences listed in Tables 3-9.

In one embodiment, the second nucleic acid encodes the second polypeptide chain comprising a second peptide linker comprising the amino acid sequence of one of SEQ ID NO:32-40 or comprising any one of the amino acid sequences listed in Tables 3-9.

In one embodiment, the first nucleic acid encodes the first polypeptide chain comprising an antibody heavy chain variable region (VH) which comprises an anti-EGFR heavy chain variable region sequence having an amino acid sequence having at least 92%, or at least 93%, or at least 94%, or at least 95%, or at least 96%, or at least 97%, or at least 98%, or at least 99% sequence identity, or having 100% sequence identity, to a portion or full-length of SEQ ID NO:2 or 4. In one embodiment, the first nucleic acid encodes the first polypeptide chain comprising an antibody heavy chain variable region (VH) which comprises an anti-CD38 heavy chain variable region sequence having an amino acid sequence having at least 92%, or at least 93%, or at least 94%, or at least 95%, or at least 96%, or at least 97%, or at least 98%, or at least 99% sequence identity, or having 100% sequence identity, to a portion or full-length of SEQ ID NO:7, 9, 11, 13, 14, 15, 16, 17, 18 or 20.

In one embodiment, the first nucleic acid encodes the first polypeptide chain comprising an antibody light chain variable region (VL) which comprises an anti-EGFR light chain variable region sequence having an amino acid sequence having at least 92%, or at least 93%, or at least 94%, or at least 95%, or at least 96%, or at least 97%, or at least 98%, or at least 99% sequence identity, or having 100% sequence identity, to a portion or full-length of SEQ ID NO:3 or 5. In one embodiment, the first nucleic acid encodes the first polypeptide chain comprising an antibody light chain variable region (VL) which comprises an anti-CD38 light chain variable region sequence having an amino acid sequence having at least 92%, or at least 93%, or at least 94%, or at least 95%, or at least 96%, or at least 97%, or at least 98%, or at least 99% sequence identity, or having 100% sequence identity, to a portion or full-length of SEQ ID NO:8, 10, 12 or 19.

In one embodiment, the second nucleic acid encodes the second polypeptide chain comprising an antibody light chain variable region (VL) which comprises an anti-EGFR light chain variable region sequence having an amino acid sequence having at least 92%, or at least 93%, or at least 94%, or at least 95%, or at least 96%, or at least 97%, or at least 98%, or at least 99% sequence identity, or having 100% sequence identity, to a portion or full-length of SEQ ID NO:3 or 5. In one embodiment, the second nucleic acid encodes the second polypeptide chain comprising an antibody light chain variable region (VL) which comprises an anti-CD38 light chain variable region sequence having an amino acid sequence having at least 92%, or at least 93%, or at least 94%, or at least 95%, or at least 96%, or at least 97%, or at least 98%, or at least 99% sequence identity, or having 100% sequence identity, to a portion or full-length of SEQ ID NO:8, 10, 12 or 19.

In one embodiment, the second nucleic acid encodes the second polypeptide chain comprising an antibody heavy chain variable region (VH) which comprises an anti-EGFR heavy chain variable region sequence having an amino acid sequence having at least 92%, or at least 93%, or at least 94%, or at least 95%, or at least 96%, or at least 97%, or at least 98%, or at least 99% sequence identity, or having 100% sequence identity, to a portion or full-length of SEQ ID NO:2 or 4. In one embodiment, the second nucleic acid encodes the second polypeptide chain comprising an antibody heavy chain variable region (VH) which comprises an anti-CD38 heavy chain variable region sequence having an amino acid sequence having at least 92%, or at least 93%, or at least 94%, or at least 95%, or at least 96%, or at least 97%, or at least 98%, or at least 99% sequence identity, or having 100% sequence identity, to a portion or full-length of SEQ ID NO:7, 9, 11, 13, 14, 15, 16, 17, 18 or 20.

In one embodiment, the first nucleic acid encodes the first polypeptide chain comprising an antibody heavy chain constant region (CH) which comprises an anti-EGFR heavy chain constant region sequence (e.g., from SEQ ID NO:2 or 4). In one embodiment, the first nucleic acid encodes the first polypeptide chain comprising an antibody heavy chain constant region (CH) which comprises an anti-CD38 heavy chain constant region sequence (e.g., from SEQ ID NO: 7, 9, 11, 13, 14, 15, 16, 17, 18 or 20).

In one embodiment, the first nucleic acid encodes the first polypeptide chain comprising an antibody light chain constant region (CL) which comprises an anti-EGFR light chain constant region sequence (e.g., from SEQ ID NO:3 or 5). In one embodiment, the first nucleic acid encodes the first polypeptide chain comprising an antibody light chain constant region (CL) which comprises an anti-CD38 light chain constant region sequence (e.g., from SEQ ID NO:8, 10, 12 or 19).

In one embodiment, the second nucleic acid encodes the second polypeptide chain comprising an antibody light chain constant region (CL) which comprises an anti-EGFR light chain constant region sequence (e.g., from SEQ ID NO:3 or 5). In one embodiment, the second nucleic acid encodes the second polypeptide chain comprising an antibody light chain constant region (CL) which comprises an anti-CD38 light chain constant region sequence (e.g., from SEQ ID NO:8, 10, 12 or 19).

In one embodiment, the second nucleic acid encodes the second polypeptide chain comprising an antibody heavy chain constant region (CH) which comprises an anti-EGFR heavy chain constant region sequence (e.g., from SEQ ID NO:2 or 4). In one embodiment, the second nucleic acid encodes the second polypeptide chain comprising an antibody heavy chain constant region (CH) which comprises an anti-CD38 heavy chain constant region sequence (e.g., from SEQ ID NO: 7, 9, 11, 13, 14, 15, 16, 17, 18 or 20).

In one embodiment, the first nucleic acid encodes the first polypeptide chain comprising a hinge region having the amino acid sequence of any one or any combination of two or more of: a CD28 hinge sequence (SEQ ID NO:44); a CD8 hinge sequence (SEQ ID NO:45); a long hinge comprising CD28 and CD8 hinge sequences (SEQ ID NO:46); and/or a PDGFR beta hinge sequence (SEQ ID NO:41).

In one embodiment, the first nucleic acid encodes the first polypeptide chain comprising a transmembrane region having the amino acid sequence of a CD28 transmembrane sequence (SEQ ID NO:47) or a PDGFR beta transmembrane sequence (SEQ ID NO:42).

In one embodiment, the first nucleic acid encodes the first polypeptide chain comprising 1-3 intracellular signaling sequences in any order and in any combination having the amino acid sequence(s) of: a 4-1BB signaling sequence (SEQ ID NO:48); a CD28 signaling sequence (SEQ ID NO:49); a CD3zeta signaling sequence (SEQ ID NO:50); and/or a CD3zeta short signaling sequence (SEQ ID NO:51).

The present disclosure provides vectors, including expression vectors, that are operably joined to nucleic acids encoding a precursor polypeptide of any of the activatable masked antigen binding proteins comprising a dimeric antigen receptor (DAR).

In one embodiment, the expression vector comprises one or more regulatory sequences (e.g., promoter and/or enhancer) that control transcription of the nucleic acids encoding any of the precursor polypeptides of the activatable masked antigen binding proteins comprising a dimeric antigen receptor (DAR). In one embodiment, the expression vector comprises one or more regulatory sequences each operably joined to a nucleic acid encoding the precursor polypeptide.

In one embodiment, an expression vector is introduced into a host cell, wherein the expression vector within the host cell carries a promoter (and optionally an enhancer sequence), that is operably joined to a nucleic acid encoding a precursor polypeptide. Thus, the host cell can express (transcription and translation) the encoded precursor polypeptide that makes up any of the activatable masked antigen binding proteins comprising a dimeric antigen receptor (DAR) described herein.

In one embodiment, the precursor polypeptide can be processed by cleaving the precursor polypeptide at the self-cleaving sequence to release the first and second polypeptide chains and secreting the precursor, and/or anchoring the precursor in the cellular membrane of the host cell. In one embodiment, the precursor polypeptide can be cleaved at the self-cleaving sequence thereby generating first and second polypeptide chains each having a leader signal sequence at their N-terminal ends.

In one embodiment, after release of the first and second polypeptide chains, the first and second polypeptide chains can dimerize (assemble) via at least one disulfide bond between the antibody heavy chain constant region and the antibody light chain constant region.

The vectors comprise promoters that are inducible or constitutive promoters. The vectors and host cells can be selected to generate transgenic host cells that transiently or stably express any of the precursor polypeptides described herein.

The expression vector can include nucleic acid backbone sequences derived from a retrovirus, lentivirus or adenovirus. The expression vectors can include one or more regulatory sequences, such as inducible and/or constitutive promoters and enhancers. The expression vectors can include ribosomal binding sites and/or polyadenylation sites.

In one embodiment, the expression vector, which is operably linked to the nucleic acid encoding the dimeric antigen receptor (DAR) construct, can direct production of the dimeric antigen receptor (DAR) construct which can be displayed on the surface of the transgenic host cell or the dimeric antigen receptor can be secreted into the cell culture medium.

The present disclosure provides vectors that are operably joined to nucleic acids encoding a first polypeptide chain and/or encoding a second polypeptide chain, wherein the first and second polypeptide chains associate/dimerize with each other to form an the activatable masked antigen binding proteins comprising a dimeric antigen receptor (DAR).

In one embodiment, a first vector (e.g., a first expression vector) is operably joined to a first nucleic acid encoding a first polypeptide chain of any of the activatable masked antigen binding proteins comprising a dimeric antigen receptor (DAR).

In one embodiment, a second vector (e.g., a second expression vector) is operably joined to a second nucleic acid encoding a second polypeptide chain of any of the activatable masked antigen binding proteins comprising a dimeric antigen receptor (DAR).

In one embodiment, a first vector (e.g., a first expression vector) that is operably joined to a first nucleic acid encoding a first polypeptide chain of any of the activatable masked antigen binding proteins comprising a dimeric antigen receptor (DAR), and the first vector is also operably joined to a second nucleic acid encoding a second polypeptide chain of any of the activatable masked antigen binding proteins comprising a dimeric antigen receptor (DAR).

In one embodiment, the expression vector comprises one or more regulatory sequences (e.g., promoter and/or enhancer) that control transcription of the nucleic acids encoding any of the precursor polypeptides of the activatable masked antigen binding proteins comprising a dimeric antigen receptor (DAR). In one embodiment, the expression vector comprises one or more regulatory sequences each operably joined to a nucleic acid encoding the precursor polypeptide.

In one embodiment, an expression vector is introduced into a host cell, wherein the expression vector within the host cell carries a promoter (and optionally an enhancer sequence), that is operably joined to a nucleic acid encoding a precursor polypeptide. Thus, the host cell can express (transcription and translation) the encoded precursor polypeptide that makes up any of the activatable masked antigen binding proteins comprising a dimeric antigen receptor (DAR) described herein.

In one embodiment, after expression of the first and second polypeptide chains, the first and second polypeptide chains can dimerize (assemble) via at least one disulfide bond between the antibody heavy chain constant region and the antibody light chain constant region (e.g., FIG. 3A and B).

The vectors comprise promoters that are inducible or constitutive promoters. The vectors and host cells can be selected to generate transgenic host cells that transiently or stably express any of the precursor polypeptides described herein.

The expression vector can include nucleic acid backbone sequences derived from a retrovirus, lentivirus or adenovirus. The expression vectors can include one or more regulatory sequences, such as inducible and/or constitutive promoters and enhancers. The expression vectors can include ribosomal binding sites and/or polyadenylation sites.

In one embodiment, the expression vector, which is operably linked to the nucleic acid encoding the dimeric antigen receptor (DAR) construct, can direct production of the dimeric antigen receptor (DAR) construct which can be displayed on the surface of the transgenic host cell or the dimeric antigen receptor can be secreted into the cell culture medium.

The present disclosure provides a host cell, or a population of host cells, that harbor one or more expression vectors that are operably joined to any of the nucleic acids that encode the first and/or second polypeptide chains that can associate/dimerize with each other to become an activatable masked antigen binding proteins comprising a dimeric antigen receptor (DAR).

In one embodiment, a first host cell, or a population of first host cells, harbors a first vector (e.g., a first expression vector) which is operably joined to a first nucleic acid encoding a first polypeptide chain of any of the activatable masked antigen binding proteins comprising a dimeric antigen receptor (DAR).

In one embodiment, a second host cell, or a population of second host cells, harbors a second vector (e.g., a second expression vector) which is operably joined to a second nucleic acid encoding a second polypeptide chain of any of the activatable masked antigen binding proteins comprising a dimeric antigen receptor (DAR).

In one embodiment, a first host cell, or a population of first host cells, harbors a first vector (e.g., a first expression vector) which is operably joined to a first nucleic acid encoding a first polypeptide chain of any of the activatable masked antigen binding proteins comprising a dimeric antigen receptor (DAR), and the first host cell or the population of first host cells also harbors a second vector which is operably joined to a second nucleic acid encoding a second polypeptide chain of any of the activatable masked antigen binding proteins comprising a dimeric antigen receptor (DAR).

In one embodiment, a first host cell, or a population of first host cells, harbors a first vector (e.g., a first expression vector) which is operably joined to a first nucleic acid encoding a first polypeptide chain of any of the activatable masked antigen binding proteins comprising a dimeric antigen receptor (DAR), and the first vector is also operably joined to a second nucleic acid encoding a second polypeptide chain of any of the activatable masked antigen binding proteins comprising a dimeric antigen receptor (DAR).

The host cell can be a bacterial or mammalian cell. In one embodiment, the host cell comprises a Chinese hamster ovary (CHO) cell. The host cell or the population of host cells include T lymphocytes (e.g., T cells, regulatory T cells, gamma-delta T cells, and cytotoxic T cells), NK (natural killer) cells, macrophages, dendritic cells, mast cells, eosinophils, B lymphocytes, monocytes. In one embodiment, the NK cells comprise cord blood-derived NK cells, or placental derived NK cells.

In one embodiment, the first expression vector is introduced into the first host cell or the population of the first host cell, wherein the first expression vector is operably linked to a first nucleic acid encoding the first polypeptide chain of any of the activatable masked antigen binding proteins comprising a dimeric antigen receptor (DAR).

In one embodiment, the second expression vector is introduced into the second host cell or the population of the second host cell, wherein the second expression vector is operably linked to a second nucleic acid encoding the second polypeptide chain of any of the activatable masked antigen binding proteins comprising a dimeric antigen receptor (DAR).

In one embodiment, the first and the second expression vectors are introduced into the first host cell or the population of the first host cell, wherein the first expression vector is operably linked to a first nucleic acid encoding the first polypeptide chain of any of the activatable masked antigen binding proteins comprising a dimeric antigen receptor (DAR), and wherein the second expression vector is operably linked to a second nucleic acid encoding the second polypeptide chain of any of the activatable masked antigen binding proteins comprising a dimeric antigen receptor (DAR).

In one embodiment, the first expression vector is introduced into the first host cell or the population of the first host cell, wherein the first expression vector is operably linked to a first nucleic acid encoding the first polypeptide chain of any of the activatable masked antigen binding proteins comprising a dimeric antigen receptor (DAR), and wherein the first expression vector is also operably linked to a second nucleic acid encoding the second polypeptide chain of any of the activatable masked antigen binding proteins comprising a dimeric antigen receptor (DAR).

In one embodiment, at least one expression vector is introduced into the host cell via lipofection (e.g., using a lipid surfactant); electroporation; transfection employing calcium chloride, rubidium chloride, calcium phosphate, DEAE-dextran, or other substances; viral transfection; non-viral transfection; microprojectile bombardment; and infection (e.g., where the vector is an infectious agent).

The present disclosure provide methods for preparing any of the activatable masked antigen binding proteins comprising a dimeric antigen receptor (DAR) described herein, including the second and third generation DAR constructs, and including the Versions V1, V2a, V2b, V2c, V3 and V4 DAR constructs, and including any of the activatable masked antigen binding proteins comprising a dimeric antigen receptor (DAR) that can bind EGFR or CD38. Methods for preparing the activatable masked antigen binding proteins comprising a dimeric antigen receptor (DAR) comprise: culturing a population of host cells, wherein individual host cells in the population harbor an expression vector that is operably linked to a first and/or a second nucleic acid encoding a first and/or second polypeptide chain described herein, wherein the culturing is conducted under conditions suitable for expressing the first and/or second polypeptide by the population of host cells.

In one embodiment, the nucleic acids encoding the first and/or second polypeptide chain further encodes a light chain and/or heavy chain leader sequence for secretion of the expressed first and/or second polypeptides by the host cell. In one embodiment, the culturing is conducted under conditions suitable for secretion of the first and/or second polypeptide by the population of host cells.

In one embodiment, the nucleic acids encoding the first and/or second polypeptide further encodes an affinity tag sequence for enriching the expressed polypeptides. Exemplary affinity tag sequences include histidine tag, FLAG tag, myc tag, HA tag, and GST tag.

In one embodiment, the methods for preparing any of the activatable masked antigen binding proteins comprising an IgG type antibody described herein further comprise isolating the expressed heavy chains and/or light chains.

In one embodiment, the culturing is conducted under conditions that are suitable for assembly or association of the heavy chain and light chain to form the activatable masked antigen binding proteins comprising an IgG type antibody.

In one embodiment, the method for preparing any of the activatable masked antigen binding proteins comprising a DAR type antibody described herein further comprises isolating the expressed precursor DAR polypeptides. In one embodiment, the method for preparing any of the activatable masked antigen binding proteins comprising a DAR type antibody described herein further comprises isolating the processed/cleaved/assembled polypeptides that form the dimeric antigen receptors (DAR).

In one embodiment, the culturing is conducted under conditions that are suitable for expression, secretion and cleavage of the precursor DAR polypeptide, and assembly of the two resulting polypeptide chains (first and second polypeptide chains) to form the activatable masked antigen binding proteins comprising a dimeric antigen receptor (DAR).

In one embodiment, the method for preparing any of the activatable masked antigen binding proteins comprising a DAR type antibody described herein further comprises isolating the expressed first and/or second polypeptides. In one embodiment, the method for preparing any of the activatable masked antigen binding proteins comprising a DAR type antibody described herein further comprises isolating the dimeric antigen receptors (DAR) which is formed by association/dimerization of the first and second Dar polypeptide chains.

In one embodiment, the culturing is conducted under conditions that are suitable for expression of the first and/or second polypeptide chains, and assembly of the first and second polypeptide chains to form the activatable masked antigen binding proteins comprising a dimeric antigen receptor (DAR).

In one embodiment, the method further comprises isolating or recovering the assembled activatable masked antigen binding proteins comprising the dimeric antigen receptor (DAR). In one embodiment, the isolating is conducted using affinity chromatography. In one embodiment, the isolating is conducted using affinity chromatography with protein A or G from Staphylococcus aureus, glutathione S-transferase (GST), or immuno-affinity. In one embodiment, one or more additional isolating steps are conducted which includes cation exchange, anion exchange chromatography, hydrophobic interaction chromatography, mixed mode chromatography and/or hydroxyapatite chromatography.

In one embodiment, the assembled activatable masked antigen binding proteins comprising the dimeric antigen receptor (DAR) comprises a first and a second polypeptide chain, wherein the (a) first polypeptide chain comprises: (i) a first masking moiety, (ii) a first peptide linker, (iii) an antibody heavy chain variable region (VH), (iv) an antibody heavy chain constant region (CH), (v) an optional hinge region, (vi) a transmembrane region (TM), and (vii) an intracellular signaling region, wherein the (b) second polypeptide chain comprises: (i) a second masking moiety, (ii) a second peptide linker, (iii) an antibody light chain variable region (VL) (e.g., kappa or lambda), and (iv) an antibody light chain constant region (CL), wherein the antibody heavy chain variable region (VH) and the antibody light chain variable region (VL) form an antigen binding domain that binds a target antigen.

In one embodiment, the assembled activatable masked antigen binding proteins comprising the dimeric antigen receptor (DAR) comprises a first and a second polypeptide chain, wherein the (a) first polypeptide chain comprises: (i) a first masking moiety, (ii) a first peptide linker, (iii) an antibody light chain variable region (VL) (e.g., kappa or lambda), (iv) an antibody light chain constant region (CL), (v) an optional hinge region, (vi) a transmembrane region (TM), and (vii) an intracellular signaling region, wherein the (b) second polypeptide chain comprises: (i) a second masking moiety, (ii) a second peptide linker, (iii) an antibody heavy chain variable region (VH), and (iv) an antibody heavy chain constant region (CH), wherein the antibody heavy chain variable region (VH) and the antibody light chain variable region (VL) form an antigen binding domain that binds a target antigen.

The activatable masked antigen binding proteins comprising a dimeric antigen receptor (DAR) can be prepared using transgenic host cell expression, phage display, yeast display and human antibody gene transgenic mice using methods that are well known in the art. In one embodiment, the yield of antigen binding proteins using transgenic host cell expression can be about 20-80%, or about 30-90%, or about 40-95%, or about 50-99% of the total activatable masked antigen binding proteins formed.

The present disclosure provide methods for cleaving at least one peptide linker of any of the activatable masked antigen binding proteins comprising a dimeric antigen receptor (DAR) described herein, the method comprising: (a) contacting at least one protease with an activatable masked antigen binding protein DAR in an inactive form, wherein the first and second peptide linkers are in the un-cleaved state.

In one embodiment, the activatable masked antigen binding protein is contacted with an one or any combination of two or more proteases selected from: a matrix metalloprotease (MMP), MMP1, MMP2, MMP3, MMP8, MMP9, MMP11, MMP13, MMP14, MT1-MMP (membrane type 1 matrix metalloproteinase), a disintegrin and metalloproteinase (ADAM) protease, ADAM10, ADAM12, ADAM17, urokinase plasminogen activator (uPA), serine proteases, cysteine proteases, aspartate proteases, threonine proteases, cathepsins (e.g., cysteine or aspartic cathepsins), cathepsin B, cathepsin C, cathepsin D, cathepsin E, cathepsin K or cathepsin L.

In one embodiment, the activatable masked antigen binding protein is contacted essentially simultaneously (at the same time) with two or more proteases, or sequentially contacted with two or more proteases in any order.

In one embodiment, at least one of the peptide linkers of the activatable masked antigen binding protein used in step (a) comprises a cleavable site that is cleavable with one or more proteases, including a matrix metalloprotease (MMP), MMP1, MMP2, MMP3, MMP8, MMP9, MMP11, MMP13, MMP14, MT1-MMP (membrane type 1 matrix metalloproteinase), a disintegrin and metalloproteinase (ADAM) protease, ADAM10, ADAM12, ADAM17, urokinase plasminogen activator (uPA), serine proteases, cysteine proteases, aspartate proteases, threonine proteases, cathepsins (e.g., cysteine or aspartic cathepsins), cathepsin B, cathepsin C, cathepsin D, cathepsin E, cathepsin K or cathepsin L.

In one embodiment, the method further comprises: (b) cleaving at least one of the peptide linkers to convert the un-cleaved activatable masked antigen binding protein to an activated form. In one embodiment, the activated form can bind a target antigen.

In one embodiment, the method further comprises: (c) binding the activatable masked antigen binding protein (now in the activated state) to a target antigen.

In one embodiment, the steps of contacting, cleaving and binding are conducted in an in vitro or in vivo condition.

In one embodiment, the target antigen comprises a soluble antigen or a surface antigen. In one embodiment, the target antigen is expressed by a healthy or diseased cell.

In one embodiment, the diseased cell (e.g., tumor or cancer cell) that expresses the target antigen also expresses one or more proteases that cleaves the peptide linker. In one embodiment, the diseased cell expresses one or a combination of two or more proteases comprising a matrix metalloprotease (MMP), MMP1, MMP2, MMP3, MMP8, MMP9, MMP11, MMP13, MMP14, MT1-MMP (membrane type 1 matrix metalloproteinase), a disintegrin and metalloproteinase (ADAM) protease, ADAM10, ADAM12, ADAM17, urokinase plasminogen activator (uPA), serine proteases, cysteine proteases, aspartate proteases, threonine proteases, cathepsins (e.g., cysteine or aspartic cathepsins), cathepsin B, cathepsin C, cathepsin D, cathepsin E, cathepsin K and/or cathepsin L.

In one embodiment, method further comprises: (d) killing the diseased cell (e.g., tumor or cancer cell) by binding the activated masked antigen binding protein to the diseased cell.

In one embodiment, at least one of the peptide linkers is cleavable with a protease that is present in a tumor microenvironment.

In one embodiment, the tumor microenvironment comprise a protease selected from a group consisting of a matrix metalloprotease (MMP), MMP1, MMP2, MMP3, MMP8, MMP9, MMP11, MMP13, MMP14, MT1-MMP (membrane type 1 matrix metalloproteinase), a disintegrin and metalloproteinase (ADAM) protease, ADAM10, ADAM12, ADAM17, urokinase plasminogen activator (uPA), serine proteases, cysteine proteases, aspartate proteases, threonine proteases, cathepsins (e.g., cysteine or aspartic cathepsins), cathepsin B, cathepsin C, cathepsin D, cathepsin E, cathepsin K and cathepsin L.

The present disclosure provide methods treating a subject having a disease associated with expression or over-expression of a tumor-associated antigen, the method comprising: administering to the subject an effective amount of a therapeutic composition comprising a population of host cells expressing any of the activatable masked antigen binding proteins comprising a dimeric antigen receptor (DAR) described herein. In one embodiment, the population of host cells express the activatable masked antigen binding proteins comprising a dimeric antigen receptor (DAR) in an inactive form having the first and second peptide linkers in the un-cleaved state (e.g., activatable). In one embodiment, the population of host cells can express any of the activatable masked antigen binding proteins comprising a dimeric antigen receptor (DAR), including the second and third generation DAR constructs, and including the Versions V1,V2a, V2b, V2c, V3 and V4 DAR constructs, and including any of the activatable masked antigen binding proteins comprising a dimeric antigen receptor (DAR) that can bind EGFR or CD38. In one embodiment, the population of host cells that express any of the activatable masked antigen binding proteins comprising a dimeric antigen receptor (DAR) described herein are administered to the subject in an amount that is sufficient to effect a measurable improvement or prevention of a disease or disorder associated with tumor or cancer antigen expression.

In one embodiment, the host cell or population of host cells used to treat the subject are autologous and are derived from the subject receiving the treatment. In one embodiment, whole blood can be obtained from the subject and the desired cells (e.g., T lymphocytes, NK cells or macrophages) can be recovered from the whole blood.

In one embodiment, the host cell or population of host cells used to treat the subject are allogenic and are derived from a different subject. Allogenic cells can be obtained from whole blood from a different subject in the same manner employed for the autologous cells. In one embodiment, the allogenic cells are derived from placenta or chord tissue after pregnancy.

In one embodiment, the desired cells are obtained from the subject to receive treatment, or from a different subject, and are engineered to harbor one or more expression vectors that direct expression of any of the first or second polypeptides, or the precursor polypeptides, thereby generating transgenic host cells. The transgenic host cells can express the first or second polypeptides, or the precursor polypeptide. The host cells can express the first and second polypeptide chains which dimerize to form a dimeric antigen receptor that binds specifically to the tumor antigen in the subject. The host cells can express the precursor polypeptide chain which can be cleaved to form first and second polypeptide chains that dimerize to form a dimeric antigen receptor that binds specifically to the tumor antigen in the subject. The transgenic host cells (e.g., harboring the expression vector(s) or expressing the polypeptide chains) can be administered to the subject to treat the disease, disorder or condition associated with over- expression of a tumor antigen.

The present disclosure provides a method of treating a subject having a disease, disorder or condition associated with detrimental expression of a tumor antigen, wherein the disorder is cancer, including, but not limited to hematologic cancer, breast cancer, ovarian cancer, prostate cancer, head and neck cancer, lung cancer, bladder cancer, melanoma, colorectal cancer, pancreatic cancer, lung cancer, liver cancer, renal cancer, esophageal cancer, leiomyoma, leiomyosarcoma, glioma, and glioblastoma, wherein the method for treating the subject comprises administering to the subject an effective amount of a therapeutic composition comprising a population of host cells expressing any of the activatable masked antigen binding proteins comprising a dimeric antigen receptor (DAR) described herein.

In one embodiment, the cancer is a hematologic cancer selected from the group consisting of non-Hodgkin’s lymphoma (NHL), Burkitt’s lymphoma (BL), B chronic lymphocytic leukemia (B-CLL), B and T acute lymphocytic leukemia (ALL), T cell lymphoma (TCL), acute myeloid leukemia (AML), hairy cell leukemia (HCL), Hodgkin’s Lymphoma (HL), chronic myeloid leukemia (CML) and multiple myeloma (MM).

The present disclosure provides an in vitro cleavage-based method to detect protease activity and specificity for detecting, diagnosing, monitoring and/or staging a diseased tissue such as a cancer or tumor. A tumor or cancer mass can be extracted from a subject and contacted with one or any combination of two or more activatable masked antigen binding proteins comprising a dimeric antigen receptor (DAR) described herein, each having at least one peptide linker with a known protease cleavage profile. The tumor or cancer mass from the subject produces one or more proteases. The method comprises: (a) contacting the diseased tissue with one or more activatable masked antigen binding proteins under conditions suitable for a protease(s) to cleave at least one peptide linker on the activatable masked antigen binding protein(s) to generate a cleaved peptide product. The method further comprises: (b) detecting the cleaved peptide product, for example using any method. Thus, the type of protease produced by the tumor or cancer mass can be identified. In one embodiment, the diseased tissue is contacted with one or more of an activatable masked antigen binding proteins comprising a dimeric antigen receptor (DAR), including the second and third generation DAR constructs, and including the Versions V1,V2a, V2b, V2c, V3 and V4 DAR constructs, and including any of the activatable masked antigen binding proteins comprising a dimeric antigen receptor (DAR) that can bind EGFR or CD38.

In one embodiment, the peptide linker is cleavable with any one or any combination of two or more protease selected from a group consisting of a matrix metalloprotease (MMP), MMP1, MMP2, MMP3, MMP8, MMP9, MMP11, MMP13, MMP14, MT1-MMP (membrane type 1 matrix metalloproteinase), ADAM protease, urokinase plasminogen activator (uPA), serine proteases, cysteine proteases, aspartate proteases, threonine proteases, cathepsins (e.g., cysteine or aspartic cathepsins), cathepsin B, cathepsin C, cathepsin D, cathepsin E, cathepsin K and cathepsin L.

The present disclosure provides a method for detecting the presence of a protease produced by a tumor from a subject, the method comprising: (a) contacting (i) a tumor obtained from the subject with (ii) at least one of the activatable masked antigen binding proteins comprising a dimeric antigen receptor (DAR) described herein, wherein the tumor sample produces a protease.

In one embodiment, the first peptide linker includes a first cleavable site and the second peptide linker includes a second cleavable site. In one embodiment, the amino acid sequence of the first and/or second cleavable sites may or may not be a substrate for cleavage by the protease produced by the tumor sample. In one embodiment, the contacting in step (a) is performed under conditions suitable for the protease to cleave the first and/or second cleavable site to generate one or more cleavage products when the protease cleaves the first and/or second cleavable site.

In one embodiment, the method further comprises: (b) detecting the first and/or second cleavage product. In one embodiment, the method further comprises: (c) identifying the type of protease produced by the tumor from the subject by detecting the first and/or second cleavage product and correlating any of the cleavage products with the amino acid sequence of the first and/or second cleavable site.

In one embodiment, by identifying the type of protease produced by the tumor in the subject, the cancer in the subject can be diagnosed. In one embodiment, the first and/or second cleavage product can be detected by gel electrophoresis, Western blot analysis, immunology, immunohistochemistry, colorimetrically, spectrophotometrically, mass spectrometry, liquid chromatography, or by any combination thereof. In one embodiment, the tumor or cancer mass can be obtained from a prostate, breast, ovary, head and neck, bladder, skin, colorectal, anus, rectum, pancreas, lung (including non-small cell lung and small cell lung cancers), leiomyoma, brain, glioma, glioblastoma, esophagus, liver, kidney, stomach, colon, cervix, uterus, endometrium, vulva, larynx, vagina, bone, nasal cavity, paranasal sinus, nasopharynx, oral cavity, oropharynx, larynx, hypolarynx, salivary glands, ureter, urethra, penis, and testis. In one embodiment, the subject is a human, non-human primate, simian, ape, murine (e.g., mice and rats), bovine, porcine, equine, canine, feline, caprine, lupine, ranine or piscine. In one embodiment, the in vitro cleavage-based method can be used for detecting, diagnosing, monitoring and/or staging a cancer in the subject.

The present disclosure provides a kit comprising: at least one of the activatable masked antigen binding proteins comprising a dimeric antigen receptor (DAR), and/or at least one nucleic acid encoding a precursor polypeptide that can be cleaved and assembled to become an activatable masked antigen binding proteins comprising a dimeric antigen receptor (DAR). In one embodiment, the kit includes at least one of: a second or third generation DAR constructs, a Version V1,V2a, V2b, V2c, V3 or V4 DAR construct, and/or an activatable masked antigen binding proteins comprising a dimeric antigen receptor (DAR) that can bind EGFR or CD38. In one embodiment, the kit comprises one or more adjunct compounds selected from a group consisting of Tris, phosphate, carbonate, stabilizers, excipients, biocides and bovine serum albumin. In one embodiment, the kit comprises one or more adjunct compounds selected from a group consisting of Tris, phosphate, carbonate, stabilizers, excipients, biocides and bovine serum albumin. In one embodiment, the kit comprises one container which contains at least one activatable masked antigen binding proteins comprising a dimeric antigen receptor (DAR) (or nucleic acid encoding the protein thereof) and optionally one or more adjunct compound. In one embodiment, the kit comprises two or more containers, wherein one container contains at least one activatable masked antigen binding proteins comprising a dimeric antigen receptor (DAR) (or nucleic acid encoding the protein thereof) and a separate container contains one or more adjunct compounds.

### Activatable Masked Antigen Binding Proteins

### IgG Type Antibodies, Bispecific Antibodies and Dimeric Antigen Receptors (DARs)

The present disclosure provides any of the activatable masked antigen binding proteins described herein, including the IgG class antibodies, bispecific antibodies and dimeric antigen receptors (DARs), comprising paired hetero-dimeric masking moieties having amino acid sequences from Table 1. In one embodiment, the activatable masked antigen binding proteins comprising the IgG class antibodies or bispecific antibodies have first and second and/or third and fourth masking moieties having the amino acid sequence of any of the paired masking moieties listed in Table 1 below. In one embodiment, the activatable masked antigen binding proteins comprising the dimeric antigen receptor (DAR) comprise first and second masking moieties having the amino acid sequence of any of the paired masking moieties listed in Table 1.

TABLE 1

Paired hetero-dimeric masking moieties

| | | |
|---|---|---|
| Paired masking moieties | Masking moiety | Masking moiety |
| Ig γ1 CH1 constant domain and Ig λ pair | Ig γ1 CH1 domain SEQ ID NO:21 | Ig CL λ constant domain SEQ ID NO:22 |
| | ASTKGPSVFPLAPSSKSTSGGTAA | GQPKAAPSVTLFPPSSEELQANKATLV |
| | LGCLVKDYFPEPVTVSWNSGALTS | CLISDFYPGAVTVAWKADSSPVKAGVE |
| | GVHTFPAVLQSSGLYSLSSVVTVP | TTTPSKQSNNKYAASSYLSLTPEQWKS |
| | SSSLGTQTYICNVNHKPSNTKVDK | HRSYSCQVTHEGSTVEKTVAPTESS |
| | RVEPKSS | |
| Ig γ1 CH1 constant domain and Ig κ pair | Ig γ1 CH1 domain SEQ ID NO:21 | Ig CL κ constant domain SEQ ID NO:23 |
| | ASTKGPSVFPLAPSSKSTSGGTAA | RTVAAPSVFIFPPSDEQLKSGTASVVC |
| | LGCLVKDYFPEPVTVSWNSGALTS | LLNNFYPREAKVQWKVDNALQSGNSQE |
| | GVHTFPAVLQSSGLYSLSSVVTVP | SVTEQDSKDSTYSLSSTLTLSKADYEK |
| | SSSLGTQTYICNVNHKPSNTKVDK | HKVYACEVTHQGLSSPVTKSFNRGES |
| | RVEPKSS | |
| Ig γ1 CH3 knob into hole domain pair | Ig γ1 CH3 domain hole SEQ ID NO:24 | Ig γ CH3 domain knob SEQ ID NO:25 |
| | GQPREPQVYTLPPSREEMTKNQVS | GQPREPQVYTLPPSREEMTKNQVSLW C |
| | LS CA VKGFYPSDIAVEWESNGQPE | L VKGFYPSDIAVEWESNGQPENNYKTT |
| | NNYKTTPPVLDSDGSFFLVSKLTV | PPVLDSDGSFFLYSKLTVDKSRWQQGN |
| | DKSRWQQGNVFSCSVMHEALHNHY | VFSCSVMHEALHNHYTQKSLSLSPGK |
| | TQKSLSLSPGK | |
| 38C2 IgG1 VH and VL domain pair | Heavy chain variable domain 38C2 SEQ ID NO:26 | Heavy chain variable domain 38C2 SEQ ID NO:27 |
| | EVQLVESGGGLVQPGGSLRLSCAA | ELQMTQSPSSLSASVGDRVTITCRSSQ |
| | SGFTFSNYWMSWVRQSPEKGLEWV | SLLHTYGSPYLNWYLQKPGQSPKLLIY |
| | SEIRLRSDNYATHYAESVKGRFTI | KVSNRFSGVPSRFSGSGSGTDFTLTIS |
| | SRDNSKNTLYLQMNSLRAEDTGIY | SLQPEDFAVYFCSQGTHLPYTFGGGTK |
| | YCKTYFYSFSYWGQGTLVTVSS | VEIK |
| TCR αβ constant domains pair | TCR α constant domain SEQ ID NO:28 | TCR β constant domain SEQ ID NO:29 |
| | IQNPDPAVYQLRDSKSSDKSVCLF | KVFPPEVAVFEPSEAEISHTQKATLVC |
| | TDFDSQTNVSQSKDSDVYITDKTV | LATGFFPDHVELSWWVNGKEVHSGVST |
| | LDMRSMDFKSNSAVAWSNKSDFAC | DPQPLKEQPALNDSRYsLSSRLRVSAT |
| | ANAFNNSIIPEDTFFPSP | FWQNPRNHFRCQVQFNGQIVSAEAWGR |
| | | ADS |

The present disclosure provides any of the activatable masked antigen binding proteins described herein, including the IgG class antibodies, bispecific antibodies and dimeric antigen receptors (DARs), comprising paired homo-dimeric masking moieties having amino acid sequences from Table 2. In one embodiment, the activatable masked antigen binding proteins comprising the IgG class antibodies or bispecific antibodies have first and second and/or third and fourth masking moieties having the amino acid sequence of any of the paired masking moieties listed in Table 2 below. In one embodiment, the activatable masked antigen binding proteins comprising the dimeric antigen receptor (DAR) comprise first and second masking moieties having the amino acid sequence of any of the paired masking moieties listed in Table 2.

TABLE 2

Paired homo-dimeric masking moieties

| Paired masking moieties | Masking moiety | Masking moiety |
|---|---|---|
| IgG1 CH3:CH3 pair | IgG1 CH3 constant domain SEQ ID NO:30 | IgG1 CH3 constant domain SEQ ID NO:30 |
| | GQPREPQVYTLPPSREEMTKNQVS | GQPREPQVYTLPPSREEMTKNQVSLTC |
| | LTCLVKGFYPSDIAVEWESNGQPE | LVKGFYPSDIAVEWESNGQPENNYKTT |
| | NNYKTTPPVLDSDGSFFLYSKLTV | PPVLDSDGSFFLYSKLTVDKSRWQQGN |
| | DKSRWQQGNVFSCSVMHEALHNHY | VFSCSVMHEALHNHYTQKSLSLSPGK |
| | TQKSLSLSPGK | |
| IgG4 CH3:CH3 pair | IgG4 CH3 constant domain SEQ ID NO:31 | IgG4 CH3 constant domain SEQ ID NO:31 |
| | GQPREPQVYTLPPSQEEMTKNQVS | GQPREPQVYTLPPSQEEMTKNQVSLTC |
| | LTCLVKGFYPSDIAVEWESNGQPE | LVKGFYPSDIAVEWESNGQPENNYKTT |
| | NNYKTTPPVLDSDGSFFLYSRLTV | PPVLDSDGSFFLYSRLTVDKSRWQEGN |
| | DKSRWQEGNVFSCSVMHEALHNHY | VFSCSVMHEALHNHYTQKSLSLSPGK |
| | TQKSLSLSPGK | |

The present disclosure provides any activatable masked antigen binding proteins described herein, including the IgG class antibodies, bispecific antibodies and dimeric antigen receptors (DARs), comprising at least first and/or second peptide linkers, and optionally further comprising third and/or fourth peptide linkers, carrying a cleavable site. In one embodiment, the cleavable site is cleavable with a cleaving condition which includes a protease, esterase, reductive condition, or oxidative condition. In one embodiment, the cleavable site is cleavable with a protease that is present in a tumor microenvironment or is cleavable with a reductive or oxidative condition that is present in a tumor microenvironment (Rakashanda et al., 2012 Biotechnology and Molecular Biology Review 7(4):90-101). In one embodiment, the cleaving condition comprises one protease or any combination of two or more proteases, including serine proteases, cysteine proteases, aspartate proteases, threonine proteases, glutamic acid proteases, metalloproteases, asparagine peptide lyases, serum proteases, matrix metalloproteinase (MMP), MMP1, MMP2, MMP3, MMP7, MMP8, MMP9, MMP11, MMP13, MMP14, MT1-MMP (membrane type 1 matrix metalloproteinase), urokinase plasminogen activator (uPA), enterokinase, prostate-specific antigen (PSA, hK3), interleukin-ḯβ converting enzyme, thrombin, FAP (FAP-a), dipeptidyl peptidase, meprins, granzymes (e.g., granzyme B), dipeptidyl peptidase IV (DPPIV/CD26), a disintegrin and metalloproteinase (e.g., ADAM proteases), ADAM10, ADAM12, ADAM17, hepsin, cathepsins (e.g., cysteine or aspartic cathepsins), cathepsin B, cathepsin C, cathepsin D, cathepsin E, cathepsin K, cathepsin L, kallikreins, hKl, hK10, hK15, plasmin, collagenase, Type IV collagenase, stromelysin, lysosomal enzyme, Factor Xa, chymotrypsin-like protease, trypsin-like protease, elastase- like protease, subtili sin-like protease, actinidain, bromelain, calpain, caspases, caspase-1, caspase-2, caspase-3, caspase-8, caspase-9, caspase-10, caspase-11, caspase-12, caspase-13, caspase-14, herpes simplex virus protease, HIV protease, CMV protease, Mirl- CP, papain, HIV-1 protease, HSV protease, CMV protease, chymosin, renin, pepsin, matriptase (e.g., matriptase 2, human ST14, or TMPRSS6), legumain, plasmepsin, nepenthesin, metalloexopeptidases, and/or metalloendopeptidases.

In one embodiment, the peptide linker comprises a cleavable site having an amino acid sequence that is at least 95%, or at least 96%, or at least 97%, or at least 98%, or at least 99% identical to a peptide that is cleavable with a matrix metalloprotease protease, for example the amino acid sequence of GGSGSGSGGSSGGGSGGGGS, TSGSGGSGGSV, TSGSGGSPLGMGGSGSV, TSGSGGSPLGVGGSGSV, TSGSGGSPAALGGSGSV, TSGSGGSPAGLGGSGSV, TSGSGGSPLGMVGV, TSGSGGSPLGVVGV, TSGSGGSPAALVGV, TSGSGGSPAGLVGV, TSGSGGSPLGMVLV, TSGSGGSPLGVVLV, TSGSGGSPAALVLV or TSGSGGSPAGLVLV.

In one embodiment, the cleavable site comprises the amino acid sequence LEATA which is recognized and cleaved by MMP9. In one embodiment, the cleavable site comprises the amino acid sequence PR(S/T)(L/I)(S/T) which is recognized and cleaved by MMP9. In one embodiment, the cleavable site comprises the amino acid sequence SGSGGSPLGMGGSGSVD, SGSGGSPAGLGGSCSVD, or SGSGGSPAGLVGVD. In one embodiment, the cleavable site comprises the amino acid sequence GGAANLVRGG which is recognized and cleaved by MMP11.

The present disclosure provides any activatable masked antigen binding proteins described herein, including the IgG class antibodies, bispecific antibodies and dimeric antigen receptors (DARs), comprising at least a first and/or second peptide linker, and optionally further comprising a third and/or fourth peptide linker, carrying a cleavable site having an amino acid sequence according to any of the peptide linkers listed in Table 3, 4, 5, 6, 7, 8 and/or 9.

TABLE 3

Amino acid sequences of peptide linkers cleavable with a matrix metalloproteinase
Amino acid sequence
SRPLALR
SRPANLR
X1X2X3X4X5X6X7, wherein X1 is S, V or R, X2 is any amino acid, X3 is P or A, X4 is L, M, A, R or Y, X5 is A, S, N, G, H or M, X6 is L, X7 is R, L, Q or M.

TABLE 4

Amino acid sequences of peptide linkers cleavable with matrix metalloproteinase 1 (MMP1)
Amino acid sequence
DVAQFVLT
VLVPMAMMAS
X1X2X3X4X5X6X7X8, wherein X1 is any amino acid, X2 is P or A, X3 and X4 are any amino acids, X5 is L or I, X6, X7, X8 are any amino acids.

TABLE 5

Amino acid sequences of peptide linkers cleavable with matrix metalloproteinase 2 (MMP2)
Amino acid sequence
PLGLAG
HRPRGX1TN
X1X2X3X4X5X6X7X8, wherein X1 is any amino acid, X2 is P, X3 and X4 are any amino acids, X5 is L or I, X6, X7 and X8 are any amino acids

TABLE 6

Amino acid sequences of peptide linkers cleavable with matrix metalloproteinase 9 (MMP9)
Amino acid sequence
VPLVLYS (MMP9)
VPLSLYS
GGPLGVRGG (MMP2/9)
GGPLGVRGGGGG (extended)
PLGVRGG (deleted)
HRPRGX1TN, wherein X1 is V or W
X1X2X3X4X5X6X7X8, wherein X1 is G, X2 is P or A, X3 is any amino acids, X4 is G or A, X5 is L, X6 is any amino acid, X7 is G, X8 is any amino acid
GPLGIAGQ
PVGLIG
HPVGLLAR

TABLE 7

Amino acid sequences of peptide linkers cleavable with matrix metalloproteinase 2 or 9
Amino acid sequence
GGPLGVRGG (MMP2/9)
GGPLGVRGGGGG (extended)
PLGVRGGG (deleted)

TABLE 8

Amino acid sequences of peptide linkers cleavable with urinary-type plasminogen activator (uPA)
Amino acid sequence
GGGGRRGGGG (uPA1)
SGRSAN (uPA2)
X1SGRSA, wherein X1 is G or K
LSGRSX1NH, wherein X1 is D, V or A
X1X2X3X4X5X6X7X8, wherein X1 is any amino acid, X2 is S, X3 is G or S, X4 is R or K, X5 is any amino acid, X6 is R or V, X7 is any amino acid, X8 is any amino acid
SGRSA
X9X10X11X12X13X14X15X16, wherein X9 is L, I, A or K, X10 is S, G, or Q, X11 is K, L, A, G, or S, X12 is R, X13 is S, K, G, A or V, X14 is A, V or L, X15 is N, X16 is H, G, V, or L
RKSSIIIRMRDWL

TABLE 9

Amino acid sequences of peptide linkers cleavable with matriptase
Amino acid sequence
AGPR
RQARVV
LSGX1, wherein X1 is R or K
X1SGRSA, wherein X1 is G or K
LSGRSX1NH, wherein X1 is D, V or A

Amino acid sequence
X1X2X3X4X5X6X7X8, wherein X1 is R, X2 and X3 are any amino acid, X4 is R, X5 is K, X6 is V, X7 is any amino acid, X8 is G
X9X10X11X12X13, wherein X9 is K or R, X10 is R, K, H or Q, X11 is G, A or S, X12 is R or K, X13 is A
X14X15X16X17X18X19X20X21, wherein X14 is L, I, A or K, X15 is S, G, or Q, X16 is K, L, A, G, or S, X17 is R, X18 is S, K, G, A or V, X19 is A, V or L, X20 is N, X21 is H, G, V, or L
X22X23X24X25X26, wherein X22 is R or K, X23 is non-basic amino acid (G,A,S,T,C,V,L,I, M,P,F,Y,W,D,E,N, or Q), X24 is S, X25 is R, X26 is A
X27X28X29X30X31, wherein X27 is non-basic amino acid (G,A,S,T,C,V,L,I, M,P,F,Y,W,D,E,N,Q), X28 is R or K, X29 is S, X30 is R, X31 is A

The present disclosure provides any activatable masked antigen binding proteins described herein, including the dimeric antigen receptors (DARs), wherein the hinge region comprises any one or any combination of two or more regions comprising an upper, core or lower hinge sequences from an IgG1, IgG2, IgG3 or IgG4 immunoglobulin molecule. In one embodiment, the hinge region comprises an IgG1 upper hinge sequence EPKSCDKTHT. In one embodiment, the hinge region comprises an IgG1 core hinge sequence CPXC, wherein X is P, R or S. In one embodiment, the hinge region comprises a lower hinge/CH2 sequence PAPELLGGP. In one embodiment, the hinge is joined to an Fc region (CH2) having the amino acid sequence SVFLFPPKPKDT. In one embodiment, the hinge region includes the amino acid sequence of an upper, core and lower hinge and comprises EPKSCDKTHTCPPCPAP ELLGGP. In one embodiment, the hinge region comprises one, two, three or more cysteines that can form at least one, two, three or more interchain disulfide bonds.

The present disclosure provides any activatable masked antigen binding proteins described herein, including the IgG class antibodies, bispecific antibodies and dimeric antigen receptors (DARs), wherein the first, second, third and/or fourth peptide linker permits dimerization of the paired masking moieties (e.g., dimerization of first and second masking moieties, and/or dimerization of third and fourth masking moieties). In one embodiment, any of the peptide linkers have a length of 1-50 amino acids which optionally can include at least one amino acid analog. In one embodiment, at least one of the linkers is a flexible linker and comprises predominantly any combination of two or more amino acids glycine, serine, alanine and/or threonine. In one embodiment, at least one of the peptide linkers comprise at least one and up to four polymers of glycine-alanine, or alanine-serine, or other flexible linkers sequences. In one embodiment, at least one of the peptide linkers comprise an amino acid sequence selected from a group consisting of: (SG)n, (SGG)n, (SGGG)n, (SSG)n, (GS)n, (GGG)n, (GSGGS)n, (GSG)n, (GGGGS)n, (GGGS)n, (GGGGSGS)n, (GGSGGGSGG)n, (GGGGSGGS)n, and (GGS)n, where n is an integer of 1-6. In one embodiment, at least one of the peptide linkers comprise an amino acid sequence selected from a group consisting of: AKTTPKLEEGEFSEAR, AKTTPKLEEGEFSEARV, AKTTPKLGG, SAKTTPKLGG, AKTTPKLEEGEFSEARV, SAKTTP, SAKTTPKLGG, RADAAP, RADAAPTVS, RADAAAAGGPGS, RADAAAA(G₄S)₄ SAKTTP, SAKTTPKLGG, SAKTTPKLEEGEFSEARV, ADAAP, ADAAPTVSIFPP, TVAAP, TVAAPSVFIFPP, QPKAAP, QPKAAPSVTLFPP, AKTTPP, AKTTPPSVTPLAP, AKTTAP, AKTTAPSVYPLAP, ASTKGP, ASTKGPSVFPLAP, GENKVEYAPALMALS, GPAKELTPLKEAKVS and GHEAAAVMQVQYPAS.

The present disclosure provides any activatable masked antigen binding proteins described herein, including the IgG class antibodies, bispecific antibodies and dimeric antigen receptors (DARs), that bind an epitope or an antigen from a human. In one embodiment, the activatable masked antigen binding proteins binds an epitope or an antigen from a human and can bind (e.g., cross-react) with an epitope or antigen (e.g., homologous antigen) from any one or any combination of non-human animals such as dog, cat, mouse, rat, goat, rabbit, hamster and/or monkey (e.g., cynomolgus, rhesus or macaque). In one embodiment, the activatable masked antigen binding proteins bind an EFGR antigen comprising the amino acid sequence of SEQ ID NO:1.

### EGFR Target Antigen - Extracellular Domain: SEQ ID NO:1

LEEKKVCQGTSNKLTQLGTFEDHFLSLQRMFNNCEVVLGNLEITYVQRNY
DLSFLKTIQEVAGYVLIALNTVERIPLENLQIIRGNMYYENSYALAVLSN
YDANKTGLKELPMRNLQEILHGAVRFSNNPALCNVESIQWRDIVSSDFLS
NMSMDFQNHLGSCQKCDPSCPNGSCWGAGEENCQKLTKIICAQQCSGRCR
GKSPSDCCHNQCAAGCTGPRESDCLVCRKFRDEATCKDTCPPLMLYNPTT
YQMDVNPEGKYSFGATCVKKCPRNYWTDHGSCVRACGADSYEMEEDGVRK
CKKCEGPCRKVCNGIGIGEFKDSLSINATNIKHFKNCTSISGDLHILPVA
FRGDSFTHTPPLDPQELDILKTVKEITGFLLIQAWPENRTDLHAFENLEI
IRGRTKQHGQFSLAVVSLNITSLGLRSLKEISDGDVIISGNKNLCYANTI
NWKKLFGTSGQKTKIISNRGENSCKATGQVCHALCSPEGCWGPEPRDCVS
CRNVSRGRECVDKCNLLEGEPREFVENSECIQCHPECLPQAMNITCTGRG
PDNCIQCAHYIDGPHCVKTCPAGVMGENNTLVWKYADAGHVCHLCHPNCT
YGCTGPGLEGCPTNGPKIPS

The present disclosure provides any activatable masked antigen binding proteins described herein, including the IgG class antibodies, bispecific antibodies and dimeric antigen receptors (DARs), that bind an EGFR antigen, wherein the activatable masked antigen binding proteins comprises an antibody heavy chain having the amino acid sequence of SEQ ID NO:2 or 4, and comprising an antibody light chain having the amino acid sequence of SEQ ID NO:3 or 5, in any heavy/light chain combination. In one embodiment, the heavy chain variable region of SEQ ID NO:2 and 4 is underlined. In one embodiment, the light chain variable region of SEQ ID NO:3 and 5 is underlined.

### Human Anti-EGFR Antibody Heavy Chain; Control Anti-EGFR Antibody And Activatable Masked Antibody: SEQ ID NO:2

QVQLKQSGPGLVQPSQSLSITCTVSGFSLTNYGVHWVRQSPGKGLEWLGV IWSGGNTDYNTPETSRLSINKDNSKSQVFFKMNSLQSNDTAIYYCARALT YYDYEFAYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK

### Human Anti-EGFR Antibody Light Chain; Control Anti-EGFR Antibody And Activatable Masked Antibody: SEQ ID NO:3

DILLTQSPVILSVSPGERVSFSCRASQSIGTNIHWYQQRTNGSPRLLIKY ASESISGIPSRFGSGSGTDFTLSINSVESEDIADYYCQQNNNWPTTFGAG TKLELKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC

### Human Anti-EGFR Antibody Heavy Chain; Cetuximab SEQ ID NO:4

QVQLKQSGPGLVQPSQSLSITCTVSGFSLTNYGVHWVRQSPGKGLEWLGV IWSGGNTDYNTPETSRLSINKDNSKSQVFFKMNSLQSNDTAIYYCARALT YYDYEFAYWGQGTLVTVSAASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK

### Human Anti-EGFR Antibody Light Chain; Cetuximab SEQ ID NO:5

DILLTQSPVILSVSPGERVSFSCRASQSIGTNIHWYQQRTNGSPRLLIKY ASESISGIPSRFSGSGSGTDFTLSINSVESEDIADYYCQQNNNWPTTFGA GTKLELKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

The present disclosure provides any activatable masked antigen binding proteins described herein, including the IgG class antibodies, bispecific antibodies and dimeric antigen receptors (DARs), that bind an epitope or an antigen from a human. In one embodiment, the activatable masked antigen binding proteins binds an epitope or an antigen from a human and can bind (e.g., cross-react) with an epitope or antigen (e.g., homologous antigen) from any one or any combination of non-human animals such as dog, cat, mouse, rat, goat, rabbit, hamster and/or monkey (e.g., cynomolgus, rhesus or macaque). In one embodiment, the activatable masked antigen binding proteins bind a CD38 antigen comprising the amino acid sequence of SEQ ID NO:6.

### CD38 Target Antigen - Extracellular Domain: SEQ ID NO: 6

VPRWRQQWSGPGTTKRFPETVLARCVKYTEIHPEMRHVDCQSVWDAFKGA
FISKHPCNITEEDYQPLMKLGTQTVPCNKILLWSRIKDLAHQFTQVQRDM
FTLEDTLLGYLADDLTWCGEF

NTSKINYQSCPDWRKDCSNNPVSVFWKTVSRRFAEAACDVVHVMLNGSRS
KIFDKNSTFGSVEV

HNLQPEKVQTLEAWVIHGGREDSRDLCQDPTIKELESIISKRNIQFSCKN
IYRPDKFLQCVKNP

EDSSCTSEI

The present disclosure provides any activatable masked antigen binding proteins described herein, including the IgG class antibodies, bispecific antibodies and dimeric antigen receptors (DARs), that bind a CD38 antigen, wherein the activatable masked antigen binding proteins comprises an antibody heavy chain having the amino acid sequence of SEQ ID NO:7, 9, 11, 13, 14, 15, 16, 17, 18, or 20, and comprising an antibody light chain having the amino acid sequence of SEQ ID NO:8, 10, 12 or 19, in any heavy/light chain combination (see Table 10 below). In one embodiment, the heavy chain variable region of SEQ ID NO:7 is underlined. In one embodiment, the light chain variable region of SEQ ID NO:8 is underlined.

### Anti-CD38 Heavy Chain: SEQ ID NO:7

QVQLVESGGGLVKPGGSLRLSCAASGFTFSDDYMSWIRQAPGKGLEWVAS VSNGRPTTYYADSVRGRFTISRDNAKNSLYLQMNSLRAEDTAVYYCARED WGGEFTDWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK

### Anti-CD38 Light Chain: SEQ ID NO:8

QAGLTQPPSASGTSGQRVTISCSGSSSNIGINFVYWYQHLPGTAPKLLIY KNNQRPSGVPDRFSGSKSGNSASLAISGLRSEDEADYYCAAWDDSLSGYV FGSGTKVTVLGQPKAAPSVTLFPPSSEELQANKATLVCLISDFYPGAVTV
AWKADSSPVKAGVETTTPSKQSNNKYAASSYLSLTPEQWKSHRSYSCQVT
HEGSTVEKTVAPTECS

TABLE 10

| | |
|---|---|
| Anti-CD38 heavy chain variable region | Anti-CD38 light chain variable region |
| A2 parental heavy chain SEQ ID NO:9 | A2 parental light chain SEQ ID NO:10 |
| QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSS | QAGLTQPPSASGTSGQRVTISCSGSSSNIGINFVYWYQHLPGTAPKLLIYKNNQRPSGVPDRFSGSKSGNSASLAISGLRSEDEADYYCAAWDDSLSGYVFGSGTKVTVL |
| 3H10m1 SEQ ID NO:11 | 3H10m1 SEQ ID NO:12 |
| QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREGWSGEFTDWGQGTLVTVSS | QAGLTQPPSASGTSGQRVTISCSGSSSNIGFHFVYWYQHLPGTAPKLLIYKNNQRPSGVPDRFSGSKSGNSASLAISGLRSEDEADYYCAAWDDSLSGYVFGSGTKVTVL |
| 3G8m1 SEQ ID NO:13 | 3G8ml SEQIDNO:12 |
| QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREAWGGEFTNWGQGTLVTVSS | QAGLTQPPSASGTSGQRVTISCSGSSSNIGFHFVYWYQHLPGTAPKLLIYKNNQRPSGVPDRFSGSKSGNSASLAISGLRSEDEADYYCAAWDDSLSGYVFGSGTKVTVL |
| 3E3m1 SEQ ID NO:14 | 3D3m1 SEQ ID NO:12 |
| QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREAWGGEFTDWGQGTLVTVSS | QAGLTQPPSASGTSGQRVTISCSGSSSNIGFHFVYWYQHLPGTAPKLLIYKNNQRPSGVPDRFSGSKSGNSASLAISGLRSEDEADYYCAAWDDSLSGYVFGSGTKVTVL |
| 3G3 SEQ ID NO:15 | 3G3 SEQ ID NO:10 |
| QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREAWSGEFTDWGQGTLVTVSS | QAGLTQPPSASGTSGQRVTISCSGSSSNIGINFVYWYQHLPGTAPKLLIYKNNQRPSGVPDRFSGSKSGNSASLAISGLRSEDEADYYCAAWDDSLSGYVFGSGTKVTVL |
| 3E11 SEQ ID NO:16 | 3E11 SEQIDNO:10 |
| QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREGWGGEFTDWGQGTLVTVSS | QAGLTQPPSASGTSGQRVTISCSGSSSNIGINFVYWYQHLPGTAPKLLIYKNNQRPSGVPDRFSGSKSGNSASLAISGLRSEDEADYYCAAWDDSLSGYVFGSGTKVTVL |
| 3H10 SEQIDNO:17 | 3H10 SEQIDNO:10 |
| QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREGWSGEFTDWGQGTLVTVSS | QAGLTQPPSASGTSGQRVTISCSGSSSNIGINFVYWYQHLPGTAPKLLIYKNNQRPSGVPDRFSGSKSGNSASLAISGLRSEDEADYYCAAWDDSLSGYVFGSGTKVTVL |
| 3H10N SEQIDNO:18 | 3H10N SEQ ID NO:19 |
| QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGQGTLVTVSS | QSVLTQPPSASGTSGQRVTISCSGSSSNIGFHFVYWYQHLPGTAPKLLIYKNNQRPSGVPDRFSGSKSGNSASLAISGLRSEDEADYYCAAWDDSLSGYVFGSGTKVTVL |
| 3H10NS SEQ ID NO:20 | 3H10NS SEQIDNO:19 |
| QVQLVESGGGLVKPGGSLRLSCAASGFTFSDDYMSWIRQAPGKGLEWVASVSSGRPTTYYADSVRGRFTISRDNAKNSLYLQMNSLRAEDTAVYYCAREDWGGEFTDWGQGTLVTVSS | QSVLTQPPSASGTSGQRVTISCSGSSSNIGFHFVYWYQHLPGTAPKLLIYKNNQRPSGVPDRFSGSKSGNSASLAISGLRSEDEADYYCAAWDDSLSGYVFGSGTKVTVL |
| 3E10 SEQIDNO:9 | 3E10 SEQIDNO:12 |
| QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSS | QAGLTQPPSASGTSGQRVTISCSGSSSNIGFHFVYWYQHLPGTAPKLLIYKNNQRPSGVPDRFSGSKSGNSASLAISGLRSEDEADYYCAAWDDSLSGYVFGSGTKVTVL |
| 3H10m1g SEQ ID NO:11 | 3H10m1g SEQIDNO:19 |
| QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREGWSGEFTDWGQGTLVTVSS | QSVLTQPPSASGTSGQRVTISCSGSSSNIGFHFVYWYQHLPGTAPKLLIYKNNQRPSGVPDRFSGSKSGNSASLAISGLRSEDEADYYCAAWDDSLSGYVFGSGTKVTVL |

### EXAMPLES

The following examples are meant to be illustrative and can be used to further understand embodiments of the present disclosure and should not be construed as limiting the scope of the present teachings in any way.

### Example 1: Designation of Activatable Masked Antibodies

Various activatable masked IgG type antibodies that bind EGFR or CD3 8 antigens were prepared which comprises a first heavy chain variable region and a first light chain variable region, wherein (i) the N-terminal end of the first heavy chain variable region is joined to a first masking moiety via a first peptide linker having a first cleavable site, and (ii) the N-terminal end of the first light chain variable region is joined to a second masking moiety via a second peptide linker having a second cleavable site, and (iii) the first and second masking moieties associate with each other to form a first dimerized masking complex, and (iv) the first cleavable site is cleavable with a first protease, and (v) the second cleavable site is cleavable with a second protease, wherein the first and second cleavable sites are cleavable with the same or different proteases.

The various activatable masked IgG type antibodies also comprise a second heavy chain variable region and a second light chain variable region, wherein (i) the N-terminal end of the second heavy chain variable region is joined to a third masking moiety via a third peptide linker having a third cleavable site, and (ii) the N-terminal end of the second light chain variable region is joined to a fourth masking moiety via a fourth peptide linker having a fourth cleavable site, and (iii) the third and fourth masking moieties associate with each other to form a second dimerized masking complex, and (iv) the third cleavable site is cleavable with a third protease, and (v) the fourth cleavable site is cleavable with a fourth protease, wherein the third and fourth cleavable sites are cleavable with the same or different proteases.

The naming convention (designation) of the various activatable masking moieties is listed in Table 11 below.

TABLE 11

| | | | |
|---|---|---|---|
| Designation: | Masking moieties | Hole or knob in masking moiety | Peptide linkers |
| HC-CH3 hole MMP2/9, LC-CH3 knob MMP2/9 | Heavy chain: Human Ig gamma 1 CH3 | hole | MMP2/9 |
| | Light chain: Human Ig gamma 1 CH3 | knob | MMP2/9 |
| HC-CH3 hole uPA1, LC-CH3 knob MMP2/9 | Heavy chain: Human Ig gamma 1 CH3 | hole | uPA1 |
| | Light chain: Human Ig gamma 1 CH3 | knob | MMP2/9 |
| HC-CH3 hole uPA1, LC-CH3 knob uPA1 | Heavy chain: Human Ig gamma 1 CH3 | hole | uPA1 |
| | Light chain: Human Ig gamma 1 CH3 | knob | uPA1 |
| HC-CH1 MMP2/9, LC-CL kappa MMP2/9 | Heavy chain: Human Ig gamma 1 CH1 | None | MMP2/9 |
| | Light chain: Human Ig kappa CL | None | MMP2/9 |
| HC-CH1 MMP2/9, LC-CL lambda MMP2/9 | Heavy chain: Human Ig gamma 1 CH1 | None | MMP2/9 |
| | Light chain: Human Ig lambda CL | None | MMP2/9 |
| HC-G4CH3 MMP2/9, LC-G4CH3 MMP2/9 | Heavy chain: Human Ig gamma 4 CH3 | None | MMP2/9 |
| | Light chain: Human Ig gamma 4 CH3 | None | MMP2/9 |
| HC-CH3 hole MMP2/9, LC-CH3 knob deleted | Heavy chain: Human Ig gamma 1 CH3 | hole | MMP2/9 |
| | Light chain: Human Ig gamma 1 CH3 | knob | deleted |
| HC-CH3 hole MMP2/9, LC-CH3 knob non-cleavable | Heavy chain: Human Ig gamma 1 CH3 | hole | MMP2/9 |
| | Light chain: Human Ig gamma 1 CH3 | knob | Non-cleavable |
| HC-CH3 hole MMP2/9, LC-CH3 knob extended | Heavy chain: Human Ig gamma 1 CH3 | hole | MMP2/9 |
| | Light chain: Human Ig gamma 1 CH3 | knob | Extended MMP2/9 |
| HC-CH3 hole deleted, LC-CH3 knob deleted | Heavy chain: Human Ig gamma 1 CH3 | hole | Deleted |
| | Light chain: Human Ig gamma 1 CH3 | knob | Deleted |
| HC-TCR alpha MMP2/9 LC-TCR beta MMP2/9 | Heavy chain: TCR alpha | None | MMP2/9 |
| | Light chain: TCR beta | None | MMP2/9 |
| HC-CH3 hole MMP9 LC-CH3 knob G4S | Heavy chain: CH3 | hole | MMP2/9 |
| | Light chain: CH3 | knob | G4S |

The naming convention of the peptide linkers in the various activatable masked antibodies, and their amino acid sequences, is listed in Table 12 below. The peptide linker sequences are underlined in Table 12 below. The non-underlined sequences represent the C-terminal amino acid sequence of the adjoining masking moiety CH3 (e.g., see SEQ ID NOS:24 and 25).

TABLE 12

| Peptide linkers: | Amino acid sequence: | SEQ ID NO |
|---|---|---|
| MMP2/9 | LSLSPGKGGPLGVRGG | 32 |
| Deleted (minus GKGG) | LSLSP----PLGVRGG | 33 |
| Non-cleavable | LSLSPGKGGSGGGSGG | 34 |
| Extended (3 extra Gs) | LSLSPGKGGPLGVRGGGGG | 35 |
| MMP9 | VPLVLYS | 36 |
| G4S | GGGGS | 37 |
| uPA1 (serine protease) | LSLSPGKGGGGRRGGGG | 38 |
| uPA2 | SGRSAN | 39 |
| Tandem | GGPLGVRGGPLGVRGG | 40 |

For the “Deleted” peptide linker sequence, the C-terminal amino acids GK of the human Ig-gamma CH3 and the N-terminal amino acids GG of the MMP2/9 peptide linker are deleted (see Table 12 above).

### Example 2: Expression, Purification and Yield of Various IgG-Type Activatable Masked Antibodies

CHO-S cells (or CHO-K1 cells) were incubated in a CO₂ incubator to a cell density of 1- 2×10⁶ cells/ml with viability no less than 90%. Separate expression vectors for heavy and light chain pairs were prepared using a commercial plasmid DNA extraction kit (e.g. Qiagen Maxi plasmid DNA extraction kit). The expression vectors used are similar to pEGFP (e.g., from CloneTech). The expression vector pCDNA (from Invitrogen) can also be used. A mouse Ig gamma leader peptide sequence (MEWSWVFLFFLSVTTGVHS) was used as the secretory peptide sequence for both heavy and light chain expression. DNA PEI complex for transfection was formed by mixing DNA and PEI (Polyethylenimine, from Polyscience catalog # 24765) at a ratio between 1:2-3 (weight/volume concentrations). CHO cells to DNA ratio was approximately 10⁶ cells per microgram DNA. DNA and PEI complex was formed in OptiPRO media (Thermo Fisher) and added to the CHO cell cultures in shaker flasks and incubated at 37° C. with rotation overnight. On Day 2 the culture was expanded by doubling the culture media with CHO culturing media containing penicillin/streptomycin and placed in 30° C. incubator with rotation for 1 to 2 weeks depending on IgG titer and viability of the CHO cells. The activatable masked anti-EGFR antibodies were purified by batch type capturing of the molecules using commercial Protein A resins.

The expression levels of Protein A titer and IgG yield from 25 mL CHO transfections of various anti-EGFR activatable masked IgG type antibodies was determined for various activatable masked antibodies and control anti-EGFR antibody with no masking moieties, which are listed in Table 13:

TABLE 13

| Anti-EGFR activatable masked antibodies: | Protein A titer (ug/mL) | IgG yield (ug) |
|---|---|---|
| HC-CH3 hole MMP2/9, LC-CH3 knob MMP2/9 | 3.1 | 150 |
| HC-CH3 hole uPA1, LC-CH3 knob MMP2/9 | 3.07 | 100 |
| HC-CH3 hole uPA1, LC-CH3 knob uPA1 | 2.19 | 70 |
| HC-CH1 MMP2/9, LC-CL kappa MMP2/9 | 10.4 | 240 |
| HC-CH1 MMP2/9, LC-CL lambda MMP2/9 | 8.39 | 200 |
| HC-G4CH3 MMP2/9, LC-G4CH3 MMP2/9 | 0.69 | 10 |
| Control anti-EGFR antibody with no masking moieties | 16.06 | 350 |

### Example 3: Cleaving MMP2/9 or uPA1peptide Linkers of Various IgG-Type Activatable Masked Antibodies, and Gel Analysis

Various activatable masked antibodies (anti-EGFR) having peptide linkers that are cleavable with either MMP2, MMP9 or uPA, were subjected to digestion with MMP2, MMP9 or uPA proteases, for various time ranges (1, 3, 6.5 or 23 hours) and the resulting digestion products were analyzed by gel electrophoresis on 4-20% gradient Tris-Glycine SDS-PAGE gels under non-reducing or reducing conditions (reducing agent is NUPAGE 10X Sample Reducing Agent from Invitrogen).

Digestion reactions contained 0.1 ug of proteases MMP2 (recombinant human MMP2 from RnD Systems, catalog # 902-MP-010) or MMP9 (recombinant human MMP9 from Origeen CAT#: TP302872, or RnD Systems CAT # 911-MP-010) per 10 or 20 ug of uncleaved activatable masked antibody were incubated in DPBS buffer at room temperature or at 37° C. for 1, 3, 6.5 or 23 hours. The final concentration of the uncleaved activatable masked antibody was 0.25 ug/uL or 0.5 ug/uL, with 0.4 ug of MMP9 protease or 0.75 ug of uPA protease. APMA activation was not conducted.

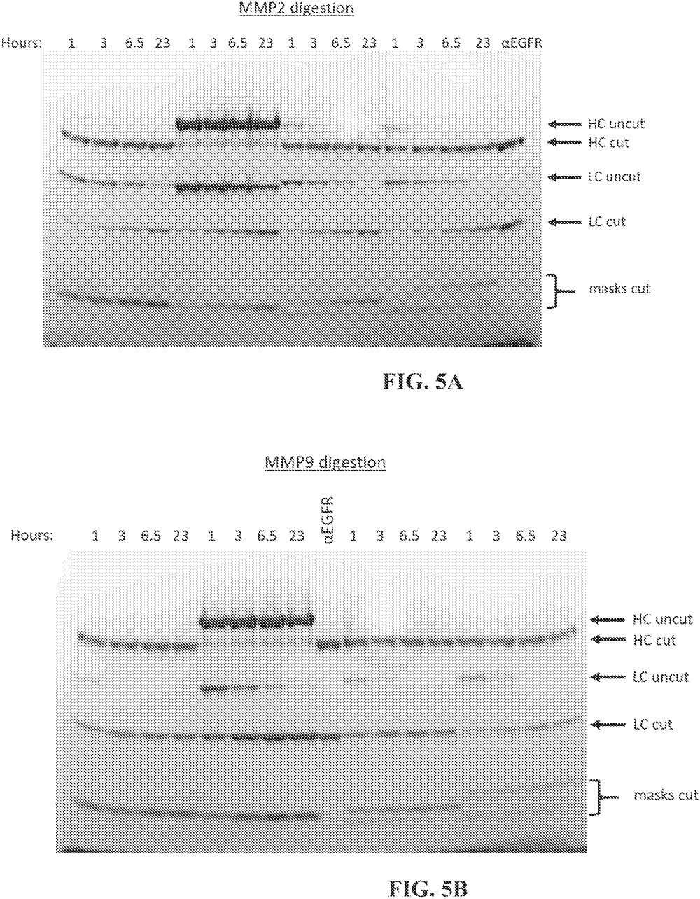

The anti-EGFR activatable masked antibodies digested with MMP2 are shown in FIG. 5A. The order of the loaded samples for FIG. 5A, from left to right, is listed in Table 14 below. The marker “M” shown in FIGS. 5A-B is SPECTRA Multicolor Broad Range Protein Ladder (from Thermo Fisher Scientific, catalog # 26623).

FIG. 5A: Digestion of the anti-EGFR activatable masked antibodies with MMP2 generates the predicted cleavage products, including cut heavy chain, cut light chain and cut masks. When comparing the cleavage products generated from the 1, 3, 6.5 and 23 hours digestion conditions the level of intact (uncut) light chains decreases and the level of cleaved light chain and cleaved masks (cut) increases for all masked antibodies tested.

TABLE 14

See FIG. 5A

| Lane: | Antibody: | Time (hours): |
|---|---|---|
| 1 | HC-CH3 hole MMP2/9, LC-CH3 knob MMP2/9 | 1 |
| 2 | HC-CH3 hole MMP2/9, LC-CH3 knob MMP2/9 | 3 |
| 3 | HC-CH3 hole MMP2/9, LC-CH3 knob MMP2/9 | 6.5 |
| 4 | HC-CH3 hole MMP2/9, LC-CH3 knob MMP2/9 | 23 |
| 5 | HC-CH3 hole uPA1, LC-CH3 knob uPA1 | 1 |
| 6 | HC-CH3 hole uPA1, LC-CH3 knob uPA1 | 3 |
| 7 | HC-CH3 hole uPA1, LC-CH3 knob uPA1 | 6.5 |
| 8 | HC-CH3 hole uPA1, LC-CH3 knob uPA1 | 23 |
| 9 | HC-CH1 MMP2/9, LC-CL kappa MMP2/9 | 1 |
| 10 | HC-CH1 MMP2/9, LC-CL kappa MMP2/9 | 3 |
| 11 | HC-CH1 MMP2/9, LC-CL kappa MMP2/9 | 6.5 |
| 12 | HC-CH1 MMP2/9, LC-CL kappa MMP2/9 | 23 |
| 13 | HC-CH1 MMP2/9, LC-CL lambda MMP2/9 | 1 |
| 14 | HC-CH1 MMP2/9, LC-CL lambda MMP2/9 | 3 |
| 15 | HC-CH1 MMP2/9, LC-CL lambda MMP2/9 | 6.5 |
| 16 | HC-CH1 MMP2/9, LC-CL lambda MMP2/9 | 23 |
| 17 | αEGFR antibody | 0 |

The anti-EGFR activatable masked antibodies digested with MMP9 are shown in FIG. 5B. The order of the loaded samples for FIG. 5B, from left to right, is listed in Table 15 below.

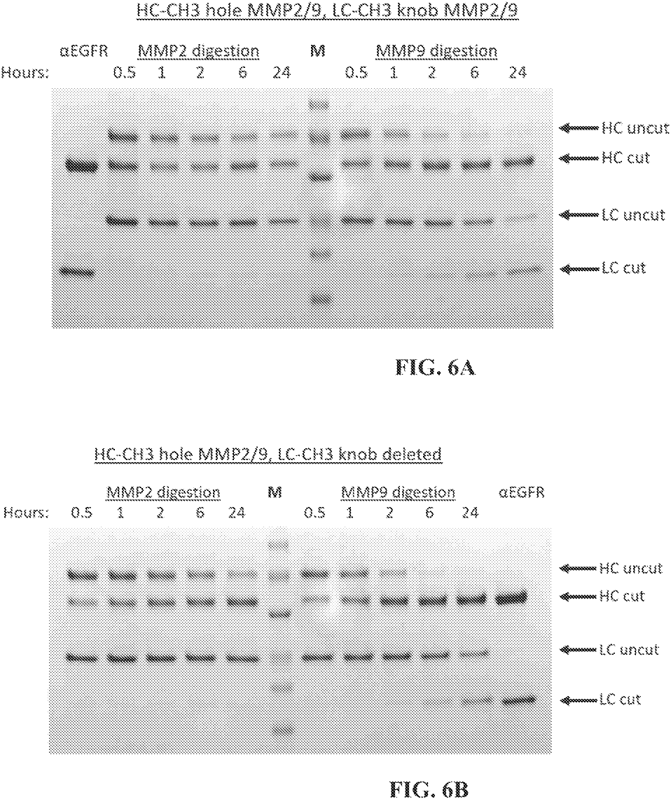

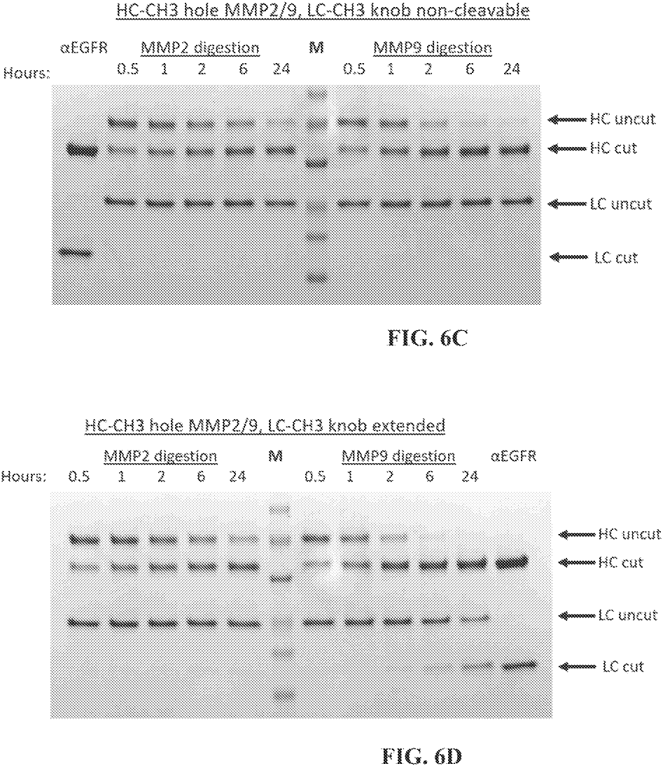

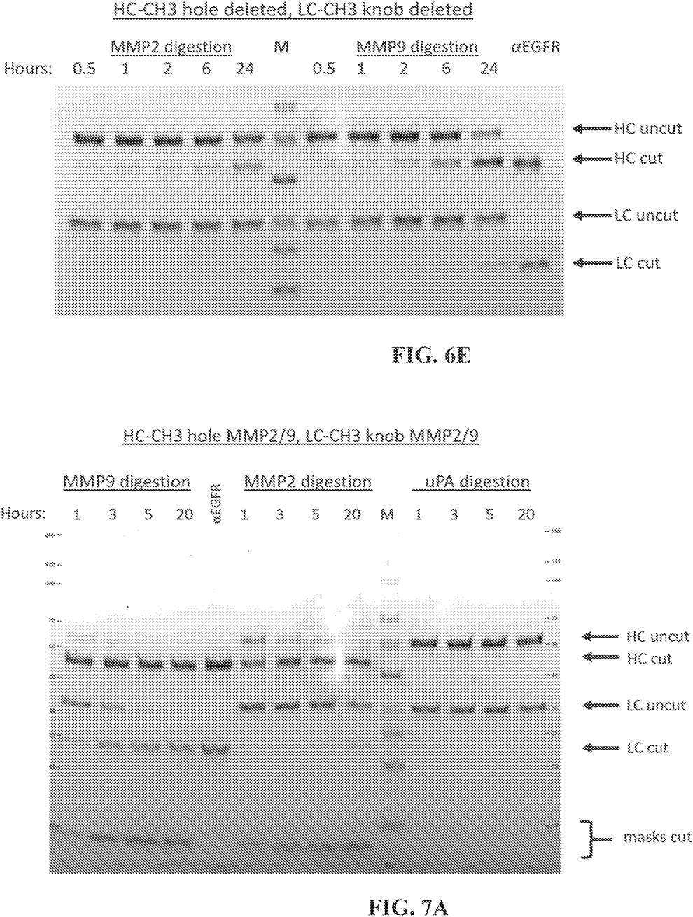

FIG. 5B: Digestion of the anti-EGFR activatable masked antibodies with MMP9 generates the predicted cleavage products, including cut heavy chain, cut light chain and cut masks (FIG. 6B). When comparing the cleavage products generated from the 1, 3, 6.5 and 23 hours digestion conditions the level of intact (uncut) light chains decreases and the level of cleaved light chain and cleaved masks (cut) increases for all masked antibodies tested.

TABLE 15

See FIG. 5B

| Lane: | Antibody: | Time (hours): |
|---|---|---|
| 1 | HC-CH3 hole MMP2/9, LC-CH3 knob MMP2/9 | 1 |
| 2 | HC-CH3 hole MMP2/9, LC-CH3 knob MMP2/9 | 3 |
| 3 | HC-CH3 hole MMP2/9, LC-CH3 knob MMP2/9 | 6.5 |
| 4 | HC-CH3 hole MMP2/9, LC-CH3 knob MMP2/9 | 23 |
| 5 | HC-CH3 hole uPA1, LC-CH3 knob uPA1 | 1 |
| 6 | HC-CH3 hole uPA1, LC-CH3 knob uPA1 | 3 |
| 7 | HC-CH3 hole uPA1, LC-CH3 knob uPA1 | 6.5 |
| 8 | HC-CH3 hole uPA1, LC-CH3 knob uPA1 | 23 |
| 9 | αEGFR antibody | 0 |
| 10 | HC-CH1 MMP2/9, LC-CL kappa MMP2/9 | 1 |
| 11 | HC-CH1 MMP2/9, LC-CL kappa MMP2/9 | 3 |
| 12 | HC-CH1 MMP2/9, LC-CL kappa MMP2/9 | 6.5 |
| 13 | HC-CH1 MMP2/9, LC-CL kappa MMP2/9 | 23 |
| 14 | HC-CH1 MMP2/9, LC-CL lambda MMP2/9 | 1 |
| 15 | HC-CH1 MMP2/9, LC-CL lambda MMP2/9 | 3 |
| 16 | HC-CH1 MMP2/9, LC-CL lambda MMP2/9 | 6.5 |
| 17 | HC-CH1 MMP2/9, LC-CL lambda MMP2/9 | 23 |

The amino acid sequences of the heavy and light chains of the various anti-EGFR IgG type activatable masked antibodies shown in FIG. 5A and B are listed below.

### HC-CH3 Hole MMP2/9, LC-CH3 Knob MMP2/9

The masking moiety is a human Ig-gamma CH3 (bolded font) which includes a hole structure, joined to an MMP2/9 peptide linker (underlined italic font), joined to an anti-EGFR IgG antibody heavy chain:

GQPREPQVYTLPPSREEMTKNQVSLSCAVKGFYPSDIAVEWESNGQPENNYKTTPPVLDSDG SFFLVSKLTVDKSRWQQGNVFSCSVMHEALHNHYTQKSLSLSPGKGGPLGVRGGQVQLKQSG
PGLVQPSQSLSITCTVSGFSLTNYGVHWVRQSPGKGLEWLGVIWSGGNTDYNTPFTSRLSIN
KDNSKSQVFFKMNSLQSN DTAIYYCARALTYYDYEFAYWGQGTLVTVSSASTKGPSVFPLAP
SSKSTSGGTAALGCLVKDYFPEPVTVSWNSGALTSGVHTFPAVLQSSGLYSLSSVVTVPSSS
LGTQTYICNVNHKPSNTKVDKRVEPKSCDKTHTCPPCPAPELLGGPSVFLFPPKPKDTLMIS
RTPEVTCVWDVSHEDPEVKFNWYVDGVEVHNAKTKPREEQYN STYRWSVLTVLHQDWLNGKE
YKCKVSNKALPAPIEKTISKAKGQPREPQVYTLPPSREEMTKNQVSLT CL VKGFYPSDIAVE
WESNGQPENNYKTTPPVLDSDGSFFLYSKLTVDKSRWQQGNVFSCSVMHEALHNHYTQKSLS
LSPGK

### HC-CH3 Hole MMP2/9, LC-CH3 Knob MMP2/9

The masking moiety is a human Ig-gamma CH3 (bolded font) which includes a knob structure, joined to an MMP2/9 peptide linker (underlined italic font), joined to an anti-EGFR IgG antibody light chain:

GQPREPQVYTLPPSREEMTKNQVSLWCLVKGFYPSDIAVEWESNGQPENNYKTTPPVLDSDG SFFLYSKLTVDKSRWQQGNVFSCSVMHEALHNHYTQKSLSLSPGKGGPLGVRGGDILLTQSP
VILSVSPGERVSFSCRASQSIGTNIHWYQQRTNGSPRLLIKYASESISGIPSRFSGSGSGTD
FTLSINSVESEDIADYYCQQNNNWPTTFGAGTKLELKRTVAAPSVFIFPPSDEQLKSGTASV
VCLLNNFYPREAKVQWKVDNALQSGNSQESVTEQDSKDSTYSLSSTLTLSKADYEKHKVYAC
EVTHQGLSSPVTKSFNRGEC

The predicted amino acid sequence of the heavy chain cleavage product of [HC-CH3 hole MMP2/9, LC-CH3 knob MMP2/9] after MMP2 or MMP9 cleavage (cleavage products confirmed by mass spectrometry as described in Example 6 below):

VRGGQVQLKQSGPGLVQPSQSLSITCTVSGFSLTNYGVHWVRQSPGKGLEWLGVIWSGGNTD
YNTPFTSRLSINKDNSKSQVFFKMNSLQSN DTAIYYCARALTYYDYEFAYWGQGTLVTVSSA
TKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCPAPELLGGPSVFLF
PPKPKDTLMISRTPEVTCVVVDVSHEDPEVKFNWYVDGVEVHNAKTKPREEQYN STYRVVSV
LTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPGK

The predicted amino acid sequence of the light chain cleavage product of [HC-CH3 hole MMP2/9, LC-CH3 knob MMP2/9] after MMP2 or MMP9 cleavage (cleavage products confirmed by mass spectrometry as described in Example 6 below):

VRGGDILLTQSPVILSVSPGERVSFSCRASQSIGTNIHWYQQRTNGSPRLLIKYASESISGI
PSRFSGSGSGTDFTLSINSVESEDIADYYCQQNNNWPTTFGAGTKLELKRTVAAPSVFIFPP
SDEQLKSGTASVVCLLNNFYPREAKVQWKVDNALQSGNSQESVTEQDSKDSTYSLSSTLTLS
KADYEKHKVYACEVTHQGLSSPVTKSFNRGEC

### HC-CH3 Hole uPA1, LC-CH3 Knob uPA1

The masking moiety is a human Ig-gamma CH3 (bolded font) which includes a hole structure, joined to a uPA1 peptide linker (underlined italic font), joined to an anti-EGFR IgG antibody heavy chain:

GQPREPQVYTLPPSREEMTKNQVSLSCAVKGFYPSDIAVEWESNGQPENNYKTTPPVLDSDG SFFLVSKLTVDKSRWQQGNVFSCSVMHEALHNHYTQKSLSLSPGKGGGGRRGGGGQVQLKQS
GPGLVQPSQSLSITCTVSGFSLTNYGVHWVRQSPGKGLEWLGVIWSGGNTDYNTPFTSRLSI
NKDNSKSQVFFKMNSLQSN DTAIYYCARALTYYDYEFAYWGQGTLVTVSSASTKGPSVFPLA
PSSKSTSGGTAALGCLVKDYFPEPVTVSWNSGALTSGVHTFPAVLQSSGLYSLSSVVTVPSS
SLGTQTYICNVNHKPSNTKVDKRVEPKSCDKTHTCPPCPAPELLGGPSVFLFPPKPKDTLMI
SRTPEVTCVVVDVSHEDPEVKFNWYVDGVEVHNAKTKPREEQYN STYRVVSVLTVLHQDWLN
GKEYKCKVSNKALPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSVMHEALHNHYTQK
SLSLSPGK

### HC-CH3 Hole uPA1, LC-CH3 Knob uPA1

The masking moiety is a human Ig-gamma CH3 (bolded font) which includes a knob structure, joined to a uPA1 peptide linker (underlined italic font), joined to an anti-EGFR IgG antibody light chain:

GQPREPQVYTLPPSREEMTKNQVSLWCLVKGFYPSDIAVEWESNGQPENNYKTTPPVLDSDG SFFLYSKLTVDKSRWQQGNVFSCSVMHEALHNHYTQKSLSLSPGKGGGGRRGGGGDILLTQS
PVILSVSPGERVSFSCRASQSIGTNIHWYQQRTNGSPRLLIKYASESISGIPSRFSGSGSGT
DFTLSINSVESEDIADYYCQQNNNWPTTFGAGTKLELKRTVAAPSVFIFPPSDEQLKSGTAS
VVCLLNNFYPREAKVQWKVDNALQSGNSQESVTEQDSKDSTYSLSSTLTLSKADYEKHKVYA
CEVTHQGLSSPVTKSFNRGEC

### HC-CH1 MMP2/9, LC-CL Kappa MMP2/9

The masking moiety is a human Ig-gamma CH1 (bolded font), joined to an MMP2/9 peptide linker (underlined italic font), joined to an anti-EGFR antibody IgG antibody heavy chain:

ASTKGPSVFPLAPSSKSTSGGTAALGCLVKDYFPEPVTVSWNSGALTSGVHTFPAVLQSSGL YSLSSVVTVPSSSLGTQTYICNVNHKPSNTKVDKRVEPKSSGGPLGVRGGQVQLKQSGPGLV
QPSQSLSITCTVSGFSLTNYGVHWVRQSPGKGLEWLGVIWSGGNTDYNTPFTSRLSINKDNS
KSQVFFKMNSLQSN DTAIYYCARALTYYDYEFAYWGQGTLVTVSSASTKGPSVFPLAPSSKS
TSGGTAALGCLVKDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPKPKDTLMISRTPE
VTCVVVDVSHEDPEVKFNWYVDGVEVHNAKTKPREEQYN STYRVVSVLTVLHQDWLNGKEYK
CKVSNKALPAPIEKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEALHNHYTQKSLSLS
PGK

### HC-CH1 MMP2/9, LC-CL Kappa MMP2/9

The masking moiety is a human Ig-gamma CL kappa (bolded font), joined to an MMP2/9 peptide linker (underlined italic font), joined to an anti-EGFR antibody IgG antibody light chain:

RTVAAPSVFIFPPSDEQLKSGTASVVCLLNNFYPREAKVQWKVDNALQSGNSQESVTEQDSK DSTYSLSSTLTLSKADYEKHKVYACEVTHQGLSSPVTKSFNRGESGGPLGVRGGDILLTQSP
VILSVSPGERVSFSCRASQSIGTNIHWYQQRTNGSPRLLIKYASESISGIPSRFSGSGSGTD
FTLSINSVESEDIADYYCQQNNNWPTTFGAGTKLELKRTVAAPSVFIFPPSDEQLKSGTASV
VCLLNNFYPREAKVQWKVDNALQSGNSQESVTEQDSKDSTYSLSSTLTLSKADYEKHKVYAC
EVTHQGLSSPVTKSFNRGEC

The predicted amino acid sequence of heavy chain cleavage product of [HC-CH1 MMP2/9, LC-CL kappa MMP2/9] after MMP2 or MMP9 cleavage:

RGGQVQLKQSGPGLVQPSQSLSITCTVSGFSLTNYGVHWVRQSPGKGLEWLGVIWSGGNTDY
NTPFTSRLSINKDNSKSQVFFKMNSLQSN DTAIYYCARALTYYDYEFAYWGQGTLVTVSSAS
TKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCPAPELLGGPSVFLF
PPKPKDTLMISRTPEVTCVVVDVSHEDPEVKFNWYVDGVEVHNAKTKPREEQYN STYRVVSV
LTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPGK

The predicted amino acid sequence of light chain cleavage product of [HC-CH1 MMP2/9, LC-CL kappa MMP2/9] after MMP2 or MMP9 cleavage:

VRGGDILLTQSPVILSVSPGERVSFSCRASQSIGTNIHWYQQRTNGSPRLLIKYASESISGI
PSRFSGSGSGTDFTLSINSVESEDIADYYCQQNNNWPTTFGAGTKLELKRTVAAPSVFIFPP
SDEQLKSGTASVVCLLNNFYPREAKVQWKVDNALQSGNSQESVTEQDSKDSTYSLSSTLTLS
KADYEKHKVYACEVTHQGLSSPVTKSFNRGEC

### HC-CH1 MMP2/9, LC-CL Lambda MMP2/9

The masking moiety is a human Ig-gamma CH1 (bolded font), joined to an MMP2/9 peptide linker (underlined italic font), joined to an anti-EGFR antibody IgG antibody heavy chain:

ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKRVEPKSSGGPLGVRGGQVQLKQSGPGLV
QPSQSLSITCTVSGFSLTNYGVHWVRQSPGKGLEWLGVIWSGGNTDYNTPFTSRLSINKDNS
KSQVFFKMNSLQSN DTAIYYCARALTYYDYEFAYWGQGTLVTVSSASTKGPSVFPLAPSSKS
TSGGTAALGCLVKDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPKPKDTLMISRTPE
VTCVVVDVSHEDPEVKFNWYVDGVEVHNAKTKPREEQYN STYRVVSVLTVLHQDWLNGKEYK
CKVSNKALPAPIEKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEALHNHYTQKSLSLS
PGK

### HC-CH1 MMP2/9, LC-CL Lambda MMP2/9

The masking moiety is a human Ig-gamma CL lambda (bolded font), joined to an MMP2/9 peptide linker (underlined italic font), joined to an anti-EGFR antibody IgG antibody light chain:

GQPKAAPSVTLFPPSSEELQANKATLVCLISDFYPGAVTVAWKADSSPVKAGVETTTPSKQS NNKYAASSYLSLTPEQWKSHRSYSCQVTHEGSTVEKTVAPTESSGGPLGVRGGDILLTQSPV
ILSVSPGERVSFSCRASQSIGTNIHWYQQRTNGSPRLLIKYASESISGIPSRFSGSGSGTDF
TLSINSVESEDIADYYCQQNNNWPTTFGAGTKLELKRTVAAPSVFIFPPSDEQLKSGTASVV
CLLNNFYPREAKVQWKVDNALQSGNSQESVTEQDSKDSTYSLSSTLTLSKADYEKHKVYACE
VTHQGLSSPVTKSFNRGEC

The predicted amino acid sequence of heavy chain cleavage product of [HC-CH1 MMP2/9, LC-CL lambda MMP2/9] after MMP2 or MMP9 cleavage:

VRGGQVQLKQSGPGLVQPSQSLSITCTVSGFSLTNYGVHWVRQSPGKGLEWLGVIWSGGNTD
YNTPFTSRLSINKDNSKSQVFFKMNSLQSN DTAIYYCARALTYYDYEFAYWGQGTLVTVSSA
STKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPELLGGPSVFL
FPPKPKDTLMISRTPEVTCVVVDVSHEDPEVKFNWYVDGVEVHNAKTKPREEQYN STYRVVS
VLTVLHQDWLNGKEYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKSRWQQGNVFSCSVM
HEALHNHYTQKSLSLSPGK

The predicted amino acid sequence of light chain cleavage product of [HC-CH1 MMP2/9, LC-CL lambda MMP2/9] after MMP2 or MMP9 cleavage:

VRGGDILLTQSPVILSVSPGERVSFSCRASQSIGTNIHWYQQRTNGSPRLLIKYASESISGI
PSRFSGSGSGTDFTLSINSVESEDIADYYCQQNNNWPTTFGAGTKLELKRTVAAPSVFIFPP
SDEQLKSGTASVVCLLNNFYPREAKVQWKVDNALQSGNSQESVTEQDSKDSTYSLSSTLTLS
KADYEKHKVYACEVTHQGLSSPVTKSFNRGEC

### HC-TCR Alpha MMP2/9, LC-TCR Beta MMP2/9

The masking moiety is a TCR alpha (bolded font), joined to an MMP2/9 peptide linker (underlined italic font), joined to an anti-CD38 IgG antibody heavy chain:

IQNPDPAVYQLRDSKSSDKSVCLFTDFDSQTNVSQSKDSDVYITDKTVLDMRSMDFKSNSAV AWSNKSDFACANAFNNSIIPEDTFFPSPGGPLGVRGGPLGVRGGQVQLVESGGGLVKPGGSL
RLSCAASGFTFSDDYMSWIRQAPGKGLEWVASVSNGRPTTYYADSVRGRFTISRDNAKNSLY
LQMNSLRAEDTAVYYCAREDWGGEFTDWGQGTLVTVSSASTKGPSVFPLAPSSKSTSGGTAA
LGCLVKDYFPEPVTVSWNSGALTSGVHTFPAVLQSSGLYSLSSVVTVPSSSLGTQTYICNVN
HKPSNTKVDKRVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQKSLSLSPGK

The masking moiety is a TCR beta (bolded font), joined to an MMP2/9 peptide linker (underlined italic font), joined to an anti-CD38 IgG antibody light chain:

KVFPPEVAVFEPSEAEISHTQKATLVCLATGFFPDHVELSWWVNGKEVHSGVSTDPQPLKEQ PALNDSRYSLSSRLRVSATFWQNPRNHFRCQVQFNGQIVSAEAWGRADSGGPLGVRGGPEGV
RGGQAGLTQPPSASGTSGQRVTISCSGSSSNIGINFVYWYQHLPGTAPKLLIYKNNQRPSGV
PDRFSGSKSGNSASLAISGLRSEDEADYYCAAWDDSLSGYVFGSGTKVTVLGQPKAAPSVTL
FPPSSEELQANKATLVCLISDFYPGAVTVAWKADSSPVKAGVETTTPSKQSNNKYAASSYLS
LTPEQWKSHRSYSCQVTHEGSTVEKTVAPTECS

### Example 4: Cleaving Modified-Sequence Peptide Linkers and Gel Analysis

Various activatable masked antibodies (anti-EGFR) having peptide linkers with modified MMP2/9 cleavage sequences were subjected to digestion with MMP2 or MMP9 proteases as described in Example 3 above except the digestion time was conducted at 0.5, 1, 2, 6 or 24 hours. The resulting digestion products were analyzed by gel electrophoresis on 4-20% gradient Tris-Glycine SDS-PAGE gels under reducing conditions (reducing agent is NUPAGE 10X Sample Reducing Agent from Invitrogen). The results are shown in FIGS. 6A-E. The marker “M” shown in FIGS. 6A-E is SPECTRA Multicolor Broad Range Protein Ladder (from Thermo Fisher Scientific, catalog # 26623).

FIG. 6A: Digestion of the activatable masked antibody [HC-CH3 hole MMP2/9, LC-CH3 knob MMP2/9] with MMP9 generates the predicted cleavage products, including cut heavy chain and cut light chain. The cleaved mask products are not shown. When comparing the cleavage products generated from the 0.5, 1, 2, 6 or 24 hours digestion conditions, the level of intact (uncut) heavy chains decreases and the level of cleaved heavy chain (cut) slightly increases, and the level of intact (uncut) light chains decreases and the level of cleaved light chain (cut) increases (FIG. 6A, lanes 8-12). Digestion with MMP2 generates little detectable cleavage products (FIG. 6A, lanes 2-6). Lane 1 is control anti-EGFR antibody, and lane 7 “M” a is molecular weight marker.

FIG. 6B: Digestion of the activatable masked antibody [HC-CH3 hole MMP2/9, LC-CH3 knob deleted] with MMP9 generates the predicted cleavage products, including cut heavy chain and cut light chain. The cleaved masks are not shown. When comparing the cleavage products generated from the 0.5, 1, 2, 6 or 24 hours digestion conditions, the level of intact (uncut) heavy chains decreases and the level of cleaved heavy chain (cut) increases, and the level of intact (uncut) light chains decreases and the level of cleaved light chain (cut) increases (FIG. 6B, lanes 7-11). Digestion with MMP2 generates little detectable cleavage products (FIG. 6B, lanes 1-5). Lane 12 is control anti-EGFR antibody, and lane 6 “M” is a molecular weight marker.

FIG. 6C: Digestion of the activatable masked antibody [HC-CH3 hole MMP2/9, LC-CH3 knob non-cleavable] with MMP9 generates the predicted cleavage products, including cut heavy chain. The cleaved masks are not shown. When comparing the cleavage products generated from the 0.5, 1, 2, 6 or 24 hours digestion conditions, the level of intact (uncut) heavy chains decreases and the level of cleaved heavy chain (cut) increases, and the level of intact (uncut) light chains does not change and cleaved light chain (cut) is not detectable (FIG. 6C, lanes 8-12). Digestion with MMP2 generates a slight decrease in the level of intact (uncut) heavy chains and a slight increase in the level of the cleaved heavy chain (cut), and the level of intact (uncut) light chains does not change and cleaved light chain (cut) is not detectable (FIG. 6C, lanes 2-6). Lane 1 is control anti-EGFR antibody, and lane 67″M″ is a molecular weight marker.

FIG. 6D: Digestion of the activatable masked antibody [HC-CH3 hole MMP2/9, LC-CH3 knob extended] with MMP9 generates the predicted cleavage products, including cut heavy chain and cut light chain. The cleaved masks are not shown. When comparing the cleavage products generated from the 0.5, 1, 2, 6 or 24 hours digestion conditions, the level of intact (uncut) heavy chains decreases and the level of cleaved heavy chain (cut) increases, and the level of intact (uncut) light chains decreases and the level of cleaved light chain (cut) increases (FIG. 6D, lanes 7-11). Digestion with MMP2 generates a slight decrease in the level of intact (uncut) heavy chains and a slight increase in the level of the cleaved heavy chain (cut), and the level of intact (uncut) light chains exhibits no detectable change and cleaved light chain (cut) is not detectable (FIG. 6D, lanes 1-5). Lane 12 is control anti-EGFR antibody, and lane 6 “M” is a molecular weight marker.

FIG. 6E: Digestion of the activatable masked antibody [HC-CH3 hole deleted, LC-CH3 knob deleted] with MMP9 generates the predicted cleavage products, including cut heavy chain and cut light chain. The cleaved masks are not shown. When comparing the cleavage products generated from the 0.5, 1, 2, 6 or 24 hours digestion conditions, the level of intact (uncut) heavy chains decreases and the level of cleaved heavy chain (cut) increases, and the level of intact (uncut) light chains decreases and the level of cleaved light chain (cut) increases (FIG. 6E, lanes 7-11). Digestion with MMP2 generates a slight decrease in the level of intact (uncut) heavy chains and a slight increase in the level of the cleaved heavy chain (cut), and the level of intact (uncut) light chains exhibits no detectable change and cleaved light chain (cut) increases very slightly (FIG. 6E, lanes 1-5). Lane 12 is control anti-EGFR antibody, and lane 6 “M” is a molecular weight marker.

### Example 5: Cleaving Modified-Sequence Peptide Linkers and Gel Analysis

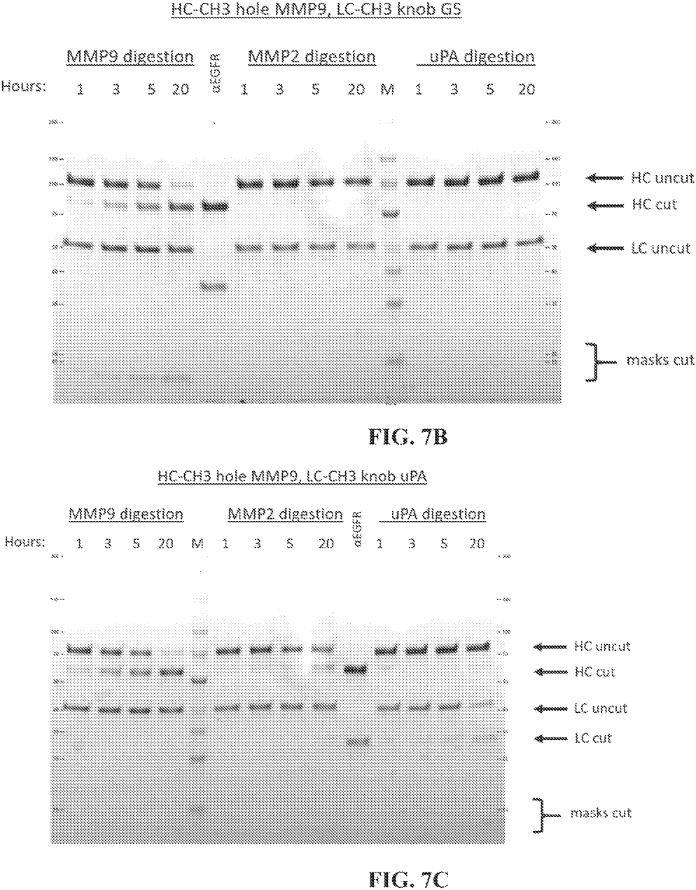

Various activatable masked antibodies (anti-EGFR) having peptide linkers with modified MMP2/9 cleavage sequences or uPA1cleavage sequences were subjected to digestion with MMP2, MMP9 or uPA proteases as described in Example 3 above except the digestion time was conducted at 1, 3, 5 or 20 hours. The resulting digestion products were analyzed by gel electrophoresis on 4-20% gradient Tris-Glycine SDS-PAGE gels under reducing conditions (reducing agent is NUPAGE 10X Sample Reducing Agent from Invitrogen). The results are shown in FIGS. 7A-C. The marker “M” shown in FIGS. 7A-C is SPECTRA Multicolor Broad Range Protein Ladder (from Thermo Fisher Scientific, catalog # 26623).

FIG. 7A: Digestion of the activatable masked antibody [HC-CH3 hole MMP2/9, LC-CH3 knob MMP2/9] with MMP9 generates the predicted cleavage products, including cut heavy chain and cut light chain. When comparing the cleavage products generated from the 1, 3, 5 or 20 hours digestion conditions, the level of intact (uncut) heavy chains disappears after 3 hours and the level of cleaved heavy chain (cut) appears after 1 hour digestion, and the level of intact (uncut) light chains decreases and the level of cleaved light chain (cut) increases, and the level of cut masking moieties increases (FIG. 7A, lanes 1-4). Digestion with MMP2 generates the predicted cleavage products, but at lower levels compared to digestion with MMP9. When comparing the cleavage products generated from the 1, 3, 5 or 20 hours digestion conditions, the level of intact (uncut) heavy chains decreases and the level of cleaved heavy chain (cut) appears after 1 hour digestion, and the level of intact (uncut) light chains decreases slightly and the level of cleaved light chain (cut) increases slightly, and the level of cut masking moieties increases (FIG. 7A, lanes 6-9). Digestion with uPA does not generate any detectable cleavage products (FIG. 7A, lanes 11-14). Lane 10 “M” a is molecular weight marker.

FIG. 7B: Digestion of the activatable masked antibody [HC-CH3 hole MMP9, LC-CH3 knob GS] with MMP9 generates the predicted cleavage products, including cut heavy chain and cut light chain. When comparing the cleavage products generated from the 1, 3, 5 or 20 hours digestion conditions, the level of intact (uncut) heavy chains decreases and the level of cleaved heavy chain (cut) appears after 1 hour digestion, and the level of intact (uncut) light chains decreases and the level of cleaved light chain (cut) increases, and the level of cut masking moieties increases (FIG. 7B, lanes 1-4). Digestion with MMP2 generates no detectable cleavage products. (FIG. 7B, lanes 6-9). Digestion with uPA generates no detectable cleavage products (FIG. 7B, lanes 11-14). Lane 10 “M” a is molecular weight marker.

FIG. 7BC: Digestion of the activatable masked antibody [HC-CH3 hole MMP9, LC-CH3 knob uPA1] with MMP9 generates the predicted heavy chain cleavage products. When comparing the cleavage products generated from the 1, 3, 5 or 20 hours digestion conditions, the level of intact (uncut) heavy chains decreases and the level of cleaved heavy chain (cut) appears after 1 hour digestion, and a low level of cut masking moieties is detectable, however no cleaved light chain products are detectable (FIG. 7C, lanes 1-4). Digestion with MMP2 generates a decrease in the level of intact heavy chain and an increase in the level of cut heavy chain, and a low level of cut masking moieties is detectable, however no cleaved light chain products are detectable (FIG. 7C, lanes 6-9). Digestion with uPA generates no detectable heavy chain cleavage products, but the level of intact light chains decreases and the level of cut light chain increases, and a low level of cut masking moieties is detectable (FIG. 7C, lanes 11-14). Lane 5 “M” a is molecular weight marker.

### Example 6: Mass Spectroscopy Analysis of Protease Cleavage Products

The mass of protease-digested anti-EGFR activatable masked antibodies were determined by subjecting the antibodies to a de-glycosylation reaction and then analyzed by mass spectroscopy.

The activatable masked antibodies were either non-MMP9 digested or digested with MMP9 according to the procedure described in Example 3 above. These antibodies were not digested to yield Fab and Fc domains. Ten micrograms of the MMP9-digested activatable masked IgG type antibody was mixed with water in total volume of 8 uL, then 2 uL of 5X PNGase F Buffer (non-reducing format, New England BioLabs, Catalog # B0717S) was added to make a 10 uL total reaction volume, which was incubated for 5 minutes at 75° C. The mixture was allowed to cool to room temperature. The de-glycosylation reaction was conducted by adding 1 uL Rapid PNGase F (non-reducing format, New England BioLabs, Catalog # P0711) which was incubated for 10 minutes at 50° C.

Liquid chromatography mass spectrometry (LC-MS) was conducted by analyzing approximately 2 ug of the de-glycosylated sample on a VANQUISH HORIZON (from Thermo Fisher Scientific, Waltham, MA) with a HESI source connected to a Q EXACTIVE Plus BioPharma mass spectrometer (Thermo Fisher Scientific, Waltham, MA) in the positive ion mode. The intact antibodies were separated by UPLC Waters BEH C4 column (2.1 x 150 mm, 300 Angstrom pore size) at a flow rate of 0.35 mL/min with the column temperature maintained at 80° C. Mobile phase A was 0.1% (v/v) formic acid in water, and mobile phase B was 0.1% (v/v) formic acid in 100% acetonitrile. Beginning with 10% mobile phase B and hold at 10% B for 5 minutes, phase B linearly increased to 25% after 1 min and to 60% at 14 min, and then to 90% B at 15 minutes. MS data was acquired in HMR mode. Acquired MS data was analyzed using Thermo BioPharma Finder software.

The anti-EGFR IgG heavy chain contains arginine residues at position 88 (e.g., in the Fab region) and 299 (e.g, in the Fc region) which are potential post-translational glycosylation sites. The “N” residue is bolded and underlined in the heavy chain amino acid sequences in Example 3 above.

It is known that CHO cells expressing antibody heavy chains will cleave off the C-terminal lysine reside. Thus, expressed IgG-type antibodies (e.g., the activatable masked IgG type antibodies described herein) may contain a heterogeneous population of antibodies having the lysine residues removed from both heavy chains, or removed from one of the heavy chains, or both heavy chains retain the C-terminal lysine residue. The activatable masked IgG type antibodies described in the Examples herein were expressed in CHO cells. In the amino acid sequences of the activatable masked antibodies described in Example 3 above, the C-terminal lysine of the heavy chains is shown in bold underlined font.

The theoretical molecular weights for de-glycosylated forms of MMP-digested or intact activatable masked antibodies [HC-CH3 hole MMP2/9, LC-CH3 knob MMP2/9] and [HC-CH1 MMP2/9, LC-CL lambda MMP2/9] were calculated using the predicted amino acid sequences of the MMP9-cleaved masked antibodies listed in Example 3 above, and compared with the experimentally determined molecular weights.

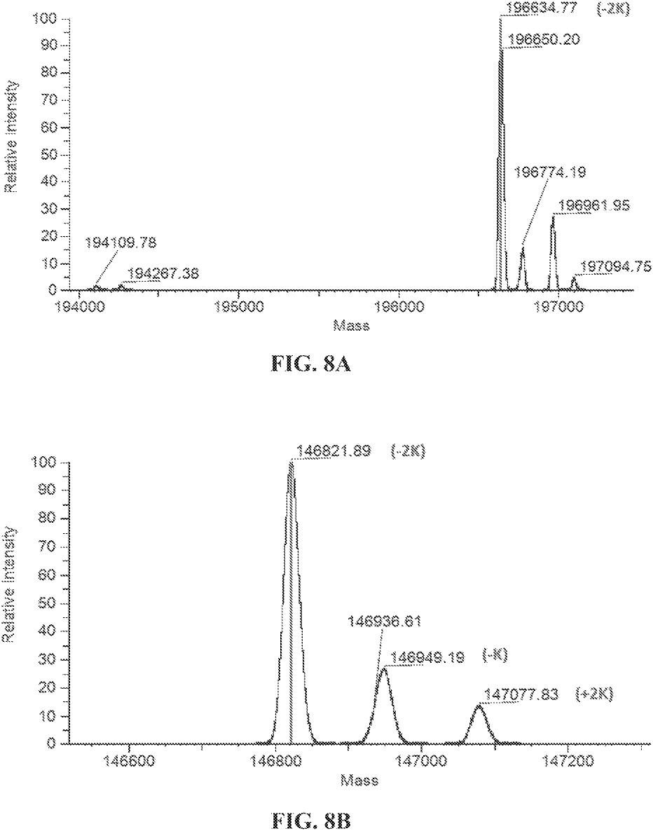

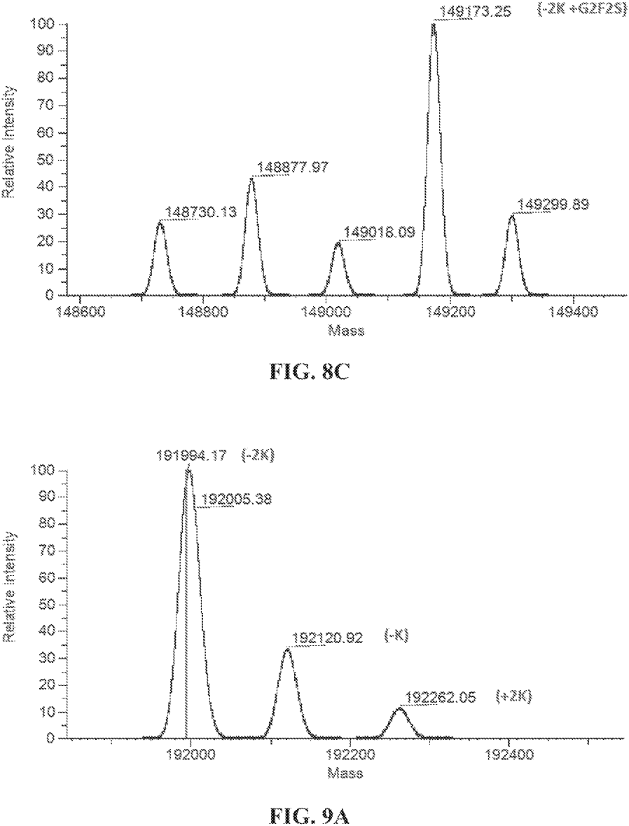

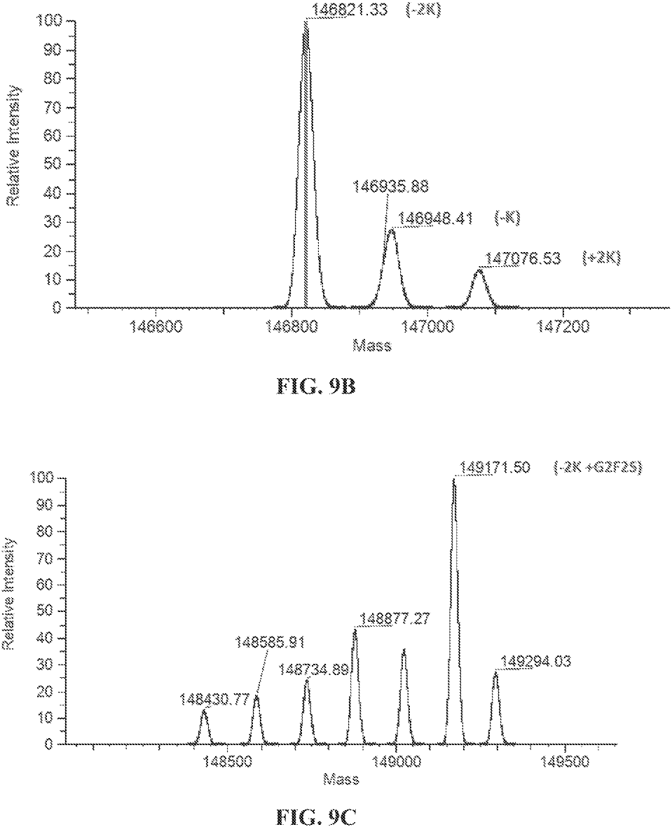

In FIGS. 8A-9C, and Tables 16-19, the term “-2K” refers to IgG molecules having the N-terminal lysine removed from both heavy chains, the term “-K″ refers to IgG molecules having the N-terminal lysine on one of the heavy chains, the term “+2K” refers to IgG molecules having the N-terminal lysine retained on both heavy chains, and the term “+G2F2S” refers to a glycan.

The LC-MS data for un-digested [HC-CH3 hole MMP2/9, LC-CH3 knob MMP2/9] is shown in FIG. 8A. The theoretical molecular weight is very close to the experimentally determined molecular weights as shown in Table 16 below.

TABLE 16

See FIG. 8A
Un-digested: HC-CH3 hole MMP2/9, LC-CH3 knob MMP2/9

| Theoretical calculated MW (Da) | Experimental MW (Da) |
|---|---|
| 196632.8 [ 2*(HC uncut + LC uncut), -2K] | 196634.77 |

The LC-MS data for MMP9 digested [HC-CH3 hole MMP2/9, LC-CH3 knob MMP2/9] is shown in FIGS. 8B and C which are split into two separate figures to accommodate the mass range of the detected molecules. The theoretical molecular weights are very close to the experimentally determined molecular weights as shown in Table 17 below.

TABLE 17

See FIGS. 8B and C
MMP9-digested: HC-CH3 hole MMP2/9, LC-CH3 knob MMP2/9

| Theoretical calculated MW (Da) | Experimental MW (Da) |
|---|---|
| 147075.1 [2*(HC short + LC short), +2K] | 147077.8 |
| 146947.1 [2*(HC short + LC short), -K] | 146949.2 |
| 146819.1 [ 2*(HC short + LC short), -2K] | 146821.9 |
| 149171.3 [ 2*(HC short + LC short), -2K,+G2F2S] | 149173.3 |

The LC-MS data for un-digested [HC-CH1 MMP2/9, LC-CL lambda MMP2/9] is shown in FIG. 9A. The theoretical molecular weight is very close to the experimentally determined molecular weights as shown in Table 18 below.

TABLE 18

See FIG. 9A
Un-digested: HC-CH1 MMP2/9, LC-CL lambda MMP2/9

| Theoretical calculated MW (Da) | Experimental MW (Da) |
|---|---|
| 192251.6 [2*(HC uncut + LC uncut), +2K] | 192262.1 |
| 192123.6 [2*(HC uncut + LC uncut), -K] | 192120.9 |
| 191995.6 [ 2*(HC uncut + LC uncut), -2K] | 191994.2 |

The LC-MS data for MMP9 digested [HC-CH1 MMP2/9, LC-CL lambda MMP2/9] is shown in FIGS. 9B and C which are split into two separate figures to accommodate the mass range of the detected molecules. The theoretical molecular weights are very close to the experimentally determined molecular weights as shown in Table 19 below.

TABLE 19

See FIGS. 9B and C
MMP9-digested: HC-CH1 MMP2/9, LC-CL lambda MMP2/9

| Theoretical calculated MW (Da) | Experimental MW (Da) |
|---|---|
| 147075.1 [2*(HC short + LC short), +2K] | 147076.5 |
| 146947.1 [2*(HC short + LC short), -K] | 146948.4 |
| 146819.1 [ 2*(HC short + LC short), -2K] | 146821.3 |
| 149171.3 [ 2*(HC short + LC short), -2K,+G2F2S] | 149171.5 |

### Example 7: FACS Analysis of Cell Binding Assays of Various IgG-Type Activatable Masked Antibodies

Cell binding assays were conducting using flow cytometry to determine EC50 values of anti-EGFR activatable masked antibodies [HC-CH3 hole MMP2/9, LC-CH3 knob MMP2/9] that were uncut (solid circles) or cut (solid squares) with MMP9, compared to a control anti-EGFR antibody (solid triangles). Additionally, uncut and cut anti-EGFR activatable masked antibodies [HC-CH3 hole uPA1, LC-CH3 knob MMP2/9] were tested and compared to the control anti-EGFR antibody.

The activatable masked antibodies were digested with MMP9 according to the protocol described in Example 3 above. EGFR positive tumor cell lines MDA-MB-231 (low EGFR expression), A431 (high EGFR expression) and MDA-MB-468 (high EGFR expression) were used. Cells were washed and resuspended in FACS buffer. The cells were counted and diluted to 1 × 10⁶ cells/mL in FACS buffer containing 0.01% sodium azide and 50 uL (~ 50,000 cells) were aliquoted into clear V-bottom 96-well plates and spun for 5 minutes at 1200 RPM. The supernatant was removed and 30 uL of serially-diluted antibody was added. The antibody was diluted to a starting concentration between 10 and 50 ug/mL and then serially diluted 3-fold using FACS buffer. Each antibody concentration was added to cells in duplicate or triplicate. After incubating one hour at 4° C., 110 uL of FACS buffer was added to each well and the plate was spun for 5 minutes at 1200 RPM. After removing the supernatant, an anti-human IgG-PE or -APC or -FITZ/Alexa488 secondary antibody was added at a 500-fold dilution in FACS buffer + 0.01% azide, and incubated for 25 minutes at 4° C. Cells were washed with 110 uL of FACS buffer and spun. The cells were resuspended in 30 uL of FACS buffer and analyzed by FACS for binding using the Intellicyt FACS. Data was analyzed using Flo-Jo and GraphPad Prism.

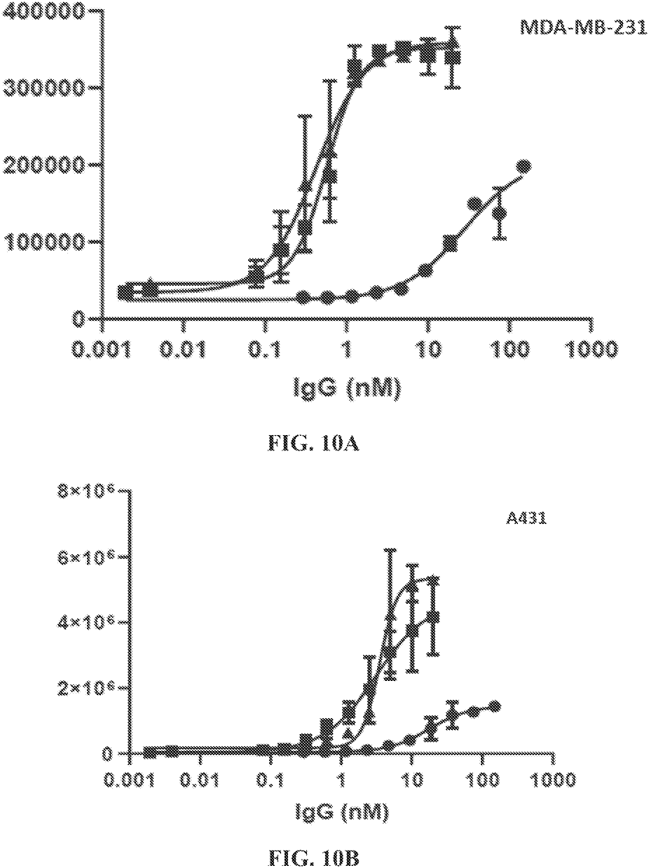

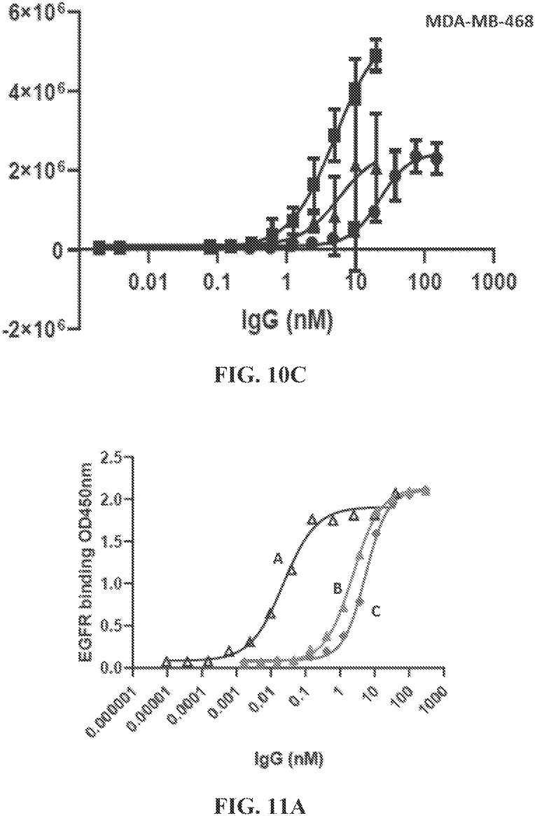

FIG. 10A shows the results of activatable masked antibody binding to MDA-MB-231 cells. The MMP9-digested antibody binds to MDA-MB-231 cells at a level similar to the control anti-EGFR antibody, while uncut antibody exhibits poor cell binding.

FIG. 10B shows the results of activatable masked antibody binding to A431 cells. The MMP9-digested antibody binds to A431 cells at a level similar to the control anti-EGFR antibody, while uncut antibody exhibits poor cell binding.

FIG. 10C shows the results of activatable masked antibody binding to MDA-MB-468 cells. The MMP9-digested antibody binds to MDA-MB-468 cells at a higher level compared to the control anti-EGFR antibody, while uncut antibody exhibits poor cell binding.

Cell binding assays were conducted with an activatable masked antibody having a first heavy chain variable region joined to a first peptide linker, and a first light chain variable region joined to a second peptide linker, wherein the first and second peptide linkers comprise different lengths and sequences and are cleavable with different proteases. For example, the cell binding ability of activatable masked antibody [HC-CH3 hole uPA1; LC-CH3 knob MMP2/9], either uncut or digested with MMP9 protease, was tested for MDA-MB-231, A431 and MDA-MB-468 cells. The EC50 values are listed in Table 20 below (see the uncut and cut antibodies designated with **).

The EC50 values obtained from these FACs assays and calculated EC50 ratios are listed in Table 20 below:

TABLE 20

See FIGS. 10A-C

| | | | |
|---|---|---|---|
| Cell line: | Antibody uncut or cut with MMP9: | EC50: | EC50 ratio: |
| MDA-MB-231 | Control anti-EGFR antibody | 0.43 nM | N/A |
| | Uncut [HC-CH3 hole MMP2/9, LC-CH3 knob MMP2/9] | 28 nM | 65X |
| | Cut [HC-CH3 hole MMP2/9, LC-CH3 knob MMP2/9] | 0.58 nM | |
| A431 | Control anti-EGFR antibody | 3.5 nM | N/A |
| | Uncut [HC-CH3 hole MMP2/9, LC-CH3 knob MMP2/9] | 17.5 nM | 5X |
| | Cut [HC-CH3 hole MMP2/9, LC-CH3 knob MMP2/9] | 3.1 nM | |
| MDA-MB-468 | Control anti-EGFR antibody | 5.8 nM | N/A |
| | Uncut [HC-CH3 hole MMP2/9, LC-CH3 knob MMP2/9] | 22.5 nM | 3.9X |
| | Cut [HC-CH3 hole MMP2/9, LC-CH3 knob MMP2/9] | 5.1 nM | |
| MDA-MB-231 | Control anti-EGFR antibody | 0.43 nM | N/A |
| | Uncut [HC-CH3 hole uPA1, LC-CH3 knob MMP2/9] ** | 16.4 nM | 38X |
| | Cut [HC-CH3 hole uPA1, LC-CH3 knob MMP2/9] ** | 0.4 nM | |
| A432 | Control anti-EGFR antibody | 3.5 nM | N/A |
| | Uncut [HC-CH3 hole uPA1, LC-CH3 knob MMP2/9] ** | 21 nM | 6X |
| | Cut [HC-CH3 hole uPA1, LC-CH3 knob MMP2/9] ** | 6 nM | |
| MDA-MB-468 | Control anti-EGFR antibody | 5.8 nM | N/A |
| | Uncut [HC-CH3 hole uPA1, LC-CH3 knob MMP2/9] ** | 38.9 nM | 6.7X |
| | Cut [HC-CH3 hole uPA1, LC-CH3 knob MMP2/9] ** | 2 nM | |

### Example 8: ELISA Analysis of Various IgG-Type Activatable Masked Antibodies

ELISA assays were conducted to compare binding characteristics of uncut and cut activatable masked anti-EGFR IgG type antibodies [HC-CH3 hole MMP2/9, LC-CH3 knob MMP2/9] and [HC-CH1 MMP2/9, LC-CL lambda MMP2/9] and compared to control anti-EGFR antibody.

The activatable masked antibodies were digested with MMP9 according to the protocol described in Example 3 above. Recombinant EGFR protein extracellular domain was adsorbed onto a microtiter plate followed by blocking with BLOCKER Casein in PBS (from Thermo Fisher Scientific, catalog #37528). The test or control antibody was added in a series of dilutions. An enzyme-labeled anti-Human IgG reagent that binds the test antibody was added. The chromogenic substrate 3,3′,5,5′-Tetramethylbenzidine (TMB) was added. A plate reader was used to detect color change. Wash steps are included between steps to remove excess unbound reagents.

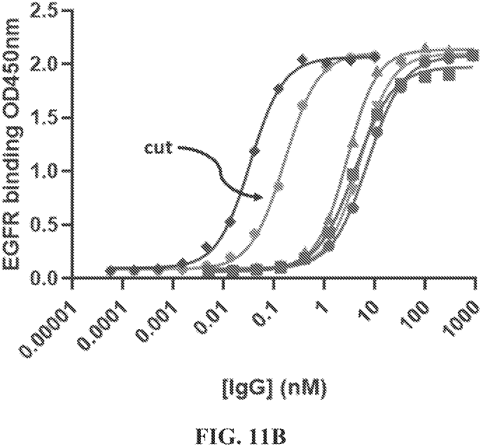

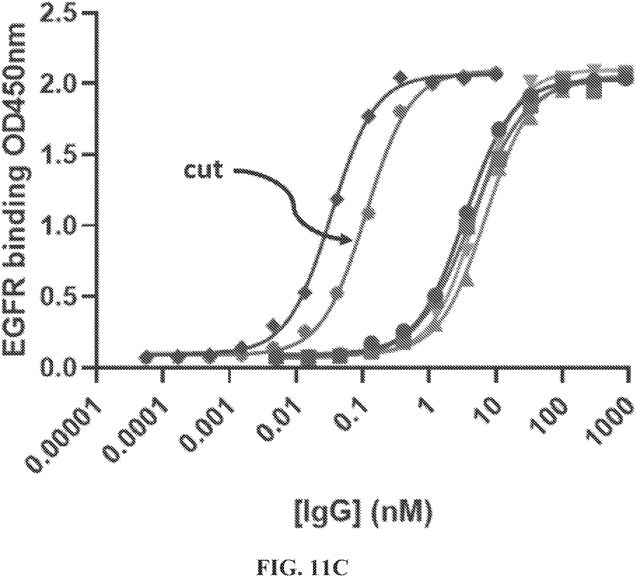

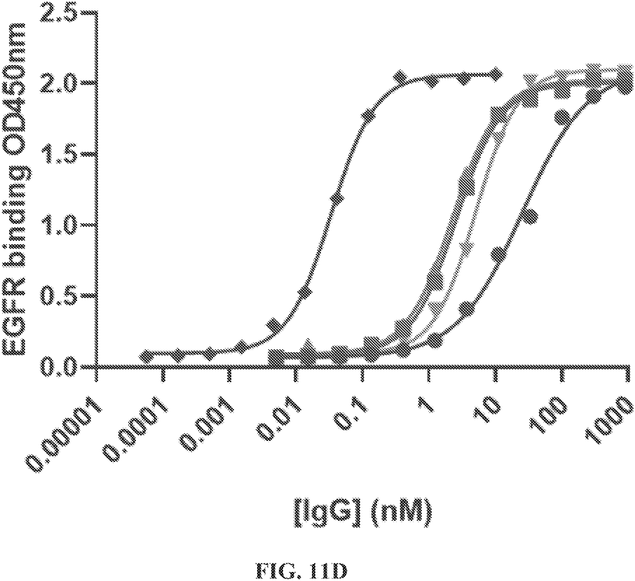

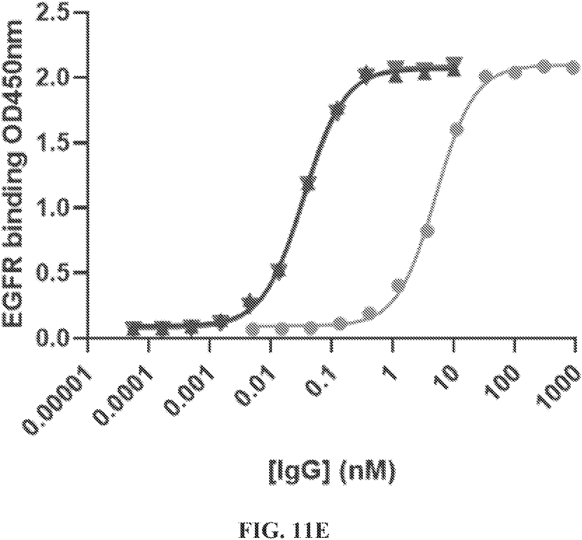

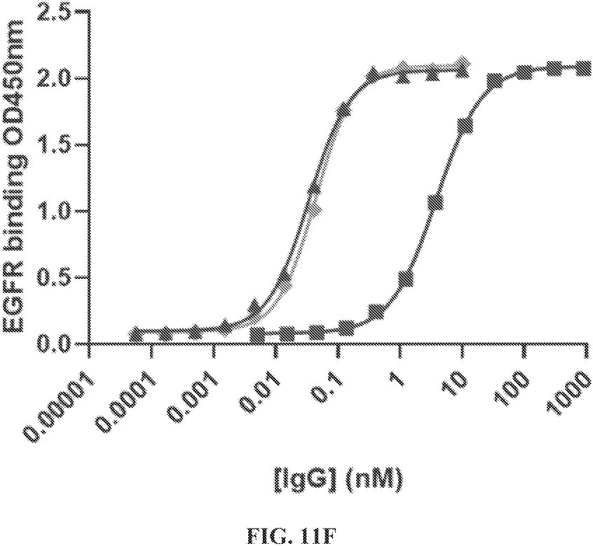

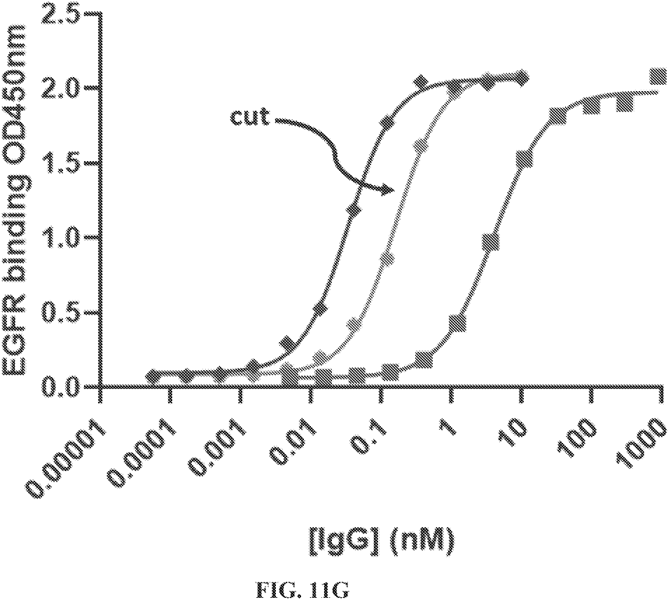

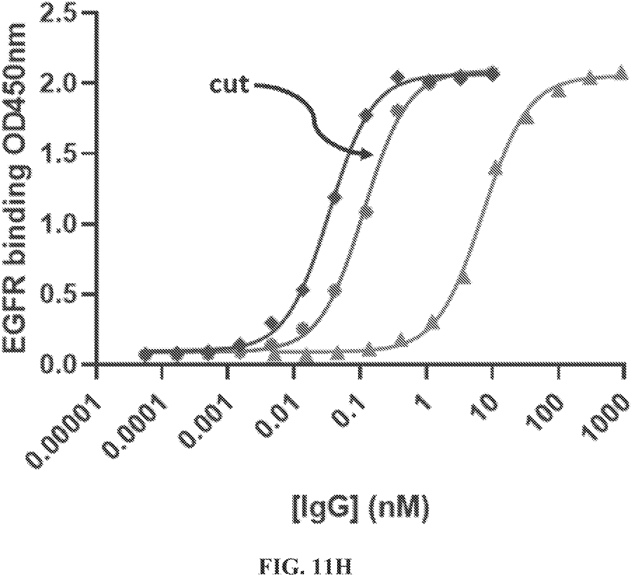

The ELISA results are shown in FIGS. 11A-H, and the EC50 and ratios are listed in Tables 21-28 below. In all cases the anti-EGFR antibody is uncut.

FIG. 11A: The binding curves of binding recombinant EGFR protein with uncut control anti-EGFR antibody (trace A, open triangle), uncut anti-EGFR [HC-CH1 MMP2/9, LC-CL lambda MMP2/9] (trace B, solid triangle), and uncut anti-EGFR [HC-CH3 hole MMP2/9, LC-CH3 knob MMP2/9] (trace C, solid diamonds). The EC50 values and ratios (EC50 of uncut control anti-EGFR antibody/EC50 value of uncut masked antibody) are listed in Table EE below. The binding curves and EC50 values indicate that the masking capability of uncut anti-EGFR [HC-CH3 hole MMP2/9, LC-CH3 knob MMP2/9] is slightly better compared to uncut anti-EGFR [HC-CH1 MMP2/9, LC-CL lambda MMP2/9].

TABLE 21

See FIG. 11A

| Antibody: | EC50: | Ratio: |
|---|---|---|
| Control anti-EGFR antibody | 22.9 pM | N/A |
| Uncut: [HC-CH3 hole MMP2/9, LC-CH3 knob MMP2/9] | 5.5 nM | 240 |
| Uncut: [HC-CH1 MMP2/9, LC-CL lambda MMP2/9] | 2.3 nM | 100 |

FIG. 11B: The binding curves of binding recombinant EGFR protein with uncut control anti-EGFR antibody (solid diamonds) and uncut activatable masked anti-EGFR antibodies [HC-CH3 hole MMP2/9, LC-CH3 knob deleted] (solid circles) and [HC-CH3 hole MMP2/9, LC-CH3 knob non-cleavable] (solid squares) and [HC-CH3 hole MMP2/9, LC-CH3 knob extended] (solid triangles) and [HC-CH3 hole MMP2/9, LC-CH3 knob MMP2/9] (solid upside-down triangles). The binding curve for [HC-CH3 hole MMP2/9, LC-CH3 knob non-cleavable] cut with MMP9 is also shown (“cut”). The EC50 values are listed in Table 22 below. The binding curves and EC50 values indicate that the masking capability of uncut masked antibody [HC-CH3 hole MMP2/9, LC-CH3 knob deleted] is slightly better compared to the other uncut masked antibodies shown in FIG. 11B, and that cleaving [HC-CH3 hole MMP2/9, LC-CH3 knob non-cleavable] with MMP9 partially restores binding capability to its target antigen compared to the binding capability of the control anti-EGFR antibody.

TABLE 22

See FIG. 11B

| | |
|---|---|
| Antibody: | EC50: |
| Uncut: Control anti-EGFR antibody | 22.9 pM |
| Uncut: [HC-CH3 hole MMP2/9, LC-CH3 knob MMP2/9] | 5.1 nM |
| Uncut: [HC-CH3 hole MMP2/9, LC-CH3 knob deleted] | 7.3 nM |
| Uncut: [HC-CH3 hole MMP2/9, LC-CH3 knob non-cleavable] | 4.1 nM |
| Uncut: [HC-CH3 hole MMP2/9, LC-CH3 knob extended] | 2.8 nM |
| Cut: [HC-CH3 hole MMP2/9, LC-CH3 knob non-cleavable] | 163.5 pM |

FIG. 11C: The binding curves of binding recombinant EGFR protein with uncut control anti-EGFR antibody (solid diamonds) and uncut activatable masked anti-EGFR antibodies [HC-CH3 hole non-cleavable, LC-CH3 knob deleted] (solid circles) and [HC-CH3 hole non-cleavable, LC-CH3 knob non-cleavable] (solid squares) and [HC-CH3 hole non-cleavable, LC-CH3 knob extended] (solid triangles) and [HC-CH3 hole MMP2/9, LC-CH3 knob MMP2/9] (solid upside-down triangles). The binding curve for [HC-CH3 hole non-cleavable, LC-CH3 knob extended] cut with MMP9 is also shown (“cut”). The EC50 values are listed in Table 23 below. The binding curves and EC50 values indicate that the masking capability of uncut masked antibody [HC-CH3 hole non-cleavable, LC-CH3 knob extended] is slightly better compared to the other uncut masked antibodies shown in FIG. 11C, and that cleaving [HC-CH3 hole non-cleavable, LC-CH3 knob extended] with MMP9 partially restores binding capability to its target antigen compared to the binding capability of the control anti-EGFR antibody.

TABLE 23

See FIG. 11C

| | |
|---|---|
| Antibody: | EC50: |
| Uncut: Control anti-EGFR antibody | 34.1 pM |
| Uncut: [HC-CH3 hole MMP2/9, LC-CH3 knob MMP2/9] | 5.1 nM |
| Uncut: [HC-CH3 hole non-cleavable, LC-CH3 knob deleted] | 3.4 nM |
| Uncut: [HC-CH3 hole non-cleavable, LC-CH3 knob non-cleavable] | 4.4 nM |
| Uncut: [HC-CH3 hole non-cleavable, LC-CH3 knob extended] | 7.2 nM |
| Cut: [HC-CH3 hole non-cleavable, LC-CH3 knob extended] | 113.5 pM |

FIG. 11D: The binding curves of binding recombinant EGFR protein with uncut control anti-EGFR antibody (solid diamonds) and uncut activatable masked anti-EGFR antibodies [HC-CH3 hole deleted, LC-CH3 knob deleted] (solid circles) and [HC-CH3 hole deleted, LC-CH3 knob non-cleavable] (solid squares) and [HC-CH3 hole deleted, LC-CH3 knob extended] (solid triangles) and [HC-CH3 hole MMP2/9, LC-CH3 knob MMP2/9] (solid upside-down triangles). The EC50 values are listed in Table 24 below. The binding curves and EC50 values indicate that the masking capability of uncut masked antibody [HC-CH3 hole deleted, LC-CH3 knob deleted] is better compared to the other uncut masked antibodies shown in FIG. 11D.

TABLE 24

See FIG. 11D

| | |
|---|---|
| Antibody: | EC50: |
| Uncut: Control anti-EGFR antibody | 34.1 pM |
| Uncut: [HC-CH3 hole MMP2/9, LC-CH3 knob MMP2/9] | 5.1 nM |
| Uncut: [HC-CH3 hole deletion, LC-CH3 knob deleted] | 25.2 nM |
| Uncut: [HC-CH3 hole deletion, LC-CH3 knob non-cleavable] | 2.6 nM |
| Uncut: [HC-CH3 hole deletion, LC-CH3 knob extended] | 2.3 nM |

FIG. 11E: The binding curves of binding recombinant EGFR protein with uncut control anti-EGFR antibody (solid diamonds) and activatable masked anti-EGFR antibody [HC-CH3 hole MMP2/9, LC-CH3 knob MMP2/9] in uncut state(solid circles) or digested with MMP9 (solid upside-down triangles). The EC50 values are listed in Table 25 below. The binding curves and EC50 values indicate that the binding capability of cut masked antibody [HC-CH3 hole MMP2/9, LC-CH3 knob MMP2/9] to it target antigen is restored to a level nearly the same as the control anti-EGFR antibody.

TABLE 25

See FIG. 11E

| | |
|---|---|
| Antibody: | EC50: |
| Uncut: Control anti-EGFR antibody | 34.1 pM |
| Uncut: [HC-CH3 hole MMP2/9, LC-CH3 knob MMP2/9] | 5.1 nM |
| Cut: [HC-CH3 hole MMP2/9, LC-CH3 knob MMP2/9] | 35.9 pM |

FIG. 11F: The binding curves of binding recombinant EGFR protein with uncut control anti-EGFR antibody (solid triangles) and activatable masked anti-EGFR antibody [HC-CH1 MMP2/9, LC-CL lambda MMP2/9] in uncut state (solid squares) or digested with MMP9 (solid diamonds). The EC50 values are listed in Table 26 below. The binding curves and EC50 values indicate that the binding capability of cut masked antibody [HC-CH3 hole MMP2/9, LC-CH3 knob MMP2/9] to its target antigen is restored to a level similar to the control anti-EGFR antibody.

TABLE 26

See FIG. 11F

| | |
|---|---|
| Antibody: | EC50: |
| Uncut: Control anti-EGFR antibody | 34.1 pM |
| Uncut: [HC-CH1 MMP2/9, LC-CL lambda MMP2/9] | 3.8 nM |
| Cut: [HC-CH1 MMP2/9, LC-CL lambda MMP2/9] | 43.8 pM |

FIG. 11G: The binding curves of binding recombinant EGFR protein with uncut control anti-EGFR antibody (solid diamonds) and activatable masked anti-EGFR antibody [HC-CH3 hole MMP2/9, LC-CH3 knob non-cleavable] in uncut state (solid squares) or digested with MMP9 (solid circles). The EC50 values are listed in Table 27 below. The binding curves and EC50 values indicate that the binding capability of cut masked antibody [HC-CH3 hole MMP2/9, LC-CH3 knob non-cleavable] to its target antigen is partially restored to the binding level of the control anti-EGFR antibody.

TABLE 27

See FIG. 11G

| | |
|---|---|
| Antibody: | EC50: |
| Uncut: Control anti-EGFR antibody | 34.1 pM |
| Uncut: [HC-CH3 hole MMP2/9, LC-CH3 knob non-cleavable] | 4.1 nM |
| Cut: [HC-CH3 hole MMP2/9, LC-CH3 knob non-cleavable] | 163.5 pM |

FIG. 11H: The binding curves of binding recombinant EGFR protein with uncut control anti-EGFR antibody (solid diamonds) and activatable masked anti-EGFR antibody [HC-CH3 hole non-cleavable, LC-CH3 knob extended] in uncut state (solid triangles) or digested with MMP9 (solid circles). The EC50 values are listed in Table 28 below. The binding curves and EC50 values indicate that the binding capability of cut masked antibody [HC-CH3 hole non-cleavable, LC-CH3 knob extended] to its target antigen is partially restored to the binding level of the control anti-EGFR antibody.

TABLE 28

See FIG. 11H

| | |
|---|---|
| Antibody: | EC50: |
| Uncut: Control anti-EGFR antibody | 34.1 pM |
| Uncut: [HC-CH3 hole non-cleavable, LC-CH3 knob extended] | 7.2 nM |
| Cut: [HC-CH3 hole non-cleavable, LC-CH3 knob extended] | 113.5 pM |

### Example 9: Bio-Layer Interferometry to Measure Protein-Protein Binding

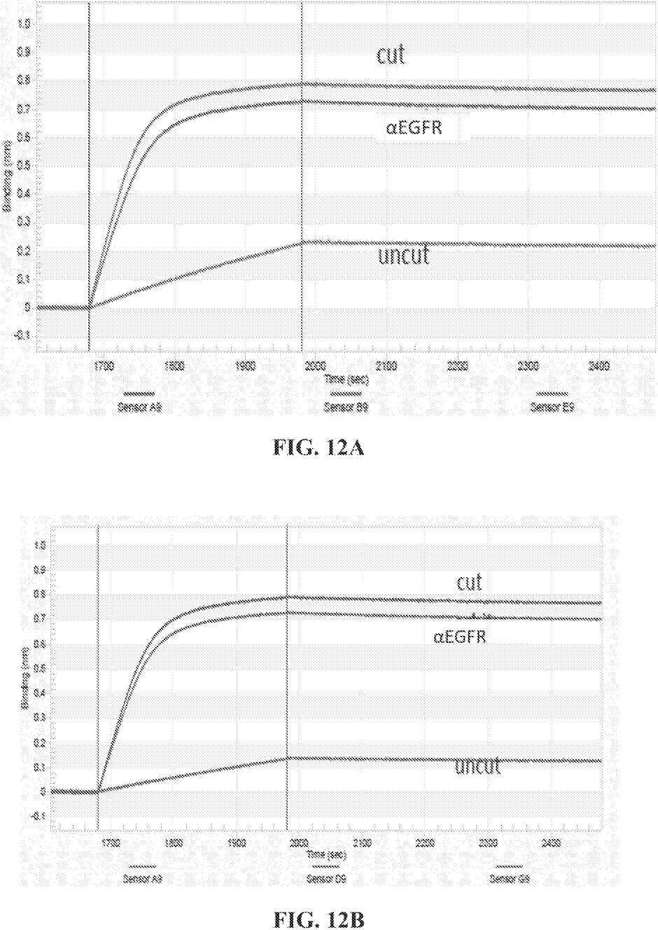

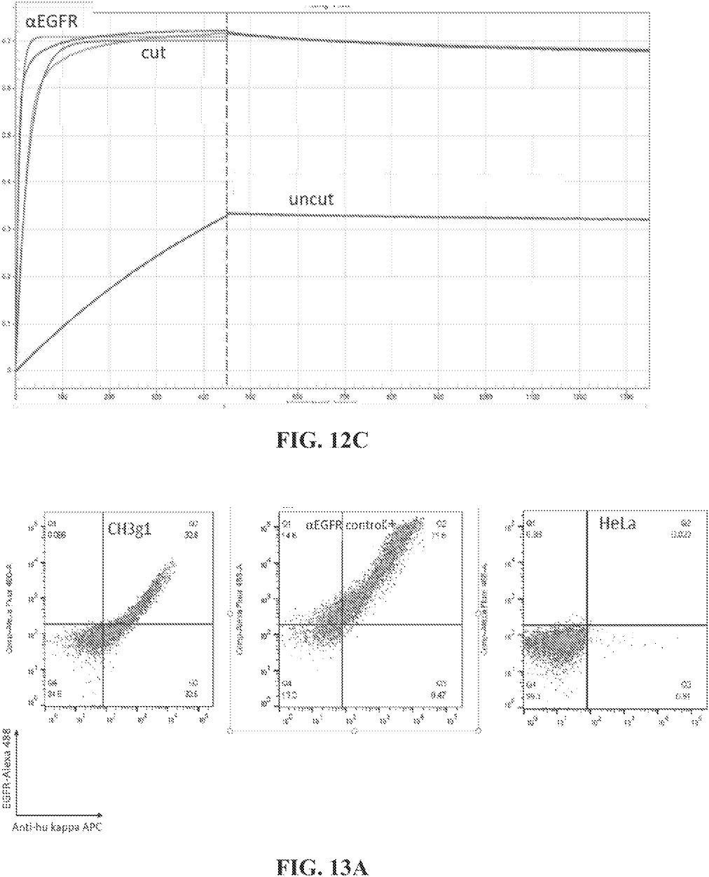

Label-free bio-layer interferometry was used to measure kinetic binding between a commercial human EGFR protein and either control anti-EGFR antibody or various anti-EGFR activatable masked antibodies. The carboxylic groups on an AR2G (amine reactive second generation) sensor were activated in aqueous solution of EDC (1-ethyl-3-(3-dimethylaminopropyl)carbodiimide hydrochloride) and NHS (N-hydroxysuccinimide). Commercial recombinant EGFR was immobilized onto the sensor in pH5.5 in acetate buffer at concentration of 5 ug/ml for 5 minutes. Time-dependent binding was detected between the EGFR protein and the control anti-EGFR antibody or activatable masked antibodies either uncut or digested with MMP9 or MMP2 protease. The control anti-EGFR antibody was not subjected to MMP9 digestion. All tested antibodies were in PBS buffer at equal molar concentration of 30 nM. Data analysis was performed according to manufacturer’s instructions. The data are shown in FIGS. 12A-C.

FIG. 12A shows that the binding capacity of the activatable masked antibody [HC-CH3 hole MMP2/9, LC-CH3 knob MMP2/9] digested with MMP9 protease is slightly better compared to the binding capacity of the control anti-EGFR antibody.

FIG. 12B shows that the binding capacity of the activatable masked antibody [HC-CH1 MMP2/9, LC-CL lambda MMP2/9] digested with MMP9 protease is slightly better compared to the binding capacity of the control anti-EGFR antibody.

FIG. 12C shows that the binding capacity of the activatable masked antibody [HC-CH1 MMP2/9, LC-CL kappa MMP2/9] digested with MMP2 protease is similar to the binding capacity of the control anti-EGFR antibody. The binding kinetic values of the [HC-CH1 MMP2/9, LC-CL kappa MMP2/9] antibody is listed in Table 29 below.

TABLE 29

See FIG. 12C

| | | | |
|---|---|---|---|
| IgG | Estimated Kd (M) | Kon (⅟Ms) | Kdis (⅟s) |
| Control anti-EGFR antibody | 6.0E-10 | 3.7E6 | 2.3E-3 |
| Uncut: [HC-CH1 MMP2/9, LC-CL kappa MMP2/9] | 1.4E-7 | 4.8E3 | 1.2E-3 |
| Cut: [HC-CH1 MMP2/9, LC-CL kappa MMP2/9] | 2.0E-9 | 1.0E6 | 2.1E-3 |

### Example 10: Determining Thermal Stability of Various IgG-Type Activatable Masked Antibodies

Intrinsic tryptophan fluorescence shift measurements were used to determine thermal stability of the various IgG-type activatable masked antibodies. UNcle from Unchained Labs (Pleasanton, California) was used to measure Tm. In a typical run, 9 µL of antibody with a concentration range of 0.1 mg/mL to 100 mg/mL was loaded. The thermal ramping was performed from 30° C. to 90° C. at a scan rate of 1° C./min, and fluorescence at full 250-720 nm spectral range was captured using a CCD digital camera. The UNcle software automatically displays the fluorescence curve calculated by BCM, and the midpoint of a thermal transition temperature (Tm, or thermal transition temperature) was also displayed. The results are listed in Tables 30-33 below:

TABLE 30

Thermal stability measurements for [HC-CH3 hole MMP2/9, LC-CH3 knob MMP2/9]:

| Sample: | Tm (°C) | Ave. Tm1 (°C) | Tagg 266 (°C) | Ave. Tagg (°C) |
|---|---|---|---|---|
| 1 - 1.23 mg/mL | 71 | 71.1 | 71.74 | 71.7 |
| 2 - 1.23 mg/mL | 70.9 | | 71.64 | |
| 3 - 1.23 mg/mL | 71.5 | | 71.62 | |

TABLE 31

Thermal stability measurements for [HC-CH1 MMP2/9, LC-CL lambda MMP2/9]:

| Sample: | Tm (°C) | Ave. Tm1 (°C) | Tagg 266 (°C) | Ave. Tagg (°C) |
|---|---|---|---|---|
| 1 - 1 mg/mL | 65.5 | 65.3 | 67.8 | 67.7 |
| 2 - 1 mg/mL | 65 | | 67.6 | |
| 3 - 1 mg/mL | 66.2 | | 67.8 | |

TABLE 32

Thermal stability measurements for [HC-CH1 MMP2/9, LC-CL kappa MMP2/9]:

| Sample: | Tm (°C) | Ave. Tm1 (°C) | Tagg 266 (°C) | Ave. Tagg (°C) |
|---|---|---|---|---|
| 1 - 1 mg/mL | 60.2 | 60 | 65.1 | 65.3 |
| 2 - 1 mg/mL | 60.3 | | 65.6 | |
| 3 - 1 mg/mL | 60 | | 65.2 | |

TABLE 33

Thermal stability measurements for additional anti-EGFR IgG-type activatable masked antibodies:

| Activatable masking moieties: | Tm 1 (°C) | Tagg 266 (°C) |
|---|---|---|
| [HC-CH3 hole MMP2/9, LC-CH3 hole uPA1] | 71 | 72.2 |
| [HC-CH3 hole MMP9, LC-CH3 knob G4S] | 71.5 | 72.9 |
| [HC-CH3 hole deleted, LC-CH3 knob deleted] | 70 | 71.2 |
| [HC-CH3 hole deleted, LC-CH3 knob non-cleavable] | 70 | 71.1 |
| [HC-CH3 hole deleted, LC-CH3 knob extended] | 69.9 | 71 |
| [HC-CH3 hole non-cleavable, LC-CH3 knob deleted] | 70.9 | 72.2 |
| [HC-CH3 hole non-cleavable, LC-CH3 knob non-cleavable] | 69.7 | 70.6 |
| [HC-CH3 hole non-cleavable, LC-CH3 knob MMP9] | 69.6 | 70.8 |
| [HC-CH3 hole MMP9, LC-CH3 knob deleted] | 69.2 | 70 |
| [HC-CH3 hole MMP9, LC-CH3 knob non-cleavable] | 70 | 70.8 |
| [HC-CH3 hole MMP9, LC-CH3 knob extended] | 69.5 | 70.5 |

### Example 11: Expression of Various Dimeric Antigen Receptor (DAR) Mimics Having Activatable Masked Antibodies

Transgenic HeLa that expressed membrane-anchored antigen binding proteins that mimicked CAR or DAR were prepared using FUGENE6 transfection reagent.

The membrane-anchored antigen binding protein DAR-mimic included (1) a first polypeptide comprising an antibody heavy chain variable region (VH), an antibody heavy chain constant region (CH), a hinge region, and a transmembrane region (TM), but lacked an intracellular signaling region, and (2) a second polypeptide comprising an antibody light chain variable region (VL) (e.g., kappa or lambda), and an antibody light chain constant region (CL). The DAR-mimic either lacked included or lacked activatable masking moieties. See the schematic of an activatable masked DAR construct in FIG. 3A and B. DAR-mimics that bind EGFR or CD38 were prepared.

The membrane-anchored antigen binding protein CAR-mimic that resembles an scFv molecule included an antibody heavy chain variable region (VH), a flexible linker, an antibody light chain variable region (VL), a hinge region, and a transmembrane region (TM), but lacked an intracellular signaling region. The CAR-mimics all lacked activatable masking moieties. CAR-mimics that bind EGFR, CD38 or BCMA antigen were prepared.

The first and second polypeptide chains of the DAR mimics were each operably joined to separate transient expression vectors that were co-introduced into HeLa cells. FUGENE HD Transfection Reagent (Promega, catalog No. E2311) was used to prepare transgenic HeLa cells that express either a CAR or DAR antigen binding protein, with or without activatable masking moieties. Transfection was conducted according the manufacturer’s instructions. Briefly, a transfection solution was prepared by mixing the FUGENE6 reagent in an EPPENDORF tube with serum free media (e.g., DMEM). The DMDM/FUGENE6 solution was added to another tube that contained the DNA encoding the CAR or DAR antigen binding proteins (e.g., expression plasmids or expression viral vectors). The quantity of the FUGENE6 and DNA used to make the DMEM/FUGENE6 and plasmid solution was according to the manufacture’s protocol. Typically, 2ug DNA with FUGENE6 reagent volume (in uL) varying between ratios of 1:2 and 1:3 for a 10 cm-well of cells (about 10⁴ or 10⁶ cells). A complex was allowed to form by incubating the DMEM media containing FUGENE6 and plasmid for 15 minutes at room temperature. The DMEM media containing the formed complex was added directly to the HeLa cells by dripping on top and swirling to mix. Cells were placed in an incubator overnight, and CAR or DAR expression was checked on day 2 or 3.

The hinge and transmembrane portions of the anti-EGFR and anti-CD38 DAR mimics (those having activatable masking moieties and those lacking activatable masking moieties) were derived from platelet-derived growth factor receptor (PDGFR).

### PDGFR Hinge: SEQ ID NO:41

AVGQDTQEVIVVPHSLPFKV

### PDGFR Transmembrane: SEQ ID NO:42

VVISAILALVVLTIISLIILIMLWQKKPR.

### PDGFR Hinge/Transmembrane: SEQ ID NO:43

AVGQDTQEVIVVPHSLPFKV VVISAILALVVLTIISLIILIMLWQKKPR.

In one embodiment, the anti-EGFR or anti-CD38 DAR or CAR (or DAR mimic or CAR mimic) having activatable masking moieties can include a hinge sequence derived from CD28 or CD8, or both CD28 and CD8.

### CD28 Hinge: SEQ ID NO:44

KIEVMYPPPYLDNEKSNGTIIHVKGKHLCPSPLFPGPSKP

### CD8 Hinge: SEQ ID NO:45

AKPTTTPAPRPPTPAPTIASQPLSLRPEACRPAAGGAVHTRGLDFAPR

### Long Hinge Sequence: CD8 and CD28 Hinge Sequences: SEQ ID NO:46

AKPTTTPAPRPPTPAPTIASQPLSLRPEACRPAAGGAVHTRGLDFAPRK
IEVMYPPPYLDNEKSNGTIIHVKGKHLCPSPLFPGPSKP

In one embodiment, the anti-EGFR or anti-CD38 DAR or CAR (or DAR mimic or CAR mimic) having activatable masking moieties can include a transmembrane sequence derived from CD28.

### CD28: Transmembrane: SEQ ID NO:47

FWVLVVVGGVLACYSLLVTVAFIIFWV

In one embodiment, the anti-EGFR or anti-CD38 DAR or CAR having activatable masking moieties can include at least one intracellular signaling sequence.

### 4-1BB Signaling Region: SEQ ID NO:48

KRGRKKLLYIFKQPFMRPVQTTQEEDGCSCRFPEEEEGGCEL

### CD28 Signaling Region: SEQ ID NO:49

RSKRSRLLHSDYMNMTPRRPGPTRKHYQPYAPPRDFAAYRS

### CD3zeta Signaling Region: SEQ ID NO:50

RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

### CD3-Zeta Signaling Region (ITAM 3): SEQ ID NO:51

RVKFSRSADKGERRRGKGHDGLYQGLSTATKDTYDALHMQALPPR

### Example 12: Cleaving Peptide Linkers of Various Dimeric Antigen Receptor (DAR) Mimics Having Activatable Masking Moieties

Transgenic HeLa cells expressing various anti-EGFR DAR mimics having activatable masking moieties were subjected to digestion with MMP9. Approximately 2 × 10⁴ transgenic HeLa cells were reacted with 0.4 ug MMP9 protease (recombinant human MMP9 from Origeen CAT#: TP302872, or RnD Systems CAT # 911-MP-010) in DPBS/2% fetal bovine serum at 25° C. for about 15 hours. APMA activation was not conducted.

### Example 13: FACS Analysis of Cell Binding Assays of Various Anti-EGFR Dimeric Antigen Receptor (DAR) Mimic Activatable Masked Antibodies

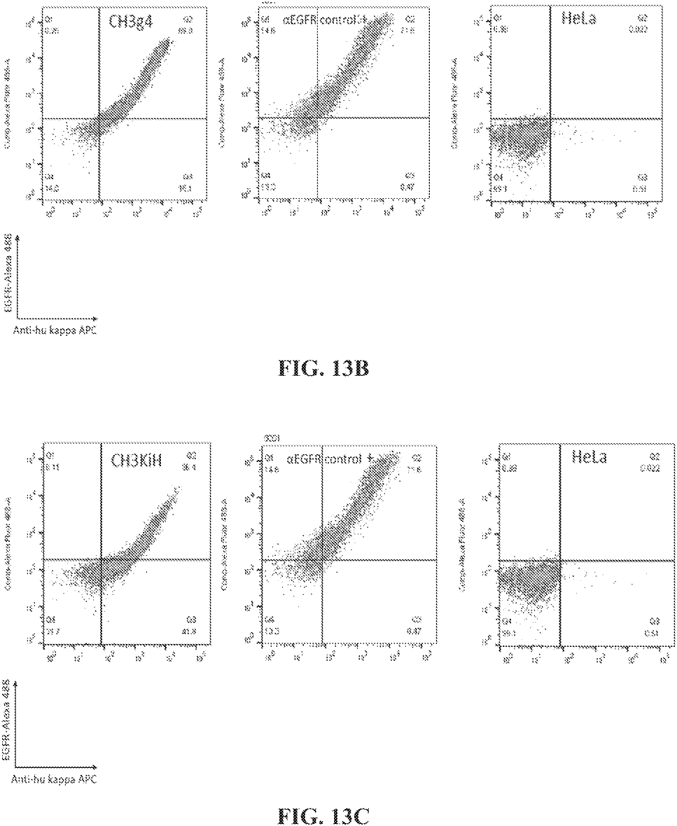

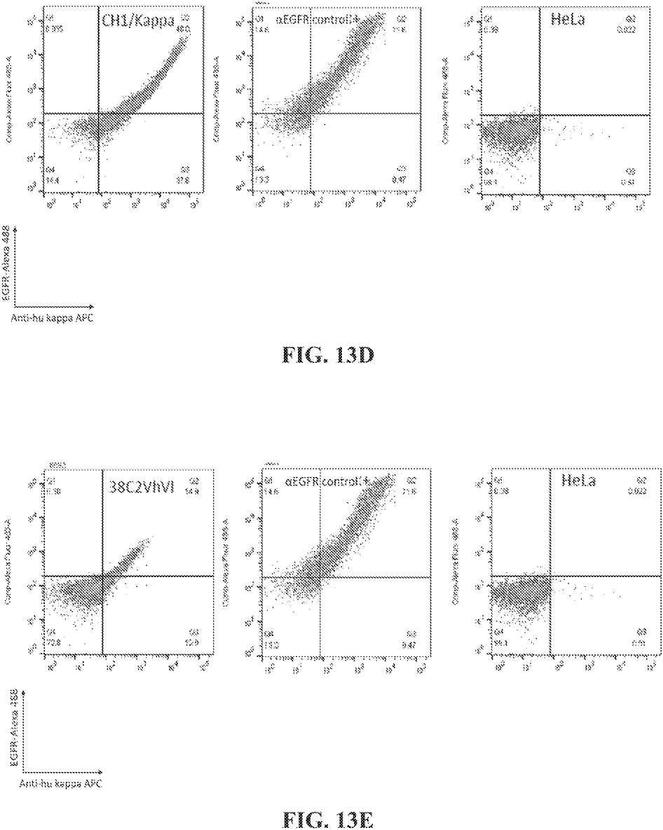

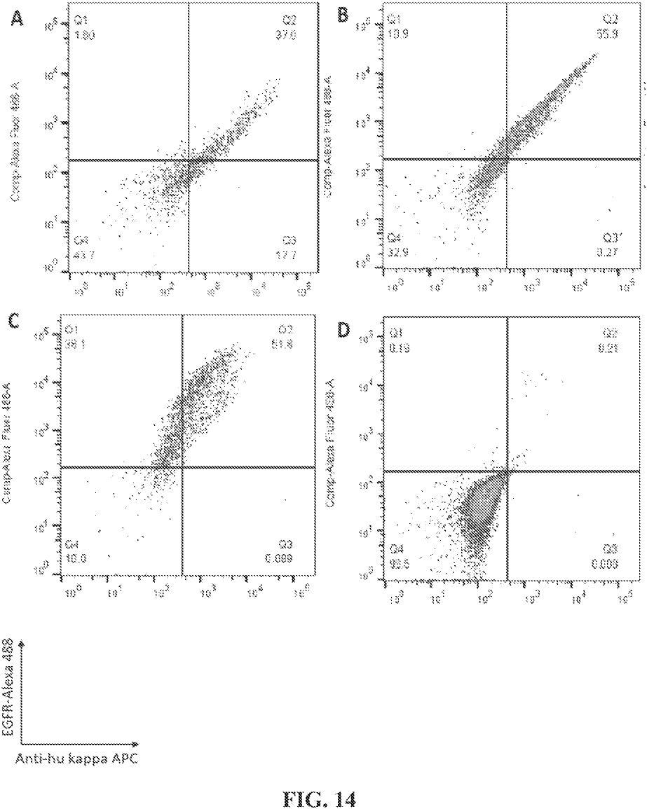

The transgenic HeLa cells expressing an anti-EGFR DAR mimic having activatable masking moieties (as described in Example 11 above) were tested in a cell binding assay using flow cytometry to assess the level of antigen binding as a reflection of masking level and to assess the level of CAR or DAR mimic expression from transgenic HeLa cells. The anti-EGFR DAR mimic having activatable masking moieties are configured as shown in the schematic of FIG. 3A. The anti-EGFR DAR mimic-expressing HeLa cells and anti-EGFR CAR mimic-expressing HeLa cells were reacted with anti-human kappa APC (from BioLegend) at 1:100 dilution and fluorophore-labeled EGFR antigen (EGFR-Alexa 488) at 1:100 dilution. The positive control transgenic HeLa cells expressed control anti-EGFR DAR mimic without peptide linkers or masking moieties, and the negative control was non-transgenic HeLa cells. The FACs results are shown in FIGS. 13A-E and FIGS. 14A-D.

FIG. 13A: Compare the Q2 quadrant showing transgenic HeLa cells expressing anti-EGFR DAR mimic having activatable masking moieties [HC-CH3(IgG1) MMP2/9, LC-CH3(IgG1) MMP2/9] in an uncut state (left; Q2 value is 32.8) binding to anti-human kappa APC and the target antigen EGFR (labeled with Alex 488), with the Q2 quadrant of positive control transgenic HeLa cells expressing the control anti-EGFR DAR mimic with no activatable masking moieties (middle; Q2 value is 71.5) binding to anti-human kappa APC and the target antigen EGFR (labeled with Alex 488), with negative control non-transgenic HeLa cells (right; Q2 value is 0.022) binding to anti-human kappa APC and the target antigen EGFR (labeled with Alex 488).

FIG. 13B: Compare the Q2 quadrant showing transgenic HeLa cells expressing anti-EGFR DAR mimic having activatable masking moieties [HC-CH3(IgG4) MMP2/9, LC-CH3(IgG4) MMP2/9] in an uncut state (left; Q2 value is 69.0) binding to anti-human kappa APC and the target antigen EGFR (labeled with Alex 488), with the Q2 quadrant of positive control transgenic HeLa cells expressing the control anti-EGFR DAR mimic with no activatable masking moieties (middle; Q2 value is 71.5) binding to anti-human kappa APC and the target antigen EGFR (labeled with Alex 488), with negative control non-transgenic HeLa cells (right; Q2 value is 0.022) binding to anti-human kappa APC and the target antigen EGFR (labeled with Alex 488).

FIG. 13C: Compare the Q2 quadrant showing transgenic HeLa cells expressing anti-EGFR DAR mimic having activatable masking moieties [HC-CH3 hole MMP2/9, LC-CH3 knob MMP2/9] in an uncut state (left; Q2 value is 36.4) binding to anti-human kappa APC and the target antigen EGFR (labeled with Alex 488), with the Q2 quadrant of positive control transgenic HeLa cells expressing the control anti-EGFR DAR mimic with no activatable masking moieties (middle; Q2 value is 71.5) binding to anti-human kappa APC and the target antigen EGFR (labeled with Alex 488), with negative control non-transgenic HeLa cells (right; Q2 value is 0.022) binding to anti-human kappa APC and the target antigen EGFR (labeled with Alex 488).

FIG. 13D: Compare the Q2 quadrant showing transgenic HeLa cells expressing anti-EGFR DAR mimic having activatable masking moieties [HC-CH1 MMP2/9, LC-CL kappa MMP2/9] in an uncut state (left; Q2 value is 46.0) binding to anti-human kappa APC and the target antigen EGFR (labeled with Alex 488), with the Q2 quadrant of positive control transgenic HeLa cells expressing the control anti-EGFR DAR mimic with no activatable masking moieties (middle; Q2 value is 71.5) binding to anti-human kappa APC and the target antigen EGFR (labeled with Alex 488), with negative control non-transgenic HeLa cells (right; Q2 value is 0.022) binding to anti-human kappa APC and the target antigen EGFR (labeled with Alex 488).

FIG. 13E: Compare the Q2 quadrant showing transgenic HeLa cells expressing anti-EGFR DAR mimic having activatable masking moieties [HC-38C2-VH MMP2/9, LC-38C2-VL MMP2/9] in an uncut state (left; Q2 value is 14.9) binding to anti-human kappa APC and the target antigen EGFR (labeled with Alex 488), with the Q2 quadrant of positive control transgenic HeLa cells expressing the control anti-EGFR DAR mimic with no activatable masking moieties (middle; Q2 value is 71.5) binding to anti-human kappa APC and the target antigen EGFR (labeled with Alex 488), with negative control non-transgenic HeLa cells (right; Q2 value is 0.022) binding to anti-human kappa APC and the target antigen EGFR (labeled with Alex 488). The paired masking moieties in the test DAR mimic construct (right) comprises a first masking moiety comprising a variable heavy chain region from an aldolase catalytic antibody 38C2, and a second masking moiety comprising a variable light chain region the aldolase catalytic antibody 38C2. The aldolase catalytic antibody 38C2 has an antigen binding domain with a reactive amino acid residue (e.g., lysine) that can catalyze an aldol reaction via an enamine mechanism to form a covalent bond/linkage with a reactive substrate molecule (e.g., ketones) (Wagner 1995 Science 270:1797; Wirshing 1995 Science 270:1775; Barbas 1997 Science 278:2085; and Hoffmann 1998 Journal of American Chemical Society 120:2768).

Compare the Q2 quadrant showing transgenic HeLa cells expressing anti-EGFR DAR mimic having activatable masking moieties [HC-CH1 MMP2/9, LC-CL kappa MMP2/9] in an uncut state (FIG. 14A; Q2 value is 37.0) binding to anti-human kappa APC and the target antigen EGFR (labeled with Alex 488), with the Q2 quadrant of transgenic HeLa cells expressing anti-EGFR DAR mimic having activatable masking moieties [HC-CH1 MMP2/9, LC-CL kappa MMP2/9] digested with MMP9 (FIG. 14B; Q2 value is 55.9) binding to anti-human kappa APC and the target antigen EGFR (labeled with Alex 488), with the Q2 quadrant of positive control transgenic HeLa cells expressing the control anti-EGFR DAR mimic with no activatable masking moieties (FIG. 14C; Q2 value is 51.8) binding to anti-human kappa APC and the target antigen EGFR (labeled with Alex 488), with the Q2 quadrant showing negative control non-transgenic HeLa cells that do not express an anti-EGFR DAR mimic and have no activatable masking moieties (FIG. 14D; Q2 value is 0.21) binding to anti-human kappa APC and the target antigen EGFR (labeled with Alex 488).

### Example 14: FACS Analysis of Cell Binding Assays of Various Anti-CD38 Dimeric Antigen Receptor (DAR) Mimic Activatable Masked Antibodies

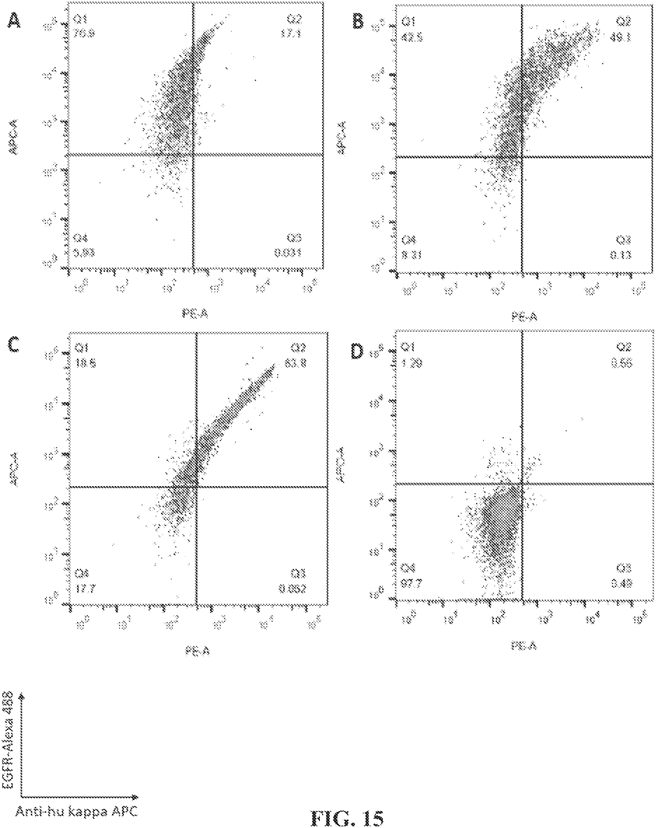

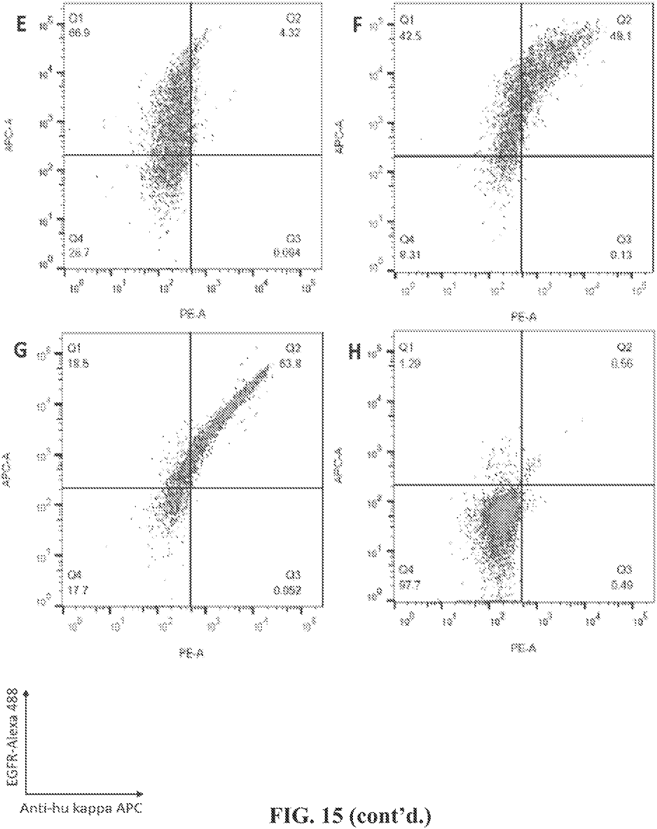

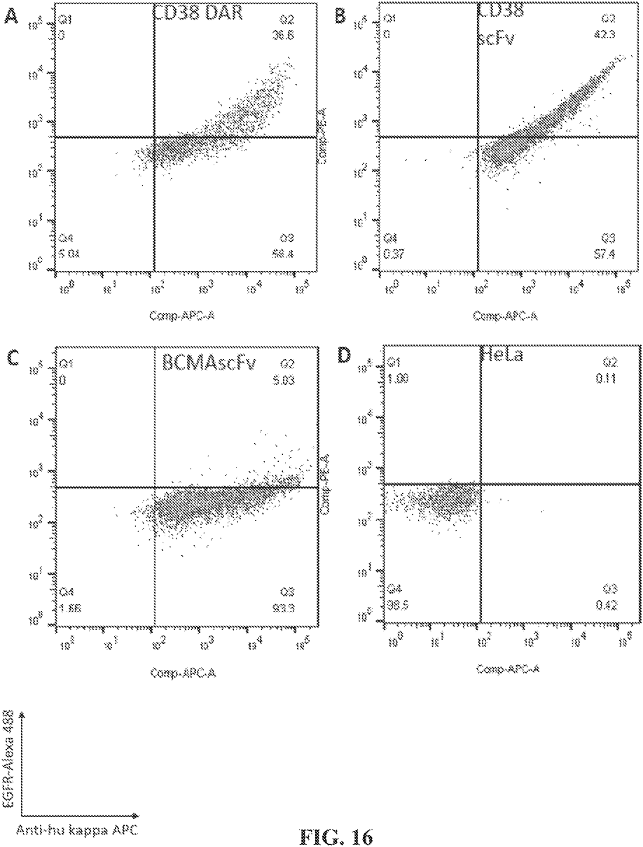

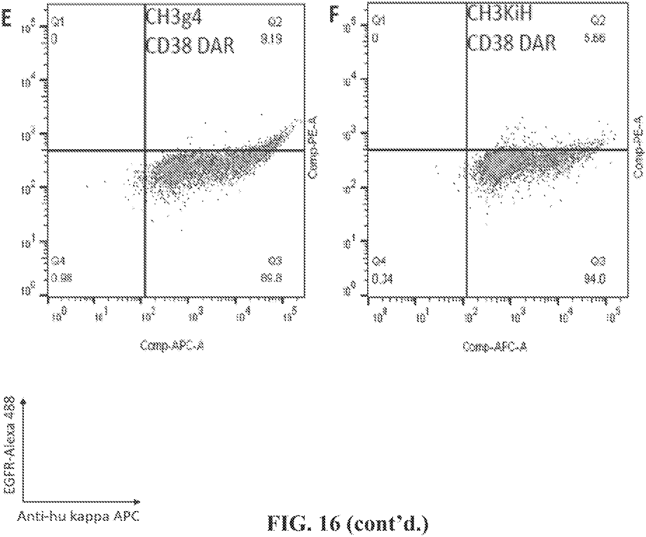

The transgenic HeLa cells expressing an anti-CD38 DAR mimic having activatable masking moieties (as described in Example 11 above) were tested in a cell binding assay using flow cytometry to assess the level of antigen binding as a reflection of masking level and to assess the level of CAR or DAR mimic expression from transgenic HeLa cells. The anti-CD38 DAR mimic having activatable masking moieties are configured as shown in the schematic of FIG. 3A. The anti-CD38 DAR mimic-expressing HeLa cells and anti-CD38 CAR mimic-expressing HeLa cells were reacted with anti-human kappa APC (from BioLegend) at 1:100 dilution and fluorophore-labeled EGFR antigen (EGFR-Alexa 488) at 1:100 dilution. The positive control transgenic HeLa cells expressed control anti-CD38 DAR mimic or CAR mimic without peptide linkers or masking moieties, and the negative control was non-transgenic HeLa cells. The FACs results are shown in FIGS. 15A-H and FIGS. 16A-F.

Compare the Q2 quadrant showing transgenic HeLa cells expressing anti-CD38 DAR mimic having activatable masking moieties [HC-CH3(IgG4) MMP2/9, LC-CH3(IgG4) MMP2/9] in an uncut state (FIG. 15A; Q2 value is 17.1) binding to CD38 Fc labeled with PE and anti-hinge antibody labeled with APC, with the Q2 quadrant showing transgenic HeLa cells expressing anti-CD38 DAR mimic having no activatable masking moieties (FIG. 15B; Q2 value is 49.1) binding to positive control CD38 Fc labeled with PE and anti-hinge antibody labeled with APC, with the Q2 quadrant showing transgenic HeLa cells expressing anti-CD38 CAR mimic but lacking activatable masking moieties (FIG. 15C; Q2 value is 63.8) binding to positive control CD38 Fc labeled with PE and anti-hinge antibody labeled with APC, with the Q2 quadrant showing negative control non-transgenic HeLa cells that do not express an anti-CD38 DAR mimic and have no activatable masking moieties (FIG. 15D; Q2 value is 0.56) binding to positive control CD38 Fc labeled with PE and anti-hinge antibody labeled with APC.

Compare the Q2 quadrant showing transgenic HeLa cells expressing anti-CD38 DAR mimic having activatable masking moieties [HC-CH3 hole MMP2/9, LC-CH3 knob MMP2/9] in an uncut state (FIG. 15E; Q2 value is 4.32) binding to CD38 Fc labeled with PE and anti-hinge antibody labeled with APC, with the Q2 quadrant showing transgenic HeLa cells expressing anti-CD38 DAR mimic having no activatable masking moieties (FIG. 15F which is the same as FIG. 15B; Q2 value is 49.1) binding to positive control CD38 Fc labeled with PE and anti-hinge antibody labeled with APC, with the Q2 quadrant showing transgenic HeLa cells expressing anti-CD38 CAR mimic but lacking activatable masking moieties (FIG. 15G which is the same as FIG. 15C; Q2 value is 63.8) binding to positive control CD38 Fc labeled with PE and anti-hinge antibody labeled with APC, with the Q2 quadrant showing negative control non-transgenic HeLa cells that do not express an anti-CD38 DAR mimic and have no activatable masking moieties (FIG. 15H which is the same as FIG. 15D; Q2 value is 0.56) binding to positive control CD38 Fc labeled with PE and anti-hinge antibody labeled with APC.

Compare the Q2 quadrant showing transgenic HeLa cells expressing anti-CD38 DAR mimic lacking activatable masking moieties (FIG. 16A; Q2 value is 36.6) binding to anti-hinge antibody labeled with APC and CD38 Fc labeled PE, with the Q2 quadrant showing transgenic HeLa cells expressing anti-CD38 CAR mimic lacking activatable masking moieties (FIG. 16B; Q2 value is 42.3) binding to anti-hinge antibody labeled with APC and CD38 Fc labeled PE, with the Q2 quadrant showing transgenic HeLa cells expressing anti-BCMA CAR mimic lacking activatable masking moieties (FIG. 16C; Q2 value is 5.03) binding to anti-hinge antibody labeled with APC and CD38 Fc labeled PE, with the Q2 quadrant showing non-transgenic HeLa cells (FIG. 16D, Q2 value is 0.11) binding to anti-hinge antibody labeled with APC and CD38 Fc labeled PE, with the Q2 quadrant showing transgenic HeLa cells expressing anti-CD38 DAR mimic having activatable masking moieties [HC-CH3(IgG4) MMP2/9, LC-CH3(IgG4) MMP2/9] in an uncut state (FIG. 16D; Q2 value is 9.19) binding to anti-hinge antibody labeled with APC and CD38 Fc labeled PE, with the Q2 quadrant showing transgenic HeLa cells expressing anti-CD38 DAR mimic having activatable masking moieties [HC-CH3 hole MMP2/9, LC-CH3 knob MMP2/9] in an uncut state (FIG. 16F; Q2 value is 5.66) binding to anti-hinge antibody labeled with APC and CD38 Fc labeled PE.

### Example 15: Imaged Cell Binding Assays of Various Anti-EGFR Dimeric Antigen Receptor (DAR) Mimics Having Activatable Masking Moieties

Incucyte imaging system (from Sartorius) was used to assess the masking and binding capability of an anti-EGFR DAR mimic having activatable masking moieties [HC-CH MMP2/9, LC-CL kappa MMP2/9] in an uncut state, in the presence or absence of conditioned media from MM1R human multiple myeloma cells.

On day one, approximately 2 x 10⁴ HeLa cells were transfected with expression vectors carrying sequences encoding either control anti-EGFR antibody lacking activatable masking moieties or encoding anti-EGFR DAR mimic with activatable masking moieties, and using the FUGENE6 reagent as described in Example 11 above. On day two, the media was replaced with MM1R conditioned media from freshly cultured tumor cell line MM1R and incubated at 37° C. for 24 hours. On day three, the HeLa cells were fixed with 4% paraformaldehyde for 15 minutes at room temperature, followed by two washes with DPBS. The cells were treated with 0.5% Triton X-100 for 10 minutes at room temperature, then washed twice with DPBS. A blocking step was conducted with 3% BSA/DPBS and double-stained with mouse anti-human LC kappa/goat anti-mouse Alexa 594 and EGFR Fc-labeled Alexa 488.

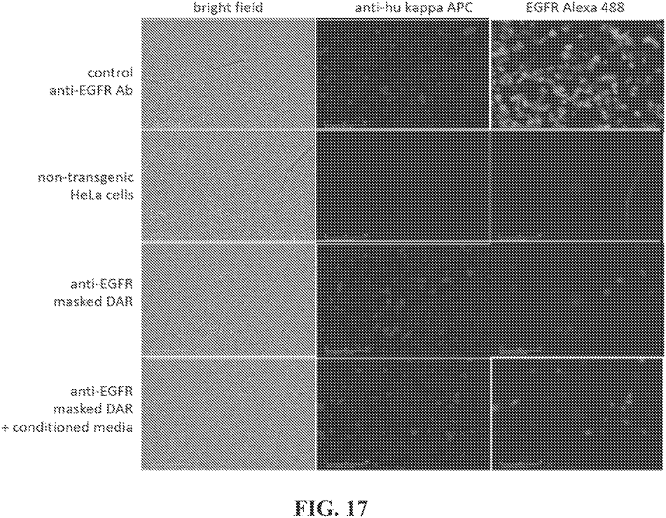

FIG. 17 shows binding/staining between transgenic HeLa cells expressing an anti-EGFR DAR mimic, and either anti-human kappa APC or Alexa 488-labeled EGFR antigen. The microscopy images in FIG. 17 (columns left to right) show bright field image, staining with anti-hu kappa APC, and staining with Alex 488-labeled EGFR antigen.

In FIG. 17, the top row shows images of positive control transgenic HeLa cells expressing the anti-EGFR DAR mimic lacking masking moieties.

The second row shows images of negative control non-transgenic HeLa cells which do not express an anti-EGFR antibody.

The third row shows images of transgenic HeLa cells expressing anti-EGFR DAR mimic having masking moieties [HC-CH MMP2/9, LC-CL kappa MMP2/9] in an uncut state, in the absence of conditioned media from MM1R human multiple myeloma cells. The APC-staining intensity is similar between the control HeLa cells expressing the DAR mimic without masking moieties (top row) and HeLa cells expressing the DAR mimic with masking moieties (third row), which indicates that these transgenic HeLa cells express similar levels of anti-EGFR DAR mimics. The Alex 488-staining intensity of the positive control transgenic HeLa cells expressing the DAR mimic lacking a masking moieties is higher compared to the staining intensity of the transgenic HeLa cells expressing the DAR mimic having the masking moieties in the uncut state, which indicates that the intact masking moieties reduced binding between the DAR mimic and EGFR antigen.

The bottom row shows images of transgenic HeLa cells expressing anti-EGFR DAR mimic having masking moieties [HC-CH MMP2/9, LC-CL kappa MMP2/9] in an uncut state, in the presence of conditioned media from MM1R human multiple myeloma cells. The Alex 488-staining intensity is similar between the HeLa cells expressing the DAR mimic having masking moieties in the absence and the presence of MM1R-conditioned media, which indicates that the conditioned media did not contain adequate levels of MMP9 protease to cleave the activatable masking moieties and thus the masking moieties are intact which reduced binding between the DAR mimic and EGFR antigen.

### Example 16 NK Cytotoxicity in an ADCC Assay

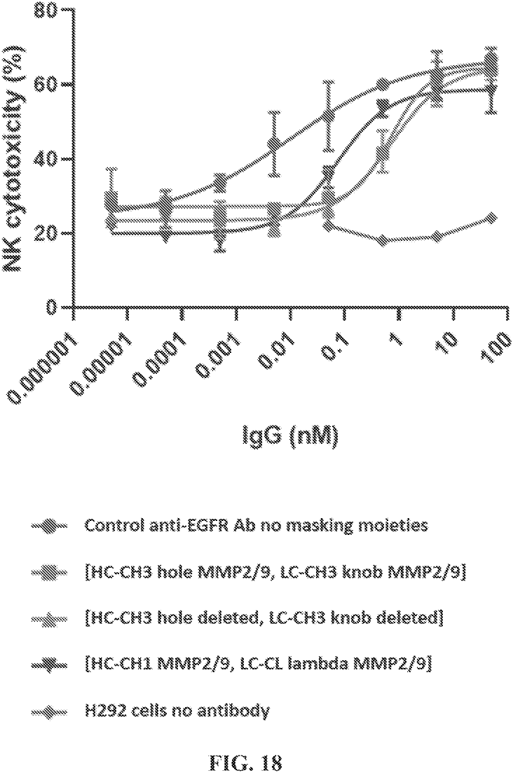

ADCC assays were conducted to determine the EC50 values of various anti-EGFR IgG-type antibodies having activatable masking moieties. NK (natural killer) cells were purified from PBMCs using EASYSEP Human NK cell enrichment kit (StemCell Technologies, catalog #19055) and incubated in RPMI 10% FBS in the presence of 20-50 IU/ml IL2. EGFR-positive tumor cell line H292 cells (which were CFSE-labeled the day before) were seeded at 15,000 cells/well in a 96-well plate. Control anti-EGFR IgG-type antibody or various anti-EGFR IgG-type antibodies having masking moieties, in the uncut state, were serially diluted in PBS buffer and added at decreasing concentration from 100 nM to 1pM (0.000001 nM) to the H292 cells in the 96-well plate then incubated 20 minutes. NK cells were added to each well such that the E:T ratio (effector cell NK : target cell H292) was 1:1, and the plate was incubated in tissue culture chamber for approximately 14 hours. FACs was used to determine the percentage of apoptotic target cells using annexin-V staining reagent while gating on the CFSE-labeled target H292 cell population (see FIG. 18). The EC50 values were determined and are listed in Table 34 below.

The results of the ADCC assay indicate that the EC50 value of anti-EGFR IgG type antibody having activatable masking moieties [HC-CH1 MMP2/9, LC-CL lambda MMP2/9] in an uncut state is about 7-fold higher compared to the control anti-EGFR IgG antibody, and the EC50 value of anti-EGFR IgG type antibody having activatable masking moieties [HC-CH3 hole MMP2/9, LC-CH3 knob MMP2/9] or [HC-CH3 hole deleted, LC-CH3 knob deleted] in an uncut state is about 70-fold higher.

TABLE 34

See FIG. 18

| | |
|---|---|
| IgG type antibody: | EC50: |
| Control anti-EGFR IgG antibody | 10.4 pM |
| [HC-CH3 hole MMP2/9, LC-CH3 knob MMP2/9] | 713.2 pM |
| [HC-CH3 hole deleted, LC-CH3 knob deleted] | 710.9 pM |
| [HC-CH1 MMP2/9, LC-CL lambda MMP2/9] | 74.5 pM |

### Example 17: Expression of Various Dimeric Antigen Receptor (DAR) Having Activatable Masked Antibodies and Carrying Intracellular Signaling Domains

Phoenix-ECO (a human embryonic kidney cell line) were transfected using FuGENE to express anti-EFGR DAR (with intracellular signaling domain) with activatable masking moieties.

Nucleic acids encoding the anti-EGFR masked DAR precursor polypeptide chains were cloned into a MFG viral vector. Two million phoenix-ECO cells were seeded in T25 flask and cultured overnight at 37° C., 5% CO₂. The next day, mixed together 8 ug DNA to 24 uL FuGENE (1:3 ratio) to phoenix-ECO cell and the cells were incubated at 37° C., 5% CO₂. At 48 hour and 96 hour post-transfection, the viral supernatant was harvested by spinning at 1600 RPM for 8 minutes. The viral supernatant was used to transduce PG13 cells.

RetroNectin coated non-tissue treated 6 well plates were used for PG13 transduction. 2 mL viral supernatant collected from phoenix-ECO was added and spun at 2500 RPM at RT for 2 hours. The viral supernatant was aspirated, then added 0.6 million PG13 cells in 2 mL viral supernatant. Spun at 2500 RPM at RT for 1 hour. The cells were incubated overnight at 37° C., 5% CO₂. The next day, the transduction was repeated. The transduction reaction was repeated 2-3 times until the DAR expression level was about 90% which required for transduction DAR into T cells. DAR expression levels were detected via flow cytometry using 1:200 APC anti-human kappa LC. The PG13 viral supernatant was harvested at 48 hours, 72 hours and 96 hours post-transduction, respectively. To transduce T cells, the PG13 viral supernatant was used freshly-prepared or was stored at -80° C. for a future T cell transduction reaction.

PBMC (either freshly-isolated from human blood or frozen) were treated with Human CD3/CD28 T Cell Activator: 3 uL of T cell Activator per million PBMC. At day 2 or 3 post-activation, the CD3 population and T cell activation marker CD25 were checked via flow cytometry. Generally, the CD3+ population was greater than 85%. Day 2 or 3 activated T cells were used for DAR transduction.

2 mL viral supernatant was collected from PG cells and added to RetroNectin coated 6 well plate, and spun at 2500 RPM at room temperature for 2 hours. The viral supernatant was aspirated, then added 1 million activated T cells in 2 mL viral supernatant. Spun at 2500 RPM for 1 hour. The transduced activated T cells were incubated for 3-4 hours, then the media was changed to T cell culture media + 300 IU/ml IL2. The transduced T cells were incubated overnight at 37° C., 5% CO₂. The T cell transduction was repeated 1-4 times until optimal DAR expression was reached.

Anti-EGFR masked DARs include: a first and a second polypeptide chain, wherein the (a) first polypeptide chain comprises: (i) a first masking moiety having the amino acid sequence of SEQ ID NO:24; (ii) a first peptide linker cleavable with MMP9 or uPA, or carrying a non-cleavable linker, and having the amino acid sequence of SEQ ID NO:32, 52 or 34, respectively; (iii) an antibody heavy chain variable region (VH) having a portion of the amino acid sequence of SEQ ID NO:2; (iv) an antibody heavy chain constant region (CH) having a portion of the amino acid sequence of SEQ ID NO:2; (v) a CD28 hinge region having the amino acid sequence of SEQ ID NO:44; (vi) a CD28 transmembrane region (TM) having the amino acid sequence of SEQ ID NO:47; and (vii) an intracellular signaling region which includes 4-1BB and CD3zeta intracellular signaling domains and having the amino acid sequence of SEQ ID NOS:48 and 50, respectively; wherein the (b) second polypeptide chain comprises: (i) a second masking moiety having the amino acid sequence of SEQ ID NO:25; (ii) a second peptide linker cleavable with MMP9 or uPA, or carrying a non-cleavable linker, and having the amino acid sequence of SEQ ID NO:32, 52 or 34, respectively; (iii) an antibody light chain variable region (VL) having a portion of the amino acid sequence of SEQ ID NO:5; and (iv) an antibody light chain constant region (CL) having a portion of the amino acid sequence of SEQ ID NO:5. These anti-EGFR masked DARs contained a first and second intracellular signaling domain, but lacked a third intracellular domain (an embodiment of the DAR molecules shown in FIG. 3A).

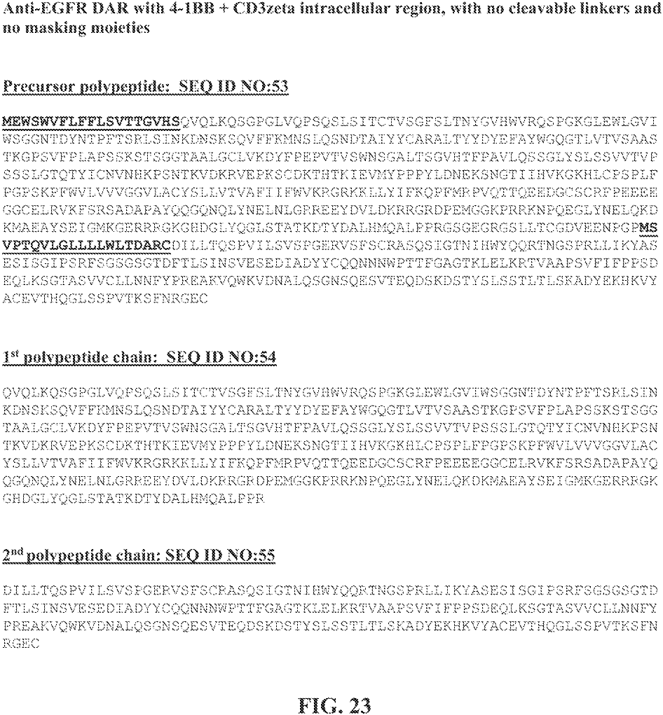

Control anti-EGFR DAR: Transgenic T cells expressed a control anti-EGFR DAR precursor polypeptide chain that contained a first polypeptide chain, a self-cleaving sequence (e.g., T2A), and a second polypeptide chain, and comprising the amino acid sequence of SEQ ID NO:53 (See FIG. 23). The T2A sequence mediated cleavage of the precursor molecule to generate first and second polypeptide chains that assembled to form a membrane-embedded DAR with intracellular signaling domains from 4-1BB and CD3zeta. The predicted first polypeptide chain of the anti-EGFR masked DAR lacking Ig gamma-1 CH3 masking moieties and lacking cleavable linkers, comprises the amino acid sequence of SEQ ID NO:54. The predicted second polypeptide chain of the anti-EGFR masked DAR lacking Ig gamma-1 CH3 masking moieties and lacking cleavable linkers, comprises the amino acid sequence of SEQ ID NO:55. The heavy and light chain leader sequences are bolded and underlined.

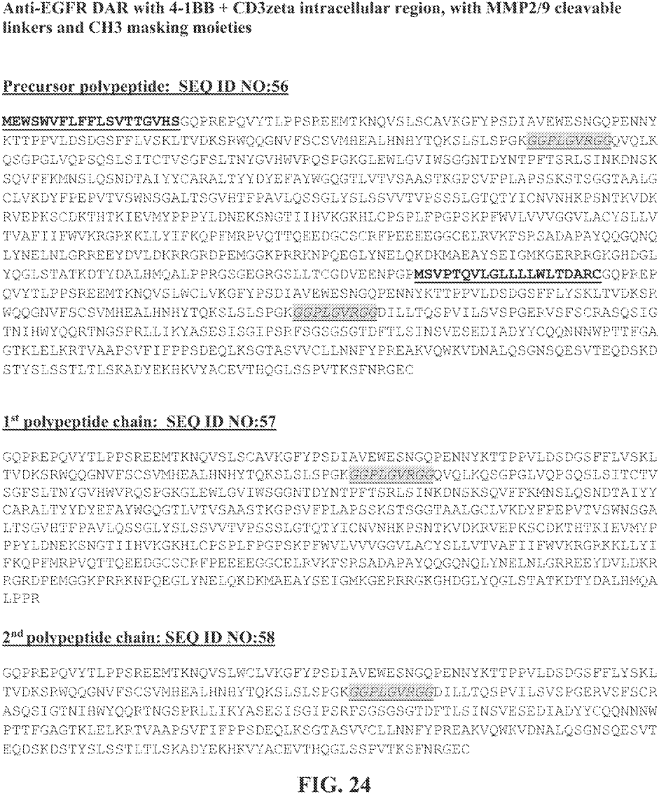

Anti-EGFR DAR with MMP2/9 cleavable linkers: Transgenic T cells expressed an anti-EGFR DAR precursor polypeptide chain that contained a first polypeptide chain, a self-cleaving sequence (e.g., T2A), and a second polypeptide chain, and comprising the amino acid sequence of SEQ ID NO:56 (See FIG. 24). The T2A sequence mediated cleavage of the precursor molecule to generate first and second polypeptide chains that assembled to form a membrane-embedded DAR with intracellular signaling domains from 4-1BB and CD3zeta. The predicted first polypeptide chain of the anti-EGFR masked DAR carrying Ig gamma-1 CH3 masking moieties linked to MMP2/9 cleavable linkers (Table 35), comprises the amino acid sequence of SEQ ID NO:57. The predicted second polypeptide chain of the anti-EGFR masked DAR carrying Ig gamma-1 CH3 masking moieties linked to MMP2/9 cleavable linkers, comprises the amino acid sequence of SEQ ID NO:58. The heavy and light chain leader sequences are bolded and underlined. The MMP2/9 cleavable linker sequences are italicized and gray-highlighted.

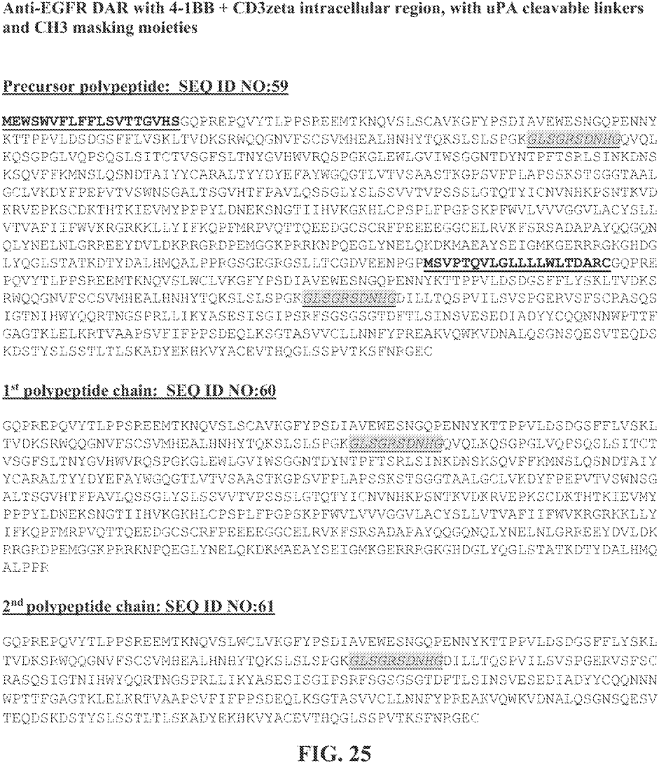

Anti-EGFR DAR with uPA cleavable linkers: Transgenic T cells expressed an anti-EGFR DAR precursor polypeptide chain that contained a first polypeptide chain, a self-cleaving sequence (e.g., T2A), and a second polypeptide chain, and comprising the amino acid sequence of SEQ ID NO:59 (See FIG. 25). The T2A sequence mediated cleavage of the precursor molecule to generate first and second polypeptide chains that assembled to form a membrane-embedded DAR with intracellular signaling domains from 4-1BB and CD3zeta. The predicted first polypeptide chain of the anti-EGFR masked DAR carrying Ig gamma-1 CH3 masking moieties linked to uPA cleavable linkers (Table 35), comprises the amino acid sequence of SEQ ID NO:60. The predicted second polypeptide chain of the anti-EGFR masked DAR carrying Ig gamma-1 CH3 masking moieties linked to uPA cleavable linkers, comprises the amino acid sequence of SEQ ID NO:61. The heavy and light chain leader sequences are bolded and underlined. The uPA cleavable linker sequences are italicized and gray-highlighted.

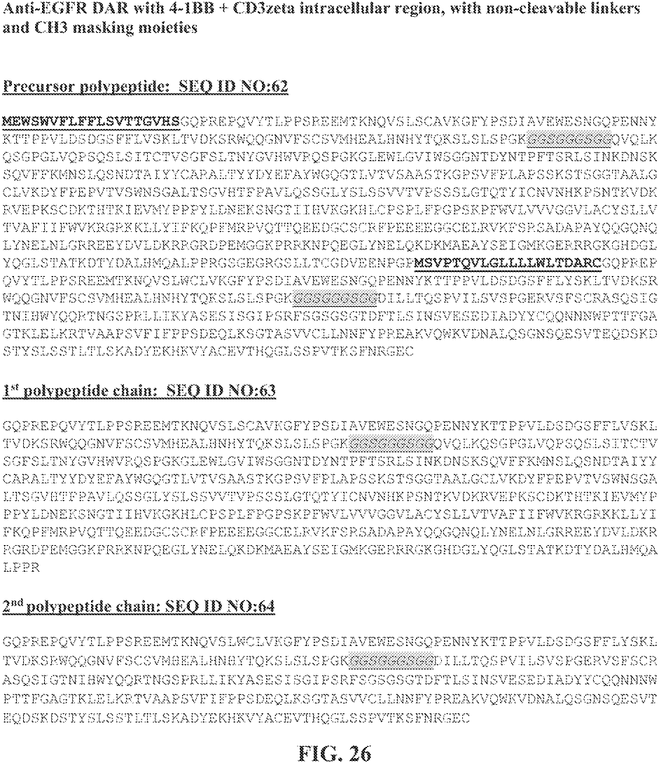

Anti-EGFR DAR with non-cleavable linkers: Transgenic T cells expressed an anti-EGFR DAR precursor polypeptide chain that contained a first polypeptide chain, a self-cleaving sequence (e.g., T2A), and a second polypeptide chain, and comprising the amino acid sequence of SEQ ID NO:62 (See FIG. 26). The T2A sequence mediated cleavage of the precursor molecule to generate first and second polypeptide chains that assembled to form a membrane-embedded DAR with intracellular signaling domains from 4-1BB and CD3zeta. The predicted first polypeptide chain of the anti-EGFR masked DAR carrying Ig gamma-1 CH3 masking moieties linked to non-cleavable linkers (Table 35), comprises the amino acid sequence of SEQ ID NO:63. The predicted second polypeptide chain of the anti-EGFR masked DAR carrying Ig gamma-1 CH3 masking moieties linked to non-cleavable linkers, comprises the amino acid sequence of SEQ ID NO:64. The heavy and light chain leader sequences are bolded and underlined. The uPA cleavable linker sequences are italicized and gray-highlighted.

TABLE 35

| | |
|---|---|
| Anti-EGFR masked DAR constructs | Peptide linker sequence: |
| [HC-CH3 MMP2/9, LC-CH3 MMP2/9] | LSLSPGKGGPLGVRGG SEQ ID NO:32 |
| [HC-CH3 uPA, LC-CH3 uPA] | GLSGRSDNHG SEQ ID NO:52 |
| [HC-CH3 NC, LC-CH3 NC] | LSLSPGKGGSGGGSGG SEQ ID NO:34 |

### Example 18: Characterizing the Anti-EGFR Dimeric Antigen Receptor (DAR) Having Activatable Masked Antibodies

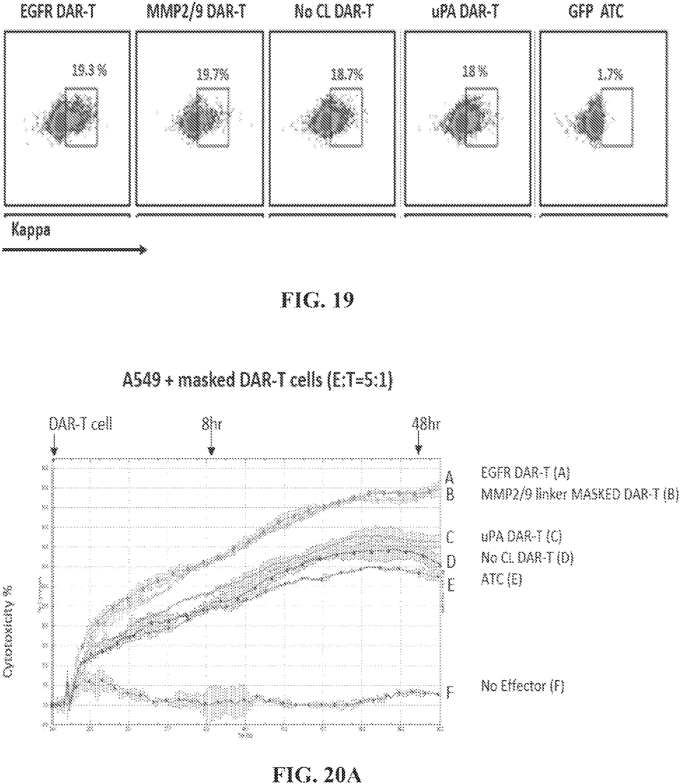

The anti-EGFR masked DAR described in Example 17 above were cultured in T cell expansion SFM media with 300 IU/mL of I-2. The media was changed every 2-3 days. In-house EGFR-his conjugated with Alexa-488 were prepared. Approximately 50k DAR-T cells or masked DAR-T cells were washed once and resuspended in 50 uL binding buffer, 1:200 APC anti-human Kappa and 1:100 EGFR-Alexa-488 was added and incubated on ice for 30 minutes. The cells were rinsed once with PBS plus 2% FBS. The cells were assay via flow cytometry using IQueScreener. The results are shown in FIG. 19.

### Example 19: Cytotoxicity Anti-EGFR Dimeric Antigen Receptor (DAR) Having Activatable Masked Antibodies Against EGFR-Expressing Tumor Cell Line

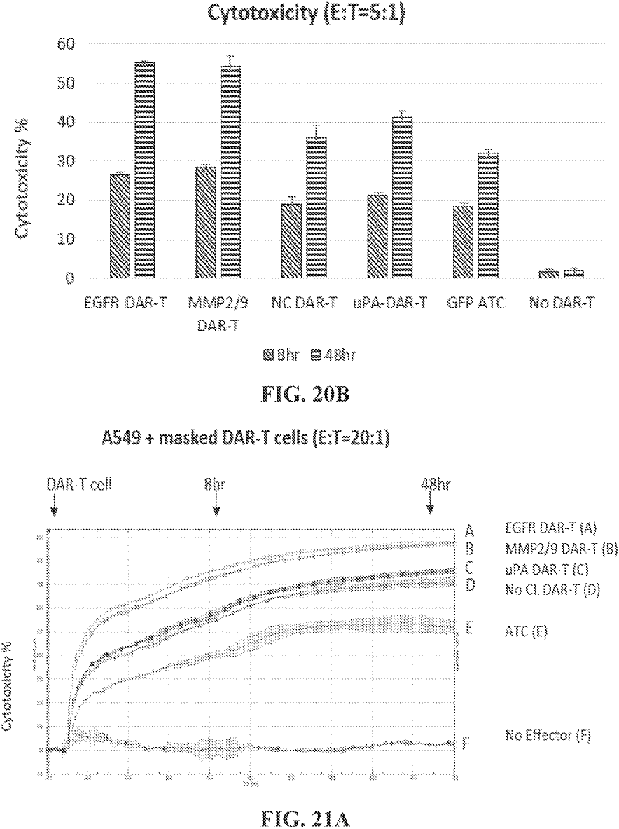

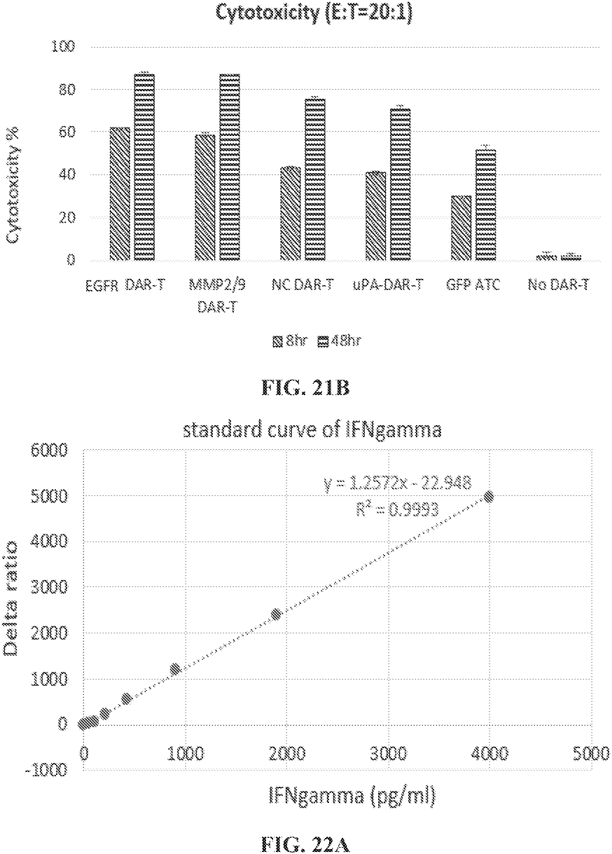

Cytotoxicity assay was conducted with a Real Time Cytotoxicity Assay (RTCA) using xCELLigence instrument. The cell lined A549 was used a target cell line. A549 is a non-small cell lung carcinoma (NSCLC) tumor cell line that expresses a high level of EGFR. 10k/well of A549 cells were seeded in E-plates and incubated for 24 hours. The control anti-EGFR DAR T cells and anti-EGFR masked DAR T cells were added at 5:1 or 20:1 of effector-to-target ratio. The cell index value was monitored kinetically for up to 72 hours. This monitoring indicates cell viability. As negative controls included no effector (A549 cells alone) and activated T cells. Normalized Cell Index was at the time point when effector cells were added (see FIGS. 20A and 21A). The results of the 5:1 and 20:1 E:T ratios are shown in FIGS. 20A and 21A, respectively.

### Example 20: Cytokine Release Assay of Anti-EGFR Dimeric Antigen Receptor (DAR) Having Activatable Masked Antibodies

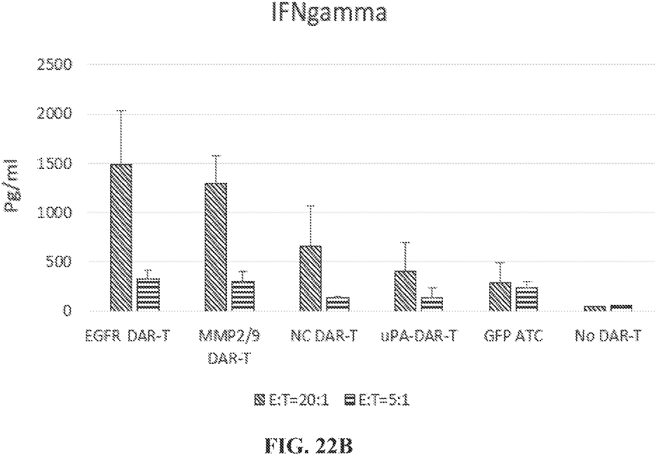

The supernatants from the cytotoxicity assay described in Example 19 above were saved and used to conduct a cytokine release assay. An IFN-gamma HTFR assay kit was used. Samples and IFN standard were diluted 1: 3 with dilution buffer. 16 uL of sample or IFN-gamma standard was added into 96W low volume, white plates, and 4 uL of mixed donor and acceptor antibody was added. The plate was sealed and incubated at room temperature for at least 2 hours then the plate was read using the HTRF setting on TECAN. The amount of IFN-gamma was calculated based on the standard curve. The results are shown in FIGS. 22A and 22B.

What is claimed:

1. An activatable masked antigen binding protein having an IgG type antibody structure, comprising:
a) a first antigen binding domain which comprises a first heavy chain variable region and a first light chain variable region and
b) a second antigen binding domain which comprises a second heavy chain variable region and a second light chain variable region, wherein
(i) the N-terminal end of the first heavy chain variable region is joined to a first masking moiety via a first peptide linker having a first cleavable site,
(ii) the N-terminal end of the first light chain variable region is joined to a second masking moiety via a second peptide linker having a second cleavable site,
(iii) the first and second masking moieties do not specifically bind the first antigen binding domain,
(iv) the first cleavable site is cleavable with a first protease, and
(v) the second cleavable site is cleavable with a second protease, and wherein
(vi) the N-terminal end of the second heavy chain variable region is joined to a third masking moiety via a third peptide linker having a third cleavable site,
(vii) the N-terminal end of the second light chain variable region is joined to a fourth masking moiety via a fourth peptide linker having a fourth cleavable site,
(viii) the third and fourth masking moieties do not specifically bind the second antigen binding domain,
(ix) the third cleavable site is cleavable with a third protease, and
(x) the fourth cleavable site is cleavable with a fourth protease, wherein the first, second, third and fourth cleavable sites are cleavable with the same protease or different proteases.

2. The activatable masked antigen binding protein of claim 1 which can bind a target antigen (e.g., monospecific antibody).

3. The activatable masked antigen binding protein of claim 1 which can bind two different target antigens (e.g., bispecific antibody).

4. The activatable masked antigen binding protein of claim 1, wherein the first and second masking moieties associate with each other to reduce the first antigen binding domain from binding to its target antigen.

5. The activatable masked antigen binding protein of claim 1, wherein the first and second masking moieties associate with each other without forming a covalent bond to reduce the first antigen binding domain from binding to its target antigen.

6. The activatable masked antigen binding protein of claim 1, wherein the amino acid sequence of the first masking moiety is mutated to form a knob or hole.

7. The activatable masked antigen binding protein of claim 1, wherein the amino acid sequence of the second masking moiety is mutated to form a hole or knob.

8. The activatable masked antigen binding protein of claim 1, wherein the first and/or second masking moieties are derived from an immunoglobulin constant region selected from a group consisting of CL (lambda), CL (kappa), CH1, CH2 and CH3.

9. The activatable masked antigen binding protein of claim 1, wherein the first and second masking moieties are derived from T cell receptors alpha (α) and beta (β) constant regions.

10. The activatable masked antigen binding protein of claim 1, wherein the first and second masking moieties associate with each other as a homodimer or heterodimer.

11. The activatable masked antigen binding protein of claim 1, wherein the first and second cleavable sites are cleavable with the same or different proteases.

12. The activatable masked antigen binding protein of claim 1, wherein the third and fourth masking moieties associate with each other to reduce the second antigen binding domain from binding to its target antigen.

13. The activatable masked antigen binding protein of claim 1, wherein the third and fourth masking moieties associate with each other without forming a covalent bond to reduce the second antigen binding domain from binding to its target antigen.

14. The activatable masked antigen binding protein of claim 1, wherein the amino acid sequence of the third masking moiety is mutated to form a knob or hole.

15. The activatable masked antigen binding protein of claim 1, wherein the amino acid sequence of the fourth masking moiety is mutated to form a hole or knob.

16. The activatable masked antigen binding protein of claim 1, wherein the third and/or fourth masking moieties are derived from an immunoglobulin constant region selected from a group consisting of CL (lambda), CL (kappa), CH1, CH2 and CH3.

17. The activatable masked antigen binding protein of claim 1, wherein the third and fourth masking moieties are derived from T cell receptors alpha (α) and beta (β) constant regions.

18. The activatable masked antigen binding protein of claim 1, wherein the third and fourth masking moieties associate with each other as a homodimer or heterodimer.

19. The activatable masked antigen binding protein of claim 1, wherein the third and fourth cleavable sites are cleavable with the same or different proteases.

20. The activatable masked antigen binding protein of claim 1, wherein the first, second, third and fourth cleavable sites are cleavable with the same or different proteases.

21. The activatable masked antigen binding protein of claim 1, comprising an inactive state activatable masked antigen binding protein, wherein the first, second, third and fourth cleavable sites are in an intact state (uncleaved), and wherein the inactive state activatable masked antigen binding protein binds its target antigen at a reduced level compared to an activated state activatable masked antigen binding protein having any one or any combination of two or more of the first, second, third and/or fourth cleavable sites in a cleaved state.

22. The activatable masked antigen binding protein of claim 1, which can bind an EGFR antigen.

23. The activatable masked antigen binding protein of claim 1, which can bind a CD38 antigen.

24. The activatable masked antigen binding protein of claim 1, wherein the first and/or second heavy chain variable region comprises an anti-EGFR heavy chain variable region comprising an amino acid sequence having at least 95% sequence identity to a portion or full-length of SEQ ID NO:2 or 4.

25. The activatable masked antigen binding protein of claim 1, wherein the first and/or second heavy chain variable region comprises an anti-CD38 heavy chain variable region comprising an amino acid sequence having at least 95% sequence identity to a portion or full-length of SEQ ID NO: 7, 9, 11, 13, 14, 15, 16, 17, 18 or 20.

26. The activatable masked antigen binding protein of claim 1, wherein the first and/or second light chain variable region comprises an anti-EGFR light chain variable region comprising an amino acid sequence having at least 95% sequence identity to a portion or full-length of SEQ ID NO:3 or 5.

27. The activatable masked antigen binding protein of claim 1, wherein the first and/or second light chain variable region comprises an anti-CD38 light chain variable region comprising an amino acid sequence having at least 95% sequence identity to a portion or full-length of SEQ ID NO: 8, 10, 12 or 19.

28. The activatable masked antigen binding protein of claim 1, wherein the first and/or third masking moiety comprises any one of the amino acid sequence of SEQ ID NO:21-31.

29. The activatable masked antigen binding protein of claim 1, wherein the second and/or fourth masking moiety comprises any one of the amino acid sequence of SEQ ID NO:21-31.

30. The activatable masked antigen binding protein of claim 1, wherein the first and/or third peptide linker comprises any one of the amino acid sequence of SEQ ID NO:32-40.

31. The activatable masked antigen binding protein of claim 1, wherein the second and/or fourth peptide linker comprises any one of the amino acid sequence of SEQ ID NO:32-40.

32. The activatable masked antigen binding protein of claim 1, further comprising a chemical linker joined to a toxin.

33. A pharmaceutical composition comprising the activatable masked antigen binding protein of claim 1 and a pharmaceutically-acceptable excipient.

34. A diagnostic agent that can detect the presence of a protease, comprising the activatable masked antigen binding protein of claim 1 joined to a detectable moiety or the activatable masked antigen binding protein of claim 1 is not joined to a detectable moiety, wherein the detectable moiety comprises a radioactive moiety, a colorimetric moiety, an antigenic moiety, an enzymatic moiety, a biotin moiety, a streptavidin moiety or a protein A moiety.

35. A kit for in vitro and/or in vivo use comprising the activatable masked antigen binding protein of claim 1.

36. A first nucleic acid encoding a first polypeptide of the activatable masked antigen binding protein of claim 1, wherein the first polypeptide comprises the first heavy chain variable region joined to the first peptide linker and the first masking moiety, wherein the first peptide linker includes the first cleavable site.

37. A second nucleic acid encoding a second polypeptide of the activatable masked antigen binding protein of claim 1, wherein the second polypeptide comprises the first light chain variable region joined to the second peptide linker and the second masking moiety, wherein the second peptide linker includes the second cleavable site.

38. A third nucleic acid encoding a third polypeptide of the activatable masked antigen binding protein of claim 1, wherein the third polypeptide comprises the second heavy chain variable region joined to the third peptide linker and the third masking moiety, wherein the third peptide linker includes the third cleavable site.

39. A fourth nucleic acid encoding a fourth polypeptide of the activatable masked antigen binding protein of claim 1, wherein the fourth polypeptide comprises the second light chain variable region joined to the fourth peptide linker and the fourth masking moiety, wherein the fourth peptide linker includes the third cleavable site.

40. An expression vector comprising the first, second, third or fourth nucleic acid of any one of claims 36-39.

41. An expression vector comprising any one or any combination of two or more of the first, second, third and/or fourth nucleic acid of any of claims 36-39.

42. A host cell, or a population of host cells, harboring the expression vector of claim 40.

43. A host cell, or a population of host cells, harboring any one or any combination of two or more of the expression vectors of claim 41.

44. A method for preparing a polypeptide, comprising: culturing the population of host cells of claim 42 under conditions suitable for expressing the first, second, third or fourth polypeptide by the population of host cells, wherein individual host cells in the population of host cells harbor an expression vector operably linked to a nucleic acid encoding the first, second, third or fourth polypeptide of the activatable masked antigen binding protein.

45. The method of claim 44, further comprising: isolating the expressed first, second, third or fourth polypeptide from the population of host cells.

46. The method of claim 45, further comprising: recovering the expressed first, second, third or fourth polypeptide.

47. A method for preparing at least one polypeptide, comprising: culturing the population of host cells of claim 43 under conditions suitable for expressing any one of or any combination of two of more of the first, second, third and/or fourth polypeptide by the population of host cells, wherein individual host cells in the population of host cells harbor one or more expression vectors each operably linked to a nucleic acid encoding any one or any combination of two or more of the first, second, third and/or fourth polypeptide of the activatable masked antigen binding protein.

48. The method of claim 47, further comprising: isolating the first, second, third and/or fourth polypeptides from the population of host cells.

49. The method of claim 47, wherein the culturing condition is suitable for associating the first and second polypeptides with each other to form a first dimerized masking complex and to form a first antigen binding domain, and/or is suitable for associating the third and fourth polypeptides with each other to form a second dimerized masking complex and to form a second antigen binding domain.

50. The method of claim 48, further comprising: recovering the associated first and second polypeptides, or recovering the associated third and fourth polypeptide, or recovering the first, second, third and fourth polypeptides.

51. A method for cleaving at least one peptide linker of the activatable masked antigen binding protein of claim 1, comprising:
a) contacting at least one protease with the activatable masked antigen binding protein in an inactive form, wherein the first, second, third and fourth peptide linkers are in the un-cleaved state; and
b) cleaving at least one of the peptide linkers to convert the activatable masked antigen binding protein to an activated form.

52. The method of claim 51, further comprising: binding the activatable masked antigen binding protein, now in the activated state, to a target antigen.

53. An in vitro method for detecting the presence of a protease produced by a tumor from a subject, the method comprising:
a) contacting (i) a tumor obtained from the subject with (ii) the activatable masked antigen binding protein of claim 1, in an uncleaved state, wherein the tumor sample produces a protease that cleaves at least one of the first, second, third and/or fourth peptide linker; and
b) detecting the cleavage product from cleaving the first, second, third and/or fourth peptide linker; and
c) identifying the type of protease produced by the tumor from the subject by detecting the first, second, third and/or fourth cleavage product and correlating the cleavage products with the amino acid sequence of the first, second, third and/or fourth peptide linker.

54. A method for treating a subject having a disease associated with expression or over-expression of a tumor-associated antigen, comprising: administering to the subject an effective amount of a therapeutic composition comprising the activatable masked antigen binding protein of claim 1, wherein the first, second, third and fourth peptide linkers are in an un-cleaved state.

55. The method of claim 54, wherein the disease is selected from a group consisting of hematologic cancer, breast cancer, ovarian cancer, prostate cancer, head and neck cancer, lung cancer, bladder cancer, melanoma, colorectal cancer, pancreatic cancer, lung cancer, liver cancer, renal cancer, esophageal cancer, leiomyoma, leiomyosarcoma, glioma, and glioblastoma.

56. The method of claim 55, wherein the hematologic cancer selected from the group consisting of non-Hodgkin’s lymphoma (NHL), Burkitt’s lymphoma (BL), B chronic lymphocytic leukemia (B-CLL), B and T acute lymphocytic leukemia (ALL), T cell lymphoma (TCL), acute myeloid leukemia (AML), hairy cell leukemia (HCL), Hodgkin’s Lymphoma (HL), chronic myeloid leukemia (CML) and multiple myeloma (MM).

57. An activatable masked antigen binding protein having a dimeric antigen receptor (DAR) structure, comprising a first and second polypeptide chain, wherein the
a) first polypeptide chain comprises: (i) a first masking moiety, (ii) a first peptide linker, (iii) an antibody heavy chain variable region (VH), (iv) an antibody heavy chain constant region (CH), (v) an optional hinge region, (vi) a transmembrane region (TM), and (vii) an intracellular signaling region, and wherein the
b) second polypeptide chain comprises: (i) a second masking moiety, (ii) a second peptide linker, (iii) an antibody light chain variable region (VL) (e.g., kappa or lambda), and (iv) an antibody light chain constant region (CL), wherein the antibody heavy chain variable region (VH) and the antibody light chain variable region (VL) form an antigen binding domain that binds a target antigen.

58. The activatable masked antigen binding protein of claim 57, wherein the first and second masking moieties associates with each other to reduce the antigen binding domain from binding to its target antigen.

59. The activatable masked antigen binding protein of claim 57, wherein the first and second masking moieties associates with each other without forming a covalent bond to reduce the antigen binding domain from binding to its target antigen.

60. The activatable masked antigen binding protein of claim 57, wherein the amino acid sequence of the first masking moiety is mutated to form a knob or hole.

61. The activatable masked antigen binding protein of claim 57, wherein the amino acid sequence of the second masking moiety is mutated to form a hole or knob.

62. The activatable masked antigen binding protein of claim 57, wherein the first and/or second masking moieties are derived from an immunoglobulin constant region selected from a group consisting of CL (lambda), CL (kappa), CH1, CH2 and CH3.

63. The activatable masked antigen binding protein of claim 57, wherein the first and second masking moieties associate with each other as a homodimer or heterodimer.

64. The activatable masked antigen binding protein of claim 57, wherein the first and second masking moieties are derived from T cell receptors alpha (α) and beta (β) constant regions.

65. The activatable masked antigen binding protein of claim 57, wherein the first and second cleavable sites are cleavable with the same or different proteases.

66. The activatable masked antigen binding protein of claim 57, which can bind an EGFR antigen.

67. The activatable masked antigen binding protein of claim 57, which can bind a CD38 antigen.

68. The activatable masked antigen binding protein of claim 57, further comprising a chemical linker j oined to a toxin.

69. A pharmaceutical composition comprising the activatable masked antigen binding protein of claim 57 and a pharmaceutically-acceptable excipient.

70. A diagnostic agent that can detect the presence of a protease, comprising the activatable masked antigen binding protein of claim 57 joined to a detectable moiety or the activatable masked antigen binding protein of claim 57 is not joined to a detectable moiety, wherein the detectable moiety comprises a radioactive moiety, a colorimetric moiety, an antigenic moiety, an enzymatic moiety, a biotin moiety, a streptavidin moiety or a protein A moiety.

71. A kit for in vitro and/or in vivo use comprising the activatable masked antigen binding protein of claim 57.

72. A first nucleic acid encoding the first polypeptide chain of the activatable masked antigen binding protein of claim 57.

73. A second nucleic acid encoding the second polypeptide of the activatable masked antigen binding protein of claim 57.

74. A first nucleic acid encoding the first polypeptide chain of the activatable masked antigen binding protein of claim 57 and a second nucleic acid encoding the second polypeptide of the activatable masked antigen binding protein of claim 57.

75. An expression vector comprising the first nucleic acid of claim 72.

76. An expression vector comprising the second nucleic acid of claim 73.

77. An expression vector comprising the first and second nucleic acids of claim 74.

78. A host cell, or a population of host cells, harboring the expression vector of claim 75.

79. A host cell, or a population of host cells, harboring the expression vector of claim 76.

80. A host cell, or a population of host cells, wherein individual host cells harbor the expression vectors of claim 75 and 76.

81. A host cell, or a population of host cells, wherein individual host cells harbor the expression vector of claim 77.

82. A method for preparing a polypeptide, comprising: culturing the population of host cells of claim 80 under conditions suitable for expressing the first or second polypeptide chains by the population of host cells.

83. The method of claim 82, further comprising: isolating the expressed first or second polypeptide chains from the population of host cells.

84. The method of claim 83, further comprising: recovering the expressed first or second polypeptide chains.

85. A method for preparing a polypeptide, comprising: culturing the population of host cells of claim 81 under conditions suitable for expressing the first and second polypeptide chains by the population of host cells.

86. The method of claim 85, further comprising: isolating the expressed first and second polypeptide chains from the population of host cells.

87. The method of claim 86, further comprising: recovering the expressed first and second polypeptide chains.

88. The method of claim 85, wherein the culturing condition is suitable for associating the first and second polypeptide chains with each other to form a dimerized masking complex and to form an antigen binding domain.

89. The method of claim 87, further comprising: recovering the associated first and second polypeptide chains.

90. A method for cleaving at least one peptide linker of the activatable masked antigen binding protein of claim 57, comprising:
a) contacting at least one protease with the activatable masked antigen binding protein in an inactive form, wherein the first and second peptide linkers are in the un-cleaved state; and
b) cleaving at least one of the peptide linkers to convert the activatable masked antigen binding protein to an activated form.

91. The method of claim 90, further comprising: binding the activatable masked antigen binding protein, now in the activated state, to a target antigen.

92. An in vitro method for detecting the presence of a protease produced by a tumor from a subject, the method comprising:
a) contacting (i) a tumor obtained from the subject with (ii) the activatable masked antigen binding protein of claim 57, wherein the tumor sample produces a protease that cleaves at least one of the first and/or second peptide linkers; and
b) detecting the cleavage product from cleaving the first and/or second peptide linker; and
c) identifying the type of protease produced by the tumor from the subject by detecting the first and/or second cleavage product and correlating the cleavage products with the amino acid sequence of the first and/or second peptide linker.

93. A method for treating a subject having a disease associated with expression or over-expression of a tumor-associated antigen, comprising: administering to the subject an effective amount of a therapeutic composition comprising the activatable masked antigen binding protein of claim 57, wherein the first and second peptide linkers are in an un-cleaved state.

94. The method of claim 93, wherein the disease is selected from a group consisting of hematologic cancer, breast cancer, ovarian cancer, prostate cancer, head and neck cancer, lung cancer, bladder cancer, melanoma, colorectal cancer, pancreatic cancer, lung cancer, liver cancer, renal cancer, esophageal cancer, leiomyoma, leiomyosarcoma, glioma, and glioblastoma.

95. The method of claim 94, wherein the hematologic cancer selected from the group consisting of non-Hodgkin’s lymphoma (NHL), Burkitt’s lymphoma (BL), B chronic lymphocytic leukemia (B-CLL), B and T acute lymphocytic leukemia (ALL), T cell lymphoma (TCL), acute myeloid leukemia (AML), hairy cell leukemia (HCL), Hodgkin’s Lymphoma (HL), chronic myeloid leukemia (CML) and multiple myeloma (MM).


SEQUENCE LISTING (176 sequences)

SEQ ID NO: 1 - protein, 621 aa, Unknown [sequence omitted]

SEQ ID NO: 2 - protein, 449 aa, Artificial Sequence
  source: /note="Description of Artificial Sequence: Synthetic polypeptide"
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 214 aa, Artificial Sequence
  source: /note="Description of Artificial Sequence: Synthetic polypeptide"
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 449 aa, Artificial Sequence
  source: /note="Description of Artificial Sequence: Synthetic polypeptide"
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 214 aa, Artificial Sequence; identical to SEQ ID NO: 3
  source: /note="Description of Artificial Sequence: Synthetic polypeptide"

SEQ ID NO: 6 - protein, 258 aa, Unknown
  source: /note="Description of Unknown: CD38 target antigen sequence"
VPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 7 - protein, 448 aa, Artificial Sequence
  source: /note="Description of Artificial Sequence: Synthetic polypeptide"
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 216 aa, Artificial Sequence
  source: /note="Description of Artificial Sequence: Synthetic polypeptide"
QAGLTQPPSASGTSGQRVTISCSGSSSNIGINFVYWYQHLPGTAPKLLIYKNNQRPSGVPDRFSGSKSGNSASLAISGLRSEDEADYYCAAWDDSLSGYVFGSGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 9 - protein, 118 aa, Artificial Sequence
  source: /note="Description of Artificial Sequence: Synthetic polypeptide"
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSS

SEQ ID NO: 10 - protein, 110 aa, Artificial Sequence
  source: /note="Description of Artificial Sequence: Synthetic polypeptide"
QAGLTQPPSASGTSGQRVTISCSGSSSNIGINFVYWYQHLPGTAPKLLIYKNNQRPSGVPDRFSGSKSGNSASLAISGLRSEDEADYYCAAWDDSLSGYVFGSGTKVTVL

SEQ ID NO: 11 - protein, 118 aa, Artificial Sequence
  source: /note="Description of Artificial Sequence: Synthetic polypeptide"
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREGWSGEFTDWGQGTLVTVSS

SEQ ID NO: 12 - protein, 110 aa, Artificial Sequence
  source: /note="Description of Artificial Sequence: Synthetic polypeptide"
QAGLTQPPSASGTSGQRVTISCSGSSSNIGFHFVYWYQHLPGTAPKLLIYKNNQRPSGVPDRFSGSKSGNSASLAISGLRSEDEADYYCAAWDDSLSGYVFGSGTKVTVL

SEQ ID NO: 13 - protein, 118 aa, Artificial Sequence
  source: /note="Description of Artificial Sequence: Synthetic polypeptide"
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREAWGGEFTNWGQGTLVTVSS

SEQ ID NO: 14 - protein, 118 aa, Artificial Sequence
  source: /note="Description of Artificial Sequence: Synthetic polypeptide"
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREAWGGEFTDWGQGTLVTVSS

SEQ ID NO: 15 - protein, 118 aa, Artificial Sequence
  source: /note="Description of Artificial Sequence: Synthetic polypeptide"
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREAWSGEFTDWGQGTLVTVSS

SEQ ID NO: 16 - protein, 118 aa, Artificial Sequence
  source: /note="Description of Artificial Sequence: Synthetic polypeptide"
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREGWGGEFTDWGQGTLVTVSS

SEQ ID NO: 17 - protein, 118 aa, Artificial Sequence; identical to SEQ ID NO: 11
  source: /note="Description of Artificial Sequence: Synthetic polypeptide"

SEQ ID NO: 18 - protein, 118 aa, Artificial Sequence
  source: /note="Description of Artificial Sequence: Synthetic polypeptide"
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGQGTLVTVSS

SEQ ID NO: 19 - protein, 110 aa, Artificial Sequence
  source: /note="Description of Artificial Sequence: Synthetic polypeptide"
QSVLTQPPSASGTSGQRVTISCSGSSSNIGFHFVYWYQHLPGTAPKLLIYKNNQRPSGVPDRFSGSKSGNSASLAISGLRSEDEADYYCAAWDDSLSGYVFGSGTKVTVL

SEQ ID NO: 20 - protein, 118 aa, Artificial Sequence
  source: /note="Description of Artificial Sequence: Synthetic polypeptide"
QVQLVESGGGLVKPGGSLRLSCAASGFTFSDDYMSWIRQAPGKGLEWVASVSSGRPTTYYADSVRGRFTISRDNAKNSLYLQMNSLRAEDTAVYYCAREDWGGEFTDWGQGTLVTVSS

SEQ ID NO: 21 - protein, 103 aa, Artificial Sequence
  source: /note="Description of Artificial Sequence: Synthetic polypeptide"
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSS

SEQ ID NO: 22 - protein, 106 aa, Artificial Sequence
  source: /note="Description of Artificial Sequence: Synthetic polypeptide"
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTESS

SEQ ID NO: 23 - protein, 107 aa, Artificial Sequence
  source: /note="Description of Artificial Sequence: Synthetic polypeptide"
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 24 - protein, 107 aa, Artificial Sequence
  source: /note="Description of Artificial Sequence: Synthetic polypeptide"
GQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 25 - protein, 107 aa, Artificial Sequence
  source: /note="Description of Artificial Sequence: Synthetic polypeptide"
GQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 118 aa, Artificial Sequence
  source: /note="Description of Artificial Sequence: Synthetic polypeptide"
EVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCKTYFYSFSYWGQGTLVTVSS

SEQ ID NO: 27 - protein, 112 aa, Artificial Sequence
  source: /note="Description of Artificial Sequence: Synthetic polypeptide"
ELQMTQSPSSLSASVGDRVTITCRSSQSLLHTYGSPYLNWYLQKPGQSPKLLIYKVSNRFSGVPSRFSGSGSGTDFTLTISSLQPEDFAVYFCSQGTHLPYTFGGGTKVEIK

SEQ ID NO: 28 - protein, 90 aa, Artificial Sequence
  source: /note="Description of Artificial Sequence: Synthetic polypeptide"
IQNPDPAVYQLRDSKSSDKSVCLFTDFDSQTNVSQSKDSDVYITDKTVLDMRSMDFKSNSAVAWSNKSDFACANAFNNSIIPEDTFFPSP

SEQ ID NO: 29 - protein, 111 aa, Artificial Sequence
  source: /note="Description of Artificial Sequence: Synthetic polypeptide"
KVFPPEVAVFEPSEAEISHTQKATLVCLATGFFPDHVELSWWVNGKEVHSGVSTDPQPLKEQPALNDSRYSLSSRLRVSATFWQNPRNHFRCQVQFNGQIVSAEAWGRADS

SEQ ID NO: 30 - protein, 107 aa, Artificial Sequence
  source: /note="Description of Artificial Sequence: Synthetic polypeptide"
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 107 aa, Artificial Sequence
  source: /note="Description of Artificial Sequence: Synthetic polypeptide"
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 32 - protein, 16 aa, Artificial Sequence
  source: /note="Description of Artificial Sequence: Synthetic peptide"
LSLSPGKGGPLGVRGG

SEQ ID NO: 33 - protein, 12 aa, Artificial Sequence
  source: /note="Description of Artificial Sequence: Synthetic peptide"
LSLSPPLGVRGG

SEQ ID NO: 34 - protein, 16 aa, Artificial Sequence
  source: /note="Description of Artificial Sequence: Synthetic peptide"
LSLSPGKGGSGGGSGG

SEQ ID NO: 35 - protein, 19 aa, Artificial Sequence
  source: /note="Description of Artificial Sequence: Synthetic peptide"
LSLSPGKGGPLGVRGGGGG

SEQ ID NO: 36 - protein, 7 aa, Artificial Sequence
  source: /note="Description of Artificial Sequence: Synthetic peptide"
VPLVLYS

SEQ ID NO: 37 - protein, 5 aa, Artificial Sequence
  source: /note="Description of Artificial Sequence: Synthetic peptide"
GGGGS

SEQ ID NO: 38 - protein, 17 aa, Artificial Sequence
  source: /note="Description of Artificial Sequence: Synthetic peptide"
LSLSPGKGGGGRRGGGG

SEQ ID NO: 39 - protein, 6 aa, Artificial Sequence
  source: /note="Description of Artificial Sequence: Synthetic peptide"
SGRSAN

SEQ ID NO: 40 - protein, 16 aa, Artificial Sequence
  source: /note="Description of Artificial Sequence: Synthetic peptide"
GGPLGVRGGPLGVRGG

SEQ ID NO: 41 - protein, 20 aa, Artificial Sequence
  source: /note="Description of Artificial Sequence: Synthetic peptide"
AVGQDTQEVIVVPHSLPFKV

SEQ ID NO: 42 - protein, 29 aa, Artificial Sequence
  source: /note="Description of Artificial Sequence: Synthetic peptide"
VVISAILALVVLTIISLIILIMLWQKKPR

SEQ ID NO: 43 - protein, 49 aa, Artificial Sequence
  source: /note="Description of Artificial Sequence: Synthetic polypeptide"
AVGQDTQEVIVVPHSLPFKVVVISAILALVVLTIISLIILIMLWQKKPR

SEQ ID NO: 44 - protein, 40 aa, Artificial Sequence
  source: /note="Description of Artificial Sequence: Synthetic polypeptide"
KIEVMYPPPYLDNEKSNGTIIHVKGKHLCPSPLFPGPSKP

SEQ ID NO: 45 - protein, 48 aa, Artificial Sequence
  source: /note="Description of Artificial Sequence: Synthetic polypeptide"
AKPTTTPAPRPPTPAPTIASQPLSLRPEACRPAAGGAVHTRGLDFAPR

SEQ ID NO: 46 - protein, 88 aa, Artificial Sequence
  source: /note="Description of Artificial Sequence: Synthetic polypeptide"
AKPTTTPAPRPPTPAPTIASQPLSLRPEACRPAAGGAVHTRGLDFAPRKIEVMYPPPYLDNEKSNGTIIHVKGKHLCPSPLFPGPSKP

SEQ ID NO: 47 - protein, 27 aa, Artificial Sequence
  source: /note="Description of Artificial Sequence: Synthetic peptide"
FWVLVVVGGVLACYSLLVTVAFIIFWV

SEQ ID NO: 48 - protein, 42 aa, Artificial Sequence
  source: /note="Description of Artificial Sequence: Synthetic polypeptide"
KRGRKKLLYIFKQPFMRPVQTTQEEDGCSCRFPEEEEGGCEL

SEQ ID NO: 49 - protein, 41 aa, Artificial Sequence
  source: /note="Description of Artificial Sequence: Synthetic polypeptide"
RSKRSRLLHSDYMNMTPRRPGPTRKHYQPYAPPRDFAAYRS

SEQ ID NO: 50 - protein, 112 aa, Artificial Sequence
  source: /note="Description of Artificial Sequence: Synthetic polypeptide"
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 45 aa, Artificial Sequence
  source: /note="Description of Artificial Sequence: Synthetic polypeptide"
RVKFSRSADKGERRRGKGHDGLYQGLSTATKDTYDALHMQALPPR

SEQ ID NO: 52 - protein, 10 aa, Artificial Sequence
  source: /note="Description of Artificial Sequence: Synthetic peptide"
GLSGRSDNHG

SEQ ID NO: 53 - protein, 722 aa, Artificial Sequence
  source: /note="Description of Artificial Sequence: Synthetic polypeptide"
MEWSWVFLFFLSVTTGVHSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMSVPTQVLGLLLLWLTDARCDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 448 aa, Artificial Sequence
  source: /note="Description of Artificial Sequence: Synthetic polypeptide"
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 214 aa, Artificial Sequence; identical to SEQ ID NO: 3
  source: /note="Description of Artificial Sequence: Synthetic polypeptide"

SEQ ID NO: 56 - protein, 954 aa, Artificial Sequence
  source: /note="Description of Artificial Sequence: Synthetic polypeptide"
MEWSWVFLFFLSVTTGVHSGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGPLGVRGGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMSVPTQVLGLLLLWLTDARCGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGPLGVRGGDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 564 aa, Artificial Sequence
  source: /note="Description of Artificial Sequence: Synthetic polypeptide"
GQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGPLGVRGGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - protein, 330 aa, Artificial Sequence
  source: /note="Description of Artificial Sequence: Synthetic polypeptide"
GQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGPLGVRGGDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 956 aa, Artificial Sequence
  source: /note="Description of Artificial Sequence: Synthetic polypeptide"
MEWSWVFLFFLSVTTGVHSGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGLSGRSDNHGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMSVPTQVLGLLLLWLTDARCGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGLSGRSDNHGDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 565 aa, Artificial Sequence
  source: /note="Description of Artificial Sequence: Synthetic polypeptide"
GQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGLSGRSDNHGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 331 aa, Artificial Sequence
  source: /note="Description of Artificial Sequence: Synthetic polypeptide"
GQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGLSGRSDNHGDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 954 aa, Artificial Sequence
  source: /note="Description of Artificial Sequence: Synthetic polypeptide"
MEWSWVFLFFLSVTTGVHSGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGSGGGSGGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMSVPTQVLGLLLLWLTDARCGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGGSGGDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 564 aa, Artificial Sequence
  source: /note="Description of Artificial Sequence: Synthetic polypeptide"
GQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGSGGGSGGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 330 aa, Artificial Sequence
  source: /note="Description of Artificial Sequence: Synthetic polypeptide"
GQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGGSGGDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 19 aa, Mus sp.
MEWSWVFLFFLSVTTGVHS

SEQ ID NO: 66 - protein, 18 aa, Artificial Sequence
  source: /note="Description of Artificial Sequence: Synthetic peptide"
EGRGSLLTCGDVEENPGP

SEQ ID NO: 67 - protein, 21 aa, Artificial Sequence
  source: /note="Description of Artificial Sequence: Synthetic peptide"
  VARIANT (1)..(3): /replace=" "
  SITE (1)..(21): /note="Variant residues given in the sequence have no preference with respect to those in the annotations for variant positions"
GSGATNFSLLKQAGDVEENPG

SEQ ID NO: 68 - protein, 22 aa, Artificial Sequence
  source: /note="Description of Artificial Sequence: Synthetic peptide"
  VARIANT (1)..(3): /replace=" "
  SITE (1)..(22): /note="Variant residues given in the sequence have no preference with respect to those in the annotations for variant positions"
GSGQCTNYALLKLAGDVESNPG

SEQ ID NO: 69 - protein, 25 aa, Artificial Sequence
  source: /note="Description of Artificial Sequence: Synthetic peptide"
  VARIANT (1)..(3): /replace=" "
  SITE (1)..(25): /note="Variant residues given in the sequence have no preference with respect to those in the annotations for variant positions"
GSGVKQTLNFDLLKLAGDVESNPGP

SEQ ID NO: 70 - protein, 20 aa, Artificial Sequence
  source: /note="Description of Artificial Sequence: Synthetic peptide"
GGSGSGSGGSSGGGSGGGGS

SEQ ID NO: 71 - protein, 11 aa, Artificial Sequence
  source: /note="Description of Artificial Sequence: Synthetic peptide"
TSGSGGSGGSV

SEQ ID NO: 72 - protein, 17 aa, Artificial Sequence
  source: /note="Description of Artificial Sequence: Synthetic peptide"
TSGSGGSPLGMGGSGSV

SEQ ID NO: 73 - protein, 17 aa, Artificial Sequence
  source: /note="Description of Artificial Sequence: Synthetic peptide"
TSGSGGSPLGVGGSGSV

SEQ ID NO: 74 - protein, 17 aa, Artificial Sequence
  source: /note="Description of Artificial Sequence: Synthetic peptide"
TSGSGGSPAALGGSGSV

SEQ ID NO: 75 - protein, 17 aa, Artificial Sequence
  source: /note="Description of Artificial Sequence: Synthetic peptide"
TSGSGGSPAGLGGSGSV

SEQ ID NO: 76 - protein, 14 aa, Artificial Sequence
  source: /note="Description of Artificial Sequence: Synthetic peptide"
TSGSGGSPLGMVGV

SEQ ID NO: 77 - protein, 14 aa, Artificial Sequence
  source: /note="Description of Artificial Sequence: Synthetic peptide"
TSGSGGSPLGVVGV

SEQ ID NO: 78 - protein, 14 aa, Artificial Sequence
  source: /note="Description of Artificial Sequence: Synthetic peptide"
TSGSGGSPAALVGV

SEQ ID NO: 79 - protein, 14 aa, Artificial Sequence
  source: /note="Description of Artificial Sequence: Synthetic peptide"
TSGSGGSPAGLVGV

SEQ ID NO: 80 - protein, 14 aa, Artificial Sequence
  source: /note="Description of Artificial Sequence: Synthetic peptide"
TSGSGGSPLGMVLV

SEQ ID NO: 81 - protein, 14 aa, Artificial Sequence
  source: /note="Description of Artificial Sequence: Synthetic peptide"
TSGSGGSPLGVVLV

SEQ ID NO: 82 - protein, 14 aa, Artificial Sequence
  source: /note="Description of Artificial Sequence: Synthetic peptide"
TSGSGGSPAALVLV

SEQ ID NO: 83 - protein, 14 aa, Artificial Sequence
  source: /note="Description of Artificial Sequence: Synthetic peptide"
TSGSGGSPAGLVLV

SEQ ID NO: 84 - protein, 5 aa, Artificial Sequence
  source: /note="Description of Artificial Sequence: Synthetic peptide"
LEATA

SEQ ID NO: 85 - protein, 17 aa, Artificial Sequence
  source: /note="Description of Artificial Sequence: Synthetic peptide"
SGSGGSPLGMGGSGSVD

SEQ ID NO: 86 - protein, 17 aa, Artificial Sequence
  source: /note="Description of Artificial Sequence: Synthetic peptide"
SGSGGSPAGLGGSCSVD

SEQ ID NO: 87 - protein, 14 aa, Artificial Sequence
  source: /note="Description of Artificial Sequence: Synthetic peptide"
SGSGGSPAGLVGVD

SEQ ID NO: 88 - protein, 10 aa, Artificial Sequence
  source: /note="Description of Artificial Sequence: Synthetic peptide"
GGAANLVRGG

SEQ ID NO: 89 - protein, 7 aa, Artificial Sequence
  source: /note="Description of Artificial Sequence: Synthetic peptide"
SRPLALR

SEQ ID NO: 90 - protein, 7 aa, Artificial Sequence
  source: /note="Description of Artificial Sequence: Synthetic peptide"
SRPANLR

SEQ ID NO: 91 - protein, 7 aa, Artificial Sequence
  source: /note="Description of Artificial Sequence: Synthetic peptide"
  VARIANT (1)..(1): /replace="V" or "R"
  MOD_RES (2)..(2): Any amino acid
  VARIANT (3)..(3): /replace="A"
  VARIANT (4)..(4): /replace="M" or "A" or "R" or "Y"
  VARIANT (5)..(5): /replace="S" or "N" or "G" or "H" or "M"
  VARIANT (7)..(7): /replace="L" or "Q" or "M"
  SITE (1)..(7): /note="Variant residues given in the sequence have no preference with respect to those in the annotations for variant positions"
SXPLALR

SEQ ID NO: 92 - protein, 8 aa, Artificial Sequence
  source: /note="Description of Artificial Sequence: Synthetic peptide"
DVAQFVLT

SEQ ID NO: 93 - protein, 10 aa, Artificial Sequence
  source: /note="Description of Artificial Sequence: Synthetic peptide"
VLVPMAMMAS

SEQ ID NO: 94 - protein, 8 aa, Artificial Sequence
  source: /note="Description of Artificial Sequence: Synthetic peptide"
  MOD_RES (1)..(1): Any amino acid
  VARIANT (2)..(2): /replace="A"
  MOD_RES (3)..(3): Any amino acid
  MOD_RES (4)..(4): Any amino acid
  VARIANT (5)..(5): /replace="I"
  MOD_RES (6)..(6): Any amino acid
  MOD_RES (7)..(7): Any amino acid
  MOD_RES (8)..(8): Any amino acid
  SITE (1)..(8): /note="Variant residues given in the sequence have no preference with respect to those in the annotations for variant positions"
XPXXLXXX

SEQ ID NO: 95 - protein, 6 aa, Artificial Sequence
  source: /note="Description of Artificial Sequence: Synthetic peptide"
PLGLAG

SEQ ID NO: 96 - protein, 8 aa, Artificial Sequence
  source: /note="Description of Artificial Sequence: Synthetic peptide"
  MOD_RES (6)..(6): Any amino acid
HRPRGXTN

SEQ ID NO: 97 - protein, 8 aa, Artificial Sequence
  source: /note="Description of Artificial Sequence: Synthetic peptide"
  MOD_RES (1)..(1): Any amino acid
  MOD_RES (3)..(3): Any amino acid
  MOD_RES (4)..(4): Any amino acid
  VARIANT (5)..(5): /replace="I"
  MOD_RES (6)..(6): Any amino acid
  MOD_RES (7)..(7): Any amino acid
  MOD_RES (8)..(8): Any amino acid
  SITE (1)..(8): /note="Variant residues given in the sequence have no preference with respect to those in the annotations for variant positions"
XPXXLXXX

SEQ ID NO: 98 - protein, 7 aa, Artificial Sequence
  source: /note="Description of Artificial Sequence: Synthetic peptide"
VPLSLYS

SEQ ID NO: 99 - protein, 9 aa, Artificial Sequence
  source: /note="Description of Artificial Sequence: Synthetic peptide"
GGPLGVRGG

SEQ ID NO: 100 - protein, 12 aa, Artificial Sequence
  source: /note="Description of Artificial Sequence: Synthetic peptide"
GGPLGVRGGGGG

SEQ ID NO: 101 - protein, 7 aa, Artificial Sequence
  source: /note="Description of Artificial Sequence: Synthetic peptide"
PLGVRGG

SEQ ID NO: 102 - protein, 8 aa, Artificial Sequence
  source: /note="Description of Artificial Sequence: Synthetic peptide"
  VARIANT (6)..(6): /replace="W"
  SITE (1)..(8): /note="Variant residues given in the sequence have no preference with respect to those in the annotations for variant positions"
HRPRGVTN

SEQ ID NO: 103 - protein, 8 aa, Artificial Sequence
  source: /note="Description of Artificial Sequence: Synthetic peptide"
  VARIANT (2)..(2): /replace="A"
  MOD_RES (3)..(3): Any amino acid
  VARIANT (4)..(4): /replace="A"
  MOD_RES (6)..(6): Any amino acid
  MOD_RES (8)..(8): Any amino acid
  SITE (1)..(8): /note="Variant residues given in the sequence have no preference with respect to those in the annotations for variant positions"
GPXGLXGX

SEQ ID NO: 104 - protein, 8 aa, Artificial Sequence
  source: /note="Description of Artificial Sequence: Synthetic peptide"
GPLGIAGQ

SEQ ID NO: 105 - protein, 6 aa, Artificial Sequence
  source: /note="Description of Artificial Sequence: Synthetic peptide"
PVGLIG

SEQ ID NO: 106 - protein, 8 aa, Artificial Sequence
  source: /note="Description of Artificial Sequence: Synthetic peptide"
HPVGLLAR

SEQ ID NO: 107 - protein, 8 aa, Artificial Sequence
  source: /note="Description of Artificial Sequence: Synthetic peptide"
PLGVRGGG

SEQ ID NO: 108 - protein, 10 aa, Artificial Sequence
  source: /note="Description of Artificial Sequence: Synthetic peptide"
GGGGRRGGGG

SEQ ID NO: 109 - protein, 6 aa, Artificial Sequence
  source: /note="Description of Artificial Sequence: Synthetic peptide"
  VARIANT (1)..(1): /replace="K"
  SITE (1)..(6): /note="Variant residues given in the sequence have no preference with respect to those in the annotations for variant positions"
GSGRSA

SEQ ID NO: 110 - protein, 8 aa, Artificial Sequence
  source: /note="Description of Artificial Sequence: Synthetic peptide"
  VARIANT (6)..(6): /replace="V" or "A"
  SITE (1)..(8): /note="Variant residues given in the sequence have no preference with respect to those in the annotations for variant positions"
LSGRSDNH

SEQ ID NO: 111 - protein, 8 aa, Artificial Sequence
  source: /note="Description of Artificial Sequence: Synthetic peptide"
  MOD_RES (1)..(1): Any amino acid
  VARIANT (3)..(3): /replace="S"
  VARIANT (4)..(4): /replace="K"
  MOD_RES (5)..(5): Any amino acid
  VARIANT (6)..(6): /replace="V"
  MOD_RES (7)..(7): Any amino acid
  MOD_RES (8)..(8): Any amino acid
  SITE (1)..(8): /note="Variant residues given in the sequence have no preference with respect to those in the annotations for variant positions"
XSGRXRXX

SEQ ID NO: 112 - protein, 5 aa, Artificial Sequence
  source: /note="Description of Artificial Sequence: Synthetic peptide"
SGRSA

SEQ ID NO: 113 - protein, 8 aa, Artificial Sequence
  source: /note="Description of Artificial Sequence: Synthetic peptide"
  VARIANT (1)..(1): /replace="I" or "A" or "K"
  VARIANT (2)..(2): /replace="G" or "Q"
  VARIANT (3)..(3): /replace="L" or "A" or "G" or "S"
  VARIANT (5)..(5): /replace="K" or "G" or "A" or "V"
  VARIANT (6)..(6): /replace="V" or "L"
  VARIANT (8)..(8): /replace="G" or "V" or "L"
  SITE (1)..(8): /note="Variant residues given in the sequence have no preference with respect to those in the annotations for variant positions"
LSKRSANH

SEQ ID NO: 114 - protein, 13 aa, Artificial Sequence
  source: /note="Description of Artificial Sequence: Synthetic peptide"
RKSSIIIRMRDWL

SEQ ID NO: 115 - protein, 4 aa, Artificial Sequence
  source: /note="Description of Artificial Sequence: Synthetic peptide"
AGPR

SEQ ID NO: 116 - protein, 6 aa, Artificial Sequence
  source: /note="Description of Artificial Sequence: Synthetic peptide"
RQARVV

SEQ ID NO: 117 - protein, 4 aa, Artificial Sequence
  source: /note="Description of Artificial Sequence: Synthetic peptide"
  VARIANT (4)..(4): /replace="K"
  SITE (1)..(4): /note="Variant residues given in the sequence have no preference with respect to those in the annotations for variant positions"
LSGR

SEQ ID NO: 118 - protein, 8 aa, Artificial Sequence
  source: /note="Description of Artificial Sequence: Synthetic peptide"
  MOD_RES (2)..(2): Any amino acid
  MOD_RES (3)..(3): Any amino acid
  MOD_RES (7)..(7): Any amino acid
RXXRKVXG

SEQ ID NO: 119 - protein, 5 aa, Artificial Sequence
  source: /note="Description of Artificial Sequence: Synthetic peptide"
  VARIANT (1)..(1): /replace="R"
  VARIANT (2)..(2): /replace="K" or "H" or "Q"
  VARIANT (3)..(3): /replace="A" or "S"
  VARIANT (4)..(4): /replace="K"
  SITE (1)..(5): /note="Variant residues given in the sequence have no preference with respect to those in the annotations for variant positions"
KRGRA

SEQ ID NO: 120 - protein, 8 aa, Artificial Sequence
  source: /note="Description of Artificial Sequence: Synthetic peptide"
  VARIANT (1)..(1): /replace="I" or "A" or "K"
  VARIANT (2)..(2): /replace="G" or "Q"
  VARIANT (3)..(3): /replace="L" or "A" or "G" or "S"
  VARIANT (5)..(5): /replace="K" or "G" or "A" or "V"
  VARIANT (6)..(6): /replace="V" or "L"
  VARIANT (8)..(8): /replace="G" or "V" or "L"
  SITE (1)..(8): /note="Variant residues given in the sequence have no preference with respect to those in the annotations for variant positions"
LSKRSANH

SEQ ID NO: 121 - protein, 5 aa, Artificial Sequence
  source: /note="Description of Artificial Sequence: Synthetic peptide"
  VARIANT (1)..(1): /replace="K"
  VARIANT (2)..(2): /replace="A" or "S" or "T" or "C" or "V" or "L" or "I" or "M" or "P" or "F" or "Y" or "W" or "D" or "E" or "N" or "Q"
  SITE (1)..(5): /note="Variant residues given in the sequence have no preference with respect to those in the annotations for variant positions"
RGSRA

SEQ ID NO: 122 - protein, 5 aa, Artificial Sequence
  source: /note="Description of Artificial Sequence: Synthetic peptide"
  VARIANT (1)..(1): /replace="A" or "S" or "T" or "C" or "V" or "L" or "I" or "M" or "P" or "F" or "Y" or "W" or "D" or "E" or "N" or "Q"
  VARIANT (2)..(2): /replace="K"
  SITE (1)..(5): /note="Variant residues given in the sequence have no preference with respect to those in the annotations for variant positions"
GRSRA

SEQ ID NO: 123 - protein, 10 aa, Artificial Sequence
  source: /note="Description of Artificial Sequence: Synthetic peptide"
EPKSCDKTHT

SEQ ID NO: 124 - protein, 9 aa, Artificial Sequence
  source: /note="Description of Artificial Sequence: Synthetic peptide"
PAPELLGGP

SEQ ID NO: 125 - protein, 12 aa, Artificial Sequence
  source: /note="Description of Artificial Sequence: Synthetic peptide"
SVFLFPPKPKDT

SEQ ID NO: 126 - protein, 23 aa, Artificial Sequence
  source: /note="Description of Artificial Sequence: Synthetic peptide"
EPKSCDKTHTCPPCPAPELLGGP

SEQ ID NO: 127 - protein, 12 aa, Artificial Sequence
  source: /note="Description of Artificial Sequence: Synthetic peptide"
  SITE (1)..(12): /note="This sequence may encompass 1-6 'Ser Gly' repeating units"
SGSGSGSGSGSG

SEQ ID NO: 128 - protein, 18 aa, Artificial Sequence
  source: /note="Description of Artificial Sequence: Synthetic peptide"
  SITE (1)..(18): /note="This sequence may encompass 1-6 'Ser Gly Gly' repeating units"
SGGSGGSGGSGGSGGSGG

SEQ ID NO: 129 - protein, 24 aa, Artificial Sequence
  source: /note="Description of Artificial Sequence: Synthetic peptide"
  SITE (1)..(24): /note="This sequence may encompass 1-6 'Ser Gly Gly Gly' repeating units"
SGGGSGGGSGGGSGGGSGGGSGGG

SEQ ID NO: 130 - protein, 18 aa, Artificial Sequence
  source: /note="Description of Artificial Sequence: Synthetic peptide"
  SITE (1)..(18): /note="This sequence may encompass 1-6 'Ser Ser Gly' repeating units"
SSGSSGSSGSSGSSGSSG

SEQ ID NO: 131 - protein, 12 aa, Artificial Sequence
  source: /note="Description of Artificial Sequence: Synthetic peptide"
  SITE (1)..(12): /note="This sequence may encompass 1-6 'Gly Ser' repeating units"
GSGSGSGSGSGS

SEQ ID NO: 132 - protein, 18 aa, Artificial Sequence
  source: /note="Description of Artificial Sequence: Synthetic peptide"
  SITE (1)..(18): /note="This sequence may encompass 1-6 'Gly Gly Gly' repeating units"
GGGGGGGGGGGGGGGGGG

SEQ ID NO: 133 - protein, 30 aa, Artificial Sequence
  source: /note="Description of Artificial Sequence: Synthetic polypeptide"
  SITE (1)..(30): /note="This sequence may encompass 1-6 'Gly Ser Gly Gly Ser' repeating units"
GSGGSGSGGSGSGGSGSGGSGSGGSGSGGS

SEQ ID NO: 134 - protein, 18 aa, Artificial Sequence
  source: /note="Description of Artificial Sequence: Synthetic peptide"
  SITE (1)..(18): /note="This sequence may encompass 1-6 'Gly Ser Gly' repeating units"
GSGGSGGSGGSGGSGGSG

SEQ ID NO: 135 - protein, 30 aa, Artificial Sequence
  source: /note="Description of Artificial Sequence: Synthetic polypeptide"
  SITE (1)..(30): /note="This sequence may encompass 1-6 'Gly Gly Gly Gly Ser' repeating units"
GGGGSGGGGSGGGGSGGGGSGGGGSGGGGS

SEQ ID NO: 136 - protein, 24 aa, Artificial Sequence
  source: /note="Description of Artificial Sequence: Synthetic peptide"
  SITE (1)..(24): /note="This sequence may encompass 1-6 'Gly Gly Gly Ser' repeating units"
GGGSGGGSGGGSGGGSGGGSGGGS

SEQ ID NO: 137 - protein, 42 aa, Artificial Sequence
  source: /note="Description of Artificial Sequence: Synthetic polypeptide"
  SITE (1)..(42): /note="This sequence may encompass 1-6 'Gly Gly Gly Gly Ser Gly Ser' repeating units"
GGGGSGSGGGGSGSGGGGSGSGGGGSGSGGGGSGSGGGGSGS

SEQ ID NO: 138 - protein, 54 aa, Artificial Sequence
  source: /note="Description of Artificial Sequence: Synthetic polypeptide"
  SITE (1)..(54): /note="This sequence may encompass 1-6 'Gly Gly Ser Gly Gly Gly Ser Gly Gly' repeating units"
GGSGGGSGGGGSGGGSGGGGSGGGSGGGGSGGGSGGGGSGGGSGGGGSGGGSGG

SEQ ID NO: 139 - protein, 48 aa, Artificial Sequence
  source: /note="Description of Artificial Sequence: Synthetic polypeptide"
  SITE (1)..(48): /note="This sequence may encompass 1-6 'Gly Gly Gly Gly Ser Gly Gly Ser' repeating units"
GGGGSGGSGGGGSGGSGGGGSGGSGGGGSGGSGGGGSGGSGGGGSGGS

SEQ ID NO: 140 - protein, 18 aa, Artificial Sequence
  source: /note="Description of Artificial Sequence: Synthetic peptide"
  SITE (1)..(18): /note="This sequence may encompass 1-6 'Gly Gly Ser' repeating units"
GGSGGSGGSGGSGGSGGS

SEQ ID NO: 141 - protein, 16 aa, Artificial Sequence
  source: /note="Description of Artificial Sequence: Synthetic peptide"
AKTTPKLEEGEFSEAR

SEQ ID NO: 142 - protein, 17 aa, Artificial Sequence
  source: /note="Description of Artificial Sequence: Synthetic peptide"
AKTTPKLEEGEFSEARV

SEQ ID NO: 143 - protein, 9 aa, Artificial Sequence
  source: /note="Description of Artificial Sequence: Synthetic peptide"
AKTTPKLGG

SEQ ID NO: 144 - protein, 10 aa, Artificial Sequence
  source: /note="Description of Artificial Sequence: Synthetic peptide"
SAKTTPKLGG

SEQ ID NO: 145 - protein, 6 aa, Artificial Sequence
  source: /note="Description of Artificial Sequence: Synthetic peptide"
SAKTTP

SEQ ID NO: 146 - protein, 6 aa, Artificial Sequence
  source: /note="Description of Artificial Sequence: Synthetic peptide"
RADAAP

SEQ ID NO: 147 - protein, 9 aa, Artificial Sequence
  source: /note="Description of Artificial Sequence: Synthetic peptide"
RADAAPTVS

SEQ ID NO: 148 - protein, 12 aa, Artificial Sequence
  source: /note="Description of Artificial Sequence: Synthetic peptide"
RADAAAAGGPGS

SEQ ID NO: 149 - protein, 27 aa, Artificial Sequence
  source: /note="Description of Artificial Sequence: Synthetic peptide"
RADAAAAGGGGSGGGGSGGGGSGGGGS

SEQ ID NO: 150 - protein, 18 aa, Artificial Sequence
  source: /note="Description of Artificial Sequence: Synthetic peptide"
SAKTTPKLEEGEFSEARV

SEQ ID NO: 151 - protein, 5 aa, Artificial Sequence
  source: /note="Description of Artificial Sequence: Synthetic peptide"
ADAAP

SEQ ID NO: 152 - protein, 12 aa, Artificial Sequence
  source: /note="Description of Artificial Sequence: Synthetic peptide"
ADAAPTVSIFPP

SEQ ID NO: 153 - protein, 5 aa, Artificial Sequence
  source: /note="Description of Artificial Sequence: Synthetic peptide"
TVAAP

SEQ ID NO: 154 - protein, 12 aa, Artificial Sequence
  source: /note="Description of Artificial Sequence: Synthetic peptide"
TVAAPSVFIFPP

SEQ ID NO: 155 - protein, 6 aa, Artificial Sequence
  source: /note="Description of Artificial Sequence: Synthetic peptide"
QPKAAP

SEQ ID NO: 156 - protein, 13 aa, Artificial Sequence
  source: /note="Description of Artificial Sequence: Synthetic peptide"
QPKAAPSVTLFPP

SEQ ID NO: 157 - protein, 6 aa, Artificial Sequence
  source: /note="Description of Artificial Sequence: Synthetic peptide"
AKTTPP

SEQ ID NO: 158 - protein, 13 aa, Artificial Sequence
  source: /note="Description of Artificial Sequence: Synthetic peptide"
AKTTPPSVTPLAP

SEQ ID NO: 159 - protein, 6 aa, Artificial Sequence
  source: /note="Description of Artificial Sequence: Synthetic peptide"
AKTTAP

SEQ ID NO: 160 - protein, 13 aa, Artificial Sequence
  source: /note="Description of Artificial Sequence: Synthetic peptide"
AKTTAPSVYPLAP

SEQ ID NO: 161 - protein, 6 aa, Artificial Sequence
  source: /note="Description of Artificial Sequence: Synthetic peptide"
ASTKGP

SEQ ID NO: 162 - protein, 13 aa, Artificial Sequence
  source: /note="Description of Artificial Sequence: Synthetic peptide"
ASTKGPSVFPLAP

SEQ ID NO: 163 - protein, 15 aa, Artificial Sequence
  source: /note="Description of Artificial Sequence: Synthetic peptide"
GENKVEYAPALMALS

SEQ ID NO: 164 - protein, 15 aa, Artificial Sequence
  source: /note="Description of Artificial Sequence: Synthetic peptide"
GPAKELTPLKEAKVS

SEQ ID NO: 165 - protein, 15 aa, Artificial Sequence
  source: /note="Description of Artificial Sequence: Synthetic peptide"
GHEAAAVMQVQYPAS

SEQ ID NO: 166 - protein, 4 aa, Artificial Sequence
  source: /note="Description of Artificial Sequence: Synthetic peptide"
GKGG

SEQ ID NO: 167 - protein, 565 aa, Artificial Sequence
  source: /note="Description of Artificial Sequence: Synthetic polypeptide"
GQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGPLGVRGGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 168 - protein, 453 aa, Artificial Sequence
  source: /note="Description of Artificial Sequence: Synthetic polypeptide"
VRGGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 169 - protein, 218 aa, Artificial Sequence
  source: /note="Description of Artificial Sequence: Synthetic polypeptide"
VRGGDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 170 - protein, 566 aa, Artificial Sequence
  source: /note="Description of Artificial Sequence: Synthetic polypeptide"
GQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGRRGGGGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - protein, 331 aa, Artificial Sequence
  source: /note="Description of Artificial Sequence: Synthetic polypeptide"
GQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGRRGGGGDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 172 - protein, 561 aa, Artificial Sequence
  source: /note="Description of Artificial Sequence: Synthetic polypeptide"
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSSGGPLGVRGGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 330 aa, Artificial Sequence
  source: /note="Description of Artificial Sequence: Synthetic polypeptide"
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESGGPLGVRGGDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 174 - protein, 329 aa, Artificial Sequence
  source: /note="Description of Artificial Sequence: Synthetic polypeptide"
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTESSGGPLGVRGGDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 175 - protein, 554 aa, Artificial Sequence
  source: /note="Description of Artificial Sequence: Synthetic polypeptide"
IQNPDPAVYQLRDSKSSDKSVCLFTDFDSQTNVSQSKDSDVYITDKTVLDMRSMDFKSNSAVAWSNKSDFACANAFNNSIIPEDTFFPSPGGPLGVRGGPLGVRGGQVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 343 aa, Artificial Sequence
  source: /note="Description of Artificial Sequence: Synthetic polypeptide"
KVFPPEVAVFEPSEAEISHTQKATLVCLATGFFPDHVELSWWVNGKEVHSGVSTDPQPLKEQPALNDSRYSLSSRLRVSATFWQNPRNHFRCQVQFNGQIVSAEAWGRADSGGPLGVRGGPLGVRGGQAGLTQPPSASGTSGQRVTISCSGSSSNIGINFVYWYQHLPGTAPKLLIYKNNQRPSGVPDRFSGSKSGNSASLAISGLRSEDEADYYCAAWDDSLSGYVFGSGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS